SEQUENCE LISTING (423 sequences)

SEQ ID NO: 1 - protein, 10 aa, synthetic construct
  REGION 1..10: note = Description of Artificial Sequence: Synthetic peptide
GYAFTNYLIE

SEQ ID NO: 2 - protein, 17 aa, synthetic construct
  REGION 1..17: note = Description of Artificial Sequence: Synthetic peptide
VINPGSGGTNYNEKFKG

SEQ ID NO: 3 - protein, 12 aa, synthetic construct
  REGION 1..12: note = Description of Artificial Sequence: Synthetic peptide
WRGDGYYAYFDV

SEQ ID NO: 4 - protein, 5 aa, synthetic construct
  REGION 1..5: note = Description of Artificial Sequence: Synthetic peptide
NYLIE

SEQ ID NO: 5 - protein, 7 aa, synthetic construct
  REGION 1..7: note = Description of Artificial Sequence: Synthetic peptide
GYAFTNY

SEQ ID NO: 6 - protein, 6 aa, synthetic construct
  REGION 1..6: note = Description of Artificial Sequence: Synthetic peptide
NPGSGG

SEQ ID NO: 7 - protein, 121 aa, synthetic construct
  REGION 1..121: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGDGYYAYFDVWGQGTTVTVSS

SEQ ID NO: 8 - DNA, 363 bp, synthetic construct
  misc_feature 1..363: note = Description of Artificial Sequence: Synthetic polynucleotide
  source 1..363: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgtgcccggtggcggggagatggctactacgcctacttcgacgtgtggggccagggcaccaccgtgaccgtgtcctct

SEQ ID NO: 9 - protein, 121 aa, synthetic construct
  REGION 1..121: note = Description of Artificial Sequence: Synthetic polypeptide
QVQLVESGGGVVQPGRSLRLSCAASGYAFTNYLIEWVRQAPGKGLEWVAVINPGSGGTNYNEKFKGRFTISADKSKSTAYLQMNSLRAEDTAVYYCARWRGDGYYAYFDVWGQGTTVTVSS

SEQ ID NO: 10 - DNA, 363 bp, synthetic construct
  misc_feature 1..363: note = Description of Artificial Sequence: Synthetic polynucleotide
  source 1..363: mol_type = other DNA
caggtgcagctggtggagagcggcggcggcgtggtgcagcccggccggagcctgcggctgagctgcgccgccagcggctacgccttcaccaactacctgatcgagtgggtgcggcaggcccccggcaagggcctggagtgggtggccgtgatcaaccccggcagcggcggcaccaactacaacgagaagttcaagggccggttcaccatcagcgccgacaagagcaagagcaccgcctacctgcagatgaacagcctgcgggccgaggacaccgccgtgtactactgcgcccggtggcggggcgacggctactacgcctacttcgacgtgtggggccagggcaccaccgtgaccgtgagcagc

SEQ ID NO: 11 - protein, 10 aa, synthetic construct
  REGION 1..10: note = Description of Artificial Sequence: Synthetic peptide
GYTFSSYLIE

SEQ ID NO: 12 - protein, 17 aa, synthetic construct
  REGION 1..17: note = Description of Artificial Sequence: Synthetic peptide
VINPGSGGTNYADSVKG

SEQ ID NO: 13 - protein, 5 aa, synthetic construct
  REGION 1..5: note = Description of Artificial Sequence: Synthetic peptide
SYLIE

SEQ ID NO: 14 - protein, 7 aa, synthetic construct
  REGION 1..7: note = Description of Artificial Sequence: Synthetic peptide
GYTFSSY

SEQ ID NO: 15 - protein, 121 aa, synthetic construct
  REGION 1..121: note = Description of Artificial Sequence: Synthetic polypeptide
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGDGYYAYFDVWGQGTTVTVSS

SEQ ID NO: 16 - DNA, 363 bp, synthetic construct
  misc_feature 1..363: note = Description of Artificial Sequence: Synthetic polynucleotide
  source 1..363: mol_type = other DNA
caggtgcaattggtggaaagcggcggaggcgtggtgcagcctggaagaagcctgagactgagctgtgccgccagcggctacaccttcagcagctacctgatcgagtgggtgcgccaggcccctggcaaaggactggaatgggtggccgtgatcaaccctggcagcggcggcaccaattacgccgatagcgtgaagggccggttcaccatcagcgccgacaagagcaagaacaccgcctacctccagatgaacagcctgcgggccgaggacaccgccgtgtactattgtgctcggtggcggggagatggctactacgcctacttcgacgtgtggggccagggcaccacagtgaccgtcagctca

SEQ ID NO: 17 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGDGYYAYFDVWGQGTTVTVSS

SEQ ID NO: 18 - DNA, 363 bp, synthetic construct
  misc_feature 1..363: note = Description of Artificial Sequence: Synthetic polynucleotide
  source 1..363: mol_type = other DNA
gaagtgcagctggtgcagtctggcgctgaagtgaagaagcccggcgagtccctgaagatctcctgcaagggctccggctacgccttcaccaactacctgatcgagtgggtccgacagatgcccggcaagggcctggagtggatgggcgtgatcaaccccggctccggcggcaccaactacaacgagaagttcaagggccaagtcacaatctccgccgacaagtccatctccaccgcctacctgcagtggtcctccctgaaggcctccgacaccgccatgtactactgcgccagatggcggggagatggctactacgcctacttcgacgtgtggggccagggcaccaccgtgaccgtgtcctct

SEQ ID NO: 19 - protein, 15 aa, synthetic construct
  REGION 1..15: note = Description of Artificial Sequence: Synthetic peptide
KASQSVDYDGDSYMN

SEQ ID NO: 20 - protein, 7 aa, synthetic construct
  REGION 1..7: note = Description of Artificial Sequence: Synthetic peptide
AASNLES

SEQ ID NO: 21 - protein, 9 aa, synthetic construct
  REGION 1..9: note = Description of Artificial Sequence: Synthetic peptide
QQSNEDPYT

SEQ ID NO: 22 - protein, 11 aa, synthetic construct
  REGION 1..11: note = Description of Artificial Sequence: Synthetic peptide
SQSVDYDGDSY

SEQ ID NO: 23 - skipped

SEQ ID NO: 24 - protein, 6 aa, synthetic construct
  REGION 1..6: note = Description of Artificial Sequence: Synthetic peptide
SNEDPY

SEQ ID NO: 25 - protein, 111 aa, synthetic construct
  REGION 1..111: note = Description of Artificial Sequence: Synthetic polypeptide
AIRLTQSPSSFSASTGDRVTITCKASQSVDYDGDSYMNWYQQKPGKAPKLLIYAASNLESGVPSRFSGSGSGTDFTLTISSLQSEDFATYYCQQSNEDPYTFGGGTKVEIK

SEQ ID NO: 26 - DNA, 333 bp, synthetic construct
  misc_feature 1..333: note = Description of Artificial Sequence: Synthetic polynucleotide
  source 1..333: mol_type = other DNA
gccatcagactgacccagagcccctccagcttctccgcctccaccggcgacagagtgaccatcacatgcaaggcctcccagtccgtggactacgacggcgactcctacatgaactggtatcagcagaagcccggcaaggcccctaagctgctgatctacgccgcctccaacctggaatccggcgtgccctcccggttctccggctctggctctggcaccgacttcaccctgaccatctccagcctgcagtccgaggacttcgccacctactactgccagcagtccaacgaggacccctacaccttcggcggaggcaccaaagtggaaatcaag

SEQ ID NO: 27 - protein, 111 aa, synthetic construct
  REGION 1..111: note = Description of Artificial Sequence: Synthetic polypeptide
DIVLTQSPLSLPVTLGQPASISCKASQSVDYDGDSYMNWYQQRPGQSPRLLIYAASNLESGVPDRFSGSGSGTDFTLKISRVEAEDVGVYYCQQSNEDPYTFGGGTKVEIK

SEQ ID NO: 28 - DNA, 333 bp, synthetic construct
  misc_feature 1..333: note = Description of Artificial Sequence: Synthetic polynucleotide
  source 1..333: mol_type = other DNA
gacatcgtgctgacacagagccctctgtccctgcccgtgaccctgggccagcctgcctccatctcctgcaaggcctcccagtccgtggactacgacggcgactcctacatgaactggtatcagcagcggcctggccagtcccctcggctgctgatctacgccgcctccaacctggaatccggcgtgcccgacagattctccggctccggctctggcaccgacttcaccctgaagatctcccgggtggaagccgaggacgtgggcgtgtactactgccagcagtccaacgaggacccctacaccttcggcggaggcaccaaagtggaaatcaag

SEQ ID NO: 29 - protein, 111 aa, synthetic construct
  REGION 1..111: note = Description of Artificial Sequence: Synthetic polypeptide
EIVLTQSPATLSVSPGERATLSCKASQSVDYDGDSYMNWYQQKPGQAPRLLIYAASNLESGIPARFSGSGSGTEFTLTISSLQSEDAAVYYCQQSNEDPYTFGGGTKVEIK

SEQ ID NO: 30 - DNA, 333 bp, synthetic construct
  misc_feature 1..333: note = Description of Artificial Sequence: Synthetic polynucleotide
  source 1..333: mol_type = other DNA
gagatcgtgctgacccagagccccgccaccctgagcgtgagccccggcgagcgggccaccctgagctgcaaggccagccagagcgtggactacgacggcgacagctacatgaactggtaccagcagaagcccggccaggccccccggctgctgatctacgccgccagcaacctggagagcggcatccccgcccggttcagcggcagcggcagcggcaccgagttcaccctgaccatcagcagcctgcagagcgaggacgccgccgtgtactactgccagcagagcaacgaggacccctacaccttcggcggcggcaccaaggtggagatcaag

SEQ ID NO: 31 - protein, 15 aa, synthetic construct
  REGION 1..15: note = Description of Artificial Sequence: Synthetic peptide
RASQSVSYDGDSYMN

SEQ ID NO: 32 - protein, 7 aa, synthetic construct
  REGION 1..7: note = Description of Artificial Sequence: Synthetic peptide
AASNLAS

SEQ ID NO: 33 - protein, 11 aa, synthetic construct
  REGION 1..11: note = Description of Artificial Sequence: Synthetic peptide
SQSVSYDGDSY

SEQ ID NO: 34 - protein, 111 aa, synthetic construct
  REGION 1..111: note = Description of Artificial Sequence: Synthetic polypeptide
EIVLTQSPATLSVSPGERATLSCRASQSVSYDGDSYMNWYQQKPGQAPRLLIYAASNLASGIPARFSGSGSGTEFTLTISSLQSEDAAVYYCQQSNEDPYTFGGGTKVEIK

SEQ ID NO: 35 - DNA, 333 bp, synthetic construct
  misc_feature 1..333: note = Description of Artificial Sequence: Synthetic polynucleotide
  source 1..333: mol_type = other DNA
gaaatcgtgctgacccagagccctgccaccctgagtgtgtctccaggcgagagagccacactgagctgtagagccagccagagcgtgtcctacgacggcgacagctacatgaactggtatcagcagaagcccggccaggcccccagactgctgatctacgccgcttccaatctggccagcggcatccccgccagattttccggctctggctccggcaccgagttcaccctgacaatcagcagcctccagagcgaggacgccgccgtgtactactgccagcagagcaacgaggacccctacacctttggcggaggcaccaaggtggaaatcaag

SEQ ID NO: 36 - protein, 12 aa, synthetic construct
  REGION 1..12: note = Description of Artificial Sequence: Synthetic peptide
WRGEGYYAYFDV

SEQ ID NO: 37 - protein, 121 aa, synthetic construct
  REGION 1..121: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGEGYYAYFDVWGQGTTVTVSS

SEQ ID NO: 38 - DNA, 363 bp, synthetic construct
  misc_feature 1..363: note = Description of Artificial Sequence: Synthetic polynucleotide
  source 1..363: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgcgccagatggcggggagagggctactacgcctacttcgacgtgtggggccagggcaccaccgtgaccgtgtcctct

SEQ ID NO: 39 - protein, 12 aa, synthetic construct
  REGION 1..12: note = Description of Artificial Sequence: Synthetic peptide
WRGDAYYAYFDV

SEQ ID NO: 40 - protein, 121 aa, synthetic construct
  REGION 1..121: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGDAYYAYFDVWGQGTTVTVSS

SEQ ID NO: 41 - DNA, 363 bp, synthetic construct
  misc_feature 1..363: note = Description of Artificial Sequence: Synthetic polynucleotide
  source 1..363: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgtgcccggtggcggggagatgcctactacgcctacttcgacgtgtggggccagggcaccaccgtgaccgtgtcctct

SEQ ID NO: 42 - protein, 12 aa, synthetic construct
  REGION 1..12: note = Description of Artificial Sequence: Synthetic peptide
WRGQGYYAYFDV

SEQ ID NO: 43 - protein, 121 aa, synthetic construct
  REGION 1..121: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGQGYYAYFDVWGQGTTVTVSS

SEQ ID NO: 44 - DNA, 363 bp, synthetic construct
  misc_feature 1..363: note = Description of Artificial Sequence: Synthetic polynucleotide
  source 1..363: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgcgccagatggcggggacagggctactacgcctacttcgacgtgtggggccagggcaccaccgtgaccgtgtcctct

SEQ ID NO: 45 - protein, 12 aa, synthetic construct
  REGION 1..12: note = Description of Artificial Sequence: Synthetic peptide
WRGSGYYAYFDV

SEQ ID NO: 46 - protein, 121 aa, synthetic construct
  REGION 1..121: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGSGYYAYFDVWGQGTTVTVSS

SEQ ID NO: 47 - DNA, 363 bp, synthetic construct
  misc_feature 1..363: note = Description of Artificial Sequence: Synthetic polynucleotide
  source 1..363: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgcgccagatggcggggatctggctactacgcctacttcgacgtgtggggccagggcaccaccgtgaccgtgtcctct

SEQ ID NO: 48 - protein, 121 aa, synthetic construct
  REGION 1..121: note = Description of Artificial Sequence: Synthetic polypeptide
QVQLVESGGGVVQPGRSLRLSCAASGYAFTNYLIEWVRQAPGKGLEWVAVINPGSGGTNYNEKFKGRFTISADKSKSTAYLQMNSLRAEDTAVYYCARWRGEGYYAYFDVWGQGTTVTVSS

SEQ ID NO: 49 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSS

SEQ ID NO: 50 - DNA, 363 bp, synthetic construct
  misc_feature 1..363: note = Description of Artificial Sequence: Synthetic polynucleotide
  source 1..363: mol_type = other DNA
gaagtgcagctggtgcagtctggcgctgaagtgaagaagcccggcgagtccctgaagatctcctgcaagggctccggctacgccttcaccaactacctgatcgagtgggtccgacagatgcccggcaagggcctggagtggatgggcgtgatcaaccccggctccggcggcaccaactacaacgagaagttcaagggccaagtcacaatctccgccgacaagtccatctccaccgcctacctgcagtggtcctccctgaaggcctccgacaccgccatgtactactgcgccagatggcggggagagggctactacgcctacttcgacgtgtggggccagggcaccaccgtgaccgtgtcctct

SEQ ID NO: 51 - protein, 121 aa, synthetic construct
  REGION 1..121: note = Description of Artificial Sequence: Synthetic polypeptide
QVQLVESGGGVVQPGRSLRLSCAASGYAFTNYLIEWVRQAPGKGLEWVAVINPGSGGTNYNEKFKGRFTISADKSKSTAYLQMNSLRAEDTAVYYCARWRGDAYYAYFDVWGQGTTVTVSS

SEQ ID NO: 52 - DNA, 363 bp, synthetic construct
  misc_feature 1..363: note = Description of Artificial Sequence: Synthetic polynucleotide
  source 1..363: mol_type = other DNA
caggtgcagctggtggagagcggcggcggcgtggtgcagcccggccggagcctgcggctgagctgcgccgccagcggctacgccttcaccaactacctgatcgagtgggtgcggcaggcccccggcaagggcctggagtgggtggccgtgatcaaccccggcagcggcggcaccaactacaacgagaagttcaagggccggttcaccatcagcgccgacaagagcaagagcaccgcctacctgcagatgaacagcctgcgggccgaggacaccgccgtgtactactgcgcccggtggcggggcgacgcctactacgcctacttcgacgtgtggggccagggcaccaccgtgaccgtgagcagc

SEQ ID NO: 53 - protein, 121 aa, synthetic construct
  REGION 1..121: note = Description of Artificial Sequence: Synthetic polypeptide
QVQLVESGGGVVQPGRSLRLSCAASGYAFTNYLIEWVRQAPGKGLEWVAVINPGSGGTNYNEKFKGRFTISADKSKSTAYLQMNSLRAEDTAVYYCARWRGQGYYAYFDVWGQGTTVTVSS

SEQ ID NO: 54 - DNA, 363 bp, synthetic construct
  misc_feature 1..363: note = Description of Artificial Sequence: Synthetic polynucleotide
  source 1..363: mol_type = other DNA
caggtgcagctggtggagagcggcggcggcgtggtgcagcccggccggagcctgcggctgagctgcgccgccagcggctacgccttcaccaactacctgatcgagtgggtgcggcaggcccccggcaagggcctggagtgggtggccgtgatcaaccccggcagcggcggcaccaactacaacgagaagttcaagggccggttcaccatcagcgccgacaagagcaagagcaccgcctacctgcagatgaacagcctgcgggccgaggacaccgccgtgtactactgcgcccggtggcggggccagggctactacgcctacttcgacgtgtggggccagggcaccaccgtgaccgtgagcagc

SEQ ID NO: 55 - protein, 121 aa, synthetic construct
  REGION 1..121: note = Description of Artificial Sequence: Synthetic polypeptide
QVQLVESGGGVVQPGRSLRLSCAASGYAFTNYLIEWVRQAPGKGLEWVAVINPGSGGTNYNEKFKGRFTISADKSKSTAYLQMNSLRAEDTAVYYCARWRGSGYYAYFDVWGQGTTVTVSS

SEQ ID NO: 56 - DNA, 363 bp, synthetic construct
  misc_feature 1..363: note = Description of Artificial Sequence: Synthetic polynucleotide
  source 1..363: mol_type = other DNA
caggtgcagctggtggagagcggcggcggcgtggtgcagcccggccggagcctgcggctgagctgcgccgccagcggctacgccttcaccaactacctgatcgagtgggtgcggcaggcccccggcaagggcctggagtgggtggccgtgatcaaccccggcagcggcggcaccaactacaacgagaagttcaagggccggttcaccatcagcgccgacaagagcaagagcaccgcctacctgcagatgaacagcctgcgggccgaggacaccgccgtgtactactgcgcccggtggcggggcagcggctactacgcctacttcgacgtgtggggccagggcaccaccgtgaccgtgagcagc

SEQ ID NO: 57 - protein, 121 aa, synthetic construct
  REGION 1..121: note = Description of Artificial Sequence: Synthetic polypeptide
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGEGYYAYFDVWGQGTTVTVSS

SEQ ID NO: 58 - DNA, 363 bp, synthetic construct
  misc_feature 1..363: note = Description of Artificial Sequence: Synthetic polynucleotide
  source 1..363: mol_type = other DNA
caggtgcaattggtggaaagcggcggaggcgtggtgcagcctggaagaagcctgagactgagctgtgccgccagcggctacaccttcagcagctacctgatcgagtgggtgcgccaggcccctggcaaaggactggaatgggtggccgtgatcaaccctggcagcggcggcaccaattacgccgatagcgtgaagggccggttcaccatcagcgccgacaagagcaagaacaccgcctacctccagatgaacagcctgcgggccgaggacaccgccgtgtactattgtgctcggtggcggggagagggctactacgcctacttcgacgtgtggggccagggcaccacagtgaccgtcagctca

SEQ ID NO: 59 - protein, 121 aa, synthetic construct
  REGION 1..121: note = Description of Artificial Sequence: Synthetic polypeptide
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGDAYYAYFDVWGQGTTVTVSS

SEQ ID NO: 60 - DNA, 363 bp, synthetic construct
  misc_feature 1..363: note = Description of Artificial Sequence: Synthetic polynucleotide
  source 1..363: mol_type = other DNA
caggtgcaattggtggaaagcggcggaggcgtggtgcagcctggaagaagcctgagactgagctgtgccgccagcggctacaccttcagcagctacctgatcgagtgggtgcgccaggcccctggcaaaggactggaatgggtggccgtgatcaaccctggcagcggcggcaccaattacgccgatagcgtgaagggccggttcaccatcagcgccgacaagagcaagaacaccgcctacctccagatgaacagcctgcgggccgaggacaccgccgtgtactattgtgctcggtggcggggagatgcctactacgcctacttcgacgtgtggggccagggcaccacagtgaccgtcagctca

SEQ ID NO: 61 - protein, 121 aa, synthetic construct
  REGION 1..121: note = Description of Artificial Sequence: Synthetic polypeptide
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGQGYYAYFDVWGQGTTVTVSS

SEQ ID NO: 62 - DNA, 363 bp, synthetic construct
  misc_feature 1..363: note = Description of Artificial Sequence: Synthetic polynucleotide
  source 1..363: mol_type = other DNA
caggtgcaattggtggaaagcggcggaggcgtggtgcagcctggaagaagcctgagactgagctgtgccgccagcggctacaccttcagcagctacctgatcgagtgggtgcgccaggcccctggcaaaggactggaatgggtggccgtgatcaaccctggcagcggcggcaccaattacgccgatagcgtgaagggccggttcaccatcagcgccgacaagagcaagaacaccgcctacctccagatgaacagcctgcgggccgaggacaccgccgtgtactattgtgctcggtggcggggacagggctactacgcctacttcgacgtgtggggccagggcaccacagtgaccgtcagctca

SEQ ID NO: 63 - protein, 121 aa, synthetic construct
  REGION 1..121: note = Description of Artificial Sequence: Synthetic polypeptide
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGSGYYAYFDVWGQGTTVTVSS

SEQ ID NO: 64 - DNA, 363 bp, synthetic construct
  misc_feature 1..363: note = Description of Artificial Sequence: Synthetic polynucleotide
  source 1..363: mol_type = other DNA
caggtgcaattggtggaaagcggcggaggcgtggtgcagcctggaagaagcctgagactgagctgtgccgccagcggctacaccttcagcagctacctgatcgagtgggtgcgccaggcccctggcaaaggactggaatgggtggccgtgatcaaccctggcagcggcggcaccaattacgccgatagcgtgaagggccggttcaccatcagcgccgacaagagcaagaacaccgcctacctccagatgaacagcctgcgggccgaggacaccgccgtgtactattgtgctcggtggcggggaagcggctactacgcctacttcgacgtgtggggccagggcaccacagtgaccgtcagctca

SEQ ID NO: 65 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGDAYYAYFDVWGQGTTVTVSS

SEQ ID NO: 66 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagatgcctactacgcctacttcgacgtgtggggccagggcaccacagtgaccgtcagctca

SEQ ID NO: 67 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGQGYYAYFDVWGQGTTVTVSS

SEQ ID NO: 68 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggacagggctactacgcctacttcgacgtgtggggccagggcaccacagtgaccgtcagctca

SEQ ID NO: 69 - protein, 15 aa, synthetic construct
  REGION 1..15: note = Description of Artificial Sequence: Synthetic peptide
KASQSVDYQGDSYMN

SEQ ID NO: 70 - protein, 111 aa, synthetic construct
  REGION 1..111: note = Description of Artificial Sequence: Synthetic polypeptide
AIRLTQSPSSFSASTGDRVTITCKASQSVDYQGDSYMNWYQQKPGKAPKLLIYAASNLESGVPSRFSGSGSGTDFTLTISSLQSEDFATYYCQQSNEDPYTFGGGTKVEIK

SEQ ID NO: 71 - DNA, 333 bp, synthetic construct
  misc_feature 1..333: note = Description of Artificial Sequence: Synthetic polynucleotide
  source 1..333: mol_type = other DNA
gccatcagactgacccagagcccctccagcttctccgcctccaccggcgacagagtgaccatcacatgcaaggcctcccagtccgtggactaccagggcgactcctacatgaactggtatcagcagaagcccggcaaggcccctaagctgctgatctacgccgcctccaacctggaatccggcgtgccctcccggttctccggctctggctctggcaccgacttcaccctgaccatctccagcctgcagtccgaggacttcgccacctactactgccagcagtccaacgaggacccctacaccttcggcggaggcaccaaagtggaaatcaag

SEQ ID NO: 72 - protein, 15 aa, synthetic construct
  REGION 1..15: note = Description of Artificial Sequence: Synthetic peptide
KASQSVDYDADSYMN

SEQ ID NO: 73 - protein, 111 aa, synthetic construct
  REGION 1..111: note = Description of Artificial Sequence: Synthetic polypeptide
AIRLTQSPSSFSASTGDRVTITCKASQSVDYDADSYMNWYQQKPGKAPKLLIYAASNLESGVPSRFSGSGSGTDFTLTISSLQSEDFATYYCQQSNEDPYTFGGGTKVEIK

SEQ ID NO: 74 - DNA, 333 bp, synthetic construct
  misc_feature 1..333: note = Description of Artificial Sequence: Synthetic polynucleotide
  source 1..333: mol_type = other DNA
gacatcgtgctgacacagagccctctgtccctgcccgtgaccctgggccagcctgcctccatctcctgcaaggcctcccagtccgtggactacgacgccgactcctacatgaactggtatcagcagcggcctggccagtcccctcggctgctgatctacgccgcctccaacctggaatccggcgtgcccgacagattctccggctccggctctggcaccgacttcaccctgaagatctcccgggtggaagccgaggacgtgggcgtgtactactgccagcagtccaacgaggacccctacaccttcggcggaggcaccaaagtggaaatcaag

SEQ ID NO: 75 - protein, 11 aa, synthetic construct
  REGION 1..11: note = Description of Artificial Sequence: Synthetic peptide
SQSVDYQGDSY

SEQ ID NO: 76 - protein, 111 aa, synthetic construct
  REGION 1..111: note = Description of Artificial Sequence: Synthetic polypeptide
EIVLTQSPATLSVSPGERATLSCKASQSVDYQGDSYMNWYQQKPGQAPRLLIYAASNLESGIPARFSGSGSGTEFTLTISSLQSEDAAVYYCQQSNEDPYTFGGGTKVEIK

SEQ ID NO: 77 - DNA, 333 bp, synthetic construct
  misc_feature 1..333: note = Description of Artificial Sequence: Synthetic polynucleotide
  source 1..333: mol_type = other DNA
gaaatcgtgctgacccagagccctgccaccctgagtgtgtctccaggcgagagagccacactgagctgtagagccagccagagcgtgtcctaccagggcgacagctacatgaactggtatcagcagaagcccggccaggcccccagactgctgatctacgccgcttccaatctggccagcggcatccccgccagattttccggctctggctccggcaccgagttcaccctgacaatcagcagcctccagagcgaggacgccgccgtgtactactgccagcagagcaacgaggacccctacacctttggcggaggcaccaaggtggaaatcaag

SEQ ID NO: 78 - protein, 11 aa, synthetic construct
  REGION 1..11: note = Description of Artificial Sequence: Synthetic peptide
SQSVDYDADSY

SEQ ID NO: 79 - protein, 111 aa, synthetic construct
  REGION 1..111: note = Description of Artificial Sequence: Synthetic polypeptide
DIVLTQSPLSLPVTLGQPASISCKASQSVDYQGDSYMNWYQQRPGQSPRLLIYAASNLESGVPDRFSGSGSGTDFTLKISRVEAEDVGVYYCQQSNEDPYTFGGGTKVEIK

SEQ ID NO: 80 - DNA, 333 bp, synthetic construct
  misc_feature 1..333: note = Description of Artificial Sequence: Synthetic polynucleotide
  source 1..333: mol_type = other DNA
gagatcgtgctgacccagagccccgccaccctgagcgtgagccccggcgagcgggccaccctgagctgcaaggccagccagagcgtggactaccagggcgacagctacatgaactggtaccagcagaagcccggccaggccccccggctgctgatctacgccgccagcaacctggagagcggcatccccgcccggttcagcggcagcggcagcggcaccgagttcaccctgaccatcagcagcctgcagagcgaggacgccgccgtgtactactgccagcagagcaacgaggacccctacaccttcggcggcggcaccaaggtggagatcaag

SEQ ID NO: 81 - protein, 111 aa, synthetic construct
  REGION 1..111: note = Description of Artificial Sequence: Synthetic polypeptide
DIVLTQSPLSLPVTLGQPASISCKASQSVDYDADSYMNWYQQRPGQSPRLLIYAASNLESGVPDRFSGSGSGTDFTLKISRVEAEDVGVYYCQQSNEDPYTFGGGTKVEIK

SEQ ID NO: 82 - DNA, 333 bp, synthetic construct
  misc_feature 1..333: note = Description of Artificial Sequence: Synthetic polynucleotide
  source 1..333: mol_type = other DNA
gagatcgtgctgacccagagccccgccaccctgagcgtgagccccggcgagcgggccaccctgagctgcaaggccagccagagcgtggactacgacgccgacagctacatgaactggtaccagcagaagcccggccaggccccccggctgctgatctacgccgccagcaacctggagagcggcatccccgcccggttcagcggcagcggcagcggcaccgagttcaccctgaccatcagcagcctgcagagcgaggacgccgccgtgtactactgccagcagagcaacgaggacccctacaccttcggcggcggcaccaaggtggagatcaag

SEQ ID NO: 83 - skipped

SEQ ID NO: 84 - protein, 111 aa, synthetic construct
  REGION 1..111: note = Description of Artificial Sequence: Synthetic polypeptide
EIVLTQSPATLSVSPGERATLSCKASQSVDYDADSYMNWYQQKPGQAPRLLIYAASNLESGIPARFSGSGSGTEFTLTISSLQSEDAAVYYCQQSNEDPYTFGGGTKVEIK

SEQ ID NO: 85 - DNA, 333 bp, synthetic construct
  misc_feature 1..333: note = Description of Artificial Sequence: Synthetic polynucleotide
  source 1..333: mol_type = other DNA
gaaatcgtgctgacccagagccctgccaccctgagtgtgtctccaggcgagagagccacactgagctgtagagccagccagagcgtgtcctacgacgccgacagctacatgaactggtatcagcagaagcccggccaggcccccagactgctgatctacgccgcttccaatctggccagcggcatccccgccagattttccggctctggctccggcaccgagttcaccctgacaatcagcagcctccagagcgaggacgccgccgtgtactactgccagcagagcaacgaggacccctacacctttggcggaggcaccaaggtggaaatcaag

SEQ ID NO: 86 - protein, 15 aa, synthetic construct
  REGION 1..15: note = Description of Artificial Sequence: Synthetic peptide
RASQSVSYQGDSYMN

SEQ ID NO: 87 - protein, 11 aa, synthetic construct
  REGION 1..11: note = Description of Artificial Sequence: Synthetic peptide
SQSVSYQGDSY

SEQ ID NO: 88 - protein, 111 aa, synthetic construct
  REGION 1..111: note = Description of Artificial Sequence: Synthetic polypeptide
EIVLTQSPATLSVSPGERATLSCRASQSVSYQGDSYMNWYQQKPGQAPRLLIYAASNLASGIPARFSGSGSGTEFTLTISSLQSEDAAVYYCQQSNEDPYTFGGGTKVEIK

SEQ ID NO: 89 - DNA, 333 bp, synthetic construct; identical to SEQ ID NO: 77
  misc_feature 1..333: note = Description of Artificial Sequence: Synthetic polynucleotide
  source 1..333: mol_type = other DNA

SEQ ID NO: 90 - protein, 15 aa, synthetic construct
  REGION 1..15: note = Description of Artificial Sequence: Synthetic peptide
RASQSVSYDADSYMN

SEQ ID NO: 91 - protein, 111 aa, synthetic construct
  REGION 1..111: note = Description of Artificial Sequence: Synthetic polypeptide
EIVLTQSPATLSVSPGERATLSCRASQSVSYDADSYMNWYQQKPGQAPRLLIYAASNLASGIPARFSGSGSGTEFTLTISSLQSEDAAVYYCQQSNEDPYTFGGGTKVEIK

SEQ ID NO: 92 - DNA, 333 bp, synthetic construct; identical to SEQ ID NO: 85
  misc_feature 1..333: note = Description of Artificial Sequence: Synthetic polynucleotide
  source 1..333: mol_type = other DNA

SEQ ID NO: 93 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - DNA, 990 bp, synthetic construct
  misc_feature 1..990: note = Description of Artificial Sequence: Synthetic polynucleotide
  source 1..990: mol_type = other DNA
gcgtcgaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 95 - protein, 326 aa, synthetic construct
  REGION 1..326: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - DNA, 978 bp, synthetic construct
  misc_feature 1..978: note = Description of Artificial Sequence: Synthetic polynucleotide
  source 1..978: mol_type = other DNA
gccagcaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcaacttcggcacccagacctacacctgcaacgtggaccacaagcccagcaacaccaaggtggacaagaccgtggagaggaagtgctgcgtggagtgccccccctgcccagcccccccagtggccggaccctccgtgttcctgttcccccccaagcccaaggacaccctgatgatcagcaggacccccgaggtgacctgcgtggtggtggacgtgagccacgaggacccagaggtgcagttcaactggtacgtggacggcgtggaggtgcacaacgccaagaccaagcccagagaggaacagtttaacagcaccttcagggtggtgtccgtgctgaccgtggtgcaccaggactggctgaacggcaaagagtacaagtgcaaggtctccaacaagggcctgccagcccccatcgagaaaaccatcagcaagaccaagggccagccacgggagccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaag

SEQ ID NO: 97 - protein, 377 aa, synthetic construct
  REGION 1..377: note = Description of Artificial Sequence: Synthetic polypeptide
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 98 - DNA, 1131 bp, synthetic construct
  misc_feature 1..1131: note = Description of Artificial Sequence: Synthetic polynucleotide
  source 1..1131: mol_type = other DNA
gccagcaccaagggccccagcgtgttccccctggccccctgcagccggagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacacctgcaacgtgaaccacaagcccagcaacaccaaggtggacaagcgggtggagctgaagacccccctgggcgacaccacccacacctgcccccggtgccccgagcccaagagctgcgacaccccccccccctgcccccggtgccccgagcccaagagctgcgacaccccccccccctgcccccggtgccccgagcccaagagctgcgacaccccccccccctgcccccggtgccccgcccccgagctgctgggcggccccagcgtgttcctgttcccccccaagcccaaggacaccctgatgatcagccggacccccgaggtgacctgcgtggtggtggacgtgagccacgaggaccccgaggtgcagttcaagtggtacgtggacggcgtggaggtgcacaacgccaagaccaagccccgggaggagcagtacaacagcaccttccgggtggtgagcgtgctgaccgtgctgcaccaggactggctgaacggcaaggagtacaagtgcaaggtgagcaacaaggccctgcccgcccccatcgagaagaccatcagcaagaccaagggccagccccgggagccccaggtgtacaccctgccccccagccgggaggagatgaccaagaaccaggtgagcctgacctgcctggtgaagggcttctaccccagcgacatcgccgtggagtgggagagcagcggccagcccgagaacaactacaacaccaccccccccatgctggacagcgacggcagcttcttcctgtacagcaagctgaccgtggacaagagccggtggcagcagggcaacatcttcagctgcagcgtgatgcacgaggccctgcacaaccggttcacccagaagagcctgagcctgagccccggcaag

SEQ ID NO: 99 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - DNA, 981 bp, synthetic construct
  misc_feature 1..981: note = Description of Artificial Sequence: Synthetic polynucleotide
  source 1..981: mol_type = other DNA
gcctctaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcagcctgggcaccaagacctacacctgcaacgtggaccacaagcccagcaacaccaaggtggacaagagggtggagagcaagtacggcccaccctgcccctcttgcccagcccccgagttcctgggcggaccctccgtgttcctgttcccccccaagcccaaggacaccctgatgatcagcaggacccccgaggtgacctgcgtggtggtggacgtgagccaggaagatccagaggtccagttcaactggtacgtggacggcgtggaggtgcacaacgccaagaccaagcccagagaggaacagtttaacagcacctacagggtggtgtccgtgctgaccgtgctgcaccaggactggctgaacggcaaagagtacaagtgcaaggtctccaacaagggcctgcccagctccatcgagaaaaccatcagcaaggccaagggccagccacgggagccccaggtgtacaccctgccaccctcccaggaagagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaggctgaccgtggacaagtccaggtggcaggaaggcaacgtctttagctgcagcgtgatgcacgaggccctgcacaaccactacacccagaagagcctgagcctgtccctgggcaag

SEQ ID NO: 101 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - DNA, 990 bp, synthetic construct
  misc_feature 1..990: note = Description of Artificial Sequence: Synthetic polynucleotide
  source 1..990: mol_type = other DNA
gcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 103 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 990 bp, synthetic construct
  misc_feature 1..990: note = Description of Artificial Sequence: Synthetic polynucleotide
  source 1..990: mol_type = other DNA
gctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacgcctccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 105 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - DNA, 990 bp, synthetic construct
  misc_feature 1..990: note = Description of Artificial Sequence: Synthetic polynucleotide
  source 1..990: mol_type = other DNA
gctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggccgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctggcagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 107 - protein, 107 aa, synthetic construct [sequence omitted]

SEQ ID NO: 108 - DNA, 321 bp, synthetic construct
  misc_feature 1..321: note = Description of Artificial Sequence: Synthetic polynucleotide
  source 1..321: mol_type = other DNA
cgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 109 - protein, 153 aa, synthetic construct
  REGION 1..153: note = Description of Artificial Sequence: Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 110 - protein, 134 aa, synthetic construct
  REGION 1..134: note = Description of Artificial Sequence: Synthetic polypeptide
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 111 - protein, 121 aa, synthetic construct [sequence omitted]

SEQ ID NO: 112 - DNA, 363 bp, synthetic construct
  misc_feature 1..363: note = Description of Artificial Sequence: Synthetic polynucleotide
  source 1..363: mol_type = other DNA
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatggaggggggatggttactacgcgtacttcgatgtctggggcgcagggaccacggtcaccgtctcctca

SEQ ID NO: 113 - protein, 111 aa, synthetic construct
  REGION 1..111: note = Description of Artificial Sequence: Synthetic polypeptide
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 114 - DNA, 333 bp, synthetic construct
  misc_feature 1..333: note = Description of Artificial Sequence: Synthetic polynucleotide
  source 1..333: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaa

SEQ ID NO: 115 - protein, 451 aa, synthetic construct
  REGION 1..451: note = Description of Artificial Sequence: Synthetic polypeptide
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 116 - DNA, 1353 bp, synthetic construct
  misc_feature 1..1353: note = Description of Artificial Sequence: Synthetic polynucleotide
  source 1..1353: mol_type = other DNA
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatggaggggggatggttactacgcgtacttcgatgtctggggcgcagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 117 - protein, 218 aa, synthetic construct
  REGION 1..218: note = Description of Artificial Sequence: Synthetic polypeptide
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118 - DNA, 654 bp, synthetic construct
  misc_feature 1..654: note = Description of Artificial Sequence: Synthetic polynucleotide
  source 1..654: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 119 - protein, 5 aa, synthetic construct
  REGION 1..5: note = Description of Artificial Sequence: Synthetic peptide
  VARIANT 1: note = Asn or Ser
XYLIE

SEQ ID NO: 120 - protein, 17 aa, synthetic construct
  REGION 1..17: note = Description of Artificial Sequence: Synthetic peptide
  VARIANT 12: note = Asn or Ala
  VARIANT 13: note = Glu or Asp
  VARIANT 14: note = Lys or Ser
  VARIANT 15: note = Phe or Val
VINPGSGGTNYXXXXKG

SEQ ID NO: 121 - protein, 12 aa, synthetic construct
  REGION 1..12: note = Description of Artificial Sequence: Synthetic peptide
  VARIANT 4: note = Glu, Asp, Ala, Gly or Thr
  VARIANT 5: note = Gly, Ala, Thr or Ser
WRGXXYYAYFDV

SEQ ID NO: 122 - protein, 15 aa, synthetic construct
  REGION 1..15: note = Description of Artificial Sequence: Synthetic peptide
  VARIANT 1: note = Arg or Lys
  VARIANT 7: note = Ser or Asp
  VARIANT 9: note = Asp, Gln, Ala, Gly or Thr
  VARIANT 10: note = Gly, Thr or Ser
XASQSVXYXXDSYMN

SEQ ID NO: 123 - protein, 7 aa, synthetic construct
  REGION 1..7: note = Description of Artificial Sequence: Synthetic peptide
  VARIANT 6: note = Glu or Ala
AASNLXS

SEQ ID NO: 124 - protein, 218 aa, synthetic construct
  REGION 1..218: note = Description of Artificial Sequence: Synthetic polypeptide
DIVLTQSPLSLPVTLGQPASISCKASQSVDYDGDSYMNWYQQRPGQSPRLLIYAASNLESGVPDRFSGSGSGTDFTLKISRVEAEDVGVYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - DNA, 654 bp, synthetic construct
  misc_feature 1..654: note = Description of Artificial Sequence: Synthetic polynucleotide
  source 1..654: mol_type = other DNA
gacatcgtgctgacacagagccctctgtccctgcccgtgaccctgggccagcctgcctccatctcctgcaaggcctcccagtccgtggactacgacggcgactcctacatgaactggtatcagcagcggcctggccagtcccctcggctgctgatctacgccgcctccaacctggaatccggcgtgcccgacagattctccggctccggctctggcaccgacttcaccctgaagatctcccgggtggaagccgaggacgtgggcgtgtactactgccagcagtccaacgaggacccctacaccttcggcggaggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 126 - protein, 451 aa, synthetic construct
  REGION 1..451: note = Description of Artificial Sequence: Synthetic polypeptide
QVQLVQSGAEVKKPGSSVKVSCKASGYAFTNYLIEWVRQAPGQGLEWMGVINPGSGGTNYNEKFKGRVTITADKSTSTAYMELSSLRSEDTAVYYCARWRGDGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - DNA, 1353 bp, synthetic construct
  misc_feature 1..1353: note = Description of Artificial Sequence: Synthetic polynucleotide
  source 1..1353: mol_type = other DNA
caagtgcagctggtgcagtctggcgctgaagtgaagaaacccggctcctccgtgaaagtgtcctgcaaggcctccggctacgccttcaccaactacctgatcgagtgggtccgacaggccccaggccagggcctggagtggatgggcgtgatcaaccctggctccggcggcaccaactacaacgagaagttcaagggcagagtgaccatcaccgccgacaagtccacctccaccgcctacatggaactgtcctccctgcggagcgaggacaccgccgtgtactactgtgcccggtggcggggagatggctactacgcctacttcgacgtgtggggccagggcaccaccgtgaccgtgtcctctgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacgcctccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 128 - protein, 218 aa, synthetic construct
  REGION 1..218: note = Description of Artificial Sequence: Synthetic polypeptide
AIRLTQSPSSFSASTGDRVTITCKASQSVDYDGDSYMNWYQQKPGKAPKLLIYAASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - DNA, 654 bp, synthetic construct
  misc_feature 1..654: note = Description of Artificial Sequence: Synthetic polynucleotide
  source 1..654: mol_type = other DNA
gccatcagactgacccagagcccctccagcttctccgcctccaccggcgacagagtgaccatcacatgcaaggcctcccagtccgtggactacgacggcgactcctacatgaactggtatcagcagaagcccggcaaggcccctaagctgctgatctacgccgcctccaacctggaatccggcgtgccctcccggttctccggctctggctctggcaccgacttcaccctgaccatctccagcctgcagtccgaggacttcgccacctactactgccagcagtccaacgaggacccctacaccttcggcggaggcaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 130 - protein, 451 aa, synthetic construct
  REGION 1..451: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGDGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 1353 bp, synthetic construct
  misc_feature 1..1353: note = Description of Artificial Sequence: Synthetic polynucleotide
  source 1..1353: mol_type = other DNA
gaagtgcagctggtgcagtctggcgctgaagtgaagaagcccggcgagtccctgaagatctcctgcaagggctccggctacgccttcaccaactacctgatcgagtgggtccgacagatgcccggcaagggcctggagtggatgggcgtgatcaaccccggctccggcggcaccaactacaacgagaagttcaagggccaagtcacaatctccgccgacaagtccatctccaccgcctacctgcagtggtcctccctgaaggcctccgacaccgccatgtactactgcgccagatggcggggagatggctactacgcctacttcgacgtgtggggccagggcaccaccgtgaccgtgtcctctgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacgcctccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 132 - protein, 52 aa, synthetic construct
  REGION 1..52: note = Description of Artificial Sequence: Synthetic polypeptide
GINNYKNPKLTRMLTFKFYMPKKATELKHLQCLEEELKPLEEVLNLAQSKNF

SEQ ID NO: 133 - protein, 10 aa, synthetic construct
  REGION 1..10: note = Description of Artificial Sequence: Synthetic peptide
GYAFSNYLIE

SEQ ID NO: 134 - protein, 17 aa, synthetic construct
  REGION 1..17: note = Description of Artificial Sequence: Synthetic peptide
VINPGSGGTYYNEKFKG

SEQ ID NO: 135 - protein, 12 aa, synthetic construct
  REGION 1..12: note = Description of Artificial Sequence: Synthetic peptide
WRGEGYYAYYDV

SEQ ID NO: 136 - protein, 5 aa, synthetic construct
  REGION 1..5: note = Description of Artificial Sequence: Synthetic peptide
NYLIE

SEQ ID NO: 137 - protein, 17 aa, synthetic construct
  REGION 1..17: note = Description of Artificial Sequence: Synthetic peptide
VINPGSGGTYYNEKFKG

SEQ ID NO: 138 - protein, 12 aa, synthetic construct
  REGION 1..12: note = Description of Artificial Sequence: Synthetic peptide
WRGEGYYAYYDV

SEQ ID NO: 139 - protein, 7 aa, synthetic construct
  REGION 1..7: note = Description of Artificial Sequence: Synthetic peptide
GYAFSNY

SEQ ID NO: 140 - protein, 6 aa, synthetic construct
  REGION 1..6: note = Description of Artificial Sequence: Synthetic peptide
NPGSGG

SEQ ID NO: 141 - protein, 12 aa, synthetic construct
  REGION 1..12: note = Description of Artificial Sequence: Synthetic peptide
WRGEGYYAYYDV

SEQ ID NO: 142 - protein, 8 aa, synthetic construct
  REGION 1..8: note = Description of Artificial Sequence: Synthetic peptide
GYAFSNYL

SEQ ID NO: 143 - protein, 8 aa, synthetic construct
  REGION 1..8: note = Description of Artificial Sequence: Synthetic peptide
INPGSGGT

SEQ ID NO: 144 - protein, 14 aa, synthetic construct
  REGION 1..14: note = Description of Artificial Sequence: Synthetic peptide
ARWRGEGYYAYYDV

SEQ ID NO: 145 - protein, 121 aa, synthetic construct
  REGION 1..121: note = Description of Artificial Sequence: Synthetic polypeptide
EVQLVQSGAEVKKPGESLKISCKGSGYAFSNYLIEWVRQMPGKGLEWMGVINPGSGGTYYNEKFKGQVTISADKSISTAYLQWSSLKASDTAMYYCARWRGEGYYAYYDVWGQGTTVTVSS

SEQ ID NO: 146 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctca

SEQ ID NO: 147 - protein, 451 aa, synthetic construct
  REGION 1..451: note = Description of Artificial Sequence: Synthetic polypeptide
EVQLVQSGAEVKKPGESLKISCKGSGYAFSNYLIEWVRQMPGKGLEWMGVINPGSGGTYYNEKFKGQVTISADKSISTAYLQWSSLKASDTAMYYCARWRGEGYYAYYDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 149 - protein, 10 aa, synthetic construct
  REGION 1..10: note = Description of Artificial Sequence: Synthetic peptide
GYAFTNYLIE

SEQ ID NO: 150 - protein, 17 aa, synthetic construct
  REGION 1..17: note = Description of Artificial Sequence: Synthetic peptide
VINPGSGGTYYNEKFKG

SEQ ID NO: 151 - protein, 12 aa, synthetic construct
  REGION 1..12: note = Description of Artificial Sequence: Synthetic peptide
WRGEGYYAYYDV

SEQ ID NO: 152 - protein, 5 aa, synthetic construct
  REGION 1..5: note = Description of Artificial Sequence: Synthetic peptide
NYLIE

SEQ ID NO: 153 - protein, 17 aa, synthetic construct
  REGION 1..17: note = Description of Artificial Sequence: Synthetic peptide
VINPGSGGTYYNEKFKG

SEQ ID NO: 154 - protein, 12 aa, synthetic construct
  REGION 1..12: note = Description of Artificial Sequence: Synthetic peptide
WRGEGYYAYYDV

SEQ ID NO: 155 - protein, 7 aa, synthetic construct
  REGION 1..7: note = Description of Artificial Sequence: Synthetic peptide
GYAFTNY

SEQ ID NO: 156 - protein, 6 aa, synthetic construct
  REGION 1..6: note = Description of Artificial Sequence: Synthetic peptide
NPGSGG

SEQ ID NO: 157 - protein, 12 aa, synthetic construct
  REGION 1..12: note = Description of Artificial Sequence: Synthetic peptide
WRGEGYYAYYDV

SEQ ID NO: 158 - protein, 8 aa, synthetic construct
  REGION 1..8: note = Description of Artificial Sequence: Synthetic peptide
GYAFTNYL

SEQ ID NO: 159 - protein, 8 aa, synthetic construct
  REGION 1..8: note = Description of Artificial Sequence: Synthetic peptide
INPGSGGT

SEQ ID NO: 160 - protein, 14 aa, synthetic construct
  REGION 1..14: note = Description of Artificial Sequence: Synthetic peptide
ARWRGEGYYAYYDV

SEQ ID NO: 161 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTYYNEKFKGQVTISADKSISTAYLQWSSLKASDTAMYYCARWRGEGYYAYYDVWGQGTTVTVSS

SEQ ID NO: 162 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctca

SEQ ID NO: 163 - protein, 451 aa, synthetic construct
  REGION 1..451: note = Description of Artificial Sequence: Synthetic polypeptide
EVQLVQSGAEVKKPGESLKISCKGSGYAFTNYLIEWVRQMPGKGLEWMGVINPGSGGTYYNEKFKGQVTISADKSISTAYLQWSSLKASDTAMYYCARWRGEGYYAYYDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 165 - protein, 10 aa, synthetic construct
  REGION 1..10: note = Description of Artificial Sequence: Synthetic peptide
GYAFSNYLIE

SEQ ID NO: 166 - protein, 17 aa, synthetic construct
  REGION 1..17: note = Description of Artificial Sequence: Synthetic peptide
VINPGSGGTNYNEKFKG

SEQ ID NO: 167 - protein, 12 aa, synthetic construct
  REGION 1..12: note = Description of Artificial Sequence: Synthetic peptide
WRGEGYYAYYDV

SEQ ID NO: 168 - protein, 5 aa, synthetic construct
  REGION 1..5: note = Description of Artificial Sequence: Synthetic peptide
NYLIE

SEQ ID NO: 169 - protein, 17 aa, synthetic construct
  REGION 1..17: note = Description of Artificial Sequence: Synthetic peptide
VINPGSGGTNYNEKFKG

SEQ ID NO: 170 - protein, 12 aa, synthetic construct
  REGION 1..12: note = Description of Artificial Sequence: Synthetic peptide
WRGEGYYAYYDV

SEQ ID NO: 171 - protein, 7 aa, synthetic construct
  REGION 1..7: note = Description of Artificial Sequence: Synthetic peptide
GYAFSNY

SEQ ID NO: 172 - protein, 6 aa, synthetic construct
  REGION 1..6: note = Description of Artificial Sequence: Synthetic peptide
NPGSGG

SEQ ID NO: 173 - protein, 12 aa, synthetic construct
  REGION 1..12: note = Description of Artificial Sequence: Synthetic peptide
WRGEGYYAYYDV

SEQ ID NO: 174 - protein, 8 aa, synthetic construct
  REGION 1..8: note = Description of Artificial Sequence: Synthetic peptide
GYAFSNYL

SEQ ID NO: 175 - protein, 8 aa, synthetic construct
  REGION 1..8: note = Description of Artificial Sequence: Synthetic peptide
INPGSGGT

SEQ ID NO: 176 - protein, 14 aa, synthetic construct
  REGION 1..14: note = Description of Artificial Sequence: Synthetic peptide
ARWRGEGYYAYYDV

SEQ ID NO: 177 - protein, 121 aa, synthetic construct
  REGION 1..121: note = Description of Artificial Sequence: Synthetic polypeptide
EVQLVQSGAEVKKPGESLKISCKGSGYAFSNYLIEWVRQMPGKGLEWMGVINPGSGGTNYNEKFKGQVTISADKSISTAYLQWSSLKASDTAMYYCARWRGEGYYAYYDVWGQGTTVTVSS

SEQ ID NO: 178 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctca

SEQ ID NO: 179 - protein, 451 aa, synthetic construct
  REGION 1..451: note = Description of Artificial Sequence: Synthetic polypeptide
EVQLVQSGAEVKKPGESLKISCKGSGYAFSNYLIEWVRQMPGKGLEWMGVINPGSGGTNYNEKFKGQVTISADKSISTAYLQWSSLKASDTAMYYCARWRGEGYYAYYDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 181 - protein, 10 aa, synthetic construct
  REGION 1..10: note = Description of Artificial Sequence: Synthetic peptide
GYAFTNYLIE

SEQ ID NO: 182 - protein, 17 aa, synthetic construct
  REGION 1..17: note = Description of Artificial Sequence: Synthetic peptide
VINPGSGGTNYNEKFKG

SEQ ID NO: 183 - protein, 12 aa, synthetic construct
  REGION 1..12: note = Description of Artificial Sequence: Synthetic peptide
WRGEGYYAYYDV

SEQ ID NO: 184 - protein, 5 aa, synthetic construct
  REGION 1..5: note = Description of Artificial Sequence: Synthetic peptide
NYLIE

SEQ ID NO: 185 - protein, 17 aa, synthetic construct
  REGION 1..17: note = Description of Artificial Sequence: Synthetic peptide
VINPGSGGTNYNEKFKG

SEQ ID NO: 186 - protein, 12 aa, synthetic construct
  REGION 1..12: note = Description of Artificial Sequence: Synthetic peptide
WRGEGYYAYYDV

SEQ ID NO: 187 - protein, 7 aa, synthetic construct
  REGION 1..7: note = Description of Artificial Sequence: Synthetic peptide
GYAFTNY

SEQ ID NO: 188 - protein, 6 aa, synthetic construct
  REGION 1..6: note = Description of Artificial Sequence: Synthetic peptide
NPGSGG

SEQ ID NO: 189 - protein, 12 aa, synthetic construct
  REGION 1..12: note = Description of Artificial Sequence: Synthetic peptide
WRGEGYYAYYDV

SEQ ID NO: 190 - protein, 8 aa, synthetic construct
  REGION 1..8: note = Description of Artificial Sequence: Synthetic peptide
GYAFTNYL

SEQ ID NO: 191 - protein, 8 aa, synthetic construct
  REGION 1..8: note = Description of Artificial Sequence: Synthetic peptide
INPGSGGT

SEQ ID NO: 192 - protein, 14 aa, synthetic construct
  REGION 1..14: note = Description of Artificial Sequence: Synthetic peptide
ARWRGEGYYAYYDV

SEQ ID NO: 193 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYYDVWGQGTTVTVSS

SEQ ID NO: 194 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctca

SEQ ID NO: 195 - protein, 451 aa, synthetic construct
  REGION 1..451: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYYDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagagggctactacgcctactacgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 197 - protein, 10 aa, synthetic construct
  REGION 1..10: note = Description of Artificial Sequence: Synthetic peptide
GYAFSNYLIE

SEQ ID NO: 198 - protein, 17 aa, synthetic construct
  REGION 1..17: note = Description of Artificial Sequence: Synthetic peptide
VINPGSGGTYYNEKFKG

SEQ ID NO: 199 - protein, 12 aa, synthetic construct
  REGION 1..12: note = Description of Artificial Sequence: Synthetic peptide
WRGEGYYAYFDV

SEQ ID NO: 200 - protein, 5 aa, synthetic construct
  REGION 1..5: note = Description of Artificial Sequence: Synthetic peptide
NYLIE

SEQ ID NO: 201 - protein, 17 aa, synthetic construct
  REGION 1..17: note = Description of Artificial Sequence: Synthetic peptide
VINPGSGGTYYNEKFKG

SEQ ID NO: 202 - protein, 12 aa, synthetic construct
  REGION 1..12: note = Description of Artificial Sequence: Synthetic peptide
WRGEGYYAYFDV

SEQ ID NO: 203 - protein, 7 aa, synthetic construct
  REGION 1..7: note = Description of Artificial Sequence: Synthetic peptide
GYAFSNY

SEQ ID NO: 204 - protein, 6 aa, synthetic construct
  REGION 1..6: note = Description of Artificial Sequence: Synthetic peptide
NPGSGG

SEQ ID NO: 205 - protein, 12 aa, synthetic construct
  REGION 1..12: note = Description of Artificial Sequence: Synthetic peptide
WRGEGYYAYFDV

SEQ ID NO: 206 - protein, 8 aa, synthetic construct
  REGION 1..8: note = Description of Artificial Sequence: Synthetic peptide
GYAFSNYL

SEQ ID NO: 207 - protein, 8 aa, synthetic construct
  REGION 1..8: note = Description of Artificial Sequence: Synthetic peptide
INPGSGGT

SEQ ID NO: 208 - protein, 14 aa, synthetic construct
  REGION 1..14: note = Description of Artificial Sequence: Synthetic peptide
ARWRGEGYYAYFDV

SEQ ID NO: 209 - protein, 121 aa, synthetic construct
  REGION 1..121: note = Description of Artificial Sequence: Synthetic polypeptide
EVQLVQSGAEVKKPGESLKISCKGSGYAFSNYLIEWVRQMPGKGLEWMGVINPGSGGTYYNEKFKGQVTISADKSISTAYLQWSSLKASDTAMYYCARWRGEGYYAYFDVWGQGTTVTVSS

SEQ ID NO: 210 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctacttcgacgtgtggggccagggcaccacagtgaccgtcagctca

SEQ ID NO: 211 - protein, 451 aa, synthetic construct
  REGION 1..451: note = Description of Artificial Sequence: Synthetic polypeptide
EVQLVQSGAEVKKPGESLKISCKGSGYAFSNYLIEWVRQMPGKGLEWMGVINPGSGGTYYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcagcaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctacttcgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 213 - protein, 10 aa, synthetic construct
  REGION 1..10: note = Description of Artificial Sequence: Synthetic peptide
GYAFTNYLIE

SEQ ID NO: 214 - protein, 17 aa, synthetic construct
  REGION 1..17: note = Description of Artificial Sequence: Synthetic peptide
VINPGSGGTYYNEKFKG

SEQ ID NO: 215 - protein, 12 aa, synthetic construct
  REGION 1..12: note = Description of Artificial Sequence: Synthetic peptide
WRGEGYYAYFDV

SEQ ID NO: 216 - protein, 5 aa, synthetic construct
  REGION 1..5: note = Description of Artificial Sequence: Synthetic peptide
NYLIE

SEQ ID NO: 217 - protein, 17 aa, synthetic construct
  REGION 1..17: note = Description of Artificial Sequence: Synthetic peptide
VINPGSGGTYYNEKFKG

SEQ ID NO: 218 - protein, 12 aa, synthetic construct
  REGION 1..12: note = Description of Artificial Sequence: Synthetic peptide
WRGEGYYAYFDV

SEQ ID NO: 219 - protein, 7 aa, synthetic construct
  REGION 1..7: note = Description of Artificial Sequence: Synthetic peptide
GYAFTNY

SEQ ID NO: 220 - protein, 6 aa, synthetic construct
  REGION 1..6: note = Description of Artificial Sequence: Synthetic peptide
NPGSGG

SEQ ID NO: 221 - protein, 12 aa, synthetic construct
  REGION 1..12: note = Description of Artificial Sequence: Synthetic peptide
WRGEGYYAYFDV

SEQ ID NO: 222 - protein, 8 aa, synthetic construct
  REGION 1..8: note = Description of Artificial Sequence: Synthetic peptide
GYAFTNYL

SEQ ID NO: 223 - protein, 8 aa, synthetic construct
  REGION 1..8: note = Description of Artificial Sequence: Synthetic peptide
INPGSGGT

SEQ ID NO: 224 - protein, 14 aa, synthetic construct
  REGION 1..14: note = Description of Artificial Sequence: Synthetic peptide
ARWRGEGYYAYFDV

SEQ ID NO: 225 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTYYNEKFKGQVTISADKSISTAYLQWSSLKASDTAMYYCARWRGEGYYAYFDVWGQGTTVTVSS

SEQ ID NO: 226 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctacttcgacgtgtggggccagggcaccacagtgaccgtcagctca

SEQ ID NO: 227 - protein, 451 aa, synthetic construct
  REGION 1..451: note = Description of Artificial Sequence: Synthetic polypeptide
EVQLVQSGAEVKKPGESLKISCKGSGYAFTNYLIEWVRQMPGKGLEWMGVINPGSGGTYYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcacctactacaacgagaagttcaagggccaagtgaccatcagcgccgacaagagcatcagcaccgcctacctccagtggtccagcctgaaggccagcgacaccgccatgtactactgcgccaggtggcggggagagggctactacgcctacttcgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 229 - protein, 451 aa, synthetic construct
  REGION 1..451: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - DNA, 1353 bp, synthetic construct
  misc_feature 1..1353: note = Description of Artificial Sequence: Synthetic polynucleotide
  source 1..1353: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggagagggctactacgcctacttcgacgtgtggggccagggcaccacagtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 231 - protein, 15 aa, synthetic construct
  REGION 1..15: note = Description of Artificial Sequence: Synthetic peptide
KASQSVDYQGDSYMN

SEQ ID NO: 232 - protein, 7 aa, synthetic construct
  REGION 1..7: note = Description of Artificial Sequence: Synthetic peptide
SASNLES

SEQ ID NO: 233 - protein, 9 aa, synthetic construct
  REGION 1..9: note = Description of Artificial Sequence: Synthetic peptide
QQSNEDPYT

SEQ ID NO: 234 - protein, 15 aa, synthetic construct
  REGION 1..15: note = Description of Artificial Sequence: Synthetic peptide
KASQSVDYQGDSYMN

SEQ ID NO: 235 - protein, 7 aa, synthetic construct
  REGION 1..7: note = Description of Artificial Sequence: Synthetic peptide
SASNLES

SEQ ID NO: 236 - protein, 9 aa, synthetic construct
  REGION 1..9: note = Description of Artificial Sequence: Synthetic peptide
QQSNEDPYT

SEQ ID NO: 237 - protein, 11 aa, synthetic construct
  REGION 1..11: note = Description of Artificial Sequence: Synthetic peptide
SQSVDYQGDSY

SEQ ID NO: 238 - skipped

SEQ ID NO: 239 - protein, 6 aa, synthetic construct
  REGION 1..6: note = Description of Artificial Sequence: Synthetic peptide
SNEDPY

SEQ ID NO: 240 - protein, 10 aa, synthetic construct
  REGION 1..10: note = Description of Artificial Sequence: Synthetic peptide
QSVDYQGDSY

SEQ ID NO: 241 - skipped

SEQ ID NO: 242 - protein, 9 aa, synthetic construct
  REGION 1..9: note = Description of Artificial Sequence: Synthetic peptide
QQSNEDPYT

SEQ ID NO: 243 - protein, 111 aa, synthetic construct
  REGION 1..111: note = Description of Artificial Sequence: Synthetic polypeptide
AIRLTQSPSSFSASTGDRVTITCKASQSVDYQGDSYMNWYQQKPGKAPKLLIYSASNLESGVPSRFSGSGSGTDFTLTISSLQSEDFATYYCQQSNEDPYTFGGGTKVEIK

SEQ ID NO: 244 - DNA, 333 bp, synthetic construct
  misc_feature 1..333: note = Description of Artificial Sequence: Synthetic polynucleotide
  source 1..333: mol_type = other DNA
gccatcagactgacccagagccccagcagctttagcgccagcaccggcgacagagtgaccatcacatgcaaggccagccagagcgtggactaccagggcgacagctacatgaactggtatcagcagaagcccggcaaggcccccaagctgctgatctactccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaag

SEQ ID NO: 245 - protein, 218 aa, synthetic construct
  REGION 1..218: note = Description of Artificial Sequence: Synthetic polypeptide
AIRLTQSPSSFSASTGDRVTITCKASQSVDYQGDSYMNWYQQKPGKAPKLLIYSASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - DNA, 654 bp, synthetic construct
  misc_feature 1..654: note = Description of Artificial Sequence: Synthetic polynucleotide
  source 1..654: mol_type = other DNA
gccatcagactgacccagagccccagcagctttagcgccagcaccggcgacagagtgaccatcacatgcaaggccagccagagcgtggactaccagggcgacagctacatgaactggtatcagcagaagcccggcaaggcccccaagctgctgatctactccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 247 - protein, 15 aa, synthetic construct
  REGION 1..15: note = Description of Artificial Sequence: Synthetic peptide
KASQSVDYQGDSYMN

SEQ ID NO: 248 - protein, 7 aa, synthetic construct
  REGION 1..7: note = Description of Artificial Sequence: Synthetic peptide
TASNLES

SEQ ID NO: 249 - protein, 9 aa, synthetic construct
  REGION 1..9: note = Description of Artificial Sequence: Synthetic peptide
QQSNEDPYT

SEQ ID NO: 250 - protein, 15 aa, synthetic construct
  REGION 1..15: note = Description of Artificial Sequence: Synthetic peptide
KASQSVDYQGDSYMN

SEQ ID NO: 251 - protein, 7 aa, synthetic construct
  REGION 1..7: note = Description of Artificial Sequence: Synthetic peptide
TASNLES

SEQ ID NO: 252 - protein, 9 aa, synthetic construct
  REGION 1..9: note = Description of Artificial Sequence: Synthetic peptide
QQSNEDPYT

SEQ ID NO: 253 - protein, 11 aa, synthetic construct
  REGION 1..11: note = Description of Artificial Sequence: Synthetic peptide
SQSVDYQGDSY

SEQ ID NO: 254 - skipped

SEQ ID NO: 255 - protein, 6 aa, synthetic construct
  REGION 1..6: note = Description of Artificial Sequence: Synthetic peptide
SNEDPY

SEQ ID NO: 256 - protein, 10 aa, synthetic construct
  REGION 1..10: note = Description of Artificial Sequence: Synthetic peptide
QSVDYQGDSY

SEQ ID NO: 257 - skipped

SEQ ID NO: 258 - protein, 9 aa, synthetic construct
  REGION 1..9: note = Description of Artificial Sequence: Synthetic peptide
QQSNEDPYT

SEQ ID NO: 259 - protein, 111 aa, synthetic construct
  REGION 1..111: note = Description of Artificial Sequence: Synthetic polypeptide
AIRLTQSPSSFSASTGDRVTITCKASQSVDYQGDSYMNWYQQKPGKAPKLLIYTASNLESGVPSRFSGSGSGTDFTLTISSLQSEDFATYYCQQSNEDPYTFGGGTKVEIK

SEQ ID NO: 260 - DNA, 333 bp, synthetic construct
  misc_feature 1..333: note = Description of Artificial Sequence: Synthetic polynucleotide
  source 1..333: mol_type = other DNA
gccatcagactgacccagagccccagcagctttagcgccagcaccggcgacagagtgaccatcacatgcaaggccagccagagcgtggactaccagggcgacagctacatgaactggtatcagcagaagcccggcaaggcccccaagctgctgatctacaccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaag

SEQ ID NO: 261 - protein, 218 aa, synthetic construct
  REGION 1..218: note = Description of Artificial Sequence: Synthetic polypeptide
AIRLTQSPSSFSASTGDRVTITCKASQSVDYQGDSYMNWYQQKPGKAPKLLIYTASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - DNA, 654 bp, synthetic construct
  misc_feature 1..654: note = Description of Artificial Sequence: Synthetic polynucleotide
  source 1..654: mol_type = other DNA
gccatcagactgacccagagccccagcagctttagcgccagcaccggcgacagagtgaccatcacatgcaaggccagccagagcgtggactaccagggcgacagctacatgaactggtatcagcagaagcccggcaaggcccccaagctgctgatctacaccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 263 - protein, 15 aa, synthetic construct
  REGION 1..15: note = Description of Artificial Sequence: Synthetic peptide
KASQSVDYQGDSYLN

SEQ ID NO: 264 - protein, 7 aa, synthetic construct
  REGION 1..7: note = Description of Artificial Sequence: Synthetic peptide
SASNLES

SEQ ID NO: 265 - protein, 9 aa, synthetic construct
  REGION 1..9: note = Description of Artificial Sequence: Synthetic peptide
QQSNEDPYT

SEQ ID NO: 266 - protein, 15 aa, synthetic construct
  REGION 1..15: note = Description of Artificial Sequence: Synthetic peptide
KASQSVDYQGDSYLN

SEQ ID NO: 267 - protein, 7 aa, synthetic construct
  REGION 1..7: note = Description of Artificial Sequence: Synthetic peptide
SASNLES

SEQ ID NO: 268 - protein, 9 aa, synthetic construct
  REGION 1..9: note = Description of Artificial Sequence: Synthetic peptide
QQSNEDPYT

SEQ ID NO: 269 - protein, 11 aa, synthetic construct
  REGION 1..11: note = Description of Artificial Sequence: Synthetic peptide
SQSVDYQGDSY

SEQ ID NO: 270 - skipped

SEQ ID NO: 271 - protein, 6 aa, synthetic construct
  REGION 1..6: note = Description of Artificial Sequence: Synthetic peptide
SNEDPY

SEQ ID NO: 272 - protein, 10 aa, synthetic construct
  REGION 1..10: note = Description of Artificial Sequence: Synthetic peptide
QSVDYQGDSY

SEQ ID NO: 273 - skipped

SEQ ID NO: 274 - protein, 9 aa, synthetic construct
  REGION 1..9: note = Description of Artificial Sequence: Synthetic peptide
QQSNEDPYT

SEQ ID NO: 275 - protein, 111 aa, synthetic construct
  REGION 1..111: note = Description of Artificial Sequence: Synthetic polypeptide
AIRLTQSPSSFSASTGDRVTITCKASQSVDYQGDSYLNWYQQKPGKAPKLLIYSASNLESGVPSRFSGSGSGTDFTLTISSLQSEDFATYYCQQSNEDPYTFGGGTKVEIK

SEQ ID NO: 276 - DNA, 333 bp, synthetic construct
  misc_feature 1..333: note = Description of Artificial Sequence: Synthetic polynucleotide
  source 1..333: mol_type = other DNA
gccatcagactgacccagagccccagcagctttagcgccagcaccggcgacagagtgaccatcacatgcaaggccagccagagcgtggactaccagggcgacagctacctgaactggtatcagcagaagcccggcaaggcccccaagctgctgatctactccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaag

SEQ ID NO: 277 - protein, 218 aa, synthetic construct
  REGION 1..218: note = Description of Artificial Sequence: Synthetic polypeptide
AIRLTQSPSSFSASTGDRVTITCKASQSVDYQGDSYLNWYQQKPGKAPKLLIYSASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - DNA, 654 bp, synthetic construct
  misc_feature 1..654: note = Description of Artificial Sequence: Synthetic polynucleotide
  source 1..654: mol_type = other DNA
gccatcagactgacccagagccccagcagctttagcgccagcaccggcgacagagtgaccatcacatgcaaggccagccagagcgtggactaccagggcgacagctacctgaactggtatcagcagaagcccggcaaggcccccaagctgctgatctactccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 279 - protein, 15 aa, synthetic construct
  REGION 1..15: note = Description of Artificial Sequence: Synthetic peptide
KASQSVDYQGDSYLN

SEQ ID NO: 280 - protein, 7 aa, synthetic construct
  REGION 1..7: note = Description of Artificial Sequence: Synthetic peptide
AASNLES

SEQ ID NO: 281 - protein, 9 aa, synthetic construct
  REGION 1..9: note = Description of Artificial Sequence: Synthetic peptide
QQSNEDPYT

SEQ ID NO: 282 - protein, 15 aa, synthetic construct
  REGION 1..15: note = Description of Artificial Sequence: Synthetic peptide
KASQSVDYQGDSYLN

SEQ ID NO: 283 - protein, 7 aa, synthetic construct
  REGION 1..7: note = Description of Artificial Sequence: Synthetic peptide
AASNLES

SEQ ID NO: 284 - protein, 9 aa, synthetic construct
  REGION 1..9: note = Description of Artificial Sequence: Synthetic peptide
QQSNEDPYT

SEQ ID NO: 285 - protein, 11 aa, synthetic construct
  REGION 1..11: note = Description of Artificial Sequence: Synthetic peptide
SQSVDYQGDSY

SEQ ID NO: 286-386 - skipped (101 entries)

SEQ ID NO: 387 - protein, 6 aa, synthetic construct
  REGION 1..6: note = Description of Artificial Sequence: Synthetic peptide
SNEDPY

SEQ ID NO: 388 - protein, 10 aa, synthetic construct
  REGION 1..10: note = Description of Artificial Sequence: Synthetic peptide
QSVDYQGDSY

SEQ ID NO: 389 - skipped

SEQ ID NO: 390 - protein, 9 aa, synthetic construct
  REGION 1..9: note = Description of Artificial Sequence: Synthetic peptide
QQSNEDPYT

SEQ ID NO: 391 - protein, 111 aa, synthetic construct
  REGION 1..111: note = Description of Artificial Sequence: Synthetic polypeptide
AIRLTQSPSSFSASTGDRVTITCKASQSVDYQGDSYLNWYQQKPGKAPKLLIYAASNLESGVPSRFSGSGSGTDFTLTISSLQSEDFATYYCQQSNEDPYTFGGGTKVEIK

SEQ ID NO: 392 - DNA, 333 bp, synthetic construct
  misc_feature 1..333: note = Description of Artificial Sequence: Synthetic polynucleotide
  source 1..333: mol_type = other DNA
gccatcagactgacccagagccccagcagctttagcgccagcaccggcgacagagtgaccatcacatgcaaggccagccagagcgtggactaccagggcgacagctacctgaactggtatcagcagaagcccggcaaggcccccaagctgctgatctacgccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaag

SEQ ID NO: 393 - protein, 218 aa, synthetic construct
  REGION 1..218: note = Description of Artificial Sequence: Synthetic polypeptide
AIRLTQSPSSFSASTGDRVTITCKASQSVDYQGDSYLNWYQQKPGKAPKLLIYAASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 394 - DNA, 654 bp, synthetic construct
  misc_feature 1..654: note = Description of Artificial Sequence: Synthetic polynucleotide
  source 1..654: mol_type = other DNA
gccatcagactgacccagagccccagcagctttagcgccagcaccggcgacagagtgaccatcacatgcaaggccagccagagcgtggactaccagggcgacagctacctgaactggtatcagcagaagcccggcaaggcccccaagctgctgatctacgccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 395 - protein, 218 aa, synthetic construct
  REGION 1..218: note = Description of Artificial Sequence: Synthetic polypeptide
AIRLTQSPSSFSASTGDRVTITCKASQSVDYQGDSYMNWYQQKPGKAPKLLIYAASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 396 - DNA, 654 bp, synthetic construct
  misc_feature 1..654: note = Description of Artificial Sequence: Synthetic polynucleotide
  source 1..654: mol_type = other DNA
gccatcagactgacccagagccccagcagctttagcgccagcaccggcgacagagtgaccatcacatgcaaggccagccagagcgtggactaccagggcgacagctacatgaactggtatcagcagaagcccggcaaggcccccaagctgctgatctacgccgccagcaatctggaaagcggcgtgcccagcagattcagcggctctggcagcggcaccgacttcaccctgacaatcagcagcctccagtccgaggacttcgccacctactactgccagcagagcaacgaggacccctacacctttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 397 - protein, 15 aa, synthetic construct
  REGION 1..15: note = Description of Artificial Sequence: Synthetic peptide
GGGGSGGGGSGGGGS

SEQ ID NO: 398 - protein, 20 aa, synthetic construct
  REGION 1..20: note = Description of Artificial Sequence: Synthetic peptide
GGGGSGGGGSGGGGSGGGGS

SEQ ID NO: 399 - protein, 25 aa, synthetic construct
  REGION 1..25: note = Description of Artificial Sequence: Synthetic peptide
GGGGSGGGGSGGGGSGGGGSGGGGS

SEQ ID NO: 400 - protein, 30 aa, synthetic construct
  REGION 1..30: note = Description of Artificial Sequence: Synthetic polypeptide
GGGGSGGGGSGGGGSGGGGSGGGGSGGGGS

SEQ ID NO: 401 - protein, 35 aa, synthetic construct
  REGION 1..35: note = Description of Artificial Sequence: Synthetic polypeptide
GGGGSGGGGSGGGGSGGGGSGGGGSGGGGSGGGGS

SEQ ID NO: 402 - protein, 40 aa, synthetic construct
  REGION 1..40: note = Description of Artificial Sequence: Synthetic polypeptide
GGGGSGGGGSGGGGSGGGGSGGGGSGGGGSGGGGSGGGGS

SEQ ID NO: 403 - protein, 45 aa, synthetic construct
  REGION 1..45: note = Description of Artificial Sequence: Synthetic polypeptide
GGGGSGGGGSGGGGSGGGGSGGGGSGGGGSGGGGSGGGGSGGGGS

SEQ ID NO: 404 - protein, 16 aa, synthetic construct
  REGION 1..16: note = Description of Artificial Sequence: Synthetic peptide
GGGSGGGSGGGSGGGS

SEQ ID NO: 405 - protein, 20 aa, synthetic construct
  REGION 1..20: note = Description of Artificial Sequence: Synthetic peptide
GGGSGGGSGGGSGGGSGGGS

SEQ ID NO: 406 - protein, 24 aa, synthetic construct
  REGION 1..24: note = Description of Artificial Sequence: Synthetic peptide
GGGSGGGSGGGSGGGSGGGSGGGS

SEQ ID NO: 407 - protein, 28 aa, synthetic construct
  REGION 1..28: note = Description of Artificial Sequence: Synthetic peptide
GGGSGGGSGGGSGGGSGGGSGGGSGGGS

SEQ ID NO: 408 - protein, 32 aa, synthetic construct
  REGION 1..32: note = Description of Artificial Sequence: Synthetic polypeptide
GGGSGGGSGGGSGGGSGGGSGGGSGGGSGGGS

SEQ ID NO: 409 - protein, 36 aa, synthetic construct
  REGION 1..36: note = Description of Artificial Sequence: Synthetic polypeptide
GGGSGGGSGGGSGGGSGGGSGGGSGGGSGGGSGGGS

SEQ ID NO: 410 - protein, 40 aa, synthetic construct
  REGION 1..40: note = Description of Artificial Sequence: Synthetic polypeptide
GGGSGGGSGGGSGGGSGGGSGGGSGGGSGGGSGGGSGGGS

SEQ ID NO: 411 - protein, 44 aa, synthetic construct
  REGION 1..44: note = Description of Artificial Sequence: Synthetic polypeptide
GGGSGGGSGGGSGGGSGGGSGGGSGGGSGGGSGGGSGGGSGGGS

SEQ ID NO: 412 - protein, 4 aa, synthetic construct
  REGION 1..4: note = Description of Artificial Sequence: Synthetic peptide
GGGG

SEQ ID NO: 413 - DNA, 1077 bp, synthetic construct
  misc_feature 1..1077: note = Description of Artificial Sequence: Synthetic polynucleotide
  source 1..1077: mol_type = other DNA
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcgggggaggttctggcggtgggggatcgggcggtggagggagcgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaa

SEQ ID NO: 414 - DNA, 1173 bp, synthetic construct
  misc_feature 1..1173: note = Description of Artificial Sequence: Synthetic polynucleotide
  source 1..1173: mol_type = other DNA
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggtgggggatcagggggcggaggttctggaggtggcgggtcggggggaggtgggagcgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 415 - DNA, 1188 bp, synthetic construct
  misc_feature 1..1188: note = Description of Artificial Sequence: Synthetic polynucleotide
  source 1..1188: mol_type = other DNA
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggtgggggatcagggggcggaggttctggaggtggcgggtcggggggaggtgggagcggtggcgggggatcagacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 416 - protein, 366 aa, synthetic construct
  REGION 1..366: note = Description of Artificial Sequence: Synthetic polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 417 - protein, 371 aa, synthetic construct
  REGION 1..371: note = Description of Artificial Sequence: Synthetic polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 418 - protein, 376 aa, synthetic construct
  REGION 1..376: note = Description of Artificial Sequence: Synthetic polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 419 - DNA, 363 bp, synthetic construct
  misc_feature 1..363: note = Description of Artificial Sequence: Synthetic polynucleotide
  source 1..363: mol_type = other DNA
caggtgcagctggtggagagcggcggcggcgtggtgcagcccggccggagcctgcggctgagctgcgccgccagcggctacgccttcaccaactacctgatcgagtgggtgcggcaggcccccggcaagggcctggagtgggtggccgtgatcaaccccggcagcggcggcaccaactacaacgagaagttcaagggccggttcaccatcagcgccgacaagagcaagagcaccgcctacctgcagatgaacagcctgcgggccgaggacaccgccgtgtactactgcgcccggtggcggggcgagggctactacgcctacttcgacgtgtggggccagggcaccaccgtgaccgtgagcagc

SEQ ID NO: 420 - protein, 11 aa, synthetic construct
  REGION 1..11: note = Description of Artificial Sequence: Synthetic peptide
SQSVSYDADSY

SEQ ID NO: 421 - protein, 121 aa, synthetic construct
  REGION 1..121: note = Description of Artificial Sequence: Synthetic polypeptide
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGSGYYAYFDVWGQGTTVTVSS

SEQ ID NO: 422 - DNA, 363 bp, synthetic construct
  misc_feature 1..363: note = Description of Artificial Sequence: Synthetic polynucleotide
  source 1..363: mol_type = other DNA
gaggtgcaattggtgcagagcggagccgaagtgaagaagcccggcgagagcctgaagatcagctgcaagggcagcggctacgccttcaccaactacctgatcgagtgggtgcgccagatgcccggcaagggcctggaatggatgggcgtgatcaatcctggcagcggcggcaccaattacaacgagaagttcaagggccaagtgaccatcagcgccgacaagagcatcagcaccgcctacctccagtggtccagcctgaaggccagcgacaccgccatgtactactgcgccaggtggcggggaagcggctactacgcctacttcgacgtgtggggccagggcaccacagtgaccgtcagctca

SEQ ID NO: 423 - protein, 10 aa, synthetic construct
  REGION 1..10: note = Description of Artificial Sequence: Synthetic peptide
  VARIANT 1..10: note = This sequence may encompass 1-10 Gly residues wherein some positions may be absent
GGGGGGGGGG

(19) United States
(12) Patent Application Publication
(10) Pub. No.: US 2024/0368266 A1
(43) Pub. Date: Nov. 7, 2024

(54) IMMUNE-STIMULATING HUMANIZED MONOCLONAL ANTIBODIES AGAINST HUMAN INTERLEUKIN-2, AND FUSION PROTEINS THEREOF

(71) Applicant: Universität Zürich, Zürich (CH)

(72) Inventors: Natalia Arenas-Ramirez, Zürich (CH); Iwan Beuvink, Basel (CH); Onur Boyman, Küsnacht (CH); Barbara Brannetti, Basel (CH); Andreas Katopodis, Basel (CH); Simone Popp, Basel (CH); Catherine Regnier, Basel (CH); Chao Zou, Basel (CH)

(73) Assignee: Universität Zürich, Zürich (CH)

(21) Appl. No.: 18/508,567

(22) Filed: Nov. 14, 2023

Related U.S. Application Data
(63) Continuation of application No. 17/124,930, filed on Dec. 17, 2020, now Pat. No. 11,851,484.
Application No. 17/124,930 is a continuation of application No. 16/069,146, filed on Jul. 10, 2018, now Pat. No. 10,889,643.
(60) Provisional application No. 62/277,113, filed on Jan. 11, 2016.

(51) Int. Cl. C07K 16/24; A61K 38/00; A61K 39/00; C07K 14/55

(52) U.S. Cl. CPC C07K 16/246; C07K 14/55; A61K 38/00; A61K 2039/505; C07K 2317/24; C07K 2317/34; C07K 2317/55; C07K 2317/74; C07K 2317/92; C07K 2319/00

(57) ABSTRACT

The present invention relates to antibodies binding to human interleukin-2 (hIL-2). The invention more specifically relates to humanized antibodies specifically binding a particular epitope of hIL-2 and, when bound to this epitope, displaying a unique capability of inhibiting binding of hIL-2 to CD25.

### CROSS-REFERENCE TO RELATED APPLICATIONS

This application is a continuation of U.S. application Ser. No. 17/124,930, filed Dec. 17, 2020, now U.S. Pat. No. 11,851,484, issued Dec. 6, 2023, which is a continuation of U.S. application Ser. No. 16/069,146 filed on Jul. 10, 2018, and issued as U.S. Pat. No. 1,088,964 on Dec. 22, 2020, which is a § 371 National Stage Application of PCT/IB2017/050127 filed on Jan. 11, 2017, which claims priority to U.S. 62/277,113 filed on Jan. 11, 2016, all of which are hereby incorporated by reference in their entirety.

### REFERENCE TO A SEQUENCE LISTING SUBMITTED ELECTRONICALLY

This application contains a Sequence Listing in computer readable form. The computer readable form is incorporated herein by reference. Said XML copy, created on Jan. 15, 2024, is named P31479USW3.xml and is 489,643 bytes in size.

### TECHNICAL FIELD

The present invention relates to antibodies binding to human interleukin-2 (hIL-2). The invention more specifically relates to humanized antibodies specifically binding a particular epitope of hIL-2 and, when bound to this epitope, displaying a unique capability of inhibiting binding of hIL-2 to CD25, and fusions between said antibodies and hIL-2. Furthermore, the invention relates to in vitro and in vivo therapeutic applications of the antibodies in combination with hIL-2, and in vitro and in vivo therapeutic applications of the fusions.

### BACKGROUND

Interleukin-2 (IL-2) is a cytokine able to potently stimulate cytotoxic lymphocytes against metastatic tumors. However, IL-2 is also able to stimulate so-called CD25⁺ CD4⁺ regulatory T cells (Treg cells) that are crucial for prevention of autoimmune disease. Importantly, Treg cells can significantly dampen anti-tumor responses by cytotoxic lymphocytes, thus somewhat antagonizing the beneficial anti-tumor effects of IL-2. Moreover, at doses required to achieve a clinical anti-tumor response, IL-2 can exert toxic adverse effects.

Immunotherapy using IL-2 has been used since the early 1980's for the immunotherapy of metastatic melanoma and metastatic renal cell carcinoma, leading to the approval by the FDA for these indications in 1996 and 1992, respectively. While IL-2 given at high doses has shown objective response rates in about 17% and complete regression in about 6-9% of patients suffering from these deadly metastatic cancers, IL-2 given at these doses frequently led to toxic adverse effects, such as hypotension, pulmonary edema, liver cell damage, gastrointestinal toxicity, vascular leakage syndrome (VLS) and general edema. Moreover, as mentioned above, IL-2 is able to stimulate immunosuppressive Treg cells, which in turn are able to dampen the activity of anti-tumor CD8⁺ T cells and NK cells.

Several variants of human IL-2 exist, and different strategies have been employed to find IL-2 based compounds with improved in vivo properties, such as described in Rosalia et al. Current Opinion in Chemical Biology 2014, 23:39-46.

However, no successful therapy based on this principle has yet been made available for use in patients due to the lack of appropriate anti-human IL-2 antibodies.

### SUMMARY OF THE INVENTION

The present disclosure relates generally to antibodies or fragments thereof that bind to a specific epitope of human IL-2, methods for their preparation and use, including methods for treating disorders.

The anti-IL-2 antibodies or fragments thereof disclosed herein can be used (alone or in combination with other agents or therapeutic modalities) to treat, prevent and/or diagnose disorders, such as cancerous disorders (for example solid and soft-tissue tumors, and hematological tumors), as well as infectious diseases (for example chronic infectious disorders). Thus, compositions comprising the anti-IL-2 antibodies or fragments thereof, as well as methods for treating various disorders including cancer and/or infectious diseases, using the anti-IL-2 antibodies or fragments thereof, or compositions comprising the anti-IL-2 antibodies or fragments thereof, are disclosed herein.

In a first aspect, the present disclosure provides an isolated antibody, or antigen-binding portion thereof, which binds human IL-2 according to SEQ ID NO: 109, wherein said antibody or antigen-binding portion thereof comprises a light chain variable region comprising LCDR1, a LCDR2 and a LCDR3 and a heavy chain variable region comprising a HCDR1, a HCDR2 and a HCDR3 and wherein the LCDR1 comprises SEQ ID NO: 122; wherein LCDR2 comprises SEQ ID NO: 123; wherein LCDR3 comprises SEQ ID NO: 21; wherein HCDR1 comprises SEQ ID NO: 119; wherein HCDR2 comprises SEQ ID NO: 120; and wherein HCDR3 comprises SEQ ID NO: 121.

In an embodiment, the isolated antibody or antigen-binding portion thereof according to the first aspect, wherein said antibody or antigen-binding portion thereof comprises a light variable region comprising a: LCDR1 selected from the group consisting of SEQ ID NO: 19, SEQ ID NO: 31, SEQ ID NO: 69, SEQ ID NO: 72, SEQ ID NO: 86 and SEQ ID NO: 90; LCDR2 selected from the group consisting of SEQ ID NO: 20 and SEQ ID NO: 32; LCDR3 as set forth in SEQ ID NO: 21, and a heavy variable region comprising a: HCDR1 selected from the group consisting of SEQ ID NO: 4, and SEQ ID NO: 13; HCDR2 selected from the group consisting of SEQ ID NO: 2, and SEQ ID NO: 12; and HCDR3 selected from the group consisting of SEQ ID NO: 3, SEQ ID NO: 36, SEQ ID NO: 39, SEQ ID NO: 42, and SEQ ID NO: 45.

In a further embodiment, the isolated antibody or antigen-binding portion thereof according to the first aspect, wherein the LCDR1, LCDR2 and LCDR3 are SEQ ID NO: 19, 20 and 21, respectively and the HCDR1, HCDR2 and HCDR3 are SEQ ID NO: 4, 2 and 3, respectively; or LCDR1, LCDR2 and LCDR3 are SEQ ID NO: 31, 32 and 21, respectively and the HCDR1, HCDR2 and HCDR3 are SEQ ID NO: 4, 2 and 3, respectively; or LCDR1, LCDR2 and LCDR3 are SEQ ID NO: 19, 20 and 21 and the HCDR1, HCDR2 and HCDR3 are SEQ ID NO: 13, 12 and 3; or LCDR1, LCDR2 and LCDR3 are SEQ ID NO: 31, 32 and 21 and the HCDR1, HCDR2 and HCDR3 are SEQ ID NO: 13, 12 and 3; or LCDR1, LCDR2 and LCDR3 are SEQ ID NO: 69, 20 and 21 and the HCDR1, HCDR2 and HCDR3 are SEQ ID NO: 4, 2, and 3, respectively; or LCDR1, LCDR2 and LCDR3 are SEQ ID NO: 31, 32 and 21, respectively and the HCDR1, HCDR2 and HCDR3 are SEQ ID NO: 4, 2, and 3, respectively; or LCDR1, LCDR2 and LCDR3 are SEQ ID NO: 69, 20, and 21, respectively and the HCDR1, HCDR2 and HCDR3 are SEQ ID NO: 4, 2, and 3, respectively; or LCDR1, LCDR2 and LCDR3 are SEQ ID NO: 19, 20, and 21, respectively, and the HCDR1, HCDR2 and HCDR3 are 4, 2, and 36, respectively; or the LCDR1, LCDR2 and LCDR3 are SEQ ID NO: 69, 20, and 21, respectively, and the HCDR1, HCDR2 and HCDR3 are 4, 2, and 36, respectively; or the LCDR1, LCDR2 and LCDR3 are SEQ ID NO: 19, 20, 21, respectively, and the HCDR1, HCDR2 and HCDR3 are 4, 2, and 36, respectively; and the LCDR1, LCDR2 and LCDR3 are SEQ ID NO: 69, 20, 21, respectively, and the HCDR1, HCDR2 and HCDR3 are 4, 2, and 36, respectively.

In another embodiment, the isolated antibody or antigen-binding portion thereof according to the first aspect, comprises the heavy chain variable (VH) and light chain variable (VL) regions have at least 95%, such as 95%, 96%, 97%, 98%, or 99% identity to the amino acid sequences: VL, SEQ ID NO: 25; VH, SEQ ID NO: 7, or VL, SEQ ID NO: 27; VH, SEQ ID NO: 7, or VL, SEQ ID NO: 34; VH, SEQ ID NO: 7, or VL, SEQ ID NO: 25; VH, SEQ ID NO: 15, or VL, SEQ ID NO: 27; VH, SEQ ID NO: 15, or VL, SEQ ID NO: 34; VH, SEQ ID NO: 15, or VL, SEQ ID NO: 25; VH, SEQ ID NO: 17, or VL, SEQ ID NO: 27; VH, SEQ ID NO: 17, or VL, SEQ ID NO: 34; VH, SEQ ID NO: 17, or VL, SEQ ID NO: 70; VH, SEQ ID NO: 7, or VL, SEQ ID NO: 25; VH, SEQ ID NO: 37, or VL, SEQ ID NO: 70; VH, SEQ ID NO: 37, VL, SEQ ID NO: 79; VH, SEQ ID NO: 7, or VL, SEQ ID NO: 27; VH, SEQ ID NO: 37, or VL, SEQ ID NO: 79; VH, SEQ ID NO: 37, or VL, SEQ ID NO: 70; VH, SEQ ID NO: 17, or VL, SEQ ID NO: 25; VH, SEQ ID NO: 49, or VL, SEQ ID NO: 70; VH, SEQ ID NO: 49, or VL, SEQ ID NO: 79; VH, SEQ ID NO: 17, or VL, SEQ ID NO: 27; VH, SEQ ID NO: 49, or VL, SEQ ID NO: 79; VH, SEQ ID NO: 49.

In one embodiment, the isolated antibody or antigen-binding portion thereof according to the first aspect has the heavy chain variable (VH) and light chain variable (VL) regions have the amino acid sequences: VL, SEQ ID NO: 25; VH, SEQ ID NO: 7, or VL, SEQ ID NO: 27; VH, SEQ ID NO: 7, or VL, SEQ ID NO: 34; VH, SEQ ID NO: 7, or VL, SEQ ID NO: 25; VH, SEQ ID NO: 15, or VL, SEQ ID NO: 27; VH, SEQ ID NO: 15, or VL, SEQ ID NO: 34; VH, SEQ ID NO: 15, or VL, SEQ ID NO: 25; VH, SEQ ID NO: 17, or VL, SEQ ID NO: 27; VH, SEQ ID NO: 17, or VL, SEQ ID NO: 34; VH, SEQ ID NO: 17, or VL, SEQ ID NO: 70; VH, SEQ ID NO: 7, or VL, SEQ ID NO: 25; VH, SEQ ID NO: 37, or VL, SEQ ID NO: 70; VH, SEQ ID NO: 37, VL, SEQ ID NO: 79; VH, SEQ ID NO: 7, or VL, SEQ ID NO: 27; VH, SEQ ID NO: 37, or VL, SEQ ID NO: 79; VH, SEQ ID NO: 37, or VL, SEQ ID NO: 70; VH, SEQ ID NO: 17, or VL, SEQ ID NO: 25; VH, SEQ ID NO: 49, or VL, SEQ ID NO: 70; VH, SEQ ID NO: 49, or VL, SEQ ID NO: 79; VH, SEQ ID NO: 17, or VL, SEQ ID NO: 27; VH, SEQ ID NO: 49, or VL, SEQ ID NO: 79; VH, SEQ ID NO: 49.

The isolated antibody according to previous embodiments may comprise an Fc domain selected from the group consisting of SEQ ID NO: 93, SEQ ID NO: 95, SEQ ID NO: 97, SEQ ID NO: 99, SEQ ID NO: 101, SEQ ID NO: 103 and SEQ ID NO: 105. In a preferred embodiment, the isolated antibody comprises the Fc domain according to SEQ ID NO: 93, SEQ ID NO: 101, SEQ ID NO: 103 or SEQ ID NO: 105.

In a specific embodiment, the isolated antibody comprises the light chain according to SEQ ID NO: 124 and the heavy chain according to SEQ ID NO: 126, or the light chain according to SEQ ID NO: 128 and the heavy chain according to SEQ ID NO: 130.

According to a second aspect of the invention, an isolated antibody or antigen-binding fragment thereof is provided, which binds to a human interleukin-2 (hIL-2) epitope which comprises the amino acids K52, P54, K55, T57, R58, T61, F62, K63, Q94, and K96.

In an embodiment, the isolated antibody or antigen-binding fragment thereof according to the second aspect, binds specifically to the amino acids K52, P54, K55, T57, R58, T61, F62, K63, Q94, and K96.

The isolated antibody or antigen-binding fragment thereof according to the second aspect, may bind to a human interleukin-2 (hIL-2) epitope which, in addition to the amino acids K52, P54, K55, T57, R58, T61, F62, K63, Q94, and K96, further comprises any one or more of the amino acids N50, N53, N91, L92, A93, and N97.

In one embodiment, the antibody or antigen-binding fragment thereof according to the second aspect binds specifically to the amino acids N50, K52, N53, P54, K55, T57, R58, T61, F62, K63, N91, L92, A93, Q94, K96, and N97.

In one embodiment, the antibody or antigen-binding fragment thereof comprises a light chain variable region comprising, in sequence a LCDR1, a LCDR2 and a LCDR3, and a heavy chain variable region comprising, in sequence a HCDR1, a HCDR2 and a HCDR3, wherein the LCDR1, LCDR2 and LCDR3 are SEQ ID NO: 231, 232 and 233, respectively and the HCDR1, HCDR2 and HCDR3 are SEQ ID NO: 181, 182 and 183, respectively; or the LCDR1, LCDR2 and LCDR3 are SEQ ID NO: 279, 280 and 281, respectively and the HCDR1, HCDR2 and HCDR3 are SEQ ID NO: 213, 214 and 215, respectively; or the LCDR1, LCDR2 and LCDR3 are SEQ ID NO: 231, 232 and 233, respectively and the HCDR1, HCDR2 and HCDR3 are SEQ ID NO: 213, 214 and 215, respectively; or the LCDR1, LCDR2 and LCDR3 are SEQ ID NO: 263, 264 and 265, respectively and the HCDR1, HCDR2 and HCDR3 are SEQ ID NO: 213, 214 and 215, respectively; or the LCDR1, LCDR2 and LCDR3 are SEQ ID NO: 263, 264 and 265, respectively and the HCDR1, HCDR2 and HCDR3 are SEQ ID NO: 149, 150 and 151, respectively; or the LCDR1, LCDR2 and LCDR3 are SEQ ID NO: 69, 20 and 21, respectively and the HCDR1, HCDR2 and HCDR3 are SEQ ID NO: 197, 198, and 199, respectively; or the LCDR1, LCDR2 and LCDR3 are SEQ ID NO: 231, 232 and 233, respectively and the HCDR1, HCDR2 and HCDR3 are SEQ ID NO: 197, 198, and 199, respectively.

In one embodiment, the antibody or antigen-binding fragment thereof comprises the heavy chain variable (VH) and light chain variable (VL) regions have at least 95% identity, such as 100% identity, to the amino acid sequences VL, SEQ ID NO: 243; VH, SEQ ID NO: 193, or VL, SEQ ID NO: 391; VH, SEQ ID NO: 225, or VL, SEQ ID NO: 243; VH, SEQ ID NO: 225, or VL, SEQ ID NO: 275; VH, SEQ ID NO: 225, or VL, SEQ ID NO: 275; VH, SEQ ID NO: 161, or VL, SEQ ID NO: 70; VH, SEQ ID NO: 209, or VL, SEQ ID NO: 243; VH, SEQ ID NO: 209.

In a specific embodiment, the isolated antibody comprises wherein the heavy chain and light chain regions have the amino acid sequences; heavy chain according to SEQ ID NO: 195 and light chain according to SEQ ID NO: 245, or heavy chain according to SEQ ID NO: 227 and light chain according to SEQ ID NO: 393, or heavy chain according to SEQ ID NO: 227 and light chain according to SEQ ID NO: 245, or heavy chain according to SEQ ID NO: 227 and light chain according to SEQ ID NO: 277, or heavy chain according to SEQ ID NO: 163 and light chain according to SEQ ID NO: 277, or heavy chain according to SEQ ID NO: 211 and light chain according to SEQ ID NO: 261, or heavy chain according to SEQ ID NO: 211 and light chain according to SEQ ID NO: 277.

According to a third aspect of the invention, a composition comprising the antibody according to the first or the second aspect of the invention, and optionally but preferably, human IL-2, is provided.

In an embodiment, the composition according to the third aspect comprises the human IL-2 selected from the group consisting of human IL-2 according to SEQ ID NO: 109 or aldesleukin according to SEQ ID NO: 110, preferably aldesleukin according to SEQ ID NO: 110.

According to a fourth aspect of the invention, a fusion protein is provided, comprising an antibody according to the first or second aspect of the invention, and human IL-2.

In one embodiment, the antibody and the human IL-2 is joined by a linker sequence selected from the group consisting of SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, and SEQ ID NO: 411, preferably SEQ ID NO: 405 or SEQ ID NO: 407.

In one embodiment the fusion protein comprises an antibody according to the first or second aspect of the invention, wherein the LCDR1 of the antibody comprises a residue Y27 and a residue D30 according to the Kabat definition, and wherein the residue Y27 is joined to residue N97 of human IL-2 with a GG linker, and wherein residue D30 is joined to residue K96 residue of human IL-2 with a linker according to SEQ ID NO: 412.

According to a fifth aspect of the invention, an antibody or antigen-binding fragment thereof according to the first or second aspect of the invention, or a composition according to the third aspect of the invention, or the fusion protein according to the fourth aspect of the invention, for use as a medicament is provided.

According to a sixth aspect of the invention, an antibody or antigen-binding fragment thereof according to the first or second aspect of the invention, or a composition according to the third aspect of the invention, or the fusion protein according to the fourth aspect of the invention, for use in the manufacture of a medicament is provided.

According to a seventh aspect of the invention, an antibody or antigen-binding fragment thereof according to the first or second aspect of the invention, or a composition according to the third aspect of the invention, or the fusion protein according to the fourth aspect of the invention, for use in treatment of cancer is provided.

According to an eight aspect of the invention, a method of treating cancer by administering an antibody or antigen-binding fragment thereof according to the first or second aspect of the invention, or a composition according to the third aspect of the invention, or the fusion protein according to the fourth aspect of the invention.

According to a ninth aspect of the invention, a vector is provided, comprising the nucleic acid molecule capable of encoding an antibody, or fragment thereof, according to the first or second aspects of the invention, or the fusion protein according to the fourth aspect of the invention.

According to a tenth aspect of the invention, a cell is provided, comprising the vector according to the eight aspect of the invention.

According to an eleventh aspect of the invention, a cell, able to produce a human interleukin-2 (hIL-2) specific monoclonal antibody, or antigen-binding fragment thereof, according to the first or second aspect, is provided.

According to a twelfth aspect of the invention, a monoclonal antibody-producing hybridoma cell line is provided, characterized in that said produced antibodies are those the first or second aspect of the invention, or the fusion protein according to the fourth aspect of the invention.

The antibodies according to aspects of the invention are advantageous, e.g. because they possess one or more of the following properties. Upon binding of antibody to hIL-2, the resulting mAb*hIL-2 complex cannot efficiently bind human IL-2 receptor alpha (also known as CD25) anymore, effectively reducing the binding of human CD25 to mAb*hIL-2 to background levels as compared to the binding of human CD25 to free (non-complexed) hIL-2 when measured by surface plasmon resonance. Furthermore, the antibodies may display no measurable cross-reactivity to murine IL-2.

### BRIEF DESCRIPTION OF THE DRAWINGS

FIG. 1 provides the overview of the three-dimensional structure of Proleukin®/Fab-NARA1 complex as obtained in Example 2.

FIG. 2 provides further analysis of epitope residues. The X-axis lists the amino acid sequence and numbering according to SEQ ID NO: 110. The upper side of Y-axis shows the total number of atoms of NARA1-Fab that are within 4 Å from corresponding residue from Proleukin® and the lower side of Y-axis shows the reduced solvent-accessible area (Å2) of corresponding residue from Proleukin® as a consequence of binding to NARA1-Fab, according to SEQ ID NO: 132.

FIG. 3 illustrates the most critical epitope residue recognized by the NARA1-Fab.

FIG. 4 shows the overlay of Proleukin®/NARA1-Fab complex with IL-2/CD25/CD122/CD132 quaternary complex.

FIG. 5 displays the overlay of C helices from IL-2_C145A (PDB: 3INK), the D10 IL-2-mutein (“Superkine”: PDB: 3QB1), IL-2/CD25/CD122/CD132 (PDB: 2B5l), and Proleukin®/NARA1-Fab.

FIG. 6 shows the counts of immune cells from spleens of mice receiving phosphate-buffered saline (PBS), low dose human IL-2 (IL-2 LD), high dose human IL-2 (IL-2 HD), or IL-2/antibody complexes made by using the indicated monoclonal antibodies. The values are shown in tables 14 and 15.

FIG. 7 shows representative bromodeoxyuridine (BrdU) profiles of CD8⁺ CD44⁺ CD122⁺ T cells from the spleens of mice treated as in FIG. 6.

FIG. 8 shows representative BrdU profiles of CD3-NK1.1⁺ NK cells from spleens of mice treated as in FIG. 6.

FIG. 9 and FIG. 10 is a schematic illustrating a fusion protein according to an embodiment.

FIG. 11 illustrates alignment of a fusion protein according to an embodiment. FIG. 11 discloses (G) 4 as SEQ ID NO: 412.

FIGS. 12 to 15 show phenotypic characterization of endogenous CD8⁺ T cells, CD8⁺ CD44⁺ CD122⁺ T cells, CD4⁺ T cells, CD4⁺ CD25^{high}Foxp3⁺ Treg cells and CD3-NK1.1⁺ NK cells from spleens of mice treated as in FIG. 6, according to an example.

FIGS. 16 and 17 show the counts of immune cell subsets and CD8⁺ CD44⁺ T cell-to-CD4⁺ CD25^{high}Foxp3⁺ Treg cell ratios obtained from spleens of mice treated as in FIG. 6, according to an example.

FIGS. 18 to 22 show representative BrdU profiles of CD8⁺, CD8⁺ CD44⁺ CD122⁺ T cells, CD3-NK1.1⁺ DX5⁺ NK cells, CD4⁺ T cells and CD4⁺ CD25⁺ T cells, from spleens of mice treated as in FIG. 6, according to an example.

FIG. 23 shows phenotypic characterization of endogenous CD8⁺ T cells and NK cells from spleens of mice receiving PBS, fusion proteins L15, L20 or L25, or human IL-2/NARA1 complexes, according to an example.

FIG. 24 shows the cell counts of the indicated immune cells obtained from spleens of mice treated as in FIG. 23, according to an example.

FIG. 25 shows representative BrdU profiles of CD8⁺ CD44⁺ CD122⁺ T cells and CD3-NK1.1⁺ NK cells from spleens of mice treated as in FIG. 23, according to an example.

FIG. 26 shows CTLL-2 cell proliferation curves from in vitro experiments using CTLL-2 cells stimulated with titrated doses of the fusion proteins L15, L20 or L25, of human IL-2/NARA1 complexes, or of human IL-2, according to an example.

FIG. 27 shows STAT5 phosphorylation levels of CTLL-2 cells stimulated with titrated doses of human IL-2/NARA1 complexes, of human IL-2, or of the fusion proteins L15, L20 or L25, according to an example.

### BRIEF DESCRIPTION OF THE TABLES

Table 1 is an overview of anti-IL-2 antibodies according to embodiments of the invention.

Table 2 is an overview of IL-2 muteins according to embodiments of the invention.

Table 3 represents structure statistics for a Proleukin®/NARA1-Fab complex.

Table 4 is an overview of epitope and paratope according to embodiments of the invention.

Table 5 is an overview of variable heavy regions according to embodiments of the invention.

Table 6 is an overview of variable light regions according to embodiments of the invention.

Table 7 comprises pl data of antibodies according to some embodiments.

Table 8 comprises comparison variable regions and variable germline regions.

Table 9 comprises structure-refined variable regions according to embodiments of the invention.

Table 10 comprises information about variable light chains and variable heavy chains according to embodiments of the invention.

Table 11 comprises light chain CDRs according to embodiments of the invention.

Table 12 comprises heavy chain CDRs according to embodiments of the invention.

Table 13 comprises optimized variable light chains and variable heavy chains according to embodiments of the invention.

Table 14 is an overview of VH mutation sequences.

Table 15 is an overview of VK mutation sequences.

Table 16 is an overview of plasmid sequences.

Table 17 is an overview of affinity-matured antibodies according to embodiments of the invention.

Table 18 is a sequence overview of first set of antibodies Table 19 is an overview of ELISA values according to an example.

Table 20 is an overview of EC50 values according to an example.

Table 21 is a subset of affinity matured antibodies according to embodiments of the invention.

Table 22 is a sequence overview of the subset of antibodies according to Table 21.

Table 23 represents binding affinity data.

Table 24 represents CD8⁺ T cell proliferation data.

Table 25 represents CD8⁺ T and NK cell proliferation data.

Table 26 and Table 27 represent cell count data.

Table 28 is an overview of linker sequences according to embodiments of the invention.

Table 29 is an overview of fusion proteins according to embodiments of the invention.

Table 30 represents CD8⁺ T and NK cell proliferation data.

Table 31 represents CD8⁺ T, NK and Treg cell count data.

Table 32 and Table 33 represent cell count data.

Table 34 represents ratios of cell count data.

Table 35 and Table 36 represent light region sequences of fusion proteins.

Table 37 is an overview of fusion proteins.

Table 38 represents cell count data.

Table 39 and Table 40 represent EC50 values according to an example.

Table 41 is a sequence listing comprising sequences useful for practicing the invention.

### DETAILED DESCRIPTION

The present disclosure relates to antibodies and fragments thereof that bind to human IL-2, and affect the in vivo function of this cytokine.

By “human interleukin-2” or “hIL-2” as used herein is meant human IL-2 (wildtype or wt) with UniProt ID number P60568, reproduced herein as SEQ ID NO: 109. In various embodiments of the invention, variants, isoforms, and species homologs of human wildtype IL-2 are also included. Accordingly, antibodies of this disclosure may, in certain cases, cross-react with IL-2 from species other than human. In certain embodiments, the antibodies may be completely specific for one or more human IL-2 proteins and may not exhibit species or other types of non-human cross-reactivity.

The term “mutein” means a polypeptide wherein specific substitutions to the interleukin-2 protein have been made. As used in reference to administrative modalities and treatments, the term “IL-2 mutein” means 1, 2, 3, 4, or 5 or more IL-2 muteins. For example, treatment using an IL-2 mutein may refer to treatment with a single IL-2 mutein, or a combination of multiple IL-2 muteins. An example of human IL-2 is the IL-2 mutein disclosed in WO2012/107417A1, having 3 mutations compared to wt hIL-2.

Proleukin® (aldesleukin) is another example of a variant of human wt IL-2, well known to a person skilled in the art, and represented herein by SEQ ID NO: 110.

The term “antibody” or “antibody to IL-2” and the like as used herein refers to whole antibodies that interact with (e.g., by binding, steric hindrance, stabilizing/destabilizing, spatial distribution) an IL-2 epitope and interfere with IL-2's binding to IL-2 receptor alpha (also termed CD25). A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelid antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “antigen-binding portion” of an antibody (or simply “antigen portion”), as used herein, refers to full length or one or more fragments of an antibody, such as a protein, that retain the ability to specifically bind to an antigen or epitope (e.g., a portion of IL-2).

The “Complementarity Determining Regions” (“CDRs”) are amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

The term “epitope binding domain” or “EBD” refers to portions of the antigen-binding portion (e.g., an antibody or epitope-binding fragment or derivative thereof), that specifically interacts with (e.g., by binding, steric hindrance, stabilizing/destabilizing, spatial distribution) a binding site on a target epitope. EBD also refers to one or more fragments of an antibody that retain the ability to specifically interact with (e.g., by binding, steric hindrance, stabilizing/destabilizing, spatial distribution) an IL-2 epitope and interferes with IL-2's binding to IL-2 receptor alpha (CD25). Examples of antibody fragments include, but are not limited to, an scFv, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).

Such single chain antibodies are also intended to be encompassed within the terms “fragment”, “epitope-binding fragment” or “antibody fragment”. These fragments are obtained using conventional techniques known to those of skill in the art, and the fragments are screened for utility in the same manner as are intact antibodies.

Antibody fragments can be incorporated into single chain molecules comprising a pair of tandem Fv segments (VH-CH1-VH-CH1) which, together with complementary light chain polypeptides, form a pair of antigen-binding regions (Zapata et al., (1995) Protein Eng. 8:1057-1062; and U.S. Pat. No. 5,641,870), and also include Fab fragments, F(ab′) fragments, F(ab′) 2 fragments, and anti-idiotypic (anti-Id) antibodies (including, e.g., anti-Id antibodies to antibodies of the invention), and epitope-binding fragments of any of the above.

EBDs also include single domain antibodies, maxibodies, unibodies, minibodies, triabodies, tetrabodies, v-NAR and bis-scFv, as is known in the art (see, e.g., Hollinger and Hudson, (2005) Nature Biotechnology 23:1126-1136), bispecific single chain diabodies, or single chain diabodies designed to bind two distinct epitopes. EBDs also include antibody-like molecules or antibody mimetics, which include, but not limited to minibodies, maxybodies, Fn3 based protein scaffolds, Ankrin repeats (also known as DARpins), VASP polypeptides, Avian pancreatic polypeptide (aPP), Tetranectin, Affililin, Knottins, SH3 domains, PDZ domains, Tendamistat, Neocarzinostatin, Protein A domains, Lipocalins, Transferrin, and Kunitz domains that specifically bind epitopes, which are within the scope of the invention. Antibody fragments can be grafted into scaffolds based on polypeptides such as Fibronectin type III (Fn3) (see U.S. Pat. No. 6,703,199, which describes fibronectin polypeptide monobodies).

The phrase “isolated antibody”, as used herein, refers to antibody that is substantially free of other antibodies having different antigenic specificities (e.g., an isolated antibody that specifically binds IL-2 is substantially free of antibodies that specifically bind antigens other than IL-2). An isolated antibody that specifically binds IL-2 may, however, have cross-reactivity to other antigens, such as IL-2 molecules from other species. Moreover, an isolated antibody may be substantially free of other cellular material and/or chemicals.

The term “monovalent antibody” as used herein, refers to an antibody that binds to a single epitope on a target molecule such as IL-2.

The term “bivalent antibody” as used herein, refers to an antibody that binds to two epitopes on at least two identical IL-2 target molecules. The bivalent antibody may also crosslink the target IL-2 molecules to one another. A “bivalent antibody” also refers to an antibody that binds to two different epitopes on at least two identical IL-2 target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for the IL-2, the mutivalent antibody (e.g., an IL-2 biparatopic antibody) has a binding moiety for two domains of IL-2, respectively.

The multivalent antibody mediates biological effect or which modulates a disease or disorder in a subject (e.g., by mediating or promoting cell killing, or by modulating the amount of a substance which is bioavailable.

The term “multivalent antibody” also refers to a single binding molecule that has more than one antigen-binding moieties for two separate IL-2 target molecules. For example, an antibody that binds to both an IL-2 target molecule and a second target molecule that is not IL-2. In one embodiment, a multivalent antibody is a tetravalent antibody that has four epitope binding domains. A tetravalent molecule may be bispecific and bivalent for each binding site on that target molecule.

The term “biparatopic antibody” as used herein, refers to an antibody that binds to two different epitopes on a single IL-2 target. The term also includes an antibody, which binds to two domains of at least two IL-2 targets, e.g., a tetravalent biparatopic antibody.

The term “bispecific antibody” as used herein, refers to an antibody that binds to two or more different epitopes on at least two different targets (e.g., an IL-2 and a target that is not IL-2).

The phrases “monoclonal antibody” or “monoclonal antibody composition” as used herein refers to polypeptides, including antibodies, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The term “humanized antibody” or “humanized anti-IL-2 antibody” as used herein includes antibodies in which CDR sequences derived from the germline of another mammalian species, such as a mouse, have been grafted onto human framework sequences.

Additional framework region modifications may be made within the human framework sequences as well as within the CDR sequences derived from the germline of another mammalian species.

The humanized antibodies of the invention may include amino acid residues not encoded by human sequences (e.g., mutations introduced by random or site-specific mutagenesis in vitro or by somatic mutation in vivo, or a conservative substitution to promote stability or manufacturing).

The phrase “recombinant humanized antibody” as used herein, includes all human antibodies that are prepared, expressed, created or isolated by recombinant means, such as antibodies isolated from a host cell transformed to express the humanized antibody, e.g., from a transfectoma, and antibodies prepared, expressed, created or isolated by any other means that involve splicing of all or a portion of a human immunoglobulin gene, sequences to other DNA sequences.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding site” as used herein comprises an area on an IL-2 target molecule to which an antibody or antigen-binding fragment selectively binds.

The term “fusion protein” is a fusion of two separate proteins, with or without an additional linker sequence.

The term “linker sequence” is an amino acid sequence used to link or join two proteins.

Generally, antibodies specific for a particular target antigen will bind to an epitope on the target antigen in a complex mixture of proteins and/or macromolecules.

As used herein, the term “Affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a KD of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can 10 vary for other antibody isotypes. For example, high affinity binding for an IgM isotype refers to an antibody having a KD of 10⁻⁷ M or less, or 10⁻⁸ M or less.

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein. The terms “biomarker” or “marker” are used interchangeably herein. A biomarker is a nucleic acid or polypeptide and the presence or absence of a mutation or differential expression of the polypeptide is used to determine sensitivity to any treatment comprising an anti-IL-2 antibody according to the invention. For example, a protein is a biomarker for a cancer cell when it is deficient, mutated, deleted, or decreased in post-translational modification, production, expression, level, stability and/or activity, as compared to the same protein in a normal (non-cancerous) cell or control cell.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Example vectors for libraries include bacteriophage (also known as “phage”), viruses that infect bacteria, for example, lambda phage. The library can then be probed for the specific cDNA (and thus mRNA) of interest.

The term “cell proliferative disorders” shall include dysregulation of normal physiological function characterized by abnormal cell growth and/or division or loss of function. Examples of “cell proliferative disorders” include, but are not limited to, hyperplasia, neoplasia, metaplasia, and various autoimmune disorders, e.g., those characterized by the dysregulation of T cell apoptosis.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a coope-rative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

“Gene expression” or alternatively a “gene product” refers to the nucleic acids or amino acids (e.g., peptide or polypeptide) generated when a gene is transcribed and translated.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or under expressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, under expression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non-cancerous cell or tissue) is undetectable.

A high expression level of the gene can occur because of over expression of the gene or an increase in gene copy number. The gene can also be translated into increased protein levels because of deregulation or absence of a negative regulator. Lastly, high expression of the gene can occur due to increased stabilization or reduced degradation of the protein, resulting in accumulation of the protein.

As used herein, the term “inhibit”, “inhibiting”, or “inhibit the growth” or “inhibiting the proliferation” of a cancer cell refers to slowing, interrupting, arresting or stopping the growth of the cancer cell, and does not necessarily indicate a total elimination of the cancer cell growth. The terms “inhibit” and “inhibiting”, or the like, denote quantitative differences between two states; refer to at least statistically significant differences between the two states. For example, “an amount effective to inhibit growth of cancer cells” means that the rate of growth of the cells will be at least statistically significantly different from the untreated cells. Such terms are applied herein to, for example, rates of cell proliferation.

The term “isolated” means separated from constituents, cellular and otherwise, in which the polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A “metastatic cell or tissue” means that the cell can invade and destroy neighboring body structures.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, siRNAs, shRNAs, RNAi agents, and primers. A polynucleotide can be modified or substituted at one or more base, sugar and/or phosphate, with any of various modifications or substitutions described herein or known in the art. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc.

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

“Suppressing” or “suppression” of tumor growth indicates a reduction in tumor cell growth when contacted with an Anti-IL-2 antibody according to the invention compared to tumor growth without contact with an Anti-IL-2 antibody according to the invention compound. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage. A “subject,” “individual” or “patient” is used interchangeably herein, which refers to a vertebrate, preferably a mammal, more preferably a human. Mammals include, but are not limited to, mice, simians, humans, farm animals, sport animals, and pets.

### Anti-IL-2 Antibodies

In a first aspect the present invention provides an isolated antibody, or antigen-binding portion thereof, which binds human IL-2, wherein said antibody or antigen-binding portion thereof comprises a light chain variable region comprising LCDR1, a LCDR2 and a LCDR3 according to Table 11 and a heavy chain variable region comprising a HCDR1, a HCDR2 and a HCDR3 according to Table 12.

In some embodiments the isolated antibody or antigen-binding portion thereof, comprises a light variable region according to Table 6 or Table 10 and a heavy variable region comprising a heavy variable region according to Table 5 or Table 10.

In some embodiments the isolated antibody, comprises variable light chains and variable heavy chains as set out in Table 10 or Table 13.

In another aspect the present invention provides variants of an antibody or fragment thereof that binds to human IL-2. Thus the present invention provides antibodies or fragments thereof that have an amino acid sequence of the non-CDR regions of the heavy and/or light chain variable region sequence which is at least 80% identical (having at least 80% amino acid sequence identity) to the amino acid sequence of the non-CDR regions of the heavy and/or light chain variable region sequence of the parent antibody of either the heavy or the light chain e.g. of either the heavy and light variable region sequences as Table 5 and Table 6, respectively. As well antibodies or fragments thereof that have an amino acid sequence of the non-extended CDR regions of the heavy and/or light chain variable region sequence which is at least 80% identical to the amino acid sequence of the non-extended CDR regions of the heavy and/or light chain variable region sequence of the parent antibody of either the heavy or the light chain are provided by the present invention. Preferably the amino acid sequence identity of the non-CDR regions or of the non-extended CDR regions of the heavy and/or light chain variable region sequence is at least 85%, more preferably at least 90%, and most preferably at least 95%, in particular 96%, more particular 97%, even more particular 98%, most particular 99%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%.

The present disclosure also provides an antibody or fragment thereof that binds to human IL-2 which further comprises a heavy and/or light constant region in particular a human heavy and/or a human light constant region. Human heavy constant regions may be selected from the group of human immunoglobulins consisting of IgG 1 (IGHG 1), IgG2 (IGHG2), IgG3 (IGHG3), IgG4 (IGHG4), IgAI (IGHAI), IgA2 (IGHA2), IgM (IGHM), IgD (IGHD), or IgE (IGHE), whereas the human heavy constant region IgG, in particular IgG 1 (IGHG 1) is preferred. Human light constant region may be selected from the group of human immunoglobulins consisting of kappa or lambda constant regions, whereas human kappa constant region is preferred. In a preferred embodiment the antibody or fragment thereof that binds to human IL-2 comprises a human IgG 1 (IGHG 1) heavy constant domain and a human light kappa constant domain.

In addition or alternative to modifications made within the framework regions or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody of the invention may be chemically modified (e.g., one or more Chemical moieties can be attached to the antibody) or be modified to alter its glycosylation.

The present invention provides for antibodies that specifically bind to human IL-2 which resulting in altered half-life in vivo.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dentritic cells). A variety of strategies can be used to extend the half-life of the antibodies and antigen-binding fragments thereof of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nancarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues.

To pegylate an antibody, the antibody, antigen-binding fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10)alkoxy- or aryloxy-polyethylene glycol or polyethylene glycol-maleimide. In one embodiment, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies and antigen-binding fragments thereof of the invention. See for example, EP 0 154 316 by Nishimura et al. and EP 0 401 384 by Ishikawa et al., each of which is incorporated by reference.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous.

Polysialylation is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375, each of which is incorporated by reference.

Further, antibodies can be conjugated to albumin in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622, each of which is incorporated by reference.

The strategies for increasing half-life is especially useful in nanobodies, fibronectin-based binders, and other antibodies or proteins for which increased in vivo half-life is desired.

In another embodiment, the antibody is modified to increase its biological half-life. Various approaches are possible. For example, one or more of the following mutations can be introduced: T252L, T254S, T256F, as described in U.S. Pat. No. 6,277,375 to Ward, which is incorporated by reference. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al, which is incorporated by reference.

In one embodiment, antibodies according to the invention comprises light and heavy chains according to Table 1.

TABLE 1
Heavy and light chain SEQ ID Nos for antibodies
according to an embodiment.

| | Light chain SEQ | Heavy chain SEQ |
|---|---|---|
| Antibody | ID | ID |
| 104343 | SEQ ID NO: 124 | SEQ ID NO: 126 |
| 104348 | SEQ ID NO: 128 | SEQ ID NO: 130 |

### 1. Nucleic Acids, Vectors and Host Cells

The present invention is also directed to cell lines that express an anti-IL-2 antibody of the invention or portion thereof. Creation and isolation of cell lines producing a antibody of the invention can be accomplished using standard techniques known in the art. The CHO cell line is preferred (available from public repositories such as ATCC, American Type Culture Collection, Manassas, VA).

A wide variety of host expression systems can be used to express an antibody of the present invention including prokaryotic and eukaryotic expression systems (such as yeast, baculovirus, plant, mammalian and other animal cells, transgenic animals, and hybridoma cells), as well as phage display expression systems. One example of a suitable bacterial expression vector is pUC119 and a suitable eukaryotic expression vector is a modified pcDNA3.1 vector with a weakened dhfr selection system. Other antibody expression systems are also known in the art.

An antibody of the invention can be prepared by recombinant expression of immunoglobulin light and heavy chain genes in a host cell, as is well known to a person skilled in the art. To express an antibody recombinantly, a host cell is transformed, transduced, infected or the like with one or more recombinant expression vectors carrying DNA fragments encoding the immunoglobulin light and/or heavy chains of the antibody such that the light and/or heavy chains are expressed in the host cell. The heavy chain and light chain may be expressed in the same or different host cells. Preferably, the recombinant antibodies are secreted into the medium in which the host cells are cultured, from which the antibodies can be recovered or purified.

Standard recombinant DNA methodologies are used to obtain antibody heavy and light chain genes, incorporate these genes into recombinant expression vectors, and introduce the vectors into host cells. Such standard recombinant DNA technologies are described, for example, in Green and Sambrook (Eds.), Molecular Cloning; A Laboratory Manual, Fourth Edition, Cold Spring Harbor, N.Y., 2012.

In one embodiment, the invention provides a vector, preferably (but not limited to) a plasmid, a recombinant expression vector, a yeast expression vector, or a retroviral expression vector comprising a polynucleotide encoding an anti-IL-2 antibody of the invention. The coding region(s) in the vector may be separated by a linker sequence of any size or content, preferably such linker, when present, is a polynucleotide encoding an internal ribosome entry site.

To express an antibody of the invention, a DNA encoding a partial amino acid chain, as described in Table 41, are inserted into an expression vector such that the gene is operably linked to transcriptional and translational control sequences. The expression vector and expression control sequences are chosen to be compatible with the expression host cell used. Additionally, the recombinant expression vector can encode a signal peptide that facilitates secretion of the anti-IL-2 antibody light and/or heavy chain from a host cell. The anti-IL-2 antibody light and/or heavy chain gene can be cloned into the vector such that the signal peptide is operably linked in-frame to the amino terminus of the antibody chain gene. The signal peptide can be an immunoglobulin signal peptide or a heterologous signal peptide.

For expression of the light and/or heavy chains, the expression vector(s) encoding the heavy and/or light chains is introduced into a host cell by standard techniques e.g., electroporation, calcium phosphate precipitation, DEAE-dextran transfection, transduction, infection and the like. Although it is theoretically possible to express the antibodies of the invention in either prokaryotic or eukaryotic host cells, eukaryotic cells are preferred and most preferably mammalian host cells, because such cells are more likely to assemble and secrete a properly folded and immunologically active antibody.

Preferred mammalian host cells for expressing the recombinant antibodies of the invention include Chinese Hamster Ovary (CHO cells), e.g. as described in Urlaub and Chasin, Proc. Natl. Acad. Sci. USA 77:4216-20, 1980. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown under appropriate conditions known in the art. Antibodies can be recovered from the host cell and/or the culture medium using standard purification methods.

### IL-2 Variants

In certain embodiments of the invention, human IL-2 of wildtype (wt) is used. It has UniProt ID number P60568 and is reproduced as SEQ ID NO: 109. Another example of human IL-2 is the IL-2 mutein disclosed in WO2012/107417A1, having 3 mutations compared to wt hIL-2. Aldesleukin (trade name Proleukin®) is another example of a variant of human IL-2, well known to a person skilled in the art, and represented herein by SEQ ID NO: 110. Other examples of IL-2 variants are no-alpha mutein and IL-2 superkine, as shown in Table 2.

TABLE 2
IL-2 muteins

| | | substitution at position x relative | substitution at position x |
|---|---|---|---|
| | Mutein | to full length wt IL-2 | relative to mature wt IL-2 |
| 1 | Proleukin ® | C145S | C125S |
| | (Aldesleukin) | | |
| 2 | no-alpha mutein | R58A, F62A, Y65A, E82A | R38A, F42A, Y45A, E62A |
| 3 | WO2012/107417A1 | F62A, Y65A, L92G | F42A, Y45A, L72G |
| 4 | IL-2 superkine | L100F, R101D, L105V, I106V, | L80F, R81D, L85V, I86V, |
| | | I112F | I92F |

### IL-2/Anti-IL-2 Antibody Combinations

In an embodiment, the antibodies, or antigen-binding portion thereof, as described above, are combined with human IL-2 or IL-2 mutants as described above.

The combination can be a pre-made mixture with 1:1, 2:1 or other proportion of IL-2: antibody binding site.

In one embodiment, the anti-IL-2 antibody and IL-2 are administered in sequence with a first injection of antibody, and a subsequent injection of anti-IL-2 antibody/IL-2 combination.

In another embodiment, the anti-IL-2 antibody and IL-2 are administered in sequence with a first injection of anti-IL-2 antibody/IL-2 combination, and a subsequent injection of IL-2.

### Pharmaceutical Compositions

Pharmaceutical compositions of the disclosure also can be administered in combination therapy, i.e., combined with other agents. For example, the combination therapy can include an anti-IL-2 antibody or fragment thereof according to the present disclosure combined with at least one other anti-inflammatory or another chemotherapeutic agent. Examples of therapeutic agents that can be used in combination therapy are described in greater detail below in the section on combination therapies.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route. Depending on the route of administration, the active compound, i.e., antibody, immunoconjugate, or bispecific molecule, may be coated in a material to protect the compound from the action of acids and other natural conditions that may inactivate the compound.

The pharmaceutical compositions of the disclosure may include one or more pharmaceutically acceptable salts. A “pharmaceutically acceptable salt” refers to a salt that retains the desired biological activity of the parent compound and does not impart any undesired toxicological effects (see e.g., Berge, S. M., et al. 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the disclosure also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Reviews on the development of stable protein (e.g. antibody) formulations may be found in Cleland et al. 1993, Crit. Reviews. Ther. Drug Carrier Systems 10 (4): 307-377 and Wei Wang 1999, Int. J. Pharmaceutcs 185:129-88. Additional formulation discussions for antibodies may be found, e.g., in Daugherty and Mrsny 2006, Advanced Drug Delivery Reviews 58:686-706; U.S. Pat. Nos. 6,171,586, 4,618,486, US Publication No. 20060286103, PCT Publication WO 06/044908, WO 07/095337, WO 04/016286, Colandene et al. 2007, J. Pharm. Sci 96:1598-1608; Schulman 2001, Am. J. Respir. Crit. Care Med. 164: S6-S11 and other known references, each of which is incorporated by reference.

Solutions or suspensions used for intradermal or subcutaneous application typically include one or more of the following components: a sterile diluent such as water for injection, saline solution, fixed oils, polyethylene glycols, glycerine, propylene glycol or other synthetic solvents, antibacterial agents such as benzyl alcohol or methyl parabens, antioxidants such as ascorbic acid or sodium bisulfite, chelating agents such ethylenediaminetetraacetic acid, buffers such as acetates, citrates or phosphates, and agents for the adjustment of tonicity such as sodium chloride or dextrose. The pH can be adjusted with acids or bases, such as hydrochloric acid or sodium hydroxide. Such preparations may be enclosed in ampoules, disposables syringes or multiple dose vials made of glass or plastic.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the antibodies or proteins of the disclosure into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

In one specific embodiment, the antibodies according to the disclosure were administered as a liquid formulation in a vial. The amount of drug per vial was 150 mg. The liquid contained 150 mg/mL antibody, 4.8 mM L-Histidine, 15.2 mM L-Histidine-HCl 220 mM Sucrose and 0.04% Polysorbate 20, at pH 6.0±0.5. A 20% overfill was added to permit complete removal of the intended dose.

### Therapeutic and Other Uses

The antibodies of the present invention have numerous in vitro and in vivo diagnostic and therapeutic utilities involving the diagnosis and treatment of disorders with IL-2-dependent pathophysiology. For example, these molecules can be administered to cells in culture, in vitro or ex vivo, or to human subjects, e.g., in vivo, to treat, prevent and to diagnose a variety disorders with IL-2-dependent pathophysiology.

Accordingly, in one embodiment, the invention provides a method of inhibiting growth of tumor cells in a subject, comprising administering to the subject a therapeutically effective amount of an anti-IL-2 antibody as disclosed herein. In one embodiment, the methods are suitable for the treatment of cancer in vivo. In one embodiment, the antibodies to IL-2 are administered together with IL-2, such as in combination with IL-2.

When antibodies to IL-2 are administered in combination with one or more agents, the combination can be administered in either order or simultaneously.

In another aspect, a method of treating a subject, e.g., reducing or ameliorating, a proliferative condition or disorder (e.g., a cancer), e.g., solid tumor, a soft tissue tumor, or a metastatic lesion, in a subject is provided.

The term cancer is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the antibodies molecules disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the antibody molecules described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In other embodiments, the cancer is a hematological malignancy or cancer including but is not limited to a leukemia or a lymphoma. For example, the anti-IL-2 therapy can be used to treat cancers and malignancies including, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the lymphoma (e.g., an anaplastic large-cell lymphoma or non-Hodgkin lymphoma) has, or is identified as having, an ALK translocation, e.g., an EML4-ALK fusion.

In one embodiment, the cancer is chosen from a lung cancer (e.g., a non-small cell lung cancer (NSCLC) (e.g., a NSCLC with squamous and/or non-squamous histology)), a melanoma (e.g., an advanced melanoma), a renal cancer (e.g., a renal cell carcinoma, e.g., clear cell renal cell carcinoma), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, a non-Hodgkin's lymphoma, or a leukemia (e.g., a myeloid leukemia).

In another embodiment, the cancer is chosen form a carcinoma (e.g., advanced or metastatic carcinoma), melanoma or a lung carcinoma, e.g., a non-small cell lung carcinoma.

In one embodiment, the cancer is a lung cancer, e.g., a non-small cell lung cancer (NSCLC). In certain embodiments, the lung cancer, e.g., the non-small cell lung cancer, has, or is identified as having, an ALK rearrangement or translocation, e.g., an ALK fusion, e.g., an EML4-ALK fusion.

In another embodiment, the cancer is an inflammatory myofibroblastic tumor (IMT). In certain embodiments, the inflammatory myofibroblastic tumor has, or is identified as having, an ALK rearrangement or translocation, e.g., an ALK fusion, e.g., an EML4-ALK fusion.

In other embodiments, the cancer is NSCLC wherein the NSCLC is characterized by one or more of: aberrant activation, amplification, or a mutation of epidermal growth factor receptor (EGFR). In certain embodiments the cancer is NSCLC wherein the NSCLC is characterized by harbouring an EGFR exon 20 insertion, an EGFR exon 19 deletion, EGFR L858R mutation, EGFR T790M, or any combination thereof. In some embodiments, the NSCLC is characterized by harboring L858R and T790M mutations of EGFR. In some embodiments, the NSCLC is characterized by harboring an EGFR exon 20 insertion and T790M mutations of EGFR. In some embodiments, the NSCLC is characterized by harboring an EGFR exon 19 deletion and T790M mutations of EGFR. In some embodiments, the NSCLC is characterized by harboring EGFR mutation selected from the group consisting of an exon 20 insertion, an exon 19 deletion, L858R mutation, T790M mutation, and any combination thereof.

In yet another embodiment, the cancer is a neuroblastoma.

In certain embodiments, the neuroblastoma has, or is identified as having, an ALK rearrangement or translocation, e.g., an ALK fusion, e.g., an EML4-ALK fusion. Methods and compositions disclosed herein are useful for treating metastatic lesions associated with the aforementioned cancers.

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC or clear cell renal cell carcinoma).

In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation).

In another embodiment, the cancer is an inflammatory myofibroblastic tumor (IMT). In certain embodiments, the inflammatory myofibroblastic tumor has, or is identified as having, an ALK rearrangement or translocation, e.g., an ALK fusion, e.g., an EML4-ALK fusion.

In yet another embodiment, the cancer is a neuroblastoma. In certain embodiments, the neuroblastoma has, or is identified as having, an ALK rearrangement or translocation, e.g., an ALK fusion, e.g., an EML4-ALK fusion. Methods and compositions disclosed herein are useful for treating metastatic lesions associated with the aforementioned cancers.

### Combination Therapies

The antibodies, or antigen-binding portion thereof of the disclosure may be administered as the sole active ingredient or in conjunction with, e.g. as an adjuvant to or in combination to, other drugs e.g. immunomodulating agents or cytotoxic or anti-cancer agents, e.g. for the treatment or prevention of diseases mentioned above.

### 1. Exemplary STING Agonists

In an embodiment, the combination includes a STING agonist. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

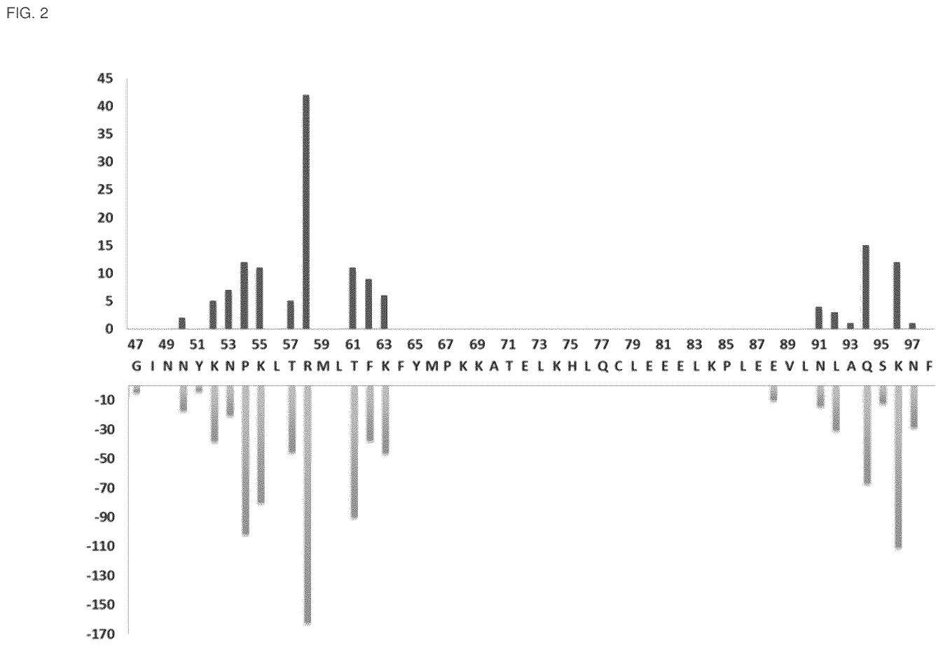

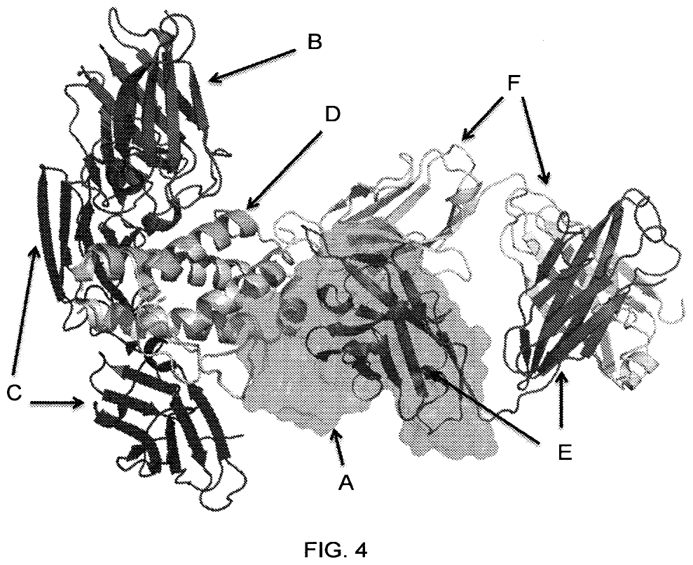

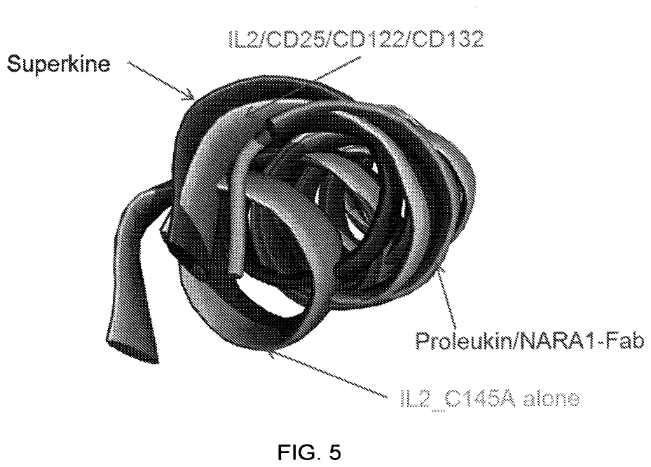

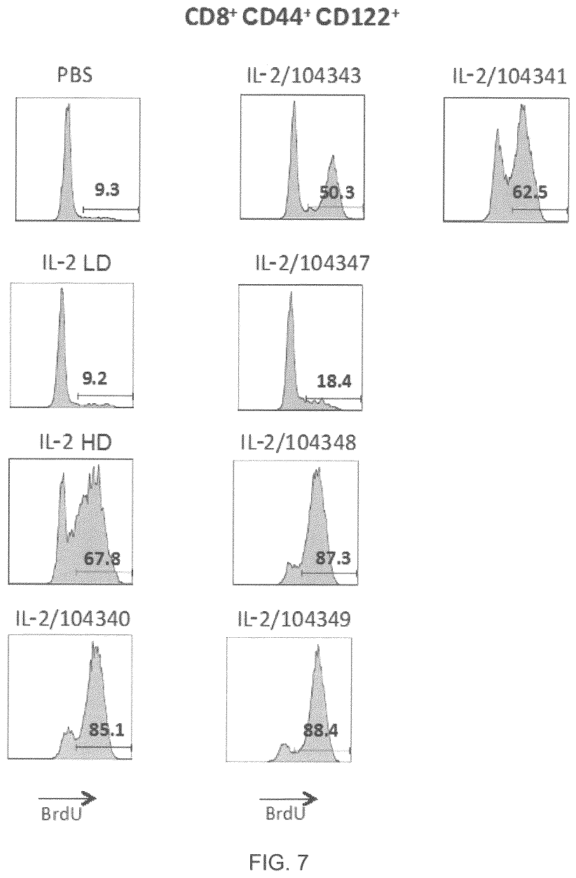

In some embodiments, the STING agonist is Rp,Rp dithio 2′,3′ c-di-AMP (e.g., Rp,Rp-dithio c-[A(2′,5′)pA(3′,5′)p]), or a cyclic dinucleotide analog thereof. In some embodiments, the STING agonist is a compound depicted in U.S. Patent Publication No. US2015/0056224 (e.g., a compound in FIG. 2c, e.g., compound 21 or compound 22). In some embodiments, the STING agonist is c-[G (2′,5′)pG(3′,5′)p], a dithio ribose O-substituted derivative thereof, or a compound depicted in FIG. 4 of PCT Publication Nos. WO 2014/189805 and WO 2014/189806, each of which is incorporated by reference. In some embodiments, the STING agonist is c-[A(2′,5′)pA(3′,5′)p] or a dithio ribose O-substituted derivative thereof, or is a compound depicted in FIG. 5 of PCT Publication Nos. WO 2014/189805 and WO 2014/189806. In some embodiments, the STING agonist is 2′-0-propargyl-cyclic-[A(2′,5′)pA(3′,5′)p] (2′-O-propargyl-ML-CDA) or a compound depicted in FIG. 7 of PCT Publication No. WO 2014/189806, which is incorporated by reference.

Other exemplary STING agonists are disclosed, e.g., in PCT Publication Nos. WO 2014/189805 and WO 2014/189806, and U.S. Publication No. 2015/0056225, each of which is incorporated by reference.

### 2. Exemplary PD-1 Inhibitors

In an embodiment, the combination includes an anti-PD-1, or anti-PD-1 ligand (PD-L1) antibody molecule. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

Exemplary non-limiting combinations and uses of the anti-PD-1 antibody molecules are disclosed in US 2015/0210769, published on Jul. 30, 2015, entitled “Antibody Molecules to PD-1 and Uses Thereof,” incorporated by reference in its entirety.

In one embodiment, the anti-PD-1 antibody molecule includes at least one or two heavy chain variable domain (optionally including a constant region), at least one or two light chain variable domain (optionally including a constant region), or both, comprising the amino acid sequence of BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1 of US 2015/0210769; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as shown in Table 4 of US 2015/0210769; or a sequence substantially identical thereto.

In yet another embodiment, the anti-PD-1 antibody molecule includes at least one, two, or three complementarity determining regions (CDRs) from a heavy chain variable region and/or a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1 of US 2015/0210769; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 1 of US 2015/0210769, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1 of US 2015/0210769, or encoded by a nucleotide sequence shown in Table 1.

In yet another embodiment, the anti-PD-1 antibody molecule includes at least one, two, or three CDRs (or collectively all of the CDRs) from a light chain variable region comprising an amino acid sequence shown in Table 1 of US 2015/0210769, or encoded by a nucleotide sequence shown in Table 1 of US 2015/0210769. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1 of US 2015/0210769. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 1 of US 2015/0210769 (e.g., SEQ ID NO: 16 or 24 for murine or chimeric, unmodified; or any of SEQ ID NOs: 34, 42, 46, 54, 58, 62, 66, 70, 74, or 78 for a modified sequence).

In another embodiment, the anti-PD-1 antibody molecule includes at least one, two, three, four, five or six CDRs (or collectively all of the CDRs) from a heavy and light chain variable region comprising an amino acid sequence shown in Table 1 of US 2015/0210769, or encoded by a nucleotide sequence shown in Table 1 of US 2015/0210769. In one embodiment, one or more of the CDRs (or collectively all of the CDRs) have one, two, three, four, five, six or more changes, e.g., amino acid substitutions or deletions, relative to the amino acid sequence shown in Table 1 of US 2015/0210769, or encoded by a nucleotide sequence shown in Table 1 of US 2015/0210769.

In one embodiment, the anti-PD-1 antibody molecule includes:
(a) a heavy chain variable region (VH) comprising a VHCDR1 amino acid sequence of SEQ ID NO: 4, a VHCDR2 amino acid sequence of SEQ ID NO: 5, and a VHCDR3 amino acid sequence of SEQ ID NO: 3; and a light chain variable region (VL) comprising a VLCDR1 amino acid sequence of SEQ ID NO: 13, a VLCDR2 amino acid sequence of SEQ ID NO: 14, and a VLCDR3 amino acid sequence of SEQ ID NO: 33, each disclosed in Table 1 of US 2015/0210769;
(b) a VH comprising a VHCDR1 amino acid sequence chosen from SEQ ID NO: 1; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32, each disclosed in Table 1 of US 2015/0210769;
(c) a VH comprising a VHCDR1 amino acid sequence of SEQ ID NO: 224, a VHCDR2 amino acid sequence of SEQ ID NO: 5, and a VHCDR3 amino acid sequence of SEQ ID NO: 3; and a VL comprising a VLCDR1 amino acid sequence of SEQ ID NO: 13, a VLCDR2 amino acid sequence of SEQ ID NO: 14, and a VLCDR3 amino acid sequence of SEQ ID NO: 33, each disclosed in Table 1 of US 2015/0210769; or
(d) a VH comprising a VHCDR1 amino acid sequence of SEQ ID NO: 224; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32, each disclosed in Table 1 of US 2015/0210769.

In the combinations herein below, in another embodiment, the anti-PD-1 antibody molecule comprises (i) a heavy chain variable region (VH) comprising a VHCDR1 amino acid sequence chosen from SEQ ID NO: 1, SEQ ID NO: 4, or SEQ ID NO: 224; a VHCDR2 amino acid sequence of SEQ ID NO: 2 or SEQ ID NO: 5; and a VHCDR3 amino acid sequence of SEQ ID NO: 3; and (ii) a light chain variable region (VL) comprising a VLCDR1 amino acid sequence of SEQ ID NO: 10 or SEQ ID NO: 13, a VLCDR2 amino acid sequence of SEQ ID NO: 11 or SEQ ID NO: 14, and a VLCDR3 amino acid sequence of SEQ ID NO: 32 or SEQ ID NO: 33, each disclosed in Table 1 of US 2015/0210769.

In other embodiments, the PD-1 inhibitor is an anti-PD-1 antibody chosen from Nivolumab, Pembrolizumab or Pidilizumab.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. In one embodiment, the inhibitor of PD-1 is Nivolumab, and having a sequence disclosed herein (or a sequence substantially identical or similar thereto, e.g., a sequence at least 85%, 90%, 95% identical or higher to the sequence specified).

In some embodiments, the anti-PD-1 antibody is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335.

In one embodiment, the inhibitor of PD-1 is Pembrolizumab disclosed in, e.g., U.S. Pat. No. 8,354,509 and WO 2009/114335, and having a sequence disclosed herein (or a sequence substantially identical or similar thereto, e.g., a sequence at least 85%, 90%, 95% identical or higher to the sequence specified).

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611.

Other anti-PD1 antibodies include AMP 514 (Amplimmune), among others, e.g., anti-PD1 antibodies disclosed in U.S. Pat. No. 8,609,089, US 2010028330, and/or US20120114649.

In some embodiments, the PD-1 inhibitor is an immunoadhesin (e.g., an immunoadhesin comprising an extracellular or PD-1 binding portion of PD-LI or PD-L2 fused to a constant region (e.g., an Fc region of an immunoglobulin sequence). In some embodiments, the PD-1 inhibitor is AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

### 3. Exemplary TIM-3 Inhibitors

In an embodiment, the combination includes a TIM-3 inhibitor. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

In one embodiment, a combination described herein includes a TIM-3 inhibitor. In some embodiments, the combination is used to treat a cancer, e.g., a cancer described herein, e.g., a solid tumor or a hematologic malignancy.

Exemplary non-limiting combinations and uses of the anti-TIM-3 antibody molecules are disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule includes at least one or two heavy chain variable domain (optionally including a constant region), at least one or two light chain variable domain (optionally including a constant region), or both, comprising the amino acid sequence of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4 of US 2015/0218274; or a sequence substantially identical (e.g., at least 80%, 85%, 90%, 92%, 95%, 97%, 98%, 99% or higher identical) to any of the aforesaid sequences. The anti-TIM-3 antibody molecule, optionally, comprises a leader sequence from a heavy chain, a light chain, or both, as shown in US 2015/0218274; or a sequence substantially identical thereto.

In yet another embodiment, the anti-TIM-3 antibody molecule includes at least one, two, or three complementarity determining regions (CDRs) from a heavy chain variable region and/or a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4 of US 2015/0218274; or a sequence substantially identical (e.g., at least 80%, 85%, 90%, 92%, 95%, 97%, 98%, 99% or higher identical) to any of the aforesaid sequences.

In yet another embodiment, the anti-TIM-3 antibody molecule includes at least one, two, or three CDRs (or collectively all of the CDRs) from a heavy chain variable region comprising an amino acid sequence shown in Tables 1-4 of US 2015/0218274, or encoded by a nucleotide sequence shown in Tables 1-4 of US 2015/0218274. In one embodiment, one or more of the CDRs (or collectively all of the CDRs) have one, two, three, four, five, six or more changes, e.g., amino acid substitutions or deletions, relative to the amino acid sequence shown in Tables 1-4 of US 2015/0218274, or encoded by a nucleotide sequence shown in Table 1-4 of US 2015/0218274.

In yet another embodiment, the anti-TIM-3 antibody molecule includes at least one, two, or three CDRs (or collectively all of the CDRs) from a light chain variable region comprising an amino acid sequence shown in Tables 1-4 of US 2015/0218274, or encoded by a nucleotide sequence shown in Tables 1-4 of US 2015/0218274. In one embodiment, one or more of the CDRs (or collectively all of the CDRs) have one, two, three, four, five, six or more changes, e.g., amino acid substitutions or deletions, relative to the amino acid sequence shown in Tables 1-4 of US 2015/0218274, or encoded by a nucleotide sequence shown in Tables 1-4 of US 2015/0218274. In certain embodiments, the anti-TIM-3 antibody molecule includes a substitution in a light chain CDR, e.g., one or more substitutions in a CDR1, CDR2 and/or CDR3 of the light chain.

In another embodiment, the anti-TIM-3 antibody molecule includes at least one, two, three, four, five or six CDRs (or collectively all of the CDRs) from a heavy and light chain variable region comprising an amino acid sequence shown in Tables 1-4 of US 2015/0218274, or encoded by a nucleotide sequence shown in Tables 1-4 of US 2015/0218274. In one embodiment, one or more of the CDRs (or collectively all of the CDRs) have one, two, three, four, five, six or more changes, e.g., amino acid substitutions or deletions, relative to the amino acid sequence shown in Tables 1-4 of US 2015/0218274, or encoded by a nucleotide sequence shown in Tables 1-4 of US 2015/0218274.

In one embodiment, the anti-TIM-3 antibody molecule includes:
(a) a heavy chain variable region (VH) comprising a VHCDR1 amino acid sequence chosen from SEQ ID NO: 9; a VHCDR2 amino acid sequence of SEQ ID NO: 10; and a VHCDR3 amino acid sequence of SEQ ID NO: 5; and a light chain variable region (VL) comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274;
(b) a VH comprising a VHCDR1 amino acid sequence chosen from SEQ ID NO: 3; a VHCDR2 amino acid sequence of SEQ ID NO: 4; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274;
(c) a VH comprising a VHCDR1 amino acid sequence chosen from SEQ ID NO: 9; a VHCDR2 amino acid sequence of SEQ ID NO: 25; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274;
(d) a VH comprising a VHCDR1 amino acid sequence chosen from SEQ ID NO: 3; a VHCDR2 amino acid sequence of SEQ ID NO: 24; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274;
(e) a VH comprising a VHCDR1 amino acid sequence chosen from SEQ ID NO: 9; a VHCDR2 amino acid sequence of SEQ ID NO: 31; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274; or
(f) a VH comprising a VHCDR1 amino acid sequence chosen from SEQ ID NO: 3; a VHCDR2 amino acid sequence of SEQ ID NO: 30; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274.

Exemplary anti-TIM-3 antibodies are disclosed in U.S. Pat. No. 8,552,156, WO 2011/155607, EP 2581113 and U.S Publication No.: 2014/044728.

### 4. Exemplary LAG-3 Inhibitors

In an embodiment, the combination includes a LAG-3 inhibitor. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

In one embodiment, a combination described herein includes a LAG-3 inhibitor. In some embodiments, the combination is used to treat a cancer, e.g., a cancer described herein, e.g., a solid tumor or a hematologic malignancy.

Exemplary non-limiting combinations and uses of the anti-LAG-3 antibody molecules are disclosed in US 2015/0259420 published on Sep. 17, 2015, entitled “Antibody Molecules to LAG-3 and Uses Thereof,” incorporated by reference in its entirety.

In one embodiment, the anti-LAG-3antibody molecule includes at least one or two heavy chain variable domain (optionally including a constant region), at least one or two light chain variable domain (optionally including a constant region), or both, comprising the amino acid sequence of any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050 (Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420, or encoded by the nucleotide sequence in Table 1 of US 2015/0259420; or a sequence substantially identical (e.g., at least 80%, 85%, 90%, 92%, 95%, 97%, 98%, 99% or higher identical) to any of the aforesaid sequences.

In yet another embodiment, the anti-LAG-3 antibody molecule includes at least one, two, or three complementarity determining regions (CDRs) from a heavy chain variable region and/or a light chain variable region of an antibody described herein, e.g., an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050 (Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420, or encoded by the nucleotide sequence in Table 1 of US 2015/0259420; or a sequence substantially identical (e.g., at least 80%, 85%, 90%, 92%, 95%, 97%, 98%, 99% or higher identical) to any of the aforesaid sequences.

In yet another embodiment, the anti-LAG-3 antibody molecule includes at least one, two, or three CDRs (or collectively all of the CDRs) from a heavy chain variable region comprising an amino acid sequence shown in Table 1 of US 2015/0259420, or encoded by a nucleotide sequence shown in Table 1 of US 2015/0259420. In one embodiment, one or more of the CDRs (or collectively all of the CDRs) have one, two, three, four, five, six or more changes, e.g., amino acid substitutions or deletions, relative to the amino acid sequence shown in Table 1 of US 2015/0259420, or encoded by a nucleotide sequence shown in Table 1 of US 2015/0259420.

In yet another embodiment, the anti-LAG-3 antibody molecule includes at least one, two, or three CDRs (or collectively all of the CDRs) from a light chain variable region comprising an amino acid sequence shown in Table 1 of US 2015/0259420, or encoded by a nucleotide sequence shown in Table 1 of US 2015/0259420. In one embodiment, one or more of the CDRs (or collectively all of the CDRs) have one, two, three, four, five, six or more changes, e.g., amino acid substitutions or deletions, relative to the amino acid sequence shown in Table 1 of US 2015/0259420, or encoded by a nucleotide sequence shown in Table 1 of US 2015/0259420. In certain embodiments, the anti-PD-L1 antibody molecule includes a substitution in a light chain CDR, e.g., one or more substitutions in a CDR1, CDR2 and/or CDR3 of the light chain.

In another embodiment, the anti-LAG-3 antibody molecule includes at least one, two, three, four, five or six CDRs (or collectively all of the CDRs) from a heavy and light chain variable region comprising an amino acid sequence shown in Table 1 of US 2015/0259420, or encoded by a nucleotide sequence shown in Table 1 of US 2015/0259420. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1 of US 2015/0259420.

In one embodiment, the anti-LAG-3 antibody molecule includes:
(i) a heavy chain variable region (VH) including a VHCDR1 amino acid sequence chosen from SEQ ID NO: 1, SEQ ID NO: 4 or SEQ ID NO: 286; a VHCDR2 amino acid sequence of SEQ ID NO: 2; and a VHCDR3 amino acid sequence of SEQ ID NO: 3, each disclosed in Table 1 of US 2015/0259420; and
(ii) a light chain variable region (VL) including a VLCDR1 amino acid sequence of SEQ ID NO: 10, a VLCDR2 amino acid sequence of SEQ ID NO: 11, and a VLCDR3 amino acid sequence of SEQ ID NO: 12, each disclosed in Table 1 of US 2015/0259420.

In another embodiment, the anti-LAG-3 antibody molecule includes:
(i) a heavy chain variable region (VH) including a VHCDR1 amino acid sequence chosen from SEQ ID NO: 1, SEQ ID NO: 4 or SEQ ID NO: 286; a VHCDR2 amino acid sequence of SEQ ID NO: 5, and a VHCDR3 amino acid sequence of SEQ ID NO: 3, each disclosed in Table 1 of US 2015/0259420; and
(ii) a light chain variable region (VL) including a VLCDR1 amino acid sequence of SEQ ID NO: 13, a VLCDR2 amino acid sequence of SEQ ID NO: 14, and a VLCDR3 amino acid sequence of SEQ ID NO: 15, each disclosed in Table 1 of US 2015/0259420.

In one embodiment, the anti-LAG-3 antibody molecule comprises the VHCDR1 amino acid sequence of SEQ ID NO: 1. In another embodiment, the anti-LAG-3 antibody molecule comprises the VHCDR1 amino acid sequence of SEQ ID NO: 4. In yet another embodiment, the anti-LAG-3 antibody molecule comprises the VHCDR1 amino acid sequence of SEQ ID NO: 286, each disclosed in Table 1 of US 2015/0259420.

In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218.

### 5. Exemplary CTLA-4 Inhibitors

In an embodiment, the combination includes a CTLA-4 inhibitor. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

In one embodiment, a combination described herein includes a CTLA-4 inhibitor. In some embodiments, the combination is used to treat a cancer, e.g., a cancer described herein, e.g., a solid tumor or a hematologic malignancy.

Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

In one embodiment, the combination includes an anti-PD-1 antibody molecule, e.g., as described herein, and an anti-CTLA-4 antibody, e.g., ipilimumab. Exemplary doses that can be use include a dose of anti-PD-1 antibody molecule of about 1 to 10 mg/kg, e.g., 3 mg/kg, and a dose of an anti-CTLA-4 antibody, e.g., ipilimumab, of about 3 mg/kg.

Other exemplary anti-CTLA-4 antibodies are disclosed, e.g., in U.S. Pat. No. 5,811,097, which is incorporated by reference.

### 6. Exemplary GITR Modulator

In an embodiment, the combination includes a GITR modulator, such as an agonist or antagonist. In an embodiment, the GITR modulator is an antagonist. In some embodiments, the combination is used to treat a cancer, e.g., a cancer described herein e.g., a solid tumor (e.g., a breast cancer, a squamous cell carcinoma, a melanoma, an ovarian cancer, a fallopian tube carcinoma, a peritoneal carcinoma, a soft tissue sarcoma, a melanoma, a breast cancer, an esophageal cancer, a head and neck cancer, an endometrial cancer, a cervical cancer, or a basal cell carcinoma), e.g., a hematologic malignancy (e.g., a leukemia (e.g., a chronic lymphocytic leukemia (CLL), or a lymphoma (e.g., a marginal zone B-cell lymphoma, a small lymphocytic lymphoma, a follicular lymphoma, Hodgkin lymphoma, non-Hodgkin lymphoma)).

Exemplary GITR modulators include, e.g., GITR fusion proteins and anti-GITR antibodies (e.g., bivalent anti-GITR antibodies), such as, a GITR fusion protein described in U.S. Pat. No. 6,111,090, European Patent No.: 0920505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, U.S. Pat. No. 8,709,424, PCT Publication No.: WO 2013/039954, International Publication No.: WO2013/039954, U.S.

Publication No.: US2014/0072566, International Publication NO.: WO2015/026684, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, U.S. Pat. No. 6,689,607, PCT Publication No.: WO2006/083289, PCT Publication No.: WO 2005/115451, U.S. Pat. No. 7,618,632, PCT Publication No.: WO 2011/051726, International Publication No.: WO2004060319, and International Publication No.: WO2014012479, each of which is incorporated by reference.

### EXAMPLES

### Example 1: Generation and Screening of Mouse Anti-Human IL-2 Antibody NARA1

A reference antibody, designated NARA1, was derived, isolated and structurally characterized according to methods well known to a person skilled in the art.

Balb/c mice were immunized with human (h) IL-2 (34-8029, eBioscience) in Freund's adjuvant (F-5881, Sigma) on days 0, 14 (subcutaneously) and 28 (intravenously). Serum was collected before the first immunization and 9-11 days after every immunization in order to check for anti-hIL-2 antibody titers. On day 35, mice were euthanized and spleen cells were collected following standard procedures. Splenocytes were mixed with myeloma cells at a 5:1 ratio with polyethylene glycol 1500 (10783641001, Roche). A feeder layer obtained from peritoneal lavage of Balb/c mice was used to grow clones in IMDM selective media (21980, Life Technologies) supplemented with 10% ultra-low IgG FBS (16250, Life Technologies), 50 UM mercaptoethanol (313050, Life technologies), 1:100 Insulin-Transferrin-Selenium (41400-045, Life Technologies), 2% IL-6-conditioned media, penicillin-streptomycin (15240, Life Technologies), gentamycin (15750, Life Technologies), and hypoxanthine-aminopterin-thymidine (HAT, H037, Sigma-Aldrich) for several days. Polyclonals were then screened for hIL-2 binding using a direct binding ELISA and for specificity using a competition ELISA, and diluted to obtain monoclonal clones. For expansion of monoclonals, HAT media was replaced by hypoxanthine-thymidine media (HT, 41065, Life Technologies). Monoclonals were then concentrated using 100 kDa centrifugal filter units according to supplier's recommendations (UFC9100, Merck Millipore). Concentrate was further tested for specificity in a dose-dependent manner using a competition ELISA and in vivo using 4 daily intraperitoneally injections of 200 μl concentrate complexed with 1.5 μg hIL-2, followed by assessment by flow cytometry of T cell subsets and natural killer (NK) cells. NARA1 was purified using Protein G agarose (20398. ThermoFisher Scientific) according to supplier's recommendations.

The full length heavy chain of NARA1 is SEQ ID NO: 115 and the full length light chain amino acid sequence of NARA1 is SEQ ID NO: 117.

The corresponding variable regions, VH and VL amino acid sequences of NARA1 are SEQ ID NO: 111 (variable heavy) and SEQ ID NO: 113 (variable light).

Full length light and heavy chain nucleotide coding sequences of NARA1 are SEQ ID NO: 116 (heavy chain coding sequence, including leader sequence) and SEQ ID NO: 118 (light chain coding sequence, including leader sequence).

Variable light and heavy chains nucleotide coding sequences of NARA1 are SEQ ID NO: 112 (variable heavy coding sequence) and SEQ ID NO: 114 (variable light coding sequence).

The CDR regions of NARA1 are delineated using the Kabat system (Kabat, E. A., et al. 1991, Sequences of Proteins of Immunological Interest, Fifth Edition, U.S. Department of Health and Human Services, NIH Publication No. 91-3242, see also Zhao&Lu 2009, Molecular Immunology 47:694-700). For the ease of reading, when CDR regions are delineated according to Kabat definition, they are called hereafter HCDR1, HCDR2, HCDR3, LCDR1, LCDR2, LCDR3 respectively. The CDR regions of NARA1 are: HCDR1 according to SEQ ID NO: 4, HCDR2 according to SEQ ID NO: 2, HCDR3 according to SEQ ID NO: 3, LCDR1 according to SEQ ID NO: 19, LCDR2 according to SEQ ID NO: 20, LCDR3 according to SEQ ID NO: 21.

### Example 2: Crystal Structure of NARA1

### (1) Material and Methods

The complex structure of a human Interleukin 2 mutant (SEQ ID NO: 110), generally known to a person skilled in the art as Proleukin® (aldesleukin), bound to the Fab fragment of antibody NARA 1 was determined. The resulting numbering of residues on Proleukin® is given according to the numbering of wt IL-2.

As will be discussed in detail below, the differences in sequence between Proleukin® and wt hIL-2 are irrelevant and Proleukin® is a valid model for structural analysis of hIL-2.

To define the epitope, X-ray crystallography was used to solve the atomic-resolution structure of the complex mentioned above. X-ray crystallography is a technology that has become routinely and widely used to generate structural data for biomolecules including antibodies and their complexes with antigens (Adams et al, (2013) Annual Review Biophysics 42:265-287; Garman, (2014) Science 343:1102-1108; Joachimiak, (2009) Current Opinion Structural Biology 19:573-584.)

The antigen, Proleukin®, is commercially available as lyophilized powder together with excipients (every 1 mg Proleukin® is mixed with approximately 50 mg mannitol, 0.18 mg sodium dodecyl sulfate, 0.173 mg sodium dihydrogen phosphate, and 0.89 mg disodium hydrogen phosphate). Before used for complex formation, Proleukin® was purified by reverse-phase HPLC to remove the excipients.

The Fab fragment of NARA1 (NARA1-Fab) was generated by papain cleavage of the full-length antibody followed by Protein A chromatography. Briefly, 6.5 ml full-length NARA1 (9 mg/ml in 50 mM citrate buffer with 90 mM sodium chloride at pH 7.0) was mixed with 5 mM DTT and 590 μg Papain (Roche). The cleavage reaction was kept at room temperature for 16h and stopped by addition of 15 ul 56 mM E64 solution (Roche). The cleavage solution was then diluted 10 times with 25 mM Tris, 25 mM NaCl, pH 8.0 and loaded onto a 5 ml Protein A column (GE Healthcare) equilibrate with 5 column volume of 25 mM Tris, 25 mM NaCl, pH 8.0 and Fab fragment was in the loading-through fraction and Fc fragment was bound to the Protein A column.

To form complex, Proleukin® powder after HPLC was dissolved in H₂O at the concentration of 5.5 mg/ml. 6.6 mg Proleukin®, in excess, was added to 11.5 mg NARA1 Fab fragment solution drop by drop. Centrifugation was used to remove the excess Proleukin® that was precipitated under current condition. The complex was then purified by gel filtration with Superdex 200 10×300 (GE Healthcare) with running buffer of 25 mM Tris, 25 mM NaCl, pH 7.4.

Proleukin®/NARA1-Fab complex after gel filtration was concentrated to 14 mg/ml and was screened by vapor diffusion method as sitting drops. The protein solution was mixed 1:1 with reservoir buffer to a total size of 0.4 ul. The experiments were set up with Phoenix robotic system (Art Robbins Instruments), stored in a RockImager hotel (Formulatrix) at 19° C., and imaged automatically. Crystals were harvested 4 days after screening under condition of 20% w/v polyethylene Glycol 3350 and 0.2M sodium nitrate. Crystals were cryo-protected with reservoir buffer containing 10% glycerol and flashed frozen in liquid nitrogen prior to data collection. Diffraction data were collected at the Swiss Light Source (Villigen, Switzerland) at beam-line PX-II with a Pilatus pixel detector using x-ray radiation wavelength of 0.99998 Å.

The dataset was processed with XDS and XSCALE (version Dec. 6, 2010) and the structure was resolved with molecular replacement method with the program PHASER by using Protein Data Bank entry “3INK” as search model for IL-2 and Protein Data Bank entry “3TTI” as search model for Fab fragment. Iterative model building and refinement were performed with the programs Coot (Crystallographic Object-Oriented Toolkit) and AUTOBUSTER (Bricogne et al., 2011). All figures were generated with the program PyMOL (Molecular Graphics System; DeLano Scientific: Palo Alto, CA; http://www.pymol.org).

Epitope residues are defined as those residues from Proleukin® that are within 4 Å distance from any atom in Fab fragment of NARA1 and are further confirmed by CCP4 program CONTACT and AREAIMOL (Collaborative Computational Project, Number 4, version 6.4.0). Similarly paratope residues are defined as those residues from NARA1-Fab that are within 4 Å distance from any atom in Proleukin®.

### (2) Results

The Proleukin®/NARA1-Fab complex was solved to 1.95 Å in space group C 1 2 1 with unit cell dimension a=201.8 Å, b=36.2 Å, c=88.7 Å, alpha=90°, beta=102.9°, gamma=90°.

Please refer to Table 3 for detailed structure statistics. In each asymmetric unit, there is one complex molecule.

TABLE 3
Structure statistics for Proleukin ®/NARA1-Fab complex

| Data collection | | |
|---|---|---|
| Space group | C1 2 1 | |
| Cell dimensions | | |
| a, b, c (Å) | 201.757, 36.233, 88.707 | |
| a, b, g (°) | 90, 102.93, 90 | |
| Resolution (Å) | 58.74-1.95 | |
| R_{merge} | 0.066 | (0.472) |
| I/σ/ | 14.18 | (2.59) |

| | |
|---|---|
| Completeness (%) | 84.8(96) |
| Redundancy | 3.19 |
| Refinement | |
| Resolution (Å) | 58.74-1.95 |
| No. reflections | 34750 |
| R_{work}/R_{free} | 0.2052/0.2872 |
| Ramachandran plot | |
| Outliners | 0.0162 |
| Allowed | 0.0378 |
| Favored | 0.9459 |
| R.m.s. deviations | |
| Bond lengths (Å) | 0.01 |
| Bond angles (°) | 1.7 |

### (3) Epitope and Paratope Analysis

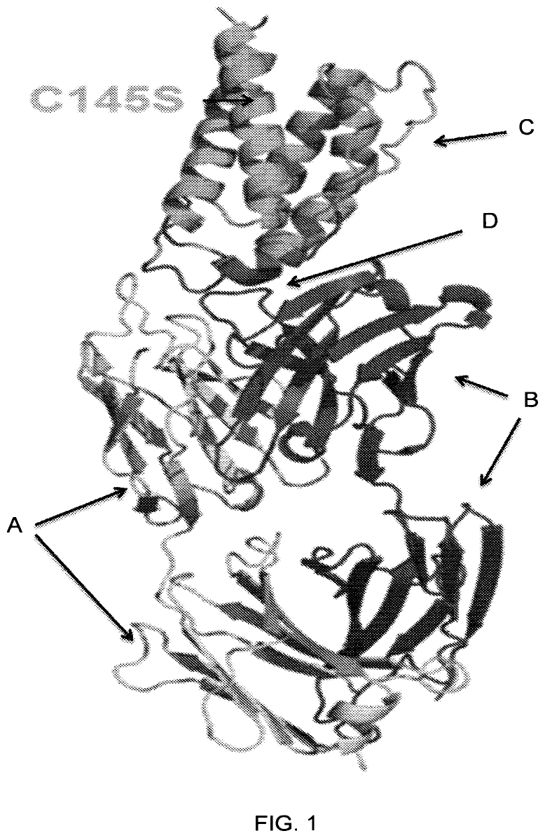

FIG. 1 provides the overview of the three-dimension structure of Proleukin®/Fab-NARA1 complex as obtained in Example 1. Light chain of Fab fragment of NARA1 is designated A, heavy chain of Fab fragment of NARA1 is shown as B, epitope residues recognized by NARA1-Fab are designated D, and Proleukin® is designated C and the mutation, C145S, is highlighted.

FIG. 2 provides further analysis of epitope residues. The X-axis lists the amino acid sequence and numbering according to SEQ ID No 110. The upper side of Y-axis demonstrates the total number of atoms of NARA1-Fab that are within 4 Å from corresponding residue from Proleukin® and the lower side of Y-axis demonstrates the reduced solvent-accessible area (Å2) after binding to NARA1-Fab.

Proleukin® used in Example 1 contains mutation of C145S. As shown in FIG. 1, C145S is far away from the epitope region. In addition the superposition of Ca atoms between Proleukin® in Example 1 with Ca atoms from wt hIL-2 in complex with CD25, CD122, and CD132 (PDB: 2B51) shows r.m.s.d of 0.447 Å, which indicates that the mutation does not disturb the over-all structure. Hence Proleukin® with C145S mutation is a valid model for structural analysis for wt hIL-2.

hIL-2 is 4-helix bundle protein and the 4 helices are named from N-terminus to C-terminus as A, B, C, and D, respectively. The epitope recognized by NARA1-Fab as shown in FIG. 1 is a conformational epitope and spans two regions as shown in FIG. 2: one region (N50-K63) comprises a loop and a short helix and connects helix A and B, and the other region (N91-N97) comprises a loop and connects helix B and C.

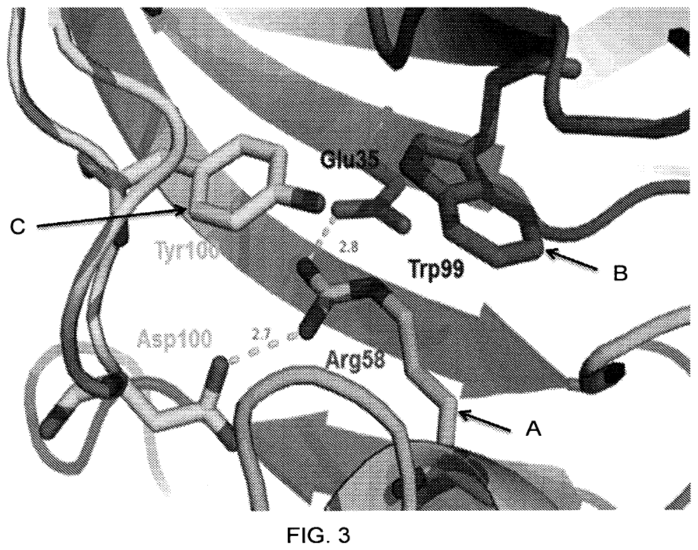

The epitope residues together with interacting paratope residues from NARA1-Fab are summarized in Table 4. Among all the epitope residues, Arg58 as shown in FIG. 2 is the most critical epitope residue for binding with NARA1-Fab, as this residue alone has 42 interacting atoms from NARA1-Fab and accounts for 17.7% of total reduced solvent-accessible surface area as a consequence of binding to NARA1-Fab. Furthermore Arg58, as shown in FIG. 3, forms two strong salt-bridges with Glu35 in HCDR1 and with Asp100 from LCDR3, respectively. Arg58 also makes IT-action interaction with the aromatic ring of Tyr100 from LCDR3. Residues K52, P54, K55, T57, T61, F62, K63, Q94, and K96 are also considered important for the binding to NARA1-Fab, since they all show equal to/more than 5 interacting atoms from NARA1-Fab and larger than 30 Å2 reduced solvent-accessible area as shown in FIG. 2.

TABLE 4
Epitope and paratope summary

| Light chain residue | Epitope residue | Heavy chain residue |
|---|---|---|
| Y31 | N50 | |
| Y31 | K52 | |
| Y31 | N53 | |
| Y31, Y36, S95, N96 | P54 | |
| | K55 | W99, G101, G103, |
| | | Y105 |
| D98 | T57 | |
| D98, Y100 | R58 | L33, E35, W47, W99 |
| | T61 | N52, S55, N59 |
| | F62 | L33, N52 |
| | K63 | S55 |
| | N91 | G101, D102, G103 |
| | L92 | W99, G101 |
| | A93 | G101 |
| | Q94 | D102, G103, Y104 |
| D32, D34 | K96 | Y104 |
| D32 | N97 | |

FIG. 3 illustrates Arg58 as the most critical epitope residue recognized the NARA1-Fab. A represents Proleukin®, B represents heavy chain, and C represents light chain. The involved residues are shown as sticks.

### (4) NARA1-Fab Binding Properties

FIG. 4 shows the overlay of Proleukin®/NARA1-Fab complex with IL-2/CD25/CD122/CD132 quaternary complex. The quaternary complex structure comes from PDB entry “2B5l” with cartoon D in pale cyan representing wt hIL-2, cartoon B in red representing CD122, cartoon C in blue representing CD132, and surface A in green representing CD25. In the Proleukin®/NARA1-Fab complex structure, cyan cartoon D overlayed with wt hIL-2 represents Proleukin®, cartoon E in magenta represents heavy chain, and cartoon F in yellow represents the light chain.

The structure overlay of the two complexes as shown in FIG. 4 clearly shows that NARA1-Fab forms direct competition against CD25 but not against CD122/CD132, which is consistent with the observation that IL-2/NARA1 complex demonstrates mainly pro-Teffector cell activity rather than pro-Treg activity.

### (5) C Helix of Proleukin® in Complex with NARA1-Fab Adopts Conformation that is Similar to that in Quaternary Complex

FIG. 5 displays the overlay of C helices from IL-2_C145A (PDB: 3INK), Superkine (PDB: 3QB1), IL-2/CD25/CD122/CD132 (PDB: 2B5), and Proleukin®/NARA1-Fab.

The polar interface between helix C in IL-2 and CD122 plays an important role in binding between the two parts (Wang et al (2005) Science 310:1159-1163). In 2012 Levin, et al have demonstrated that superkine, an IL-2 mutant, alone has a Helix C adopting confirmation similar to that in the quaternary complex and superkine showed ˜215 times higher binding affinity towards CD122 than wtIL-2 (Levin et al, (2012) Nature 484:529-533). It was observed that such a conformational change in helix C is associated with conformational stabilization, which then reduces the energetic penalties for binding to CD122. As shown in FIG. 5, The conformation of helix C from Proleukin® in complex with NARA1-Fab is also similar to that observed in superkine as well as in IL-2/CD25/CD122/CD132 quaternary complex, therefore it is possible that Proleukin®/NARA1-Fab complex may demonstrate higher binding affinity towards CD122 than wt hIL-2 does.

### Example 3: Humanization of Mouse Monoclonal Antibody NARA1

Humanizing the anti-human IL-2 mouse antibody NARA1 including selection of human acceptor frameworks, back mutations, and mutations that substantially retain and/or improve the binding properties of human CDR-grafted acceptor frameworks is described herein.

The process of humanization is well described in the art (Jones, et al 1986, Queen, et al 1989, Riechmann, et al 1988, Verhoeyen, Milstein and Winter 1988). The term humanization describes as the transfer of the antigen-binding site of a non-human antibody, e.g. a murine derived antibody, to a human acceptor framework, e.g. a human germline sequence (Retter, et al 2005). Main rationale of humanizing an antibody is seen in minimizing the risk of developing an immunogenic response to the antibody in human (Rebello, et al 1999).

The antigen-binding site comprises the complementary determining regions (CDRs) (Chothia and Lesk 1987, Kabat, et al 1991) and positions outside the CDR, i.e. in the framework region of the variable domains (VL and VH) that directly or indirectly affect binding. Framework residues that may directly affect binding can, for example, be found in the so called “outer” loop region located between CDR2 and CDR3. Residues that indirectly affect binding are for example found at so called Vernier Zones (Foote and Winter 1992). They are thought to support CDR conformation. Those positions outside the CDRs are taken into account when choosing a suitable acceptor framework to minimize the number of deviations of the final humanized antibody to the human germline acceptor sequence in the framework regions.

### 1. Sequence Optimization Affinity Maturation

Certain amino acid sequence motifs are known to undergo post-translational modification (PTM) such as glycosylation (i.e. NxS/T, x any but P), oxidation of free cysteines, deamidation (e.g. NG) or isomerization (e.g. DG). If present in the CDR regions, those motifs are ideally removed by site-directed mutagenesis in order to increase product homogeneity.

The process of affinity maturation is well described in the art. Among many display systems, phage display (Smith 1985) and display on eukaryotic cells such as yeast (Boder E. and Wittrup K. (1997). Yeast surface display for screening combinatorial polypeptide libraries. Nat Biotechnol., 15 (6), pp. 553-7.), seem to be the most commonly applied systems to select for antibody-antigen interaction. Advantages of those display systems are that they are suitable for a wide range of antigens and that the selection stringency can be easily adjusted. In phage display, scFv or Fab fragments can be displayed and in yeast display full-length IgG in addition. Those commonly applied methods allow selection of a desired antibody variant from larger libraries with diversities of more than 107. Libraries with smaller diversity, e.g. 103, may be screen by micro-expression and ELISA.

Non-targeted or random antibody variant libraries can be generated for example by error-prone PCR (Cadwell and Joyce 1994) and provide a very simple, but sometimes limited approach. Another strategy is the CDR directed diversification of an antibody candidate. One or more positions in one or more CDRs can be targeted specifically using for example degenerated oligos (Thompson, et al 1996), trinucloetide mutagenesis (TRIM) (Kayushin, et al 1996) or any other approach known to the art.

### 2. Generation of Expression Plasmids

DNA sequences coding for humanized VL and VH domains were ordered at GeneArt (Life Technologies Inc. Regensburg, Germany) including codon optimization for Homo sapiens. Sequences coding for VL and VH domains were subcloned by cut and paste from the GeneArt derived vectors into expression vectors suitable for secretion in mammalian cells. The heavy and light chains were cloned into individual expression vectors to allow co-transfection. Elements of the expression vector include a promoter (Cytomegalovirus (CMV) enhancer-promoter), a signal sequence to facilitate secretion, a polyadenylation signal and transcription terminator (Bovine Growth Hormone (BGH) gene), an element allowing episomal replication and replication in prokaryotes (e.g. SV40 origin and CoIE1 or others known in the art) and elements to allow selection (ampicilin resistance gene and zeocin marker).

### 3. Expression and Purification of Humanized Antibody Candidates

Human Embryonic Kidney cells constitutively expressing the SV40 large T antigen (HEK293-T ATCC11268) are one of the preferred host cell lines for transient expression of humanized and/or optimized IgG proteins. Transfection is performed using PEI (Polyethylenimine, MW 25.000 linear, Polysciences, USA Cat. No. 23966) as transfection reagent. The PEI stock solution is prepared by carefully dissolving 1 g of PEI in 900 ml cell culture grade water at room temperature (RT). To facilitate dissolution of PEI, the solution is acidified by addition of HCl to pH 3-5, followed by neutralization with NaOH to a final pH of 7.05. Finally, the volume is adjusted to 1L and the solution is filtered through a 0.22 μm filter, aliquoted and frozen at −80° C. until further use. Once thawed, an aliquot can be re-frozen up to 3 times at −20° C. but should not be stored long term at −20° C. HEK 293T cells are cultivated using a Novartis proprietary serum-free culture medium for transfection and propagation of the cells, and ExCell VPRO serum-free culture medium (SAFC

Biosciences, USA, Cat. No. 24561C) as production/feed medium. Cells prepared for transient transfections are cultivated in suspension culture. For small scale (<5L) transfections, cells are grown in Corning shake flasks (Corning, Tewksbury, MA) on an orbital shaker (100-120 rpm) in a humidified incubator at 5% CO2 (seed flasks). Cells in the seed cultures should be maintained in the exponential growth phase (cell densities between 5×10⁵ and 3×10⁶/mL) and display a viability of >90% for transfection. Cell densities outside of this range will result in either a lag phase after dilution or reduced transfection efficiency. For small scale (<5L) transfection an aliquot of cells is taken out of the seed cultures and adjusted to 1.4×10⁶ cells/mL in 36% of the final volume with Novartis serum-free culture medium. The DNA solution (Solution 1:0.5 mg of heavy chain and 0.5 mg of light chain expression plasmid for a 1 L transfection) is prepared by diluting the DNA to 1 mg/L (final volume) in 7% of the final culture volume followed by gentle mixing. To prevent bacterial contamination, this solution is filtered using a 0.22 μm filter (e.g. Millipore Stericup). Then 3 mg/L (final volume) of PEI solution is also diluted in 7% of final culture volume and mixed gently (Solution 2). Both solutions are incubated for 5-10 min at room temperature (RT). Thereafter solution 2 is added to solution 1 with gentle mixing and incubated for another 5-15 minutes at room temperature. The transfection mix is then added to the cells and the cultivation of cells is continued for 4 to 6 hours. Finally, the remaining 50% of total production volume are achieved by addition of ExCell® VPRO serum-free culture medium. The cell cultivation is continued for eleven days post transfection. The culture is harvested by centrifugation at 4500 rpm for 20 minutes at 4° C. (Heraeus®, Multifuge 3 S-R, Thermo Scientific, Rockford, IL). The cell supernatant recovered is sterile filtered through a stericup filter (0.22 μm) and stored at 4° C. until further processing.

Purification was performed on an “ÄKTA 100 explorer Air” chromatography system at 4° C. in a cooling cabinet, using a freshly sanitized (0.25 M NaOH) HiTrap ProtA MabSelect®SuRe, 5 ml column. The column was equilibrated with 5 CV of PBS (Gibco, Life Technologies, Carlsbad, CA), and then the sterile filtered supernatant (2 L) was loaded at 4.0 ml/min. The column was washed with 8 CV of PBS to elute the unbound sample and again washed with 5 CV of PBS. Antibody was eluted with 5 CV of 50 mM citrate, 70 mM NaCl pH 3.2. The eluate was collected in 3 ml fractions; fractions were pooled and adjusted at pH 7 with 1 M Tris HCl pH10. The pools were pooled and sterile filtered (Millipore Steriflip, 0.22 μm), the OD 280 nm was measured in a Spectrophotometer ND-1000 (NanoDrop), and the protein concentration was calculated based on the sequence data. The eluate was tested for aggregation (SEC-MALS) and purity (SDS-PAGE, LAL and MS). For the second purification step, if needed, pools from the first purification were loaded into a freshly sanitised (0.5 M NaOH) SPX (Hi Load 16/60 Superdex 200 grade 120 mL (GE-Healthcare). The column was equilibrated with PBS and the run was done with PBS buffer at 1 ml/min, the eluate was collected in 1.2 ml fractions and analyzed as described for the first purification step.

Accordingly, three humanized variable heavy regions; VH1, VH3 and VH5 were generated, as highlighted in Table 5.

TABLE 5
Variable heavy regions

| Variable heavy region | Sequence listing |
|---|---|
| VH1 | SEQ ID NO: 7 |
| VH3 | SEQ ID NO: 9 |
| VH5 | SEQ ID NO: 17 |

Also, three humanized variable light (kappa) regions; VK1, VK2 and VK3, were generated as highlighted in Table 6.

TABLE 6
Variable light regions

| Variable light region | Sequence listing |
|---|---|
| VK1 | SEQ ID NO: 25 |
| VK2 | SEQ ID NO: 27 |
| VK3 | SEQ ID NO: 29 |

### Example 4: Structure-Revised Humanization

Using the crystal structure NARA1/hIL-2 results of Example 2, the humanization design was refined.

Identity was calculated between initial humanized sequences of Example 3 and closest germline. Separately, the isoelectric point (pl) was calculated for heavy and light chains. The results are shown in the Table 7 and below.

TABLE 7
pl data

| | | | | pl Light |
|---|---|---|---|---|
| Variable | | pl Heavy | | refined |
| region | pl Heavy | refined version | pl Light | version |
| VH1 | 9.3 | 9.4 | | |
| VH3 | 9.4 | 9.4 | | |
| VH5 | 9.3 | 9.4 | | |
| VK1 | | | 5.3 | 6.6 |
| VK2 | | | 4.7 | 4.6 |
| VK3 | | | 4.7 | 5.0 |

TABLE 8
Comparison variable regions and variable germline regions

| | % ID (% sim) VH/VL | % ID (% sim) VH/VL |
|---|---|---|
| Variable region | germline | germline refined |
| VH1 | 85% (89%) | 89% (90%) |
| VH3 | 77% (84%) | 85% (88%) |
| VH5 | 86% (91%) | 89% (91%) |
| VK1 | 85% (90%) | 89% (93%) |
| VK2 | 82% (93%) | 84% (93%) |
| VK3 | 83% (87%) | 86% (89%) |

Based on this data, it was decided to refine the structures VH3 and VK3, leading to the sequences in were generated as highlighted in Table 9. VH3 was chosen because the germline refined sequence by using structure information helped to increase the % of identity with the human template till 85% from 77%.

VK3 was chosen because of the increase in pl from 4.7 to 5.0. (VK1 is already 5.3 and VK2 we are not going to increase the pl by using the structure information so we decided for VK3.)

TABLE 9
Structure-refined variable regions

| | Sequence listing |
|---|---|
| Variable light region | |
| VK3s | SEQ ID NO: 34 |
| Variable heavy region | |
| VH3s | SEQ ID NO: 15 |

Based on these six variable heavy and light regions, nine antibodies were generated using a human IgG1 Fc domain with the N297A point mutation, as represented by SEQ ID NO: 103, according to the overview in Table 10.

TABLE 10
Antibodies

| | Variable | Variable | Variable | Variable |
|---|---|---|---|---|
| | Light | Light | Heavy | Heavy |
| Antibody | region | SEQ ID | region | SEQ ID |
| 104341 | VK1 | SEQ ID NO: 25 | VH1 | SEQ ID NO: 7 |
| 104343 | VK2 | SEQ ID NO: 27 | VH1 | SEQ ID NO: 7 |
| 104344 | VK3s | SEQ ID NO: 34 | VH1 | SEQ ID NO: 7 |
| 104345 | VK1 | SEQ ID NO: 25 | VH3s | SEQ ID NO: 15 |
| 104346 | VK2 | SEQ ID NO: 27 | VH3s | SEQ ID NO: 15 |
| 104347 | VK3s | SEQ ID NO: 34 | VH3s | SEQ ID NO: 15 |
| 104348 | VK1 | SEQ ID NO: 25 | VH5 | SEQ ID NO: 17 |
| 104349 | VK2 | SEQ ID NO: 27 | VH5 | SEQ ID NO: 17 |
| 104350 | VK3s | SEQ ID NO: 34 | VH5 | SEQ ID NO: 17 |

Any Fc domain can be used to generate further antibodies, as known to a person skilled in the art. Particularly contemplated Fc domains are non Fc modified human IgG1 according to SEQ ID NO: 93, human IgG2 according to SEQ ID NO: 95, human IgG3 according to SEQ ID NO: 97, human IgG4 according to SEQ ID NO: 99, human IgG1 Fc modified with LALA mutation according to SEQ ID NO: 101, human IgG1 Fc modified with N297A mutation according to SEQ ID NO: 103, human IgG1 Fc modified with DAPA mutation according to SEQ ID NO: 105.

According to a preferred embodiment, the Fc domain is a human IgG1 according to SEQ ID NO: 93, and according to an even more preferred embodiment, the Fc domain is a human IgG1 Fc modified with N297A mutation according to SEQ ID NO: 103.

According to a specific embodiment, the full light chain sequence of antibody 104343 is according to SEQ ID NO: 124 and full the heavy chain sequence is according to SEQ ID NO: 126. According to another specific embodiment the full light chain sequence of antibody 104348 is according to SEQ ID NO: 128 and the full heavy chain sequence is according to SEQ ID NO: 130.

### Example 5: Structural Optimization

Using the crystal structure NARA1/hIL-2 results of Example 2, certain amino acid residues in the CDRs were identified for further structure optimization. Particularly, a so called DG site was identified in LCDR1, and another DG site in HCDR3. Surprisingly, some mutations in these sites dramatically reduce the affinity for human IL-2, whereas other mutations have little to no impact on affinity.

The structure of the complex was analyzed by using modeling software like PyMOL or MOE and residues on the CDR loops that are not in close contact with the antigen (i.e. more than 4 Angstrom from the antigen) have been chosen for further humanization in order to increase the % of identity between the humanized sequence and the human template.

The resulting light chain (kappa) CDRs according to the Kabat definition are found in Table 11.

TABLE 11
Light chain CDRs

| VL | LCDR1 | LCDR2 | LCDR3 |
|---|---|---|---|
| VK1 | SEQ ID NO: 19 | SEQ ID NO: 20 | SEQ ID NO: 21 |
| VK2 | SEQ ID NO: 19 | SEQ ID NO: 20 | SEQ ID NO: 21 |
| VK3 | SEQ ID NO: 19 | SEQ ID NO: 20 | SEQ ID NO: 21 |
| VK3s | SEQ ID NO: 31 | SEQ ID NO: 32 | SEQ ID NO: 21 |
| VK1_D28Q | SEQ ID NO: 69 | SEQ ID NO: 20 | SEQ ID NO: 21 |
| VK1_G29A | SEQ ID NO: 72 | SEQ ID NO: 20 | SEQ ID NO: 21 |
| VK2_D28Q | SEQ ID NO: 69 | SEQ ID NO: 20 | SEQ ID NO: 21 |
| VK2_G29A | SEQ ID NO: 72 | SEQ ID NO: 20 | SEQ ID NO: 21 |
| VK3_D28Q | SEQ ID NO: 69 | SEQ ID NO: 20 | SEQ ID NO: 21 |
| VK3_G29A | SEQ ID NO: 72 | SEQ ID NO: 20 | SEQ ID NO: 21 |
| VK3s_D28Q | SEQ ID NO: 86 | SEQ ID NO: 32 | SEQ ID NO: 21 |
| VK3s_G29A | SEQ ID NO: 90 | SEQ ID NO: 32 | SEQ ID NO: 21 |
| Consensus | SEQ ID NO: 122 | SEQ ID NO: 123 | SEQ ID NO: 21 |

The resulting heavy chain CDRs according to the Kabat definition are found in Table 12.

TABLE 12
Heavy chain CDRs

| VH | HCDR1 | HCDR2 | HCDR3 |
|---|---|---|---|
| VH1 | SEQ ID NO: 4 | SEQ ID NO: 2 | SEQ ID NO: 3 |
| VH3 | SEQ ID NO: 4 | SEQ ID NO: 2 | SEQ ID NO: 3 |
| VH3s | SEQ ID NO: 13 | SEQ ID NO: 12 | SEQ ID NO: 3 |
| VH5 | SEQ ID NO: 4 | SEQ ID NO: 2 | SEQ ID NO: 3 |
| VH1_D98E | SEQ ID NO: 4 | SEQ ID NO: 2 | SEQ ID NO: 36 |
| VH1_G99A | SEQ ID NO: 4 | SEQ ID NO: 2 | SEQ ID NO: 39 |
| VH1_D98Q | SEQ ID NO: 4 | SEQ ID NO: 2 | SEQ ID NO: 42 |
| VH1_D98S | SEQ ID NO: 4 | SEQ ID NO: 2 | SEQ ID NO: 45 |
| VH3_D98E | SEQ ID NO: 4 | SEQ ID NO: 2 | SEQ ID NO: 36 |
| VH3_G99A | SEQ ID NO: 4 | SEQ ID NO: 2 | SEQ ID NO: 39 |
| VH3_D98Q | SEQ ID NO: 4 | SEQ ID NO: 2 | SEQ ID NO: 42 |
| VH3_D98S | SEQ ID NO: 4 | SEQ ID NO: 2 | SEQ ID NO: 45 |
| VH3s_D98E | SEQ ID NO: 13 | SEQ ID NO: 12 | SEQ ID NO: 36 |
| VH3s_G99A | SEQ ID NO: 13 | SEQ ID NO: 12 | SEQ ID NO: 39 |
| VH3s_D98Q | SEQ ID NO: 13 | SEQ ID NO: 12 | SEQ ID NO: 42 |
| VH3s_D98S | SEQ ID NO: 13 | SEQ ID NO: 12 | SEQ ID NO: 45 |
| VH5_D98E | SEQ ID NO: 4 | SEQ ID NO: 2 | SEQ ID NO: 36 |
| VH5_G99A | SEQ ID NO: 4 | SEQ ID NO: 2 | SEQ ID NO: 39 |
| VH5_D98Q | SEQ ID NO: 4 | SEQ ID NO: 2 | SEQ ID NO: 42 |
| VH5_D98S | SEQ ID NO: 4 | SEQ ID NO: 2 | SEQ ID NO: 45 |
| Consensus | SEQ ID NO: 119 | SEQ ID NO: 120 | SEQ ID NO: 121 |

The VH5 mutation D98E was tolerated while D98S and D98Q were surprisingly not tolerated. The mutation G99A was also tolerated. For VK1, the mutation D28Q was tolerated, while surprisingly the mutation G29A was not tolerated.

According to a non-binding theory of the inventors, substituting the VH D98 amino acid, and/or the VL D28 amino acid, with amino acids A, G or T could also be tolerated. Also, substituting the VH G99 amino acid, or the VL G29 amino acid, with amino acids T or S, could also be tolerated.

Based on these optimized variable heavy and light regions, twelve optimized antibodies were generated using a human IgG1 Fc domain with the N297A point mutation, and variable light and heavy regions according to the overview in Table 13.

TABLE 13
Optimized antibodies

| | Variable | Variable | Variable | Variable |
|---|---|---|---|---|
| | Light | Light | Heavy | Heavy |
| Antibody | region | SEQ ID | region | SEQ ID |
| 104341_VH1_{—} | VK1_{—} | SEQ ID | VH1 | SEQ ID |
| VK1D28Q | D28Q | NO: 70 | | NO: 7 |
| 104341_VH1D98E_{—} | VK1 | SEQ ID | VH1_D98E | SEQ ID |
| VK1 | | NO: 25 | | NO: 37 |
| 104341_VH1D98E_{—} | VK1_{—} | SEQ ID | VH1_D98E | SEQ ID |
| VK1D28Q | D28Q | NO: 70 | | NO: 37 |
| 104343_VH1_{—} | VK2_{—} | SEQ ID | VH1 | SEQ ID |
| VK2D28Q | D28Q | NO: 79 | | NO: 7 |
| 104343_VH1D98E_{—} | VK2 | SEQ ID | VH1_D98E | SEQ ID |
| VK2 | | NO: 27 | | NO: 37 |
| 104343_VH1D98E_{—} | VK2_{—} | SEQ ID | VH1_D98E | SEQ ID |
| VK2D28Q | D28Q | NO: 79 | | NO: 37 |
| 104348_VH5_{—} | VK1_{—} | SEQ ID | VH5 | SEQ ID |
| VK1D28Q | D28Q | NO: 70 | | NO: 17 |
| 104348_VH5D98E_{—} | VK1 | SEQ ID | VH5_D98E | SEQ ID |
| VK1 | | NO: 25 | | NO: 49 |
| 104348_VH5D98E_{—} | VK1_{—} | SEQ ID | VH5_D98E | SEQ ID |
| VK1D28Q | D28Q | NO: 70 | | NO: 49 |
| 104349_VH5_{—} | VK2_{—} | SEQ ID | VH5 | SEQ ID |
| VK2D28Q | D28Q | NO: 79 | | NO: 17 |
| 104349_VH5D98E_{—} | VK2 | SEQ ID | VH5_D98E | SEQ ID |
| VK2 | | NO: 27 | | NO: 49 |
| 104349_VH5D98E_{—} | VK2_{—} | SEQ ID | VH5_D98E | SEQ ID |
| VK2D28Q | D28Q | NO: 79 | | NO: 49 |

Any Fc domain can be used to generate further antibodies, as known to a person skilled in the art. Particularly contemplated Fc domains are non Fc modified human IgG1 according to SEQ ID NO: 93, human IgG2 according to SEQ ID NO: 95, human IgG3 according to SEQ ID NO: 97, human IgG4 according to SEQ ID NO: 99, human IgG1 Fc modified with LALA mutation according to SEQ ID NO: 101, human IgG1 Fc modified with N297A mutation according to SEQ ID NO: 103, human IgG1 Fc modified with DAPA mutation according to SEQ ID NO: 105.

According to a preferred embodiment, the Fc domain is a human IgG1 according to SEQ ID NO: 93, and according to an even more preferred embodiment, the Fc domain is a human IgG1 Fc modified with N297A mutation according to SEQ ID NO: 103.

According to one specific embodiment, the full heavy chain sequence of antibody 104348_VH5D98E_VK1D28Q is according to SEQ ID NO: 229 and the full light chain sequence of is according to SEQ ID NO: 395

### Example 6: Affinity Maturation

A humanized NARA1 (104348_VH5D98E_VK1D28Q) was used as a starting point for an affinity maturation process based on multiple steps starting with the cloning and expression of the parental VH (SEQ ID NO: 49) and VK (SEQ ID NO: 70) as Fab on the surface of yeast and the determination of the optimal and sub-optimal binding concentrations of biotinylated Proleukin®.

In brief, parental or wildtype (WT) VH (SEQ ID NO: 49) and VK (SEQ ID NO: 70) sequences were cloned as Fab in a yeast display vector containing the aga2 sequence in-frame with the carboxyl-terminus of VH and a 6 amino acid tag derived from beta-amyloid (APP-tag) in-frame with the carboxyl-terminus of the light chain. Detection of this tag allows for the visualization of the expression level of the Fab on the surface, which is well known to a person skilled in the art. After electroporation of the vector in yeast (Benatuil L. et al. (2010). An improved yeast transformation method for the generation of very large human antibody libraries. Protein Eng Des Sel., 23 (4), pp. 155-9.), the cells were grown in CM Glucose Broth minus Uracil. At time of induction, 7.8E+4 yeast cells growing in their exponential phase were washed with 7 ml induction-medium (CM Galactose Broth minus Ura/0.05% Glucose) and pelleted by spinning the cells 10 minutes at 4000 rpm. The pellet was re-suspended in induction-medium (1E+7 cells/ml) and grown for 16 Hours (HR) at 22° C. in a shaker. Induced yeast cells (4E+7) were collected by centrifugation at 13000 rpm for 1 minute in a 4° C. pre-cooled centrifuge. Cells were washed by re-suspending the pellet in 1 ml FACS buffer (PBS+0.5% BSA) followed by 1 minute centrifugation at 13000 rpm in a 4° C. pre-cooled centrifuge. The yeast pellet was re-suspended in 1 ml FACS buffer and 50 μl was transferred to 12 tubes containing various concentration of biotinylated Proleukin® diluted in FACS buffer (0 nm/0.02 nM/0.05 nM/0.15 nM/0.45 nM/1.3 nM/4 nM/12 nM/36 nM/100 nM/333 nM/1 μM). Yeast was incubated for 1 hour at room temperature (RT) on a rotator, washed two times with 1 ml FACS buffer as described above and pellets re-suspended in 200 μl FACS buffer containing anti-APP mouse monoclonal antibody. After 30 minutes incubation at RT on a rotator, yeast was washed twice with 1 ml FACS buffer and pellets re-suspended in 200 μl labelling buffer (Allophycocyanin (APC)-conjugated Streptavidin/Phycoerythrin (PE)-labelled anti-mouse antibody/FACS buffer). After 30 minutes incubation at RT on a rotor the cells were washed twice with 1 ml FACS buffer and re-suspended in 500 μl cold FACS buffer and filtered through the cap of the FACS tube. Samples were kept in the dark until FACS analysis. The gating-strategy of the FACS analysis was chosen in such a way that PE signals (level of Fab expression on the surface of the yeast) and APC signals (binding of biotinylated Proleukin®) were measured of single yeast cells (singlets). Binding of biotinylated Proleukin® to the Fab on the surface of the yeast could be visualized as events in the FACS-plots that were positive in both PE-signal and APC-signal (data not shown). As expected, incubation of yeast with high amounts of biotinylated Proleukin® (1 μM-12 nM) resulted in the detection of large number of events positive for both PE and APC. This concentration range was considered as the optimal concentration range. Incubation of yeast with 4 nM and 1.3 nM biotinylated Proleukin® resulted in similar levels of PE signal but a dramatic drop in APC signal indicating that less biotinylated Proleukin® was bound to the Fab on the surface of the yeast. This concentration range was considered as the sub-optimal concentration. Yeast incubated with biotinylated Proleukin® concentrations below 1.3 nM (0.45 nM/0.15 nM/0.05 nM/0.02 nM) displayed background levels of APC signal indicating that no biotinylated Proleukin® was bound to the Fab.

In the next step, VH (SEQ ID NO: 49) and VK (SEQ ID NO: 70) were individually random mutagenized by error-prone PCR (Cadwell R. and Joyce G. (1994). Mutagenic PCR. PCR Methods Appl., 3 (6), pp. 136-40.) using the GeneMorph II Random Mutagenesis Kit (Agilent, Catalog #200550). From this, two yeast libraries were generated (Benatuil et al. 2010) expressing either Fabs consisting of the mutagenized VH (VHep) paired with the parental VK (VKD) (SEQ ID NO: 70) or Fabs consisting of the mutagenized VK (VKep) paired with the parental VH (VHp) (SEQ ID NO: 49). In the first round of selection, both yeast libraries (VHep/VKD and VHp/VKep) as well as the parental (VHp/VKD) expressing yeast were induced and FACS stained (1E+9 VHep/VKD and VHp/VKep: 1E+7 VHp/VKD) with an optimal concentration (10 nM) biotinylated Proleukin® using the protocol described above. At least 80 000 yeast cells positive for both PE and APC were FACS sorted in a 15 ml Falcon tube containing 1 ml CM Glucose Broth minus Uracil. This selection allows for the enrichment of yeast expressing a functional Fab on their surface.

After expansion of the FACS sorted yeast from the first round, a second round of selection was applied. Yeast (1E+7) expressing VHp/VKD, VHep/VKD and VHp/VKep were incubated without biotinylated Proleukin®, 10 nM biotinylated Proleukin® (optimal concentration) or 2 nM biotinylated Proleukin® (sub-optimal concentration) and FACS stained as described above. As observed previously, incubation of yeast expressing VHp/VKD with 10 nM Proleukin® resulted in the detection of significant number of events positive for both PE and APC. A similar result was obtained for the Yeast expressing the VHp/VKep library. Interestingly, at this concentration, the APC signal was much stronger in the VHep/VKD library whereas the PE signals were comparable to those observed in yeast expressing VHp/VKD and VHp/VKep suggesting that more biotinylated Proleukin® was bound on the Fabs expressed on the surface of the yeast. This trend became even more pronounced when yeast was incubated with sub-optimal concentrations of biotinylated Proleukin®. Under these conditions, both the VHp/VKD and the VHp/VKep expressing yeast displayed a low number of events that were positive for both PE and APC whereas yeast expressing VHep/VKD displayed similar levels of PE but much higher levels of APC. Based on these findings, all yeast expressing the VHep/VKD library incubated with 2 nM biotinylated Proleukin®, positive for both PE and APC was FACS sorted as described previously. The same was done for the yeast library expressing VHp/VKep incubated with 10 nM biotinylated Proleukin®.

After expansion of the two libraries, plasmids were extracted from the yeast by pre-incubation of the yeast-pellet with Zymolase (Zymoresearch, Catalog #E1004) followed by plasmid recovery using mini-prep spin columns (Qiagen, Catalog #27106). Recovered plasmids were electroporated into bacteria and grown on selection plates. Single colonies were picked and grown overnight in 96-well plates followed by plasmid isolation using the Nucleospin 96 Plasmid Core Kit according to the manufacturers protocol (Macherey-Nagel, Catalog #740616.24). Plasmids were sequenced and analyzed. The VH mutations are summarized in Table 14

The observed mutations were cloned in various combinations (Table 14 for VH SEQ ID Nos and Table 15 for VK SEQ ID NOs) into the parental IgG1 mammalian expression vector (Table 16).

TABLE 14
VH mutations

| Designation | VH |
|---|---|
| VHp/WT (VH5_D98E) | SEQ ID NO: 49 |
| VH-F100dY/N58Y/T30S | SEQ ID NO: 145 |
| VH-F100dY/N58Y | SEQ ID NO: 161 |
| VH-F100dY/T30S | SEQ ID NO: 177 |
| VH-F100dY | SEQ ID NO: 193 |
| VH-N58Y/T30S | SEQ ID NO: 209 |
| VH-N58Y | SEQ ID NO: 225 |

TABLE 15
VK mutations

| Designation | VL |
|---|---|
| VKp/WT(VK1_D28Q) | SEQ ID NO: 70 |
| VK-A50S | SEQ ID NO: 243 |
| VK-A50T | SEQ ID NO: 259 |
| VK-M33L/A50S | SEQ ID NO: 275 |
| VK-M33L | SEQ ID NO: 391 |

TABLE 16
Plasmids

| Plasmid | | |
|---|---|---|
| ID | Mutation/s | VH |
| SP#2764 | WT | SEQ ID NO: 49 |
| SP#3563 | F100d_Y | SEQ ID NO: 193 |
| SP#3564 | N58Y | SEQ ID NO: 225 |
| SP#3569 | F100d_Y + N58Y | SEQ ID NO: 161 |
| SP#3566 | F100d_Y + T30S | SEQ ID NO: 177 |
| SP#3567 | N58Y + T30S | SEQ ID NO: 209 |
| SP#3587 | F100d_Y + N58Y + T30S | SEQ ID NO: 145 |

| Plasmid | | |
|---|---|---|
| ID | Mutation/s | VL |
| SP#2445 | WT | SEQ ID NO: 70 |
| SP#3568 | M33L | SEQ ID NO: 391 |
| SP#3569 | A50S | SEQ ID NO: 243 |
| SP#3570 | A50T | SEQ ID NO: 259 |
| SP#3571 | M33L + A50S | SEQ ID NO: 275 |

Subsequently, a transfection matrix was designed of plasmids encoding either wild type or mutated versions of VH and VK generating 35 unique antibodies, as seen in Table 17. The plasmids were transfected into HEK293F cells grown in 6-well plates using Fugene® HD (Promega, Catalog #E2311) according to the manufacturers' protocol. Supernatants were harvested three days post transfection and the antibody titer (internal process control, IPC) in the supernatant was determined using proteinA/HPLC (Holenstein F. et al. (2015) Automated harvesting and 2-step purification of unclarified mammalian cell-culture broths containing antibodies. J Chromatogr A., 1418, pp. 103-9.).

TABLE 17
First set of antibodies

| | VH- | | | |
|---|---|---|---|---|
| Antibody | plasmid | VH- | VK- | VK- |
| No: | ID | mutations | plasmid ID | mutations |
| 1 | SP#2764 | WT | SP#2445 | WT |
| 2 | | | SP#3568 | M33L |
| 3 | | | SP#3569 | A50S |
| 4 | | | SP#3570 | A50T |
| 5 | | | SP#3571 | M33L + A50S |
| 6 | SP#3563 | F100d_Y | SP#2445 | WT |
| 7 | | | SP#3568 | M33L |
| 8 | | | SP#3569 | A50S |
| 9 | | | SP#3570 | A50T |
| 10 | | | SP#3571 | M33L + A50S |
| 11 | SP#3564 | N58Y | SP#2445 | WT |
| 12 | | | SP#3568 | M33L |
| 13 | | | SP#3569 | A50S |
| 14 | | | SP#3570 | A50T |
| 15 | | | SP#3571 | M33L + A50S |
| 16 | SP#3569 | F100d_Y + N58Y | SP#2445 | WT |
| 17 | | | SP#3568 | M33L |
| 18 | | | SP#3569 | A50S |
| 19 | | | SP#3570 | A50T |
| 20 | | | SP#3571 | M33L + A50S |
| 21 | SP#3566 | F100d_Y + T30S | SP#2445 | WT |
| 22 | | | SP#3568 | M33L |
| 23 | | | SP#3569 | A50S |
| 24 | | | SP#3570 | A50T |
| 25 | | | SP#3571 | M33L + A50S |
| 26 | SP#3567 | N58Y + T30S | SP#2445 | WT |
| 27 | | | SP#3568 | M33L |
| 28 | | | SP#3569 | A50S |
| 29 | | | SP#3570 | A50T |
| 30 | | | SP#3571 | M33L + A50S |
| 31 | SP#3587 | F100d_Y + N58Y + | SP#2445 | WT |
| 32 | | T30S | SP#3568 | M33L |
| 33 | | | SP#3569 | A50S |
| 34 | | | SP#3570 | A50T |
| 35 | | | SP#3571 | M33L + A50S |

The sequences of the antibodies are set forth in Table 41, and summarized in Table 18 below.

TABLE 18
Sequence overview of first set of antibodies

| | | | Full length | Full length |
|---|---|---|---|---|
| Antibody | VH | VL | heavy chain | light chain |
| 1 | SEQ ID NO: | SEQ ID NO: | SEQ ID NO: | SEQ ID NO: |
| | 49 | 70 | 229 | 395 |
| 2 | SEQ ID NO: | SEQ ID NO: | SEQ ID NO: | SEQ ID NO: |
| | 49 | 391 | 229 | 393 |
| 3 | SEQ ID NO: | SEQ ID NO: | SEQ ID NO: | SEQ ID NO: |
| | 49 | 243 | 229 | 245 |
| 4 | SEQ ID NO: | SEQ ID NO: | SEQ ID NO: | SEQ ID NO: |
| | 49 | 259 | 229 | 261 |
| 5 | SEQ ID NO: | SEQ ID NO: | SEQ ID NO: | SEQ ID NO: |
| | 49 | 275 | 229 | 277 |
| 6 | SEQ ID NO: | SEQ ID NO: | SEQ ID NO: | SEQ ID NO: |
| | 193 | 70 | 195 | 395 |
| 7 | SEQ ID NO: | SEQ ID NO: | SEQ ID NO: | SEQ ID NO: |
| | 193 | 391 | 195 | 393 |
| 8 | SEQ ID NO: | SEQ ID NO: | SEQ ID NO: | SEQ ID NO: |
| | 193 | 243 | 195 | 245 |
| 9 | SEQ ID NO: | SEQ ID NO: | SEQ ID NO: | SEQ ID NO: |
| | 193 | 259 | 195 | 261 |
| 10 | SEQ ID NO: | SEQ ID NO: | SEQ ID NO: | SEQ ID NO: |
| | 193 | 275 | 195 | 277 |
| 11 | SEQ ID NO: | SEQ ID NO: | SEQ ID NO: | SEQ ID NO: |
| | 225 | 70 | 227 | 395 |
| 12 | SEQ ID NO: | SEQ ID NO: | SEQ ID NO: | SEQ ID NO: |
| | 225 | 391 | 227 | 393 |
| 13 | SEQ ID NO: | SEQ ID NO: | SEQ ID NO: | SEQ ID NO: |
| | 225 | 243 | 227 | 245 |
| 14 | SEQ ID NO: | SEQ ID NO: | SEQ ID NO: | SEQ ID NO: |
| | 225 | 259 | 227 | 261 |
| 15 | SEQ ID NO: | SEQ ID NO: | SEQ ID NO: | SEQ ID NO: |
| | 225 | 275 | 227 | 277 |
| 16 | SEQ ID NO: | SEQ ID NO: | SEQ ID NO: | SEQ ID NO: |
| | 161 | 70 | 163 | 395 |
| 17 | SEQ ID NO: | SEQ ID NO: | SEQ ID NO: | SEQ ID NO: |
| | 161 | 391 | 163 | 393 |
| 18 | SEQ ID NO: | SEQ ID NO: | SEQ ID NO: | SEQ ID NO: |
| | 161 | 243 | 163 | 245 |
| 19 | SEQ ID NO: | SEQ ID NO: | SEQ ID NO: | SEQ ID NO: |
| | 161 | 259 | 163 | 261 |
| 20 | SEQ ID NO: | SEQ ID NO: | SEQ ID NO: | SEQ ID NO: |
| | 161 | 275 | 163 | 277 |
| 21 | SEQ ID NO: | SEQ ID NO: | SEQ ID NO: | SEQ ID NO: |
| | 177 | 70 | 179 | 395 |
| 22 | SEQ ID NO: | SEQ ID NO: | SEQ ID NO: | SEQ ID NO: |
| | 177 | 391 | 179 | 393 |
| 23 | SEQ ID NO: | SEQ ID NO: | SEQ ID NO: | SEQ ID NO: |
| | 177 | 243 | 179 | 245 |
| 24 | SEQ ID NO: | SEQ ID NO: | SEQ ID NO: | SEQ ID NO: |
| | 177 | 259 | 179 | 261 |
| 25 | SEQ ID NO: | SEQ ID NO: | SEQ ID NO: | SEQ ID NO: |
| | 177 | 275 | 179 | 277 |
| 26 | SEQ ID NO: | SEQ ID NO: | SEQ ID NO: | SEQ ID NO: |
| | 209 | 70 | 211 | 395 |
| 27 | SEQ ID NO: | SEQ ID NO: | SEQ ID NO: | SEQ ID NO: |
| | 209 | 391 | 211 | 393 |
| 28 | SEQ ID NO: | SEQ ID NO: | SEQ ID NO: | SEQ ID NO: |
| | 209 | 243 | 211 | 245 |
| 29 | SEQ ID NO: | SEQ ID NO: | SEQ ID NO: | SEQ ID NO: |
| | 209 | 259 | 211 | 261 |
| 30 | SEQ ID NO: | SEQ ID NO: | SEQ ID NO: | SEQ ID NO: |
| | 209 | 275 | 211 | 277 |
| 31 | SEQ ID NO: | SEQ ID NO: | SEQ ID NO: | SEQ ID NO: |
| | 145 | 70 | 147 | 395 |
| 32 | SEQ ID NO: | SEQ ID NO: | SEQ ID NO: | SEQ ID NO: |
| | 145 | 391 | 147 | 393 |
| 33 | SEQ ID NO: | SEQ ID NO: | SEQ ID NO: | SEQ ID NO: |
| | 145 | 243 | 147 | 245 |
| 34 | SEQ ID NO: | SEQ ID NO: | SEQ ID NO: | SEQ ID NO: |
| | 145 | 259 | 147 | 261 |
| 35 | SEQ ID NO: | SEQ ID NO: | SEQ ID NO: | SEQ ID NO: |
| | 145 | 275 | 147 | 277 |

Subsequently, hIL-2 binding efficiency of the antibodies present in the supernatants was determined using ELISA. In brief, ELISA plates (Maxisorp 96-well black microtiter plate) were coated overnight at 4° C. with 100 μL/well of Proleukin® (5 μg/ml PBS). ELISA plates were washed once with TBST (1×TBS/0.05% Tween 20) using a plate-washer (BioTek). Plates were blocked by adding 350 μl/well blocking buffer (TBS/1× casein (Vector laboratories 10× solution #SP-5020), filtered 0.22 μm) and incubated for 2 hours at RT under gentle agitation. After removal of the blocking buffer, plates were washed once with TBST using the plate washer. Supernatants were diluted 1:2 in blocking buffer and 50 μl transferred to the designated well of the ELISA plate. Each sample was present on three ELISA plates. The three plates were incubated at RT under gentle agitation. After 2 hours incubation one of the plates was washed three times with TBST before 100 μl/well detection antibody (TBST/goat polyclonal HRP-conjugated anti-human Fab2; Dianova/Jackson ImmunResearch, Catalog #109-036-006) was added. The other two plates were washed either every hour three times with TBST for 4 hours or washed every hour three times for 12 hours before adding the detection antibody. After 1 hour incubation at RT under gentle agitation, the wells were washed three times with washing buffer and the plates tapped dry on a stack of paper towels before adding 100 μl/well BM ChemiLuminesence ELISA Substrate (POD) (Roche Diagnostics #11582950001). Luminescence signal was measured after 5 minutes incubation in the dark. The ELISA values for the 35 antibodies are seen in Table 19. All ELISA values are mean values based on two measurements.

TABLE 19
ELISA values

| | | | wash 3× | wash 3× |
|---|---|---|---|---|
| Antibody | IPC | Normal | every hour | every hour |
| No: | (mg/L) | ELISA | for 4 HRS | for 12 HRS |
| 1 | 3.89 | 1182 | 1063 | 717 |
| 2 | 3.34 | 1355 | 1231 | 518 |
| 3 | 4.75 | 4496 | 4580 | 1313 |
| 4 | 4 | 2175 | 1987 | 705 |
| 5 | 4.17 | 4974 | 4490 | 873 |
| 6 | 2.77 | 5849 | 5115 | 1732 |
| 7 | 2.7 | 7638 | 6360 | 1553 |
| 8 | 2.75 | 9373 | 7314 | 2533 |
| 9 | 2.1 | 1944 | 1427 | 686 |
| 10 | 2.3 | 6047 | 4433 | 1630 |
| 11 | 3.78 | 12391 | 10500 | 3498 |
| 12 | 4.15 | 20047 | 17398 | 5960 |
| 13 | 3.17 | 14541 | 14591 | 4524 |
| 14 | 2.86 | 7181 | 7203 | 2538 |
| 15 | 2.71 | 11086 | 10494 | 3796 |
| 16 | 2.14 | 10215 | 9814 | 2277 |
| 17 | 1.85 | 3048 | 2815 | 1141 |
| 18 | 1.91 | 8060 | 7319 | 3656 |
| 19 | 1.89 | 5402 | 4875 | 2208 |
| 20 | 1.93 | 11719 | 11055 | 5200 |
| 21 | 2.15 | 2195 | 1839 | 892 |
| 22 | 2.27 | 3972 | 3188 | 1204 |
| 23 | 2.39 | 6168 | 4635 | 1248 |
| 24 | 2.06 | 2772 | 1877 | 674 |
| 25 | 2.16 | 3574 | 3050 | 1084 |
| 26 | 2.74 | 4029 | 4212 | 1800 |
| 27 | 3.01 | 7755 | 6997 | 2770 |
| 28 | 3.27 | 18459 | 18060 | 5872 |
| 29 | 2.65 | 6954 | 6848 | 2724 |
| 30 | 2.8 | 14920 | 14375 | 2803 |
| 31 | 2.06 | 11389 | 10263 | 3403 |
| 32 | 1.9 | 7187 | 6211 | 3332 |
| 33 | 1.82 | 3988 | 3566 | 1823 |
| 34 | 1.84 | 3857 | 3077 | 1362 |
| 35 | 1.82 | 4914 | 4045 | 1789 |

Most of the introduced mutations (e.g. Antibody No: Aug. 11, 2012/13/15/16/20/28/30/31) displayed similar or lower titers when compared to the parental antibody (Antibody No: 1) but dramatically improved the binding efficiency. Even after 12 hours excessive washing, some of the mutants still displayed high level of binding (e.g. Antibody No: Dec. 13, 2015/20/28). In order to rank the various antibodies, the supernatants were serial diluted and binding affinities determined by ELISA.

EC50 values were calculated using the titers determined by proteinA/HPLC, according to standard methods well known to a person skilled in the art. The results are summarized in Table 20.

TABLE 20
EC50 values

| Antibody | | | Cell Sups |
|---|---|---|---|
| No: | VH-mutations | VK-mutations | EC50 (nM) |
| 1 | WT | WT | 1.04 |
| 2 | | M33L | 0.79 |
| 3 | | A50S | 0.51 |
| 4 | | A50T | 0.73 |
| 5 | | M33L + A50S | 0.50 |
| 6 | F100d_Y | WT | 0.34 |
| 7 | | M33L | 0.31 |
| 8 | | A50S | 0.24 |
| 9 | | A50T | 0.50 |
| 10 | | M33L + A50S | 0.35 |
| 11 | N58Y | WT | 0.34 |
| 12 | | M33L | 0.25 |
| 13 | | A50S | 0.19 |
| 14 | | A50T | 0.36 |
| 15 | | M33L + A50S | 0.28 |
| 16 | F100d_Y + N58Y | WT | 0.38 |
| 17 | | M33L | 1.39 |
| 18 | | A50S | 0.54 |
| 19 | | A50T | 0.50 |
| 20 | | M33L + A50S | 0.27 |
| 21 | F100d_Y + T30S | WT | 0.65 |
| 22 | | M33L | 0.43 |
| 23 | | A50S | 0.26 |
| 24 | | A50T | 0.59 |
| 25 | | M33L + A50S | 0.42 |
| 26 | N58Y + T30S | WT | 0.49 |
| 27 | | M33L | 0.39 |
| 28 | | A50S | 0.16 |
| 29 | | A50T | 0.24 |
| 30 | | M33L + A50S | 0.22 |
| 31 | F100d_Y + N58Y + | WT | 0.39 |
| 32 | T30S | M33L | 0.65 |
| 33 | | A50S | 0.67 |
| 34 | | A50T | 0.65 |
| 35 | | M33L + A50S | 0.72 |

Based on binding efficiency and representation of the mutation, a panel of 7 antibodies (Table 21) were selected, expressed and purified (Holenstein et al. 2015) and further characterized.

TABLE 21
Selected antibodies

| Antibody | | | |
|---|---|---|---|
| No: | VH-mutations | VK-mutations | Designation |
| 8 | F100d_Y | A50S | 108923 |
| 12 | N58Y | M33L | 108924 |
| 13 | N58Y | A50S | 108925 |
| 15 | N58Y | M33L + A50S | 108926 |
| 20 | F100d_Y + | M33L + A50S | 108928 |
| | N58Y | | |
| 26 | N58Y + T30S | WT | 108929 |
| 28 | N58Y + T30S | A50S | 108930 |

The sequences of the selected antibodies are set forth in Table 41, and summarized in Table 22 below.

TABLE 22
Sequence overview of selected of antibodies

| | | | Full length | Full length |
|---|---|---|---|---|
| Antibody | VH | VL | heavy chain | light chain |
| 108923 | SEQ ID NO: | SEQ ID NO: | SEQ ID NO: | SEQ ID NO: |
| | 193 | 243 | 195 | 245 |
| 108924 | SEQ ID NO: | SEQ ID NO: | SEQ ID NO: | SEQ ID NO: |
| | 225 | 391 | 227 | 393 |
| 108925 | SEQ ID NO: | SEQ ID NO: | SEQ ID NO: | SEQ ID NO: |
| | 225 | 243 | 227 | 245 |
| 108926 | SEQ ID NO: | SEQ ID NO: | SEQ ID NO: | SEQ ID NO: |
| | 225 | 275 | 227 | 277 |
| 108928 | SEQ ID NO: | SEQ ID NO: | SEQ ID NO: | SEQ ID NO: |
| | 161 | 275 | 163 | 277 |
| 108929 | SEQ ID NO: | SEQ ID NO: | SEQ ID NO: | SEQ ID NO: |
| | 209 | 259 | 211 | 261 |
| 108930 | SEQ ID NO: | SEQ ID NO: | SEQ ID NO: | SEQ ID NO: |
| | 209 | 275 | 211 | 277 |

### Example 7: Binding Affinity

### 1. Enzyme-Linked Immunosorbent Assay (ELISA)

ELISA, well known to a person skilled in the art, was used to screen the candidates.

ELISA plates (Corning) were coated with human IL-2 (Proleukin®) at 10 μg/mL in PBS overnight at 4° C. The plates were washed 6 times with PBST, then blocked in 1% BSA/PBST for 2 hours. After blocking and washing, the anti-human IL-2 antibodies were added on the plate in an 11-point dilution series in 1% BSA/PBST and incubated for 2 hours. Afterwards the plates were washed again, followed by the incubation with the detection antibodies, either anti-mouse IgG-biotinylated (NARA1) or anti-human IgG-biotinylated (humanized antibodies) in an 1:10′000 dilution in 1% BSA/PBST for 2 hours. The plates were then washed again and incubated with Streptavidin-Horseradish Peroxidase in 1% BSA/PBS for 45 minutes. After washing, the substrate (R&D Systems) was added into the plates and the enzymatic reaction was stopped after 3 minutes by adding Stop Solution. The plates were read by a microplate reader at 450 nm with a wavelength correction set to 540 nm.

The whole process was performed at room temperature.

The mean of 3 independent ELISA experiments are found in Table 23.

### 2. Solution Equilibration Titration (SET) Assays

A SET assay, well known to a person skilled in the art, was conducted to determine and compare the affinity (K_{D}) of five humanized anti-IL-2 antibodies and the anti-IL-2 (NARA1)-mouse IgG2a to IL-2 protein.

The solution equilibration titration (SET) assay allows the determination of antibody-antigen interaction affinities (K_{D}) for tight binders. This technique does not require immobilization or labelling of either interaction partner and is suitable for strong interactions (K_{D}=pM to low nM range).

Mixtures of a constant concentration of antibody (concentrations at or below the expected K_{D}) are co-incubated with antigen within a suitable concentration range (well below and well above the K_{D}) until equilibrium is reached. The amount of free antibody binding sites is determined by transferring the mixtures on antigen-coated plates and a brief incubation. The free antibody consequently bind to the plate and is detected with a detection antibody. The resulting signal is plotted versus the antigen concentration. The K_{D} is accurately determined by non-linear curve fitting.

### (1) Materials and Methods

The following antibodies were used:
anti-IL-2 (NARA1)-mIgG2a: 8.7 mg/ml, 150 kDa, 58.00 μM
anti-IL-2-hsIgG1 104340 (chimeric NARA1 antibody): 4.25 mg/ml, 150 kDa, 28.33 μM
anti-IL-2-hsIgG1 104343:3.69 mg/ml, 150 kDa, 24.60 μM
anti-IL-2-hsIgG1 104347:4.03 mg/ml, 150 kDa, 26.87 M
anti-IL-2-hsIgG1 104348:2.71 mg/ml, 150 kDa, 18.07 μM
anti-IL-2-hsIgG1 104349:3.29 mg/ml, 150 kDa, 21.93 M

### Antigens

IL-2 (proleukin®): 1.00 mg/ml, 15.33 kDa, 65.24 μM
IL-2 (WT): 0.15 mg/ml, 15.55 kDa, 9.65 M

### Detection Antibodies

MSD Sulfo-tag-labeled goat anti-human antibody, Meso Scale Discovery, Cat #R32AJ-5
MSD Sulfo-tag-labeled goat anti-mouse antibody, Jackson IR, Cat #115-005-071

### Instrument and Software

MSD SECTOR Imager 6000 controlled by Discovery Workbench software
Data processing with XLfit, a MS Excel add-in software

### Assay Plates

Standard 384-well plate for SECTOR Imager 6000, Meso Scale Discovery Cat #L21XA
Polypropylene 384-well plate, Greiner Cat #781280

### Reagents

Bovine serum albumin (BSA), VWR Cat #422351S.
Phosphate-buffered saline (PBS) 10×, Teknova Cat #P0195.
MSD Read Buffer T 4×, Meso Scale Discovery Cat #R92TC-1
Tris-buffered saline (TBS) 20×, Teknova Cat #T1680
Tween-20, VWR Cat #437082Q

### Buffers

Blocking buffer: 1×PBS+5% (w/v) BSA
Coating buffer: 1×PBS
Sample buffer: 1×PBS+0.5% (w/v) BSA+0.02% (v/v) Tween-20
Wash buffer: 1×TBS+0.05% (v/v) Tween-20
Read buffer: 1×MSD Read Buffer

A 22 serial 2n dilution of the antigen was prepared in sample buffer. A constant concentration of the antibody was added. The antigen and antibody concentrations are listed below. A volume of 60 μl of each antigen: antibody mix was distributed in duplicates to a 384-well polypropylene microtiter plate (PP MTP). Sample buffer served as negative control and a sample containing no antigen as positive control (Bₘₐₓ). The plate was sealed and incubated overnight (o/n) at room temperature (RT). A 384-well standard MSD array plate was coated o/n with 2 μg/ml of IL-2. After three times washing with washing buffer, the plate was blocked with 50 μl/well blocking buffer for 1 hour at RT. After washing, a volume of 30 μl/well of the antigen: antibody mix was transferred from the PP MTP to the coated MSD plate and incubated for 20 min at RT. After an additional wash step, 30 μl of detection antibody (diluted 1:2000) in sample buffer was added to each well and incubated for 30 min at RT. The MSD plate was washed and 35 μl/well of read buffer was added and incubated for 5 min. ECL signals were measured with the MSD SECTOR Imager 6000.

### (2) Results

The results of the SET are found in Table 23. All tested anti-IL-2 antibodies showed similar affinities to IL-2 proteins in the low pM range (IL-2 wt not shown).

TABLE 23
Binding affinity data

| | EC50 from IL- | Kd from SET |
|---|---|---|
| Antibody | 2 ELISA (nM) | assay (nM) |
| NARA1 | 0.3 | 0.078 |
| 104341 | 0.4 | |
| 104343 | 0.3 | 0.075 |
| 104344 | 0.7 | |
| 104345 | 2.9 | |
| 104346 | 1.7 | |
| 104347 | 39 | 10 |
| 104348 | 0.3 | 0.105 |
| 104349 | 0.3 | 0.082 |
| 104350 | 0.8 | |
| 104343_VH1_VK2D28Q | 0.59 | 0.069 |
| 104343_VH1D98E_VK2 | 0.57 | 0.098 |
| 104343_VH1D98E_VK2D28Q | 0.75 | 0.111 |
| 104348_VH5_VK1D28Q | 0.97 | 0.155 |
| 104348_VH5D98E_VK1 | 0.77 | 0.206 |
| 104348_VH5D98E_VK1D28Q | 1.18 | 0.272 |
| 104349_VH5_VK2D28Q | 0.43 | 0.069 |
| 104349_VH5D98E_VK2 | 0.56 | 0.178 |
| 104349_VH5D98E_VK2D28Q | 0.66 | 0.188 |
| 108923 | 0.05 | |
| 108924 | 0.11 | |
| 108925 | 0.06 | |
| 108926 | 0.05 | |
| 108928 | 0.71 | |
| 108929 | 0.15 | |
| 108930 | 0.06 | |

As can be seen in Table 23, most of the humanized antibodies have similar binding affinity to human IL-2 as NARA1. However, surprisingly, some humanized antibodies have lower binding affinity, which is the case of 104347.

### Example 8: Evaluation of IL-2/anti-IL-2 mAb Complex In Vitro

The activity of the humanized anti-IL-2 antibodies was compared to NARA1 in a PBMC derived-CD8 T cell proliferation assay.

Human CD8 T cells isolated by negative magnetic separation after Ficoll from Buffy-coat were plated at 100000 cells/well in complete RPMI medium supplemented with 5% human serum. Cells were stimulated for 48 hours at 37° C. with the anti-IL-2 antibody alone (0.5 μg/ml) or with IL-2 (Proleukin®; 0.1 μg/ml) plus anti-IL-2 antibodies (0.5 μg/ml) at a 2:1 molar ratio. Cells were pulsed for the last 16 hours with 3H-Thymidine, harvested and proliferation was measured with β-counter. The experiment was run in triplicates and the counts of the antibody alone were equivalent to the background signal level of unstimulated cells.

TABLE 24
CD8 T cell proliferation data

| | | [H3] Thymidine |
|---|---|---|
| | | Incorporation (cpm) |
| | [H3] Thymidine | Proleukin ® (0.1 μg/ml) + |
| | Incorporation (cpm) | Antibody (0.5 μg/ml) |
| Antibody | Antibody (0.5 μg/ml) | 2:1 molar ratio |
| NARA1 | 453 | 2766 |
| 104340 | 491 | 3363 |
| 104341 | 671 | 3463 |
| 104343 | 639 | 3457 |
| 104344 | 532 | 3799 |
| 104345 | 577 | 4874 |
| 104346 | 626 | 4607 |
| 104347 | 645 | 4636 |
| 104348 | 543 | 3360 |
| 104349 | 560 | 3348 |
| 104350 | 653 | 3974 |

As seen in Table 24 most of the humanized antibodies have a similar ability to NARA1 for induction of human CD8 T cell proliferation in vitro.

In an alternative method, the activity of some humanized anti-IL-2 antibodies was evaluated in 7-day proliferation assay in human PBMC derived-CD8 T and NK cells. Human PBMCs purified from buffy coats by Ficoll density gradient centrifugation were subjected to magnetic bead negative selection to isolate CD8⁺ T and NK cells. Cells were labelled with the CellTrace Violet Proliferation Kit and plated in 96 U-bottom plate at 50000 cells/well in RPMI 1640 Medium supplemented 10% fetal calf serum. Cells were then stimulated with hIL-2 or hIL-2/anti-IL-2 mAb complex (at a 2:1 molar ratio, 10-fold serial dilutions) and incubated for 7 days at 37° C. Proliferation was assessed by CellTrace Violet incorporation measured by FACS. The experiment was run in triplicates and average EC50 values were calculated from 2 independent experiments.

TABLE 25
CD8 T and NK cell proliferation data

| | hCD8 T Cell | hNK Cell |
|---|---|---|
| | Proliferation | Proliferation |
| IL-2/anti-IL-2 complex | EC50 (nM) | EC50 (nM) |
| Proleukin ® alone | 3.5 | 0.3 |
| Proleukin ®/ | 8.1 | 0.5 |
| 104343_VH1D98E_VK2D28Q | | |
| Proleukin ®/ | 9.2 | 0.5 |
| 104348_VH5D98E_VK1D28Q | | |
| Proleukin ®/ | 6.9 | 0.4 |
| 104349_VH5D98E_VK2D28Q | | |
| Proleukin ®/108923 | 19.1 | 1.6 |
| Proleukin ®/108924 | 18.5 | 1.6 |
| Proleukin ®/108925 | 12.1 | 1.1 |
| Proleukin ®/108926 | 14.8 | 1.1 |
| Proleukin ®/108929 | 11.9 | 0.8 |
| Proleukin ®/108930 | 12.1 | 1.0 |

### Example 9: Evaluation of IL-2/anti-IL-2 mAb Complex In Vivo

Counts of CD8⁺ T cells, CD4⁺ T cells, and NK cells were determined in WT C57BL/6 mice receiving IL-2/anti-IL-2 mAb complex as described below. In parallel, the proliferation levels of CD8⁺ T cells and NK cells were evaluated using bromodeoxyuridine (BrdU).

### (1) Materials and Methods

The following antibodies were used: 104340 (chimeric NARA1 antibody), 104343, 104347, 104348, 104349, 104341.

Proleukin® IL-2 was used.

This experiment was performed in duplicate; the first time the humanized 104341 was not included.

Mice received 4 consecutive injections of hIL-2 at 1.5 μg (low dose; LD) or 20 μg (high dose; HD), or hIL-2/monoclonal antibody (1.5 μg and 15 μg, respectively, corresponding to a 1:1 molar ratio). The day of the last injection 5-bromo-2′-deoxyuridine (BrdU) was given in the drinking water at 0.8 mg/ml for 24 hours. The following day, mice were sacrificed and spleens and lymph nodes (LNs) were analyzed by flow cytometry. To do so, single cell suspensions of LNs and spleens were prepared according to standard protocols and 2*10⁶ cells were stained for flow cytometry analysis using PBS with 2% fetal calf serum (FCS), 2 mM EDTA and fluorochrome-conjugated antibodies (see below).

Two different stains were performed: The first staining was done in order to identify and quantify CD4⁺ CD25⁺ forkhead box P3 (FoxP3)⁺ T regulatory cells. To this end, single cell suspensions were stained using the FoxP3 staining buffer and following the supplier's recommendations (eBiosciences, 00-5523-00) and using fluorochrome-conjugated antibodies to the following markers: CD25, CD8a, CD4, CD3, FoxP3.

A second staining was performed in order to identify and quantify cell proliferation of particular cell subsets whereby a fluorochrome-conjugated anti-BrdU antibody was used to stain cells that had proliferated. The BrdU stain was performed using the FITC BrdU kit and following the supplier's recommendations (BD Pharmingen, 51-2354 AK) and using fluorochrome-conjugated antibodies to the following markers: CD44, CD8a, CD4, NK1.1, CD3, CD122, Brdu.

Data was collected using a Becton Dickinson LSR Fortessa flow cytometer, well known to a person skilled in the art.

### (2) Results

The results of the cell count data is shown in Table 26 and Table 27.

TABLE 26
Cell count data
Cell counts (×10\hat{ }6)

| | CD3⁺CD8⁺ | | CD3⁺CD4⁺ | | CD3⁻NK1.1⁺ | |
|---|---|---|---|---|---|---|
| IL-2 LD | 13.41994 | 11.89759 | 19.95176 | 16.30791 | 2.042918 | 1.484522 |
| IL-2/104340 | 23.41843 | 25.82988 | 11.81956 | 11.59474 | 5.065738 | 5.448658 |
| IL-2/104343 | 14.45484 | 20.53916 | 18.5848 | 15.89161 | 5.286026 | 7.333515 |
| IL-2/104347 | 13.79718 | 14.83423 | 18.27195 | 21.03542 | 2.442164 | 2.597084 |
| IL-2/104348 | 23.68391 | 28.30016 | 14.75169 | 19.55284 | 7.37502 | 10.09379 |
| IL-2/104349 | 21.18129 | 24.34502 | 16.2933 | 14.88262 | 5.719003 | 11.14665 |
| IL-2/104341 | 27.68841 | 25.55466 | 19.12686 | 18.45614 | 13.83036 | 9.069159 |
| IL-2 HD | 21.4074 | 15.66434 | 27.66495 | 19.26853 | 5.97563 | 3.492621 |
| PBS | 8.776036 | 11.32218 | 12.99657 | 14.99693 | 0.730655 | 1.437321 |

TABLE 27
Cell count data
Cell counts (×10\hat{ }6)

| | CD3⁺CD8⁺CD44⁺ | | CD3⁺CD4⁺CD25⁺FoxP3⁺ | |
|---|---|---|---|---|
| IL-2 LD | 1.599656 | 1.169533 | 1.701715 | 1.612555 |
| IL-2/104340 | 14.98077 | 17.97501 | 3.949957 | 3.390384 |
| IL-2/104343 | 3.928826 | 7.53171 | 2.086871 | 3.359948 |
| IL-2/104347 | 2.127526 | 1.809776 | 2.563509 | 2.514805 |
| IL-2/104348 | 12.50984 | 12.61338 | 4.282856 | 4.927512 |
| IL-2/104349 | 7.286364 | 3.648101 | 3.578653 | 3.063865 |
| IL-2/104341 | 12.83635 | 11.69892 | 5.273673 | 4.708051 |
| IL-2 HD | 4.964377 | 3.648225 | 7.348498 | 4.172373 |
| PBS | 0.581851 | 1.25563 | 0.802762 | 1.019274 |

As can be seen in the Table 26 and Table 27, the antibodies 104343, 104347, 104348, 104349 and 104341 in complex with IL-2 can stimulate CD8 and NK cells in vivo.

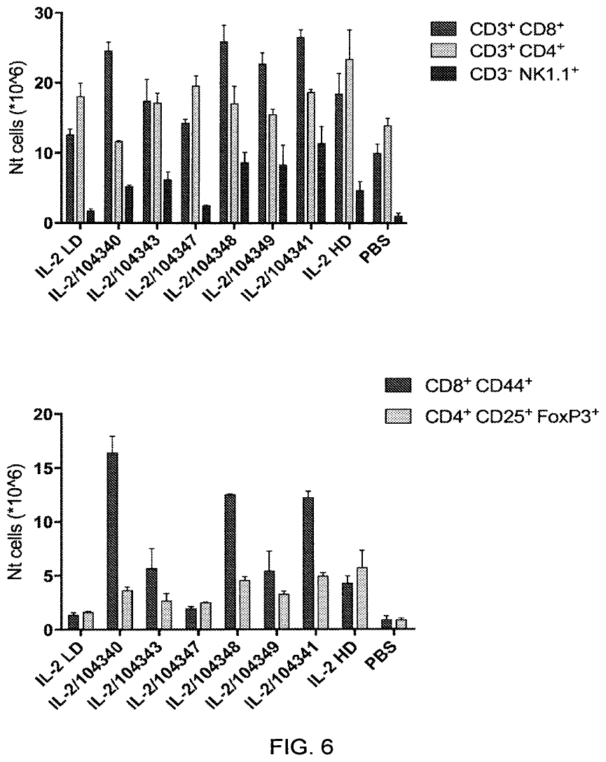

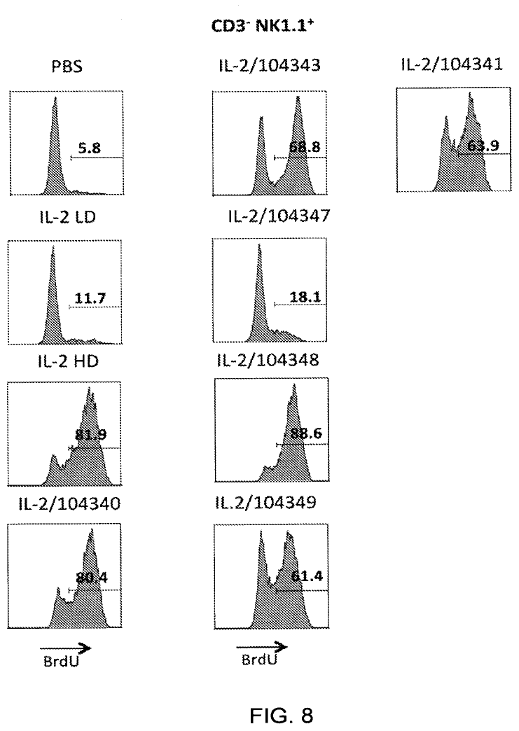

Also, FIG. 6 shows the number of immune cells obtained in the spleen of mice. Plotted values are seen in Table 23 and Table 24. FIG. 7 shows representative BrdU profiles of CD8⁺ CD44⁺ CD122⁺ T cells in the spleen of mice. FIG. 8 shows representative BrdU profiles of CD3-NK1.1⁺ NK cells in the spleen of mice.

From these results we can conclude that the humanized antibodies 104343, 104348, 104349 and 104341 like the chimeric NARA1 antibody 104340 in combination with IL-2 are able to preferentially stimulate CD8⁺ T cells and NK cells. This is not the case for the humanized 104347 antibody.

### Example 10: Generation of IL-2/Anti-IL-2 mAb Fusion Protein Using a Linker

Using the crystal structure NARA1/hIL-2 results of Example 2, ways have been identified for connecting NARA1 heavy and light chain N-terminal regions to hIL-2.

The structure of the complex was analyzed by using modeling software like PyMOL or MOE and it has been observed that the C-terminal end of hIL-2 is on opposite side of the antibody's antigen-binding site, thus linkers are needed for the connection. The resulting fusion protein is composed of IL-2 followed by a linker region followed by an antibody heavy chain region. The linker region has to cover a distance that is of least 60 Angstroms (Å), thus different linker's length and also composition may be designed and tested for optimal connection.

The fusion molecule can also be IL-2 followed by the linker followed by the light chain of an antibody. In this type of fusion, the linker region has to cover a distance of at least 50 Angstroms (Å), thus also here several linker's length and composition may be designed and tested for finding the optimal candidates.

The sequence of the antibody heavy or light chain can be any of the antibodies that were generated using a human IgG1 Fc domain, as represented by SEQ ID NO: 93, and the variable domains as reported in Table 10.

The IL-2 sequence can be wt IL-2, represented by SEQ ID NO: 109 or aldesleukin, represented by SEQ ID NO: 110.

The linker sequences that can be used to connect the C-terminal end of IL-2 and the N-terminal residue of the antibody heavy or light chains are reported in Table 28.

TABLE 28
Example of Linker's length and composition

| Linker | Sequence ID |
|---|---|
| (G4S)3 | SEQ ID NO: 397 |
| (G4S)4 | SEQ ID NO: 398 |
| (G4S)5 | SEQ ID NO: 399 |
| (G4S)6 | SEQ ID NO: 400 |
| (G4S)7 | SEQ ID NO: 401 |
| (G4S)8 | SEQ ID NO: 402 |
| (G4S)9 | SEQ ID NO: 403 |
| (G3S)4 | SEQ ID NO: 404 |
| (G3S)5 | SEQ ID NO: 405 |
| (G3S)6 | SEQ ID NO: 406 |
| (G3S)7 | SEQ ID NO: 407 |
| (G3S)8 | SEQ ID NO: 408 |
| (G3S)9 | SEQ ID NO: 409 |
| (G3S)10 | SEQ ID NO: 410 |
| (G3S)11 | SEQ ID NO: 411 |

Two specific fusion proteins were generated, according to Table 29.

TABLE 29
Fusion proteins

| Designation | IL-2 | Linker | mAb |
|---|---|---|---|
| 107348 | Aldesleukin | (G4S)5, 25 aa | VH5_D98E_VK1_{—} |
| | | (SEQ ID NO: 399) | D28Q-hIgG1 |
| 107350 | Aldesleukin | (G4S)7, 35 aa | VH5_D98E_VK1_{—} |
| | | (SEQ ID NO: 401) | D28Q-hIgG1 |

### Example 11: Generation of IL-2/Anti-IL-2 mAb Fusion Protein

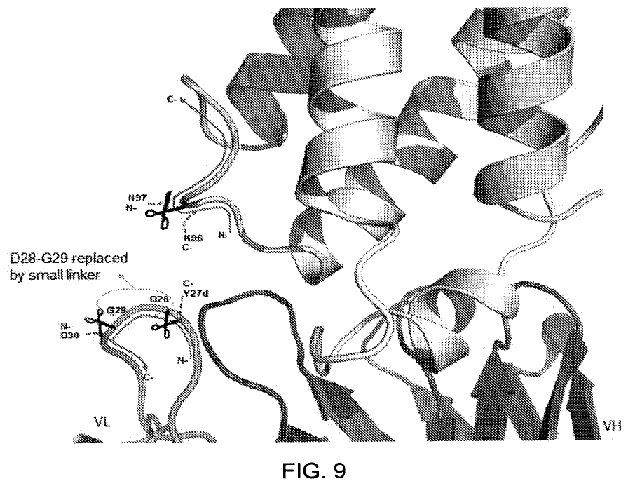

The structure of the complex NARA1/hIL-2 has been used to guide the embedding of IL-2 into the heavy or light chain of NARA1 antibody. The LCDR1 of the antibody and the region connecting hIL-2 helix B and helix C between residue K96 and N97, can be identified as regions to be used for further engineering. The LCDR1 was opened between Y27d and D28 and between G29 and D30, numbering according to Kabat definition. hIL-2 was opened between K96 and N97. See FIG. 9.

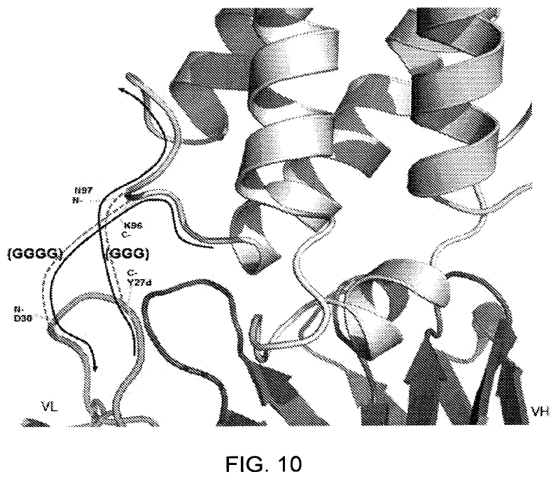

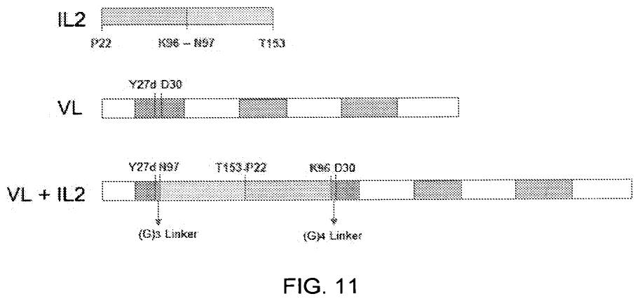

Residues D28G29 of LCDR1 of the antibody were replaced by Gs. hIL-2 new N-terminal end N97 is connected via a GGG linker to the new C-terminal end of LCDR1 Y27d. hIL-2 C-terminal end K96 is connected via a GGGG linker (SEQ ID NO: 412) to LCDR1 N-terminal D30. See FIG. 10. The two original N- and C-terminal ends of hIL-2 are connected together, i.e. IL-2-T153 C-terminal residue is directly fused to IL-2 N-terminal residue P22. FIG. 11 is a schematic overview of how the IL-2 and antibody VL sequences are fused together.

The resulting fusion protein was designated 107351.

This engineering procedure allowed hIL-2 to be completely embedded into the light chain of the NARA1 antibody. This embedding can be done by using any of the humanized sequences as reported in Table 10. The linker's sequence and length used to embed hIL-2 into the light chain may be a repetition of 1 to 10 of Glycine (G) (SEQ ID NO: 423), a fusion may be tested also in which LCDR1 Y27d and hIL-2 N97, LCDR1 D30 and hIL-2 K96 are directly connected without any linker sequence in between. The numbering of hIL-2 refers to the full length sequence as reported in SEQ ID: 109. The corresponding residues in aldesleukin, represented by SEQ ID NO: 110, can also be used for the embedding procedure.

### Example 12: Activity of IL-2/Anti-IL-2 mAb Fusion Protein

The activity of some IL-2/anti-IL-2 mAb fusion proteins was evaluated in 7-day proliferation assay in human PBMC derived-CD8 T and NK cells.

Human PBMCs purified from buffy coats by Ficoll density gradient centrifugation were subjected to magnetic bead negative selection to isolate CD8⁺ T and NK cells. Cells were labelled with the CellTrace Violet Proliferation Kit and plated in 96 U-bottom plate at 50000 cells/well in RPMI 1640 Medium supplemented 10% fetal calf serum. Cells were then stimulated with hIL-2 or hIL-2/anti-IL-2 mAb complex (at a 2:1 molar ratio, 10-fold serial dilutions) and incubated for 7 days at 37° C. Proliferation was assessed by CellTrace Violet incorporation measured by FACS. The experiment was run in triplicates and average EC50 values were calculated from 3 independent experiments, and are shown in Table 30.

TABLE 30
CD8 T and NK cell proliferation data

| | hCD8 T Cell | hNK Cell |
|---|---|---|
| IL-2/anti-IL-2 mAb | Proliferation | Proliferation |
| fusion protein | EC50 AVE (nM) | EC50 AVE (nM) |
| Proleukin ® alone | 3.24 | 0.34 |
| 107348 [L25 = (G4S)5] | >100 | 8.72 |
| (SEQ ID NO: 399) | | |
| 107350 [L35 = (G4S)7] | >100 | 2.21 |
| (SEQ ID NO: 401) | | |
| 107351 [LCDR1 graft] | 3.79 | 0.15 |

The IL-2/anti-IL-2 mAb fusion proteins using linker sequences of 25 or 35 residues have limited to no activity on NK cell proliferation and CD8 T cell proliferation, respectively. In contrast, the IL-2/anti-IL-2 mAb fusion protein grafted to the light chain of a humanized NARA1 antibody is as potent as IL-2 alone to activate CD8 T cell proliferation and more potent than IL-2 to stimulate NK cell proliferation.

This latter fusion protein was subsequently evaluated in vivo. Mice received one single dose of 107351 (360 μg/kg, 720 μg/kg or 1440 μg/kg) or PBS and were sacrificed 96h post-injection. Splenocytes were analyzed by flow cytometry following standard protocols, and the results are summarized in Table 31.

TABLE 31
CD8 T, NK and Treg cell counts

| | CD8+CD44+ | CD3−NK1.1+ | CD4+CD25+FoxP3+ |
|---|---|---|---|
| | T cell count | NK cell count | Treg cell count |
| Reagent | AVE (10⁶) | AVE (10⁶) | AVE (10⁶) |
| PBS | 1.5 | 1.3 | 0.5 |
| 107351 (360 | 41.5 | 17.3 | 1.1 |
| μg/kg) | | | |
| 107351 (720 | 57.2 | 24.8 | 1.5 |
| μg/kg) | | | |
| 107351 (1440 | 65.5 | 26.3 | 2.6 |
| μg/kg) | | | |

The fusion protein 107351 can induce in a dose-dependent manner a robust expansion of CD8⁺ and NK cells with very limited activation of regulatory T cells in vivo.

### Example 13: Evaluation of IL-2/Anti-IL-2 mAb Complex In Vivo (Humanized and Affinity-Matured Humanized Anti-hIL-2 Antibodies)

In order to evaluate the effect of IL-2/antil-IL-2 mAb Complex in vivo two experimental approaches were performed. In the first one, counts of CD8⁺ T cells, CD4⁺ T cells, and NK cells were determined in WT C57BL/6 mice receiving two injections of IL-2/anti-IL-2 mAb complex as described below. In the second experiment the proliferation levels of CD8⁺ T cells, CD4⁺ T cells and NK cells were evaluated using BrdU after one single injection of IL-2/antil-IL-2 mAb complexes.

### (1) Materials and Methods

The following antibodies were used: NARA1, 104348, 106260, 108923, 108924, 108925, 108926, 108929, and 108930.

Proleukin® IL-2 was used.

In the first experiment mice received 2 injections at days 1 and 3 of hIL-2 at 1.5 μg and hIL-2/monoclonal antibody (15 μg, corresponding to a 1:1 molar ratio). At day 5 mice were sacrificed and spleens and lymph nodes (LNs) were analyzed by flow cytometry. To do so, single cell suspensions of LNs and spleens were prepared according to standard protocols and 2-3*10⁶ cells were stained for flow cytometry analysis using PBS with 2% fetal calf serum (FCS), 2 mM EDTA and fluorochrome-conjugated antibodies. The stain was done in order to identify and quantify CD4⁺ CD25⁺ forkhead box P3 (FoxP3)⁺ T regulatory cells (Tregs). To this end, single cell suspensions were stained using the FoxP3 staining buffer and following the supplier's recommendations (eBiosciences, 00-5523-00) and using fluorochrome-conjugated antibodies to the following markers: CD25, CD8a, CD44, CD122, NK1.1, DX5, CD4, CD3, FoxP3.

In the second experiment, mice received one single injection of hIL-2 at 1.5 μg and hIL-2/monoclonal antibody (15 μg, corresponding to a 1:1 molar ratio). In parallel, BrdU was given in the drinking water at 0.8 mg/ml The following day (24 hours after injection and BrdU), stain was performed in order to identify and quantify cell proliferation of particular cell immune subsets whereby a fluorochrome-conjugated anti-BrdU antibody was used to stain cells that had proliferated. The BrdU stain was performed using the FITC BrdU kit and following the supplier's recommendations (BD Pharmingen, 51-2354 AK) and using fluorochrome-conjugated antibodies to the following markers: CD44, CD8a, CD4, NK1.1, CD3, CD122, Brdu, CD4.

Data was collected using a Becton Dickinson LSR Fortessa flow cytometer, well known to a person skilled in the art.

### (2) Results

The results of the cell count data is shown in Table 32 and Table 33.

TABLE 32
Cell count data

| | Cell counts (×10⁶) | | | | | |
|---|---|---|---|---|---|---|
| | CD3⁺ CD8⁺ | | CD3⁺ CD4⁺ | | CD3⁻ NK1.1⁺ DX5⁺ | |
| PBS | 2.495371 | 2.543511 | 3.8753 | 3.765718 | 0.936491 | 0.865639 |
| IL-2/NARA1 | 9.711202 | 24.05544 | 10.23144 | 12.94353 | 8.302392 | 27.26912 |
| (105192) | | | | | | |
| IL-2/104348 | 3.434657 | 3.326519 | 4.306137 | 4.117778 | 4.371078 | 2.262703 |
| IL-2/106260 | 4.819404 | 5.134374 | 5.958536 | 5.784579 | 4.108989 | 5.38501 |
| IL-2/108923 | 43.34794 | 24.56542 | 9.965975 | 8.123486 | 22.66742 | 20.29286 |
| IL-2/108924 | 12.17483 | 14.31066 | 6.465253 | 7.388885 | 11.60917 | 12.92698 |
| IL-2/108925 | 16.40202 | 19.57112 | 6.966743 | 7.345211 | 13.00835 | 16.15564 |
| IL-2/108926 | 15.40003 | 8.585168 | 5.453268 | 4.761522 | 15.08973 | 9.894915 |
| IL-2/108929 | 10.28953 | 10.05284 | 6.239835 | 3.892643 | 8.272205 | 10.35257 |
| IL-2/108930 | 16.0777 | 6.489082 | 6.878161 | 2.625737 | 17.55283 | 7.434181 |

TABLE 33
Cell count data

| | Cell counts (×10⁶) | | | |
|---|---|---|---|---|
| | CD3⁺ CD8⁺ CD44⁺ | | CD3⁺ CD4⁺ FoxP3⁺ | |
| PBS | 0.341866 | 0.278769 | 0.215854 | 0.1562773 |
| IL-2/NARA1 | 4.269044 | 15.16695 | 0.640488 | 1.695603 |
| (105192) | | | | |
| IL-2/104348 | 0.950713 | 0.959035 | 0.368605 | 0.3528936 |
| IL-2/106260 | 0.918578 | 1.969546 | 0.57619 | 0.6478728 |
| IL-2/108923 | 31.60932 | 15.84961 | 1.574624 | 1.186029 |
| IL-2/108924 | 6.8873 | 9.234667 | 0.724108 | 0.8127773 |
| IL-2/108925 | 10.79581 | 13.11657 | 0.766342 | 0.7146891 |
| IL-2/108926 | 9.517218 | 4.714974 | 0.504427 | 0.359971 |
| IL-2/108929 | 4.882382 | 5.851761 | 0.582177 | 0.4671171 |
| IL-2/108930 | 8.355582 | 4.038156 | 0.818501 | 0.3413458 |

TABLE 34
Ratios of Cell count data

Ratio cell counts
CD3⁺ CD8⁺ CD44⁺/CD3⁺ CD4⁺ FoxP3⁺

| | | |
|---|---|---|
| PBS | 1.583781 | 1.783809 |
| IL-2/NARA1 | 6.665295 | 8.944873 |
| (105192) | | |
| IL-2/104348 | 2.579217 | 2.717633 |
| IL-2/106260 | 1.594227 | 3.040019 |
| IL-2/108923 | 20.0742 | 13.36359 |
| IL-2/108924 | 9.511421 | 11.36187 |
| IL-2/108925 | 14.08746 | 18.35283 |
| IL-2/108926 | 18.86737 | 13.0982 |
| IL-2/108929 | 8.386427 | 12.5274 |
| IL-2/108930 | 10.20839 | 11.8301 |

As can be seen in Table 32, Table 33, and Table 34, the antibodies 104348, 106260, 108923, 108924, 108925, 108926, 108929 and 108930 in complex with IL-2 can preferentially stimulate CD8⁺ T cells and NK cells in vivo.

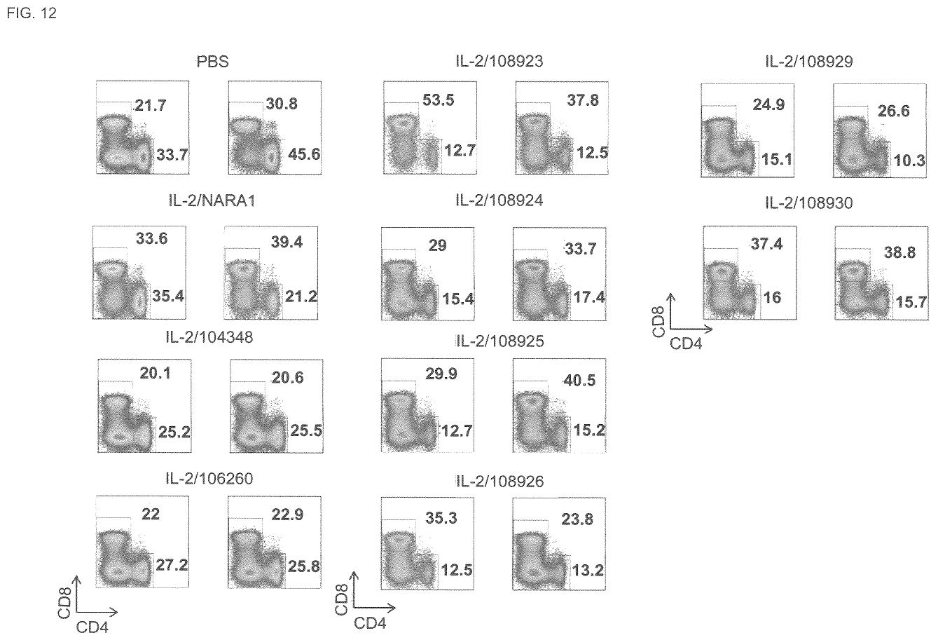

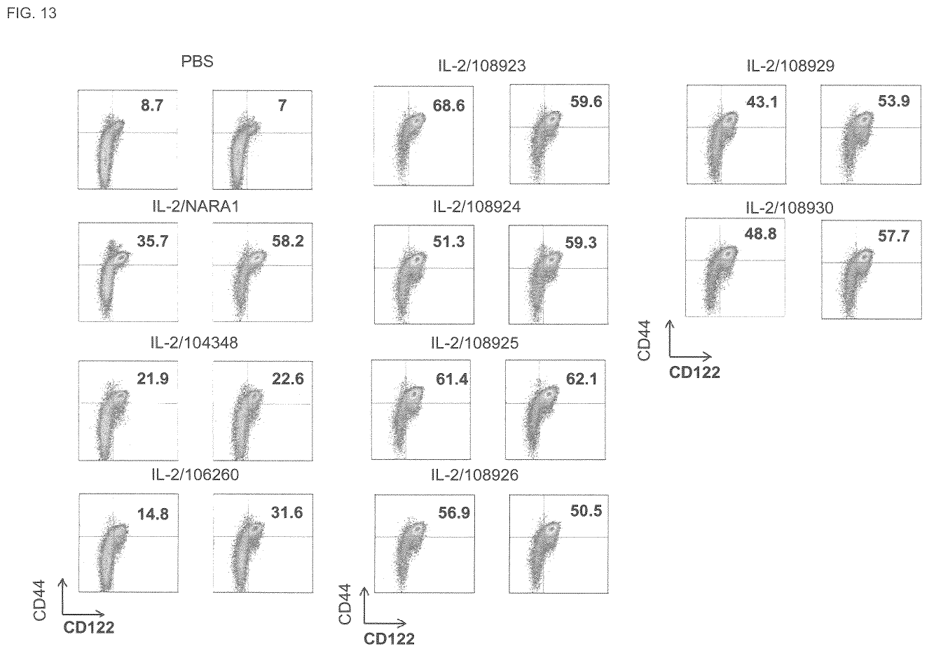

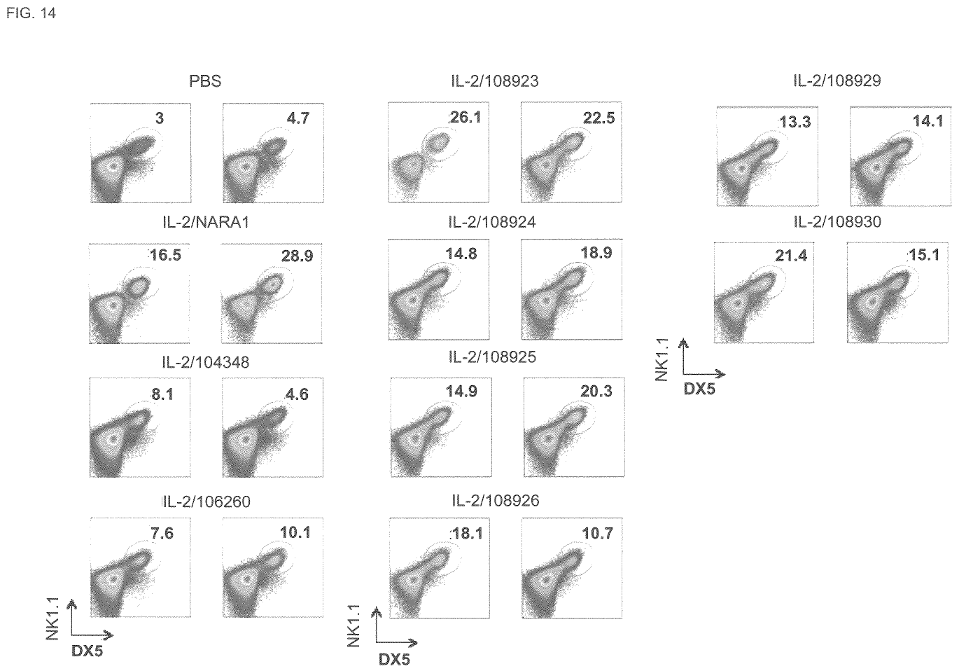

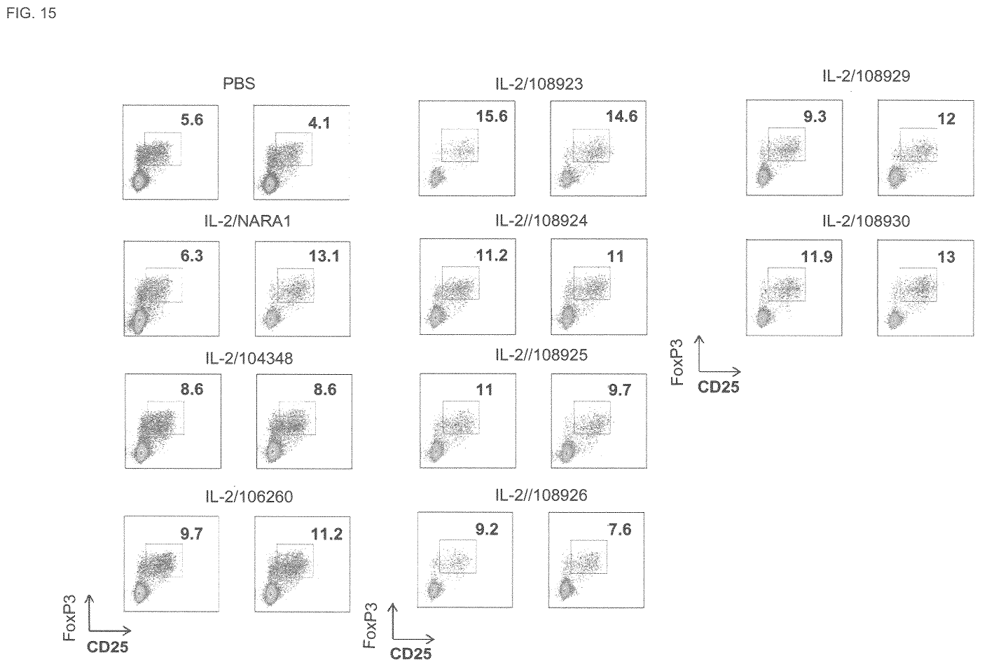

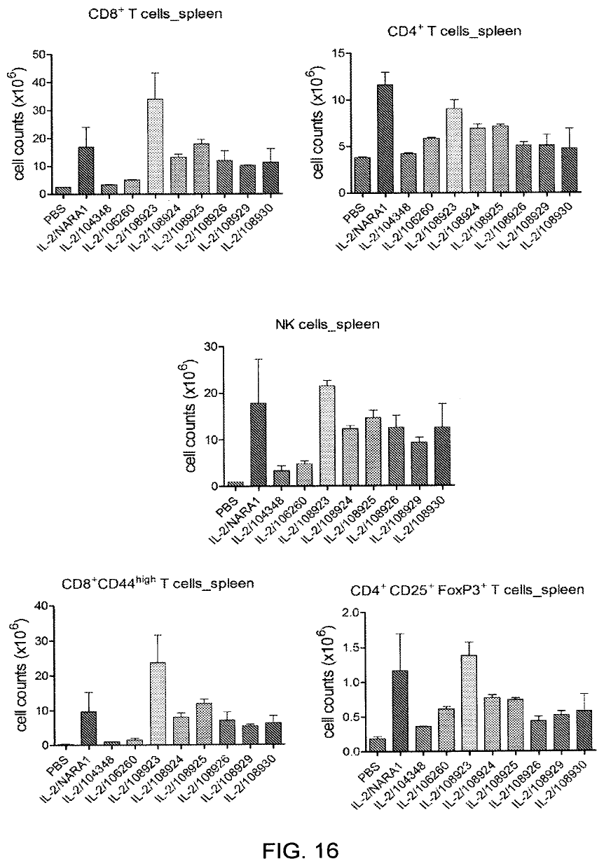

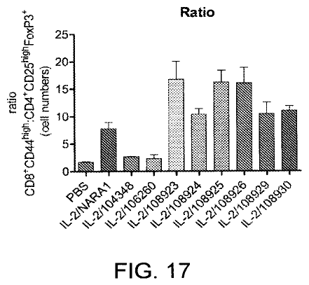

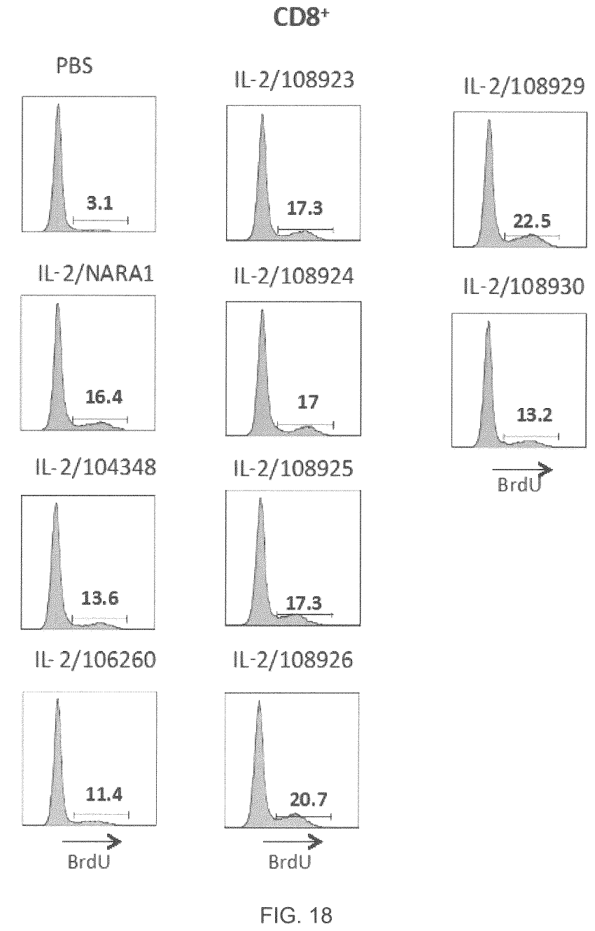

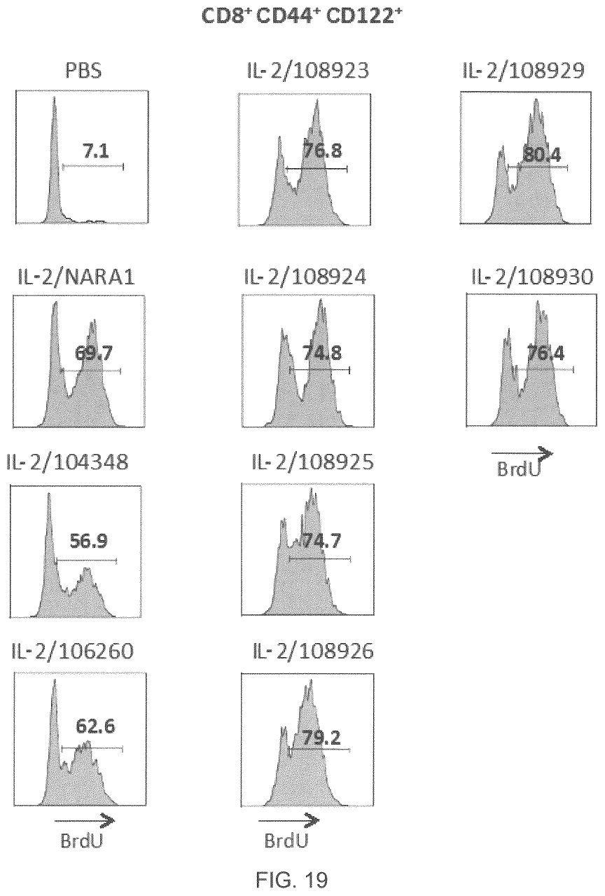

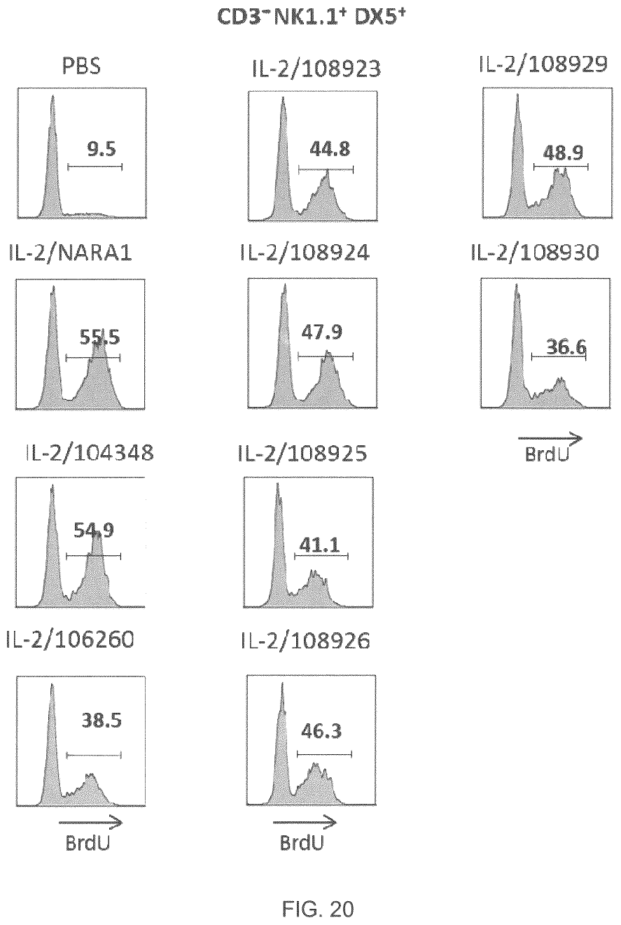

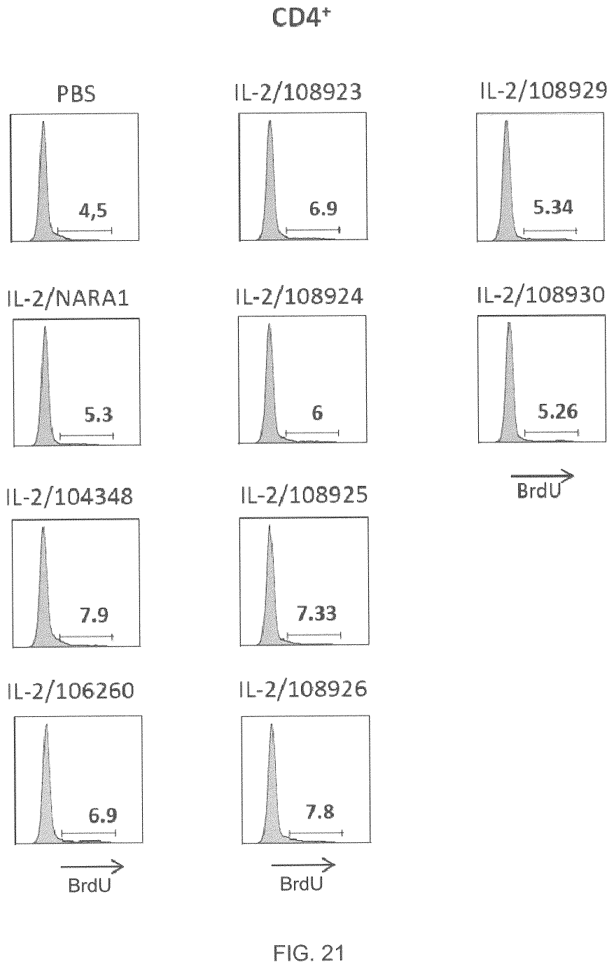

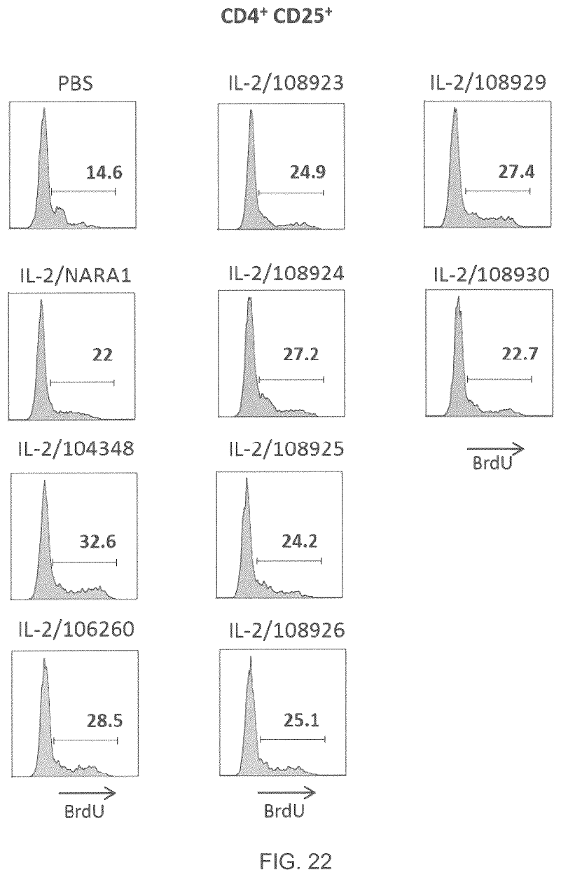

Also, FIGS. 12-15 show phenotypic characterization of endogenous CD8⁺, CD8⁺ CD44⁺ CD122⁺, CD4⁺ T cells, Treg cells and NK cells. FIGS. 16-17 show the number of immune cell subsets obtained in the spleen of mice; plotted values of Table 32, Table 33, and Table 34. FIGS. 18-22 show representative BrdU profiles of CD8⁺, CD8⁺ CD44⁺ CD122⁺ T cells, CD3-NK1.1⁺ DX5⁺ NK cells, CD4⁺ and CD4⁺ CD25⁺ T cells, in the spleen of mice. As we can see the humanized antibodies 104348, 106260 and the affinity matured humanized antibodies 108923, 108924, 108925, 108926, 108929 and 108930 in complex with IL-2 enhance the proliferation levels of CD8⁺ T cells (especially of CD8⁺ CD44⁺ CD122⁺ T cells) and NK cells in vivo over CD4⁺ and CD4⁺ CD25⁺ T cells.

From these results we can conclude that the humanized antibodies 104348, 106260, and the affinity matured humanized antibodies 108923, 108924, 108925, 108926, 108929 and 108930 similar to the parental NARA1 antibody (105192), when mixed with hIL-2 (Proleukin®), are able to preferentially stimulate CD8⁺ T cells and NK cells.

### Example 14: Generation of Expression Plasmids of NARA1-Based Fusion Proteins

DNA sequences coding for human IL-2 (pORF-hIL-2 palsmid, Invivogen, porf-hIL-2) and the light chain of NARA1 were subcloned by cut and paste and ligated using standard cloning techniques (PCR amplification/assembly using phusion polymerase, Finnzymes, F-530S) adding within the primers the linkers aimed for (e.g 15, 20 or 25 amino acid linkers as shown in tables Table 35, Table 36, and Table 37). The resulting PCR products were inserted by cut and paste into expression vectors suitable for secretion in mammalian cells. The heavy chain of NARA1 and light chain of NARA1 fused to hIL-2 were cloned into individual expression vectors to allow co-transfection. Elements of the expression vector include a promoter (Cytomegalovirus (CMV) enhancer-promoter), a signal sequence to facilitate secretion, a polyadenylation signal and transcription terminator (Bovine Growth Hormone (BGH) gene), an element allowing episomal replication and replication in prokaryotes (e.g. SV40 origin and CoIE1 or others known in the art) and elements to allow selection (ampicillin resistance gene and zeocin marker).

TABLE 35
Light regions

| Light region | Sequence listing |
|---|---|
| IL-2-(G4S)₃- L (NARA1) | SEQ ID NO: 413 (DNA) |
| IL-2-(G4S)₄- L (NARA1) | SEQ ID NO: 414 (DNA) |
| IL-2-(G4S)₅- L (NARA1) | SEQ ID NO: 415 (DNA) |

### Example 15: Expression and Purification of NARA1-Based Fusion Proteins

Chinese hamster Ovary (CHO) cells are one of the preferred host cell lines for transient expression of IgG proteins. Transfection is performed using PEI (Polyethylenimine, MW 25.000 linear, Polysciences, USA Cat. No. 23966) as transfection reagent. The PEI stock solution is prepared by carefully dissolving 1 g of PEI in 900 ml cell culture grade water at room temperature (RT). To facilitate dissolution of PEI, the solution is acidified by addition of HCl to pH 3-5, followed by neutralization with NaOH to a final pH of 7. Finally, the volume is adjusted to 1L and the solution is filtered through a 0.22 μm filter, aliquoted and frozen at −80° C. until further use. CHO cells are cultivated using Power CHO-2D medium and ProCHO-4 medium (serum-free culture mediums for propagation and transfection respectively, 12-770Q and 12-029Q Lonza) with sodium hypoxanthine and thymidine (HT, 41065012, Invitrogen), L-Glutamine (17-605C, Lonza) and antibiotic-antimycotic (15240062, ThermoFisher) as supplements. Cells prepared for transient transfections are cultivated in suspension culture on a shaker at 150 rpm. Cells in the seed cultures should be maintained in the exponential growth phase (cell densities between 1.5×10⁵ and 3×10⁶/mL) and display a viability of >98% for transfection. Cell densities outside of this range will result in either a lag phase after dilution or reduced transfection efficiency. For transfections an aliquot of cells is taken out of the seed cultures and adjusted to 2×10⁶ cells/mL in 50% of the final volume with Power CHO-4 serum-free culture medium. The DNA solution (total DNA=125 μg for 100 ml scale, adjusted to 1:1 molar ratio of the heavy chain and the light chain(s) fused to IL-2) is prepared by diluting the DNA in 150 mM NaCL solution. Then PEI solution (0.5 ml for 100 ml scale) is added to the DNA solution. The mixture is vortexed and incubated for 10⁻¹² min at room temperature. The transfection mix is then added to the cells and the cultivation of cells is continued for 3 to 4 hours. Finally, the remaining 50% of total production volume are achieved by addition of Power CHO-2D serum-free culture medium. The cell cultivation is continued for four to six days post transfection. The culture is harvested by centrifugation at 4700 rpm for 45 minutes at 4° C. (Heraeus®, Multifuge 3 S-R, Thermo Scientific, Rockford, IL). The cell supernatant recovered is sterile filtered through a stericup filter (0.22 μm) and stored at 4° C. with 2.5 mM ETDA and sodium azide (0.01%) until further processing.

Purification was performed using protein G agarose (20397, Thermo Scientific) at 4° C. Accordingly, three fusion proteins where the C terminus of IL-2 was fused to the N-terminus of the light chain of NARA1 with Glycin-Serin linkers of 15, 20 or 25 amino acids length were generated, as highlighted in Table 36. The heavy chain of NARA1 remains the same (SEQ ID NO: 115).

TABLE 36
Light regions

| Light region | Sequence listing |
|---|---|
| IL-2-(G4S)₃- L (NARA1) | SEQ ID NO: 416 |
| IL-2-(G4S)₄- L (NARA1) | SEQ ID NO: 417 |
| IL-2-(G4S)₅- L (NARA1) | SEQ ID NO: 418 |

TABLE 37
NARA1-based fusion protein

| Fusion | | | Heavy | |
|---|---|---|---|---|
| Protein | Light region | Light SEQ ID | region | Heavy SEQ ID |
| L15 | IL-2-(G4S)₃- | SEQ ID NO: 416 | H1 | SEQ ID NO: 115 |
| | L (NARA1) | (AA) | | |
| L20 | IL-2-(G4S)₄- | SEQ ID NO: 417 | H1 | SEQ ID NO: 115 |
| | L (NARA1) | (AA) | | |
| L25 | IL-2-(G4S)₅- | SEQ ID NO: 418 | H1 | SEQ ID NO: 115 |
| | L (NARA1) | (AA) | | |

### Example 16: Evaluation of the NARA1-Based Fusion Proteins In Vivo

Percentages and counts of CD8⁺ T cells, CD4⁺ T cells, and NK cells were determined in WT C57BL/6 mice receiving L15, L20, L25, or IL-2/NARA1 as described below. In parallel, the proliferation levels of CD8⁺ T cells and NK cells were evaluated using bromodeoxyuridine (BrdU).

### (1) Materials and Methods

The following antibodies and fusion proteins were used: NARA1, L15, L20 and L25.

Recombinant human IL-2 (Teceleukin®) was used.

Mice received 3 consecutive injections of hIL-2 at 2 μg (20′000 IU) with hIL-2/monoclonal antibody (NARA1, 10 μg, corresponding to a 2:1 molar ratio), L15, L20, L25 (the corresponding IL-2 amount in term of activity) or PBS. The day of the last injection BrdU was given in the drinking water at 0.8 mg/ml for 24 hours. The following day, mice were sacrificed and spleens and lymph nodes (LNs) were analyzed by flow cytometry. To do so, single cell suspensions of LNs and spleens were prepared according to standard protocols and 2*10⁶ cells were stained for flow cytometry analysis using PBS with 2% fetal calf serum (FCS), 2 mM EDTA and fluorochrome-conjugated antibodies (see below).

Two different stains were performed: The first staining was done in order to identify and quantify CD4⁺ CD25⁺ forkhead box P3 (FoxP3)⁺ T regulatory cells. To this end, single cell suspensions were stained using the FoxP3 staining buffer and following the supplier's recommendations (eBiosciences, 00-5523-00) and using fluorochrome-conjugated antibodies to the following markers: CD25, CD8a, CD4, CD3, FoxP3.

A second staining was performed in order to identify and quantify cell proliferation of particular cell subsets whereby a fluorochrome-conjugated anti-BrdU antibody was used to stain cells that had proliferated. The BrdU stain was performed using the FITC BrdU kit and following the supplier's recommendations (BD Pharmingen, 51-2354 AK) and using fluorochrome-conjugated antibodies to the following markers: CD44, CD8a, CD4, NK1.1, CD3, CD122, Brdu.

Data was collected using a Becton Dickinson LSR Fortessa flow cytometer, well known to a person skilled in the art.

### (2) Results

The results of the cell count data are shown in Table 38.

TABLE 38
Cell counts

| | CD8⁺CD44⁺ | | CD3⁻NK1.1⁺DX5⁺ | | CD4⁺CD25⁺FoxP3⁺ | |
|---|---|---|---|---|---|---|
| PBS | 2.988014 | 1.905477 | 2.636173 | 2.153964 | 1.704692 | 1.612001 |
| hIL-2/NARA1 | 11.58204 | 12.41452 | 4.885715 | 5.945634 | 3.763952 | 4.461254 |
| L15 | 21.08623 | 9.229327 | 6.761334 | 4.056121 | 8.416243 | 7.998698 |
| L20 | 12.9221 | 15.2443 | 7.870497 | 6.671924 | 8.024002 | 3.606802 |
| L25 | 12.36608 | 14.4394 | 6.9261 | 6.736377 | 7.07191 | 5.494133 |

As can be seen in the Table 38 the fusion proteins L15, L20 and L25 can stimulate CD8⁺ T cells and NK cells in vivo.

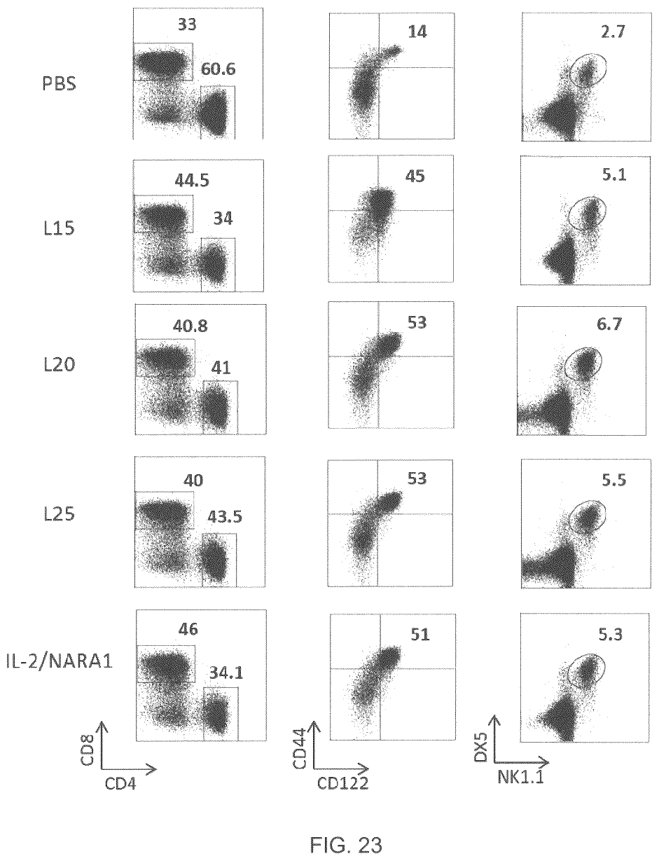

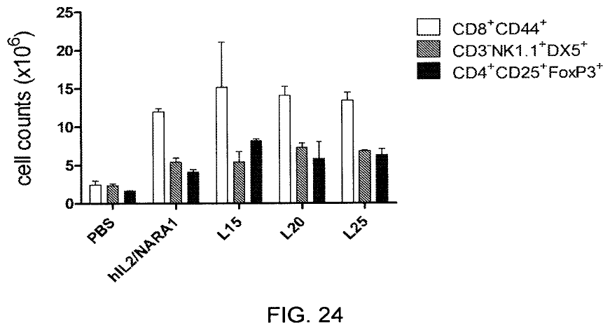

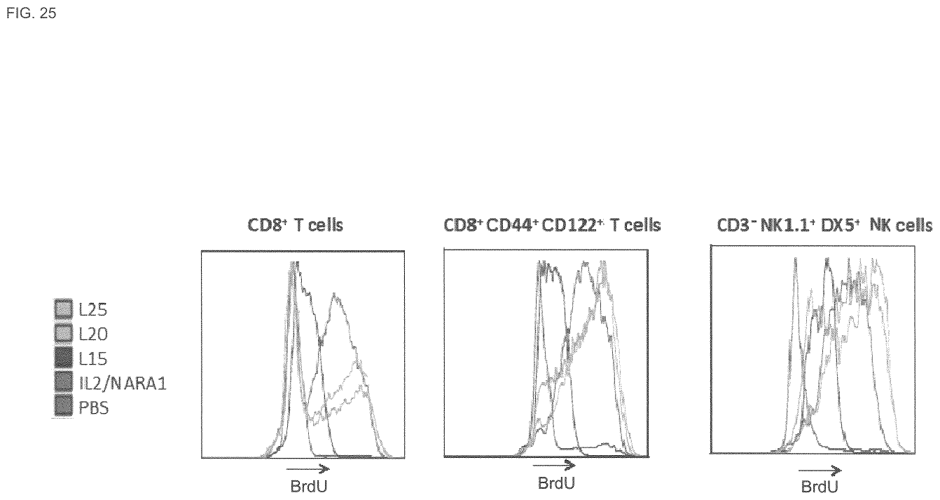

Also, FIG. 23 shows phenotypic characterization of endogenous CD8⁺ T cells and NK cells. FIG. 24 shows the number of immune cells obtained in the spleen of mice, plotted values of Table 38. FIG. 25 shows representative BrdU profiles of CD8⁺ CD44⁺ CD122⁺ T cells and CD3-NK1.1⁺ NK cells in the spleen of mice.

From these results we can conclude that the NARA1-based fusion proteins are able to preferentially stimulate CD8⁺ T cells and NK cells, similar to the parental NARA1 antibody in combination with human IL-2.

### Example 17: Evaluation of the NARA1-Based Fusion Proteins In Vitro

The activity of NARA1-based fusion proteins was compared to NARA1 and IL-2 alone in a cell proliferation assay using CTLL-2 murine cell lines responsive to human IL-2. CTLL-2 cells were seeded into 96-well plates (10000 cells/well) and stimulated using, hIL-2, or hIL-2/NARA1 complex (2:1 molar ratio), L15, L20 or L25 (the corresponding IL-2 amount) and proliferation was assessed after 48 hours of incubation at 37° C. Proliferation was assessed by adding WST-1 (Sigma-Aldrich) for the least 4 hours to the cells, followed by reading at 450 nm on an iMark microplate reader.

The experiment was run in duplicates and Table 39 provides the EC50 values obtained.

TABLE 39
CTTL-2 cell proliferation data (EC50)

| | L15 | L20 | L25 | hIL-2 | hIL-2/Nara1 |
|---|---|---|---|---|---|
| EC50 (ng/ml) | 2.7 | 1.6 | 2.6 | 0.8 | 0.7 |

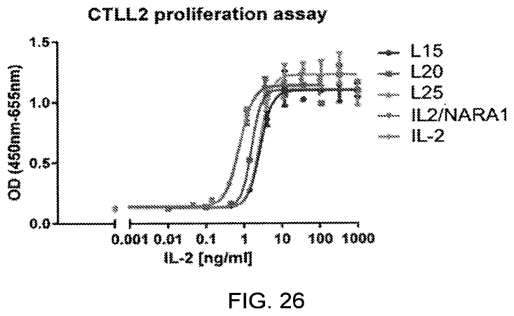

As seen in Table 39 the NARA1-based fusion proteins have the ability to induce CTLL-2 cell proliferation in vitro but to a lower extent compared to hIL-2 and hIL-2/NARA1. Also, FIG. 26 shows the proliferation curves obtained.

The activity of the NARA1-based fusion proteins was compared to NARA1 and IL-2 alone in a STAT5 phosphorylation (P-STAT5) assay using CTLL-2 murine cell lines responsive to human IL-2. CTLL-2 (200000 cells/well) were seeded in 96-well plates and stimulated using hIL-2 hIL-2/NARA1 complex (at 2:1 molar ratio), L15, L20 or L25 (the corresponding IL-2 amount). Phosphorylation of STAT5 was assessed after 15 minutes of stimulation and detected using intracellular staining with pSTAT5-specific mAbs (BD Biosciences). The experiment was run in duplicates and Table 40 provides the EC50 values obtained.

TABLE 40
P-STAT5 (EC50)

| | hIL-2 | hIL-2/NARA1 | L15 | L20 | L25 |
|---|---|---|---|---|---|
| EC50 (ng/ml) | 1.03 | 1.4 | 107.2 | ~426.6 | ~434.8 |

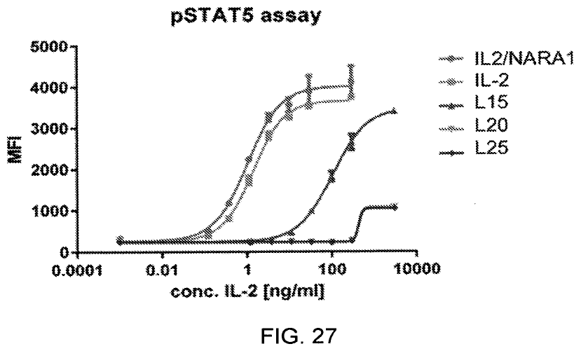

Also, FIG. 27 provides the phosphorylation levels obtained. As observed the in vitro effect obtained on cells expressing high CD25 levels such as CTLL-2 cells is lower when stimulating the cells with L15, L20 and L25 than with IL-2 or IL-2/NARA1. This difference is more prominent for a short time (such as 15 minutes stimulation for P-STAT5). Indeed, with the shortest linker: L15, IL-2 might dissociate easier to NARA1 allowing the stimulation of cells in a short time. On the other hand, for the fusions L20 and L25, IL-2 might remain bound longer to NARA1 preventing the stimulation of CTLL-2 cells.

### Sequence Table

Useful amino acids and nucleotide sequences for practicing the invention are found in Table 41. Throughout the text of this application, should there be a discrepancy between the text of the specification (e.g., Table 41) and the sequence listing, the text of the specification shall prevail.

TABLE 41
Sequence list

| | | |
|---|---|---|
| >VH1 | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 3 (Combined) | HCDR3 | WRGDGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 3 (Kabat) | HCDR3 | WRGDGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 3 (Chothia) | HCDR3 | WRGDGYYAYFDV |
| SEQ ID NO: 7 | VH | QVQLVQSGAEVKKPGSSVKVSCKASGYAFTNYLIEWVR |
| | | QAPGQGLEWMGVINPGSGGTNYNEKFKGRVTITADKS |
| | | TSTAYMELSSLRSEDTAVYYCARWRGDGYYAYFDVWG |
| | | QGTTVTVSS |
| SEQ ID NO: 8 | DNA VH | CAAGTGCAGCTGGTGCAGTCTGGCGCTGAAGTGAAG |
| | | AAACCCGGCTCCTCCGTGAAAGTGTCCTGCAAGGCCT |
| | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGTGCCCGGTG |
| | | GCGGGGAGATGGCTACTACGCCTACTTCGACGTGTGG |
| | | GGCCAGGGCACCACCGTGACCGTGTCCTCT |
| >VH3 | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 3 (Combined) | HCDR3 | WRGDGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 3 (Kabat) | HCDR3 | WRGDGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 3 (Chothia) | HCDR3 | WRGDGYYAYFDV |
| SEQ ID NO: 9 | VH | QVQLVESGGGVVQPGRSLRLSCAASGYAFTNYLIEWVR |
| | | QAPGKGLEWVAVINPGSGGTNYNEKFKGRFTISADKSKS |
| | | TAYLQMNSLRAEDTAVYYCARWRGDGYYAYFDVWGQ |
| | | GTTVTVSS |
| SEQ ID NO: 10 | DNA VH | CAGGTGCAGCTGGTGGAGAGCGGCGGCGGCGTGGTG |
| | | CAGCCCGGCCGGAGCCTGCGGCTGAGCTGCGCCGCC |
| | | AGCGGCTACGCCTTCACCAACTACCTGATCGAGTGGG |
| | | TGCGGCAGGCCCCCGGCAAGGGCCTGGAGTGGGTGG |
| | | CCGTGATCAACCCCGGCAGCGGCGGCACCAACTACAA |
| | | CGAGAAGTTCAAGGGCCGGTTCACCATCAGCGCCGAC |
| | | AAGAGCAAGAGCACCGCCTACCTGCAGATGAACAGCC |
| | | TGCGGGCCGAGGACACCGCCGTGTACTACTGCGCCCG |
| | | GTGGCGGGGCGACGGCTACTACGCCTACTTCGACGTG |
| | | TGGGGCCAGGGCACCACCGTGACCGTGAGCAGC |
| >VH3s | | |
| SEQ ID NO: 11 (Combined) | HCDR1 | GYTFSSYLIE |
| SEQ ID NO: 12 (Combined) | HCDR2 | VINPGSGGTNYADSVKG |
| SEQ ID NO: 3 (Combined) | HCDR3 | WRGDGYYAYFDV |
| SEQ ID NO: 13 (Kabat) | HCDR1 | SYLIE |
| SEQ ID NO: 12 (Kabat) | HCDR2 | VINPGSGGTNYADSVKG |
| SEQ ID NO: 3 (Kabat) | HCDR3 | WRGDGYYAYFDV |
| SEQ ID NO: 14 (Chothia) | HCDR1 | GYTFSSY |
| SEQ ID NO: 6 (Chothia) | HCDR2 | NPGSGG |
| SEQ ID NO: 3 (Chothia) | HCDR3 | WRGDGYYAYFDV |
| SEQ ID NO: 15 | VH | QVQLVESGGGVVQPGRSLRLSCAASGYTFSSYLIEWVRQ |
| | | APGKGLEWVAVINPGSGGTNYADSVKGRFTISADKSKN |
| | | TAYLQMNSLRAEDTAVYYCARWRGDGYYAYFDVWGQ |
| | | GTTVTVSS |
| SEQ ID NO: 16 | DNA VH | CAGGTGCAATTGGTGGAAAGCGGCGGAGGCGTGGTG |
| | | CAGCCTGGAAGAAGCCTGAGACTGAGCTGTGCCGCCA |
| | | GCGGCTACACCTTCAGCAGCTACCTGATCGAGTGGGT |
| | | GCGCCAGGCCCCTGGCAAAGGACTGGAATGGGTGGC |
| | | CGTGATCAACCCTGGCAGCGGCGGCACCAATTACGCC |
| | | GATAGCGTGAAGGGCCGGTTCACCATCAGCGCCGACA |
| | | AGAGCAAGAACACCGCCTACCTCCAGATGAACAGCCT |
| | | GCGGGCCGAGGACACCGCCGTGTACTATTGTGCTCGG |
| | | TGGCGGGGAGATGGCTACTACGCCTACTTCGACGTGT |
| | | GGGGCCAGGGCACCACAGTGACCGTCAGCTCA |
| >VH5 | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 3 (Combined) | HCDR3 | WRGDGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 3 (Kabat) | HCDR3 | WRGDGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 3 (Chothia) | HCDR3 | WRGDGYYAYFDV |
| SEQ ID NO: 17 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGDGYYAYFDVWGQ |
| | | GTTVTVSS |
| SEQ ID NO: 18 | DNA VH | GAAGTGCAGCTGGTGCAGTCTGGCGCTGAAGTGAAG |
| | | AAGCCCGGCGAGTCCCTGAAGATCTCCTGCAAGGGCT |
| | | CCGGCTACGCCTTCACCAACTACCTGATCGAGTGGGTC |
| | | CGACAGATGCCCGGCAAGGGCCTGGAGTGGATGGGC |
| | | GTGATCAACCCCGGCTCCGGCGGCACCAACTACAACG |
| | | AGAAGTTCAAGGGCCAAGTCACAATCTCCGCCGACAA |
| | | GTCCATCTCCACCGCCTACCTGCAGTGGTCCTCCCTGA |
| | | AGGCCTCCGACACCGCCATGTACTACTGCGCCAGATG |
| | | GCGGGGAGATGGCTACTACGCCTACTTCGACGTGTGG |
| | | GGCCAGGGCACCACCGTGACCGTGTCCTCT |
| >VK1 | | |
| SEQ ID NO: 19 (Combined) | LCDR1 | KASQSVDYDGDSYMN |
| SEQ ID NO: 20 (Combined) | LCDR2 | AASNLES |
| SEQ ID NO: 21 (Combined) | LCDR3 | QQSNEDPYT |
| SEQ ID NO: 19 (Kabat) | LCDR1 | KASQSVDYDGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 22 (Chothia) | LCDR1 | SQSVDYDGDSY |
| SEQ ID NO: 23 (Chothia) | LCDR2 | AAS |
| SEQ ID NO: 24 (Chothia) | LCDR3 | SNEDPY |
| SEQ ID NO: 25 | VL | AIRLTQSPSSFSASTGDRVTITCKASQSVDYDGDSYMNW |
| | | YQQKPGKAPKLLIYAASNLESGVPSRFSGSGSGTDFTLTIS |
| | | SLQSEDFATYYCQQSNEDPYTFGGGTKVEIK |
| SEQ ID NO: 26 | DNA VL | GCCATCAGACTGACCCAGAGCCCCTCCAGCTTCTCCGC |
| | | CTCCACCGGCGACAGAGTGACCATCACATGCAAGGCC |
| | | TCCCAGTCCGTGGACTACGACGGCGACTCCTACATGA |
| | | ACTGGTATCAGCAGAAGCCCGGCAAGGCCCCTAAGCT |
| | | GCTGATCTACGCCGCCTCCAACCTGGAATCCGGCGTG |
| | | CCCTCCCGGTTCTCCGGCTCTGGCTCTGGCACCGACTT |
| | | CACCCTGACCATCTCCAGCCTGCAGTCCGAGGACTTCG |
| | | CCACCTACTACTGCCAGCAGTCCAACGAGGACCCCTAC |
| | | ACCTTCGGCGGAGGCACCAAAGTGGAAATCAAG |
| >VK2 | | |
| SEQ ID NO: 19 (Combined) | LCDR1 | KASQSVDYDGDSYMN |
| SEQ ID NO: 20 (Combined) | LCDR2 | AASNLES |
| SEQ ID NO: 21 (Combined) | LCDR3 | QQSNEDPYT |
| SEQ ID NO: 19 (Kabat) | LCDR1 | KASQSVDYDGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 22 (Chothia) | LCDR1 | SQSVDYDGDSY |
| SEQ ID NO: 23 (Chothia) | LCDR2 | AAS |
| SEQ ID NO: 24 (Chothia) | LCDR3 | SNEDPY |
| SEQ ID NO: 27 | VL | DIVLTQSPLSLPVTLGQPASISCKASQSVDYDGDSYMNW |
| | | YQQRPGQSPRLLIYAASNLESGVPDRFSGSGSGTDFTLKI |
| | | SRVEAEDVGVYYCQQSNEDPYTFGGGTKVEIK |
| SEQ ID NO: 28 | DNA VL | GACATCGTGCTGACACAGAGCCCTCTGTCCCTGCCCGT |
| | | GACCCTGGGCCAGCCTGCCTCCATCTCCTGCAAGGCCT |
| | | CCCAGTCCGTGGACTACGACGGCGACTCCTACATGAA |
| | | CTGGTATCAGCAGCGGCCTGGCCAGTCCCCTCGGCTG |
| | | CTGATCTACGCCGCCTCCAACCTGGAATCCGGCGTGCC |
| | | CGACAGATTCTCCGGCTCCGGCTCTGGCACCGACTTCA |
| | | CCCTGAAGATCTCCCGGGTGGAAGCCGAGGACGTGG |
| | | GCGTGTACTACTGCCAGCAGTCCAACGAGGACCCCTA |
| | | CACCTTCGGCGGAGGCACCAAAGTGGAAATCAAG |
| >VK3 | | |
| SEQ ID NO: 19 (Combined) | LCDR1 | KASQSVDYDGDSYMN |
| SEQ ID NO: 20 (Combined) | LCDR2 | AASNLES |
| SEQ ID NO: 21 (Combined) | LCDR3 | QQSNEDPYT |
| SEQ ID NO: 19 (Kabat) | LCDR1 | KASQSVDYDGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 22 (Chothia) | LCDR1 | SQSVDYDGDSY |
| SEQ ID NO: 23 (Chothia) | LCDR2 | AAS |
| SEQ ID NO: 24 (Chothia) | LCDR3 | SNEDPY |
| SEQ ID NO: 29 | VL | EIVLTQSPATLSVSPGERATLSCKASQSVDYDGDSYMNW |
| | | YQQKPGQAPRLLIYAASNLESGIPARFSGSGSGTEFTLTIS |
| | | SLQSEDAAVYYCQQSNEDPYTFGGGTKVEIK |
| SEQ ID NO: 30 | DNA VL | GAGATCGTGCTGACCCAGAGCCCCGCCACCCTGAGCG |
| | | TGAGCCCCGGCGAGCGGGCCACCCTGAGCTGCAAGG |
| | | CCAGCCAGAGCGTGGACTACGACGGCGACAGCTACAT |
| | | GAACTGGTACCAGCAGAAGCCCGGCCAGGCCCCCCG |
| | | GCTGCTGATCTACGCCGCCAGCAACCTGGAGAGCGGC |
| | | ATCCCCGCCCGGTTCAGCGGCAGCGGCAGCGGCACCG |
| | | AGTTCACCCTGACCATCAGCAGCCTGCAGAGCGAGGA |
| | | CGCCGCCGTGTACTACTGCCAGCAGAGCAACGAGGAC |
| | | CCCTACACCTTCGGCGGCGGCACCAAGGTGGAGATCA |
| | | AG |
| >VK3s | | |
| SEQ ID NO: 31 (Combined) | LCDR1 | RASQSVSYDGDSYMN |
| SEQ ID NO: 32 (Combined) | LCDR2 | AASNLAS |
| SEQ ID NO: 21 (Combined) | LCDR3 | QQSNEDPYT |
| SEQ ID NO: 31 (Kabat) | LCDR1 | RASQSVSYDGDSYMN |
| SEQ ID NO: 32 (Kabat) | LCDR2 | AASNLAS |
| SEQ ID NO: 21 (Kabat) | LCDR3 | QQSNEDPYT |
| SEQ ID NO: 33 (Chothia) | LCDR1 | SQSVSYDGDSY |
| SEQ ID NO: 23 (Chothia) | LCDR2 | AAS |
| SEQ ID NO: 24 (Chothia) | LCDR3 | SNEDPY |
| SEQ ID NO: 34 | VL | EIVLTQSPATLSVSPGERATLSCRASQSVSYDGDSYMNW |
| | | YQQKPGQAPRLLIYAASNLASGIPARFSGSGSGTEFTLTIS |
| | | SLQSEDAAVYYCQQSNEDPYTFGGGTKVEIK |
| SEQ ID NO: 35 | DNA VL | GAAATCGTGCTGACCCAGAGCCCTGCCACCCTGAGTG |
| | | TGTCTCCAGGCGAGAGAGCCACACTGAGCTGTAGAGC |
| | | CAGCCAGAGCGTGTCCTACGACGGCGACAGCTACATG |
| | | AACTGGTATCAGCAGAAGCCCGGCCAGGCCCCCAGAC |
| | | TGCTGATCTACGCCGCTTCCAATCTGGCCAGCGGCATC |
| | | CCCGCCAGATTTTCCGGCTCTGGCTCCGGCACCGAGTT |
| | | CACCCTGACAATCAGCAGCCTCCAGAGCGAGGACGCC |
| | | GCCGTGTACTACTGCCAGCAGAGCAACGAGGACCCCT |
| | | ACACCTTTGGCGGAGGCACCAAGGTGGAAATCAAG |
| >VH1_D98E | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 36 (Combined) | HCDR3 | WRGEGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 36 (Kabat) | HCDR3 | WRGEGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 36 (Chothia) | HCDR3 | WRGEGYYAYFDV |
| SEQ ID NO: 37 | VH | QVQLVQSGAEVKKPGSSVKVSCKASGYAFTNYLIEWVR |
| | | QAPGQGLEWMGVINPGSGGTNYNEKFKGRVTITADKS |
| | | TSTAYMELSSLRSEDTAVYYCARWRGEGYYAYFDVWGQ |
| | | GTTVTVSS |
| SEQ ID NO: 38 | DNA VH | CAAGTGCAGCTGGTGCAGTCTGGCGCTGAAGTGAAG |
| | | AAACCCGGCTCCTCCGTGAAAGTGTCCTGCAAGGCCT |
| | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGCGCCAGAT |
| | | GGGGGGAGAGGGCTACTACGCCTACTTCGACGTGT |
| | | GGGGCCAGGGCACCACCGTGACCGTGTCCTCT |
| >VH1_G99A | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 39 (Combined) | HCDR3 | WRGDAYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 39 (Kabat) | HCDR3 | WRGDAYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 39 (Chothia) | HCDR3 | WRGDAYYAYFDV |
| SEQ ID NO: 40 | VH | QVQLVQSGAEVKKPGSSVKVSCKASGYAFTNYLIEWVR |
| | | QAPGQGLEWMGVINPGSGGTNYNEKFKGRVTITADKS |
| | | TSTAYMELSSLRSEDTAVYYCARWRGDAYYAYFDVWGQ |
| | | GTTVTVSS |
| SEQ ID NO: 41 | DNA VH | CAAGTGCAGCTGGTGCAGTCTGGCGCTGAAGTGAAG |
| | | AAACCCGGCTCCTCCGTGAAAGTGTCCTGCAAGGCCT |
| | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGTGCCCGGTG |
| | | GCGGGGAGATGCCTACTACGCCTACTTCGACGTGTGG |
| | | GGCCAGGGCACCACCGTGACCGTGTCCTCT |
| >VH1_D98Q | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 42 (Combined) | HCDR3 | WRGQGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 42 (Kabat) | HCDR3 | WRGQGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 42 (Chothia) | HCDR3 | WRGQGYYAYFDV |
| SEQ ID NO: 43 | VH | QVQLVQSGAEVKKPGSSVKVSCKASGYAFTNYLIEWVR |
| | | QAPGQGLEWMGVINPGSGGTNYNEKFKGRVTITADKS |
| | | TSTAYMELSSLRSEDTAVYYCARWRGQGYYAYFDVWG |
| | | QGTTVTVSS |
| SEQ ID NO: 44 | DNA VH | CAAGTGCAGCTGGTGCAGTCTGGCGCTGAAGTGAAG |
| | | AAACCCGGCTCCTCCGTGAAAGTGTCCTGCAAGGCCT |
| | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGCGCCAGAT |
| | | GGCGGGGACAGGGCTACTACGCCTACTTCGACGTGTG |
| | | GGGCCAGGGCACCACCGTGACCGTGTCCTCT |
| >VH1_D98S | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 45 (Combined) | HCDR3 | WRGSGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 45 (Kabat) | HCDR3 | WRGSGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 45 (Chothia) | HCDR3 | WRGSGYYAYFDV |
| SEQ ID NO: 46 | VH | QVQLVQSGAEVKKPGSSVKVSCKASGYAFTNYLIEWVR |
| | | QAPGQGLEWMGVINPGSGGTNYNEKFKGRVTITADKS |
| | | TSTAYMELSSLRSEDTAVYYCARWRGSGYYAYFDVWGQ |
| | | GTTVTVSS |
| SEQ ID NO: 47 | DNA VH | CAAGTGCAGCTGGTGCAGTCTGGCGCTGAAGTGAAG |
| | | AAACCCGGCTCCTCCGTGAAAGTGTCCTGCAAGGCCT |
| | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGCGCCAGAT |
| | | GGCGGGGATCTGGCTACTACGCCTACTTCGACGTGTG |
| | | GGGCCAGGGCACCACCGTGACCGTGTCCTCT |
| >VH3_D98E | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 36 (Combined) | HCDR3 | WRGEGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 36 (Kabat) | HCDR3 | WRGEGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 36 (Chothia) | HCDR3 | WRGEGYYAYFDV |
| SEQ ID NO: 48 | VH | QVQLVESGGGVVQPGRSLRLSCAASGYAFTNYLIEWVR |
| | | QAPGKGLEWVAVINPGSGGTNYNEKFKGRFTISADKSKS |
| | | TAYLQMNSLRAEDTAVYYCARWRGEGYYAYFDVWGQ |
| | | GTTVTVSS |
| SEQ ID NO: 419 | DNA VH | CAGGTGCAGCTGGTGGAGAGCGGCGGCGGCGTGGTG |
| | | CAGCCCGGCCGGAGCCTGCGGCTGAGCTGCGCCGCC |
| | | AGCGGCTACGCCTTCACCAACTACCTGATCGAGTGGG |
| | | TGCGGCAGGCCCCCGGCAAGGGCCTGGAGTGGGTGG |
| | | CCGTGATCAACCCCGGCAGCGGCGGCACCAACTACAA |
| | | CGAGAAGTTCAAGGGCCGGTTCACCATCAGCGCCGAC |
| | | AAGAGCAAGAGCACCGCCTACCTGCAGATGAACAGCC |
| | | TGCGGGCCGAGGACACCGCCGTGTACTACTGCGCCCG |
| | | GTGGCGGGGCGAGGGCTACTACGCCTACTTCGACGTG |
| | | TGGGGCCAGGGCACCACCGTGACCGTGAGCAGC |
| >VH3_G99A | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 39 (Combined) | HCDR3 | WRGDAYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 39 (Kabat) | HCDR3 | WRGDAYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 39 (Chothia) | HCDR3 | WRGDAYYAYFDV |
| SEQ ID NO: 51 | VH | QVQLVESGGGVVQPGRSLRLSCAASGYAFTNYLIEWVR |
| | | QAPGKGLEWVAVINPGSGGTNYNEKFKGRFTISADKSKS |
| | | TAYLQMNSLRAEDTAVYYCARWRGDAYYAYFDVWGQ |
| | | GTTVTVSS |
| SEQ ID NO: 52 | DNA VH | CAGGTGCAGCTGGTGGAGAGCGGCGGCGGCGTGGTG |
| | | CAGCCCGGCCGGAGCCTGCGGCTGAGCTGCGCCGCC |
| | | AGCGGCTACGCCTTCACCAACTACCTGATCGAGTGGG |
| | | TGCGGCAGGCCCCCGGCAAGGGCCTGGAGTGGGTGG |
| | | CCGTGATCAACCCCGGCAGCGGCGGCACCAACTACAA |
| | | CGAGAAGTTCAAGGGCCGGTTCACCATCAGCGCCGAC |
| | | AAGAGCAAGAGCACCGCCTACCTGCAGATGAACAGCC |
| | | TGCGGGCCGAGGACACCGCCGTGTACTACTGCGCCCG |
| | | GTGGCGGGGCGACGCCTACTACGCCTACTTCGACGTG |
| | | TGGGGCCAGGGCACCACCGTGACCGTGAGCAGC |
| >VH3_D98Q | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 42 (Combined) | HCDR3 | WRGQGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 42 (Kabat) | HCDR3 | WRGQGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 42 (Chothia) | HCDR3 | WRGQGYYAYFDV |
| SEQ ID NO: 53 | VH | QVQLVESGGGVVQPGRSLRLSCAASGYAFTNYLIEWVR |
| | | QAPGKGLEWVAVINPGSGGTNYNEKFKGRFTISADKSKS |
| | | TAYLQMNSLRAEDTAVYYCARWRGQGYYAYFDVWGQ |
| | | GTTVTVSS |
| SEQ ID NO: 54 | DNA VH | CAGGTGCAGCTGGTGGAGAGCGGCGGCGGCGTGGTG |
| | | CAGCCCGGCCGGAGCCTGCGGCTGAGCTGCGCCGCC |
| | | AGCGGCTACGCCTTCACCAACTACCTGATCGAGTGGG |
| | | TGCGGCAGGCCCCCGGCAAGGGCCTGGAGTGGGTGG |
| | | CCGTGATCAACCCCGGCAGCGGCGGCACCAACTACAA |
| | | CGAGAAGTTCAAGGGCCGGTTCACCATCAGCGCCGAC |
| | | AAGAGCAAGAGCACCGCCTACCTGCAGATGAACAGCC |
| | | TGCGGGCCGAGGACACCGCCGTGTACTACTGCGCCCG |
| | | GTGGCGGGGCCAGGGCTACTACGCCTACTTCGACGTG |
| | | TGGGGCCAGGGCACCACCGTGACCGTGAGCAGC |
| >VH3_D98S | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 45 (Combined) | HCDR3 | WRGSGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 45 (Kabat) | HCDR3 | WRGSGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 45 (Chothia) | HCDR3 | WRGSGYYAYFDV |
| SEQ ID NO: 55 | VH | QVQLVESGGGVVQPGRSLRLSCAASGYAFTNYLIEWVR |
| | | QAPGKGLEWVAVINPGSGGTNYNEKFKGRFTISADKSKS |
| | | TAYLQMNSLRAEDTAVYYCARWRGSGYYAYFDVWGQ |
| | | GTTVTVSS |
| SEQ ID NO: 56 | DNA VH | CAGGTGCAGCTGGTGGAGAGCGGCGGCGGCGTGGTG |
| | | CAGCCCGGCCGGAGCCTGCGGCTGAGCTGCGCCGCC |
| | | AGCGGCTACGCCTTCACCAACTACCTGATCGAGTGGG |
| | | TGCGGCAGGCCCCCGGCAAGGGCCTGGAGTGGGTGG |
| | | CCGTGATCAACCCCGGCAGCGGCGGCACCAACTACAA |
| | | CGAGAAGTTCAAGGGCCGGTTCACCATCAGCGCCGAC |
| | | AAGAGCAAGAGCACCGCCTACCTGCAGATGAACAGCC |
| | | TGCGGGCCGAGGACACCGCCGTGTACTACTGCGCCCG |
| | | GTGGCGGGGCAGCGGCTACTACGCCTACTTCGACGTG |
| | | TGGGGCCAGGGCACCACCGTGACCGTGAGCAGC |
| >VH3s_D98E | | |
| SEQ ID NO: 11 (Combined) | HCDR1 | GYTFSSYLIE |
| SEQ ID NO: 12 (Combined) | HCDR2 | VINPGSGGTNYADSVKG |
| SEQ ID NO: 36 (Combined) | HCDR3 | WRGEGYYAYFDV |
| SEQ ID NO: 13 (Kabat) | HCDR1 | SYLIE |
| SEQ ID NO: 12 (Kabat) | HCDR2 | VINPGSGGTNYADSVKG |
| SEQ ID NO: 36 (Kabat) | HCDR3 | WRGEGYYAYFDV |
| SEQ ID NO: 14 (Chothia) | HCDR1 | GYTFSSY |
| SEQ ID NO: 6 (Chothia) | HCDR2 | NPGSGG |
| SEQ ID NO: 36 (Chothia) | HCDR3 | WRGEGYYAYFDV |
| SEQ ID NO: 57 | VH | QVQLVESGGGVVQPGRSLRLSCAASGYTFSSYLIEWVRQ |
| | | APGKGLEWVAVINPGSGGTNYADSVKGRFTISADKSKN |
| | | TAYLQMNSLRAEDTAVYYCARWRGEGYYAYFDVWGQ |
| | | GTTVTVSS |
| SEQ ID NO: 58 | DNA VH | CAGGTGCAATTGGTGGAAAGCGGCGGAGGCGTGGTG |
| | | CAGCCTGGAAGAAGCCTGAGACTGAGCTGTGCCGCCA |
| | | GCGGCTACACCTTCAGCAGCTACCTGATCGAGTGGGT |
| | | GCGCCAGGCCCCTGGCAAAGGACTGGAATGGGTGGC |
| | | CGTGATCAACCCTGGCAGCGGCGGCACCAATTACGCC |
| | | GATAGCGTGAAGGGCCGGTTCACCATCAGCGCCGACA |
| | | AGAGCAAGAACACCGCCTACCTCCAGATGAACAGCCT |
| | | GCGGGCCGAGGACACCGCCGTGTACTATTGTGCTCGG |
| | | TGGCGGGGAGAGGGCTACTACGCCTACTTCGACGTGT |
| | | GGGGCCAGGGCACCACAGTGACCGTCAGCTCA |
| >VH3s_G99A | | |
| SEQ ID NO: 11 (Combined) | HCDR1 | GYTFSSYLIE |
| SEQ ID NO: 12 (Combined) | HCDR2 | VINPGSGGTNYADSVKG |
| SEQ ID NO: 39 (Combined) | HCDR3 | WRGDAYYAYFDV |
| SEQ ID NO: 13 (Kabat) | HCDR1 | SYLIE |
| SEQ ID NO: 12 (Kabat) | HCDR2 | VINPGSGGTNYADSVKG |
| SEQ ID NO: 39 (Kabat) | HCDR3 | WRGDAYYAYFDV |
| SEQ ID NO: 14 (Chothia) | HCDR1 | GYTFSSY |
| SEQ ID NO: 6 (Chothia) | HCDR2 | NPGSGG |
| SEQ ID NO: 39 (Chothia) | HCDR3 | WRGDAYYAYFDV |
| SEQ ID NO: 59 | VH | QVQLVESGGGVVQPGRSLRLSCAASGYTFSSYLIEWVRQ |
| | | APGKGLEWVAVINPGSGGTNYADSVKGRFTISADKSKN |
| | | TAYLQMNSLRAEDTAVYYCARWRGDAYYAYFDVWGQ |
| | | GTTVTVSS |
| SEQ ID NO: 60 | DNA VH | CAGGTGCAATTGGTGGAAAGCGGCGGAGGCGTGGTG |
| | | CAGCCTGGAAGAAGCCTGAGACTGAGCTGTGCCGCCA |
| | | GCGGCTACACCTTCAGCAGCTACCTGATCGAGTGGGT |
| | | GCGCCAGGCCCCTGGCAAAGGACTGGAATGGGTGGC |
| | | CGTGATCAACCCTGGCAGCGGCGGCACCAATTACGCC |
| | | GATAGCGTGAAGGGCCGGTTCACCATCAGCGCCGACA |
| | | AGAGCAAGAACACCGCCTACCTCCAGATGAACAGCCT |
| | | GCGGGCCGAGGACACCGCCGTGTACTATTGTGCTCGG |
| | | TGGCGGGGAGATGCCTACTACGCCTACTTCGACGTGT |
| | | GGGGCCAGGGCACCACAGTGACCGTCAGCTCA |
| >VH3s_D98Q | | |
| SEQ ID NO: 11 (Combined) | HCDR1 | GYTFSSYLIE |
| SEQ ID NO: 12 (Combined) | HCDR2 | VINPGSGGTNYADSVKG |
| SEQ ID NO: 42 (Combined) | HCDR3 | WRGQGYYAYFDV |
| SEQ ID NO: 13 (Kabat) | HCDR1 | SYLIE |
| SEQ ID NO: 12 (Kabat) | HCDR2 | VINPGSGGTNYADSVKG |
| SEQ ID NO: 42 (Kabat) | HCDR3 | WRGQGYYAYFDV |
| SEQ ID NO: 14 (Chothia) | HCDR1 | GYTFSSY |
| SEQ ID NO: 6 (Chothia) | HCDR2 | NPGSGG |
| SEQ ID NO: 42 (Chothia) | HCDR3 | WRGQGYYAYFDV |
| SEQ ID NO: 61 | VH | QVQLVESGGGVVQPGRSLRLSCAASGYTFSSYLIEWVRQ |
| | | APGKGLEWVAVINPGSGGTNYADSVKGRFTISADKSKN |
| | | TAYLQMNSLRAEDTAVYYCARWRGQGYYAYFDVWGQ |
| | | GTTVTVSS |
| SEQ ID NO: 62 | DNA VH | CAGGTGCAATTGGTGGAAAGCGGCGGAGGCGTGGTG |
| | | CAGCCTGGAAGAAGCCTGAGACTGAGCTGTGCCGCCA |
| | | GCGGCTACACCTTCAGCAGCTACCTGATCGAGTGGGT |
| | | GCGCCAGGCCCCTGGCAAAGGACTGGAATGGGTGGC |
| | | CGTGATCAACCCTGGCAGCGGCGGCACCAATTACGCC |
| | | GATAGCGTGAAGGGCCGGTTCACCATCAGCGCCGACA |
| | | AGAGCAAGAACACCGCCTACCTCCAGATGAACAGCCT |
| | | GCGGGCCGAGGACACCGCCGTGTACTATTGTGCTCGG |
| | | TGGCGGGGACAGGGCTACTACGCCTACTTCGACGTGT |
| | | GGGGCCAGGGCACCACAGTGACCGTCAGCTCA |
| >VH3s_D98S | | |
| SEQ ID NO: 11 (Combined) | HCDR1 | GYTFSSYLIE |
| SEQ ID NO: 12 (Combined) | HCDR2 | VINPGSGGTNYADSVKG |
| SEQ ID NO: 45 (Combined) | HCDR3 | WRGSGYYAYFDV |
| SEQ ID NO: 13 (Kabat) | HCDR1 | SYLIE |
| SEQ ID NO: 12 (Kabat) | HCDR2 | VINPGSGGTNYADSVKG |
| SEQ ID NO: 45 (Kabat) | HCDR3 | WRGSGYYAYFDV |
| SEQ ID NO: 14 (Chothia) | HCDR1 | GYTFSSY |
| SEQ ID NO: 6 (Chothia) | HCDR2 | NPGSGG |
| SEQ ID NO: 45 (Chothia) | HCDR3 | WRGSGYYAYFDV |
| SEQ ID NO: 63 | VH | QVQLVESGGGVVQPGRSLRLSCAASGYTFSSYLIEWVRQ |
| | | APGKGLEWVAVINPGSGGTNYADSVKGRFTISADKSKN |
| | | TAYLQMNSLRAEDTAVYYCARWRGSGYYAYFDVWGQ |
| | | GTTVTVSS |
| SEQ ID NO: 64 | DNA VH | CAGGTGCAATTGGTGGAAAGCGGCGGAGGCGTGGTG |
| | | CAGCCTGGAAGAAGCCTGAGACTGAGCTGTGCCGCCA |
| | | GCGGCTACACCTTCAGCAGCTACCTGATCGAGTGGGT |
| | | GCGCCAGGCCCCTGGCAAAGGACTGGAATGGGTGGC |
| | | CGTGATCAACCCTGGCAGCGGCGGCACCAATTACGCC |
| | | GATAGCGTGAAGGGCCGGTTCACCATCAGCGCCGACA |
| | | AGAGCAAGAACACCGCCTACCTCCAGATGAACAGCCT |
| | | GCGGGCCGAGGACACCGCCGTGTACTATTGTGCTCGG |
| | | TGGCGGGGAAGCGGCTACTACGCCTACTTCGACGTGT |
| | | GGGGCCAGGGCACCACAGTGACCGTCAGCTCA |
| >VH5_D98E | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 36 (Combined) | HCDR3 | WRGEGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 36 (Kabat) | HCDR3 | WRGEGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 36 (Chothia) | HCDR3 | WRGEGYYAYFDV |
| SEQ ID NO: 49 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGEGYYAYFDVWGQ |
| | | GTTVTVSS |
| SEQ ID NO: 50 | DNA VH | GAAGTGCAGCTGGTGCAGTCTGGCGCTGAAGTGAAG |
| | | AAGCCCGGCGAGTCCCTGAAGATCTCCTGCAAGGGCT |
| | | CCGGCTACGCCTTCACCAACTACCTGATCGAGTGGGTC |
| | | CGACAGATGCCCGGCAAGGGCCTGGAGTGGATGGGC |
| | | GTGATCAACCCCGGCTCCGGCGGCACCAACTACAACG |
| | | AGAAGTTCAAGGGCCAAGTCACAATCTCCGCCGACAA |
| | | GTCCATCTCCACCGCCTACCTGCAGTGGTCCTCCCTGA |
| | | AGGCCTCCGACACCGCCATGTACTACTGCGCCAGATG |
| | | GCGGGGAGAGGGCTACTACGCCTACTTCGACGTGTG |
| | | GGGCCAGGGCACCACCGTGACCGTGTCCTCT |
| >VH5_G99A | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 39 (Combined) | HCDR3 | WRGDAYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 39 (Kabat) | HCDR3 | WRGDAYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 39 (Chothia) | HCDR3 | WRGDAYYAYFDV |
| SEQ ID NO: 65 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGDAYYAYFDVWGQ |
| | | GTTVTVSS |
| SEQ ID NO: 66 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGATGCCTACTACGCCTACTTCGACGT |
| | | GTGGGGCCAGGGCACCACAGTGACCGTCAGCTCA |
| >VH5_D98Q | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 42 (Combined) | HCDR3 | WRGQGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 42 (Kabat) | HCDR3 | WRGQGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 42 (Chothia) | HCDR3 | WRGQGYYAYFDV |
| SEQ ID NO: 67 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGQGYYAYFDVWGQ |
| | | GTTVTVSS |
| SEQ ID NO: 68 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGACAGGGCTACTACGCCTACTTCGACGT |
| | | GTGGGGCCAGGGCACCACAGTGACCGTCAGCTCA |
| >VH5_D98S | | |
| SEQ ID NO: 1 (Combined) | HCDR1 | GYAFTNYLIE |
| SEQ ID NO: 2 (Combined) | HCDR2 | VINPGSGGTNYNEKFKG |
| SEQ ID NO: 45 (Combined) | HCDR3 | WRGSGYYAYFDV |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 45 (Kabat) | HCDR3 | WRGSGYYAYFDV |
| SEQ ID NO: 5 (Chothia) | HCDR1 | GYAFTNY |
| SEQ ID NO: 6 (Chothia) | HCDR2 | NPGSGG |
| SEQ ID NO: 45 (Chothia) | HCDR3 | WRGSGYYAYFDV |
| SEQ ID NO: 46 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGSGYYAYFDVWGQ |
| | | GTTVTVSS |
| SEQ ID NO: 47 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAAGCGGCTACTACGCCTACTTCGACGT |
| | | GTGGGGCCAGGGCACCACAGTGACCGTCAGCTCA |
| >VK1_D28Q | | |
| SEQ ID NO: 69 (Combined) | LCDR1 | KASQSVDYQGDSYMN |
| SEQ ID NO: 20 (Combined) | LCDR2 | AASNLES |
| SEQ ID NO: 21 (Combined) | LCDR3 | QQSNEDPYT |
| SEQ ID NO: 69 (Kabat) | LCDR1 | KASQSVDYQGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: (Chothia) | LCDR1 | SQSVDYQGDSY |
| SEQ ID NO: 23 (Chothia) | LCDR2 | AAS |
| SEQ ID NO: 24 (Chothia) | LCDR3 | SNEDPY |
| SEQ ID NO: 70 | VL | AIRLTQSPSSFSASTGDRVTITCKASQSVDYQGDSYMNW |
| | | YQQKPGKAPKLLIYAASNLESGVPSRFSGSGSGTDFTLTIS |
| | | SLQSEDFATYYCQQSNEDPYTFGGGTKVEIK |
| SEQ ID NO: 71 | DNA VL | GCCATCAGACTGACCCAGAGCCCCTCCAGCTTCTCCGC |
| | | CTCCACCGGCGACAGAGTGACCATCACATGCAAGGCC |
| | | TCCCAGTCCGTGGACTACCAGGGCGACTCCTACATGA |
| | | ACTGGTATCAGCAGAAGCCCGGCAAGGCCCCTAAGCT |
| | | GCTGATCTACGCCGCCTCCAACCTGGAATCCGGCGTG |
| | | CCCTCCCGGTTCTCCGGCTCTGGCTCTGGCACCGACTT |
| | | CACCCTGACCATCTCCAGCCTGCAGTCCGAGGACTTCG |
| | | CCACCTACTACTGCCAGCAGTCCAACGAGGACCCCTAC |
| | | ACCTTCGGCGGAGGCACCAAAGTGGAAATCAAG |
| >VK1_G29A | | |
| SEQ ID NO: 72 (Combined) | LCDR1 | KASQSVDYDADSYMN |
| SEQ ID NO: 20 (Combined) | LCDR2 | AASNLES |
| SEQ ID NO: 21 (Combined) | LCDR3 | QQSNEDPYT |
| SEQ ID NO: 72 (Kabat) | LCDR1 | KASQSVDYDADSYMN |
| SEQ ID NO: 20 (Kabat) | LCDR2 | AASNLES |
| SEQ ID NO: 21 (Kabat) | LCDR3 | QQSNEDPYT |
| SEQ ID NO: 78 (Chothia) | LCDR1 | SQSVDYDADSY |
| SEQ ID NO: 23 (Chothia) | LCDR2 | AAS |
| SEQ ID NO: 24 (Chothia) | LCDR3 | SNEDPY |
| SEQ ID NO: 73 | VL | AIRLTQSPSSFSASTGDRVTITCKASQSVDYDADSYMNW |
| | | YQQKPGKAPKLLIYAASNLESGVPSRFSGSGSGTDFTLTIS |
| | | SLQSEDFATYYCQQSNEDPYTFGGGTKVEIK |
| SEQ ID NO: 74 | DNA VL | GACATCGTGCTGACACAGAGCCCTCTGTCCCTGCCCGT |
| | | GACCCTGGGCCAGCCTGCCTCCATCTCCTGCAAGGCCT |
| | | CCCAGTCCGTGGACTACGACGCCGACTCCTACATGAA |
| | | CTGGTATCAGCAGCGGCCTGGCCAGTCCCCTCGGCTG |
| | | CTGATCTACGCCGCCTCCAACCTGGAATCCGGCGTGCC |
| | | CGACAGATTCTCCGGCTCCGGCTCTGGCACCGACTTCA |
| | | CCCTGAAGATCTCCCGGGTGGAAGCCGAGGACGTGG |
| | | GCGTGTACTACTGCCAGCAGTCCAACGAGGACCCCTA |
| | | CACCTTCGGCGGAGGCACCAAAGTGGAAATCAAG |
| >VK2_D28Q | | |
| SEQ ID NO: 69 (Combined) | LCDR1 | KASQSVDYQGDSYMN |
| SEQ ID NO: 20 (Combined) | LCDR2 | AASNLES |
| SEQ ID NO: 21 (Combined) | LCDR3 | QQSNEDPYT |
| SEQ ID NO: 69 (Kabat) | LCDR1 | KASQSVDYQGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 75 (Chothia) | LCDR1 | SQSVDYQGDSY |
| SEQ ID NO: 23 (Chothia) | LCDR2 | AAS |
| SEQ ID NO: 24 (Chothia) | LCDR3 | SNEDPY |
| SEQ ID NO: 79 | VL | DIVLTQSPLSLPVTLGQPASISCKASQSVDYQGDSYMNW |
| | | YQQRPGQSPRLLIYAASNLESGVPDRFSGSGSGTDFTLKI |
| | | SRVEAEDVGVYYCQQSNEDPYTFGGGTKVEIK |
| SEQ ID NO: 80 | DNA VL | GAGATCGTGCTGACCCAGAGCCCCGCCACCCTGAGCG |
| | | TGAGCCCCGGCGAGCGGGCCACCCTGAGCTGCAAGG |
| | | CCAGCCAGAGCGTGGACTACCAGGGCGACAGCTACAT |
| | | GAACTGGTACCAGCAGAAGCCCGGCCAGGCCCCCCG |
| | | GCTGCTGATCTACGCCGCCAGCAACCTGGAGAGCGGC |
| | | ATCCCCGCCCGGTTCAGCGGCAGCGGCAGCGGCACCG |
| | | AGTTCACCCTGACCATCAGCAGCCTGCAGAGCGAGGA |
| | | CGCCGCCGTGTACTACTGCCAGCAGAGCAACGAGGAC |
| | | CCCTACACCTTCGGCGGCGGCACCAAGGTGGAGATCA |
| | | AG |
| >VK2_G29A | | |
| SEQ ID NO: 72 (Combined) | LCDR1 | KASQSVDYDADSYMN |
| SEQ ID NO: 20 (Combined) | LCDR2 | AASNLES |
| SEQ ID NO: 21 (Combined) | LCDR3 | QQSNEDPYT |
| SEQ ID NO: 72 (Kabat) | LCDR1 | KASQSVDYDADSYMN |
| SEQ ID NO: 20 (Kabat) | LCDR2 | AASNLES |
| SEQ ID NO: 21 (Kabat) | LCDR3 | QQSNEDPYT |
| SEQ ID NO: 78 (Chothia) | LCDR1 | SQSVDYDADSY |
| SEQ ID NO: 23 (Chothia) | LCDR2 | AAS |
| SEQ ID NO: 24 (Chothia) | LCDR3 | SNEDPY |
| SEQ ID NO: 81 | VL | DIVLTQSPLSLPVTLGQPASISCKASQSVDYDADSYMNW |
| | | YQQRPGQSPRLLIYAASNLESGVPDRFSGSGSGTDFTLKI |
| | | SRVEAEDVGVYYCQQSNEDPYTFGGGTKVEIK |
| SEQ ID NO: 82 | DNA VL | GAGATCGTGCTGACCCAGAGCCCCGCCACCCTGAGCG |
| | | TGAGCCCCGGCGAGCGGGCCACCCTGAGCTGCAAGG |
| | | CCAGCCAGAGCGTGGACTACGACGCCGACAGCTACAT |
| | | GAACTGGTACCAGCAGAAGCCCGGCCAGGCCCCCCG |
| | | GCTGCTGATCTACGCCGCCAGCAACCTGGAGAGCGGC |
| | | ATCCCCGCCCGGTTCAGCGGCAGCGGCAGCGGCACCG |
| | | AGTTCACCCTGACCATCAGCAGCCTGCAGAGCGAGGA |
| | | CGCCGCCGTGTACTACTGCCAGCAGAGCAACGAGGAC |
| | | CCCTACACCTTCGGCGGCGGCACCAAGGTGGAGATCA |
| | | AG |
| >VK3_D28Q | | |
| SEQ ID NO: 69 (Combined) | LCDR1 | KASQSVDYQGDSYMN |
| SEQ ID NO: 20 (Combined) | LCDR2 | AASNLES |
| SEQ ID NO: 21 (Combined) | LCDR3 | QQSNEDPYT |
| SEQ ID NO: 69 (Kabat) | LCDR1 | KASQSVDYQGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 75 (Chothia) | LCDR1 | SQSVDYQGDSY |
| SEQ ID NO: 23 (Chothia) | LCDR2 | AAS |
| SEQ ID NO: 24 (Chothia) | LCDR3 | SNEDPY |
| SEQ ID NO: 76 | VL | EIVLTQSPATLSVSPGERATLSCKASQSVDYQGDSYMNW |
| | | YQQKPGQAPRLLIYAASNLESGIPARFSGSGSGTEFTLTIS |
| | | SLQSEDAAVYYCQQSNEDPYTFGGGTKVEIK |
| SEQ ID NO: 77 | DNA VL | GAAATCGTGCTGACCCAGAGCCCTGCCACCCTGAGTG |
| | | TGTCTCCAGGCGAGAGAGCCACACTGAGCTGTAGAGC |
| | | CAGCCAGAGCGTGTCCTACCAGGGCGACAGCTACATG |
| | | AACTGGTATCAGCAGAAGCCCGGCCAGGCCCCCAGAC |
| | | TGCTGATCTACGCCGCTTCCAATCTGGCCAGCGGCATC |
| | | CCCGCCAGATTTTCCGGCTCTGGCTCCGGCACCGAGTT |
| | | CACCCTGACAATCAGCAGCCTCCAGAGCGAGGACGCC |
| | | GCCGTGTACTACTGCCAGCAGAGCAACGAGGACCCCT |
| | | ACACCTTTGGCGGAGGCACCAAGGTGGAAATCAAG |
| >VK3_G29A | | |
| SEQ ID NO: 72 (Combined) | LCDR1 | KASQSVDYDADSYMN |
| SEQ ID NO: 20 (Combined) | LCDR2 | AASNLES |
| SEQ ID NO: 21 (Combined) | LCDR3 | QQSNEDPYT |
| SEQ ID NO: 72 (Kabat) | LCDR1 | KASQSVDYDADSYMN |
| SEQ ID NO: 20 (Kabat) | LCDR2 | AASNLES |
| SEQ ID NO: 21 (Kabat) | LCDR3 | QQSNEDPYT |
| SEQ ID NO: 78 (Chothia) | LCDR1 | SQSVDYDADSY |
| SEQ ID NO: 23 (Chothia) | LCDR2 | AAS |
| SEQ ID NO: 24 (Chothia) | LCDR3 | SNEDPY |
| SEQ ID NO: 84 | VL | EIVLTQSPATLSVSPGERATLSCKASQSVDYDADSYMNW |
| | | YQQKPGQAPRLLIYAASNLESGIPARFSGSGSGTEFTLTIS |
| | | SLQSEDAAVYYCQQSNEDPYTFGGGTKVEIK |
| SEQ ID NO: 85 | DNA VL | GAAATCGTGCTGACCCAGAGCCCTGCCACCCTGAGTG |
| | | TGTCTCCAGGCGAGAGAGCCACACTGAGCTGTAGAGC |
| | | CAGCCAGAGCGTGTCCTACGACGCCGACAGCTACATG |
| | | AACTGGTATCAGCAGAAGCCCGGCCAGGCCCCCAGAC |
| | | TGCTGATCTACGCCGCTTCCAATCTGGCCAGCGGCATC |
| | | CCCGCCAGATTTTCCGGCTCTGGCTCCGGCACCGAGTT |
| | | CACCCTGACAATCAGCAGCCTCCAGAGCGAGGACGCC |
| | | GCCGTGTACTACTGCCAGCAGAGCAACGAGGACCCCT |
| | | ACACCTTTGGCGGAGGCACCAAGGTGGAAATCAAG |
| >VK3s_D28Q | | |
| SEQ ID NO: 86 (Combined) | LCDR1 | RASQSVSYQGDSYMN |
| SEQ ID NO: 32 (Combined) | LCDR2 | AASNLAS |
| SEQ ID NO: 21 (Combined) | LCDR3 | QQSNEDPYT |
| SEQ ID NO: 86 (Kabat) | LCDR1 | RASQSVSYQGDSYMN |
| SEQ ID NO: 32 (Kabat) | LCDR2 | AASNLAS |
| SEQ ID NO: 21 (Kabat) | LCDR3 | QQSNEDPYT |
| SEQ ID NO: 87 (Chothia) | LCDR1 | SQSVSYQGDSY |
| SEQ ID NO: 23 (Chothia) | LCDR2 | AAS |
| SEQ ID NO: 24 (Chothia) | LCDR3 | SNEDPY |
| SEQ ID NO: 88 | VL | EIVLTQSPATLSVSPGERATLSCRASQSVSYQGDSYMNW |
| | | YQQKPGQAPRLLIYAASNLASGIPARFSGSGSGTEFTLTIS |
| | | SLQSEDAAVYYCQQSNEDPYTFGGGTKVEIK |
| SEQ ID NO: 89 | DNA VL | GAAATCGTGCTGACCCAGAGCCCTGCCACCCTGAGTG |
| | | TGTCTCCAGGCGAGAGAGCCACACTGAGCTGTAGAGC |
| | | CAGCCAGAGCGTGTCCTACCAGGGCGACAGCTACATG |
| | | AACTGGTATCAGCAGAAGCCCGGCCAGGCCCCCAGAC |
| | | TGCTGATCTACGCCGCTTCCAATCTGGCCAGCGGCATC |
| | | CCCGCCAGATTTTCCGGCTCTGGCTCCGGCACCGAGTT |
| | | CACCCTGACAATCAGCAGCCTCCAGAGCGAGGACGCC |
| | | GCCGTGTACTACTGCCAGCAGAGCAACGAGGACCCCT |
| | | ACACCTTTGGCGGAGGCACCAAGGTGGAAATCAAG |
| >VK3s_G29A | | |
| SEQ ID NO: 90 (Combined) | LCDR1 | RASQSVSYDADSYMN |
| SEQ ID NO: 32 (Combined) | LCDR2 | AASNLAS |
| SEQ ID NO: 21 (Combined) | LCDR3 | QQSNEDPYT |
| SEQ ID NO: 90 (Kabat) | LCDR1 | RASQSVSYDADSYMN |
| SEQ ID NO: 32 (Kabat) | LCDR2 | AASNLAS |
| SEQ ID NO: 21 (Kabat) | LCDR3 | QQSNEDPYT |
| SEQ ID NO: 420 (Chothia) | LCDR1 | SQSVSYDADSY |
| SEQ ID NO: 23 (Chothia) | LCDR2 | AAS |
| SEQ ID NO: 24 (Chothia) | LCDR3 | SNEDPY |
| SEQ ID NO: 91 | VL | EIVLTQSPATLSVSPGERATLSCRASQSVSYDADSYMNW |
| | | YQQKPGQAPRLLIYAASNLASGIPARFSGSGSGTEFTLTIS |
| | | SLQSEDAAVYYCQQSNEDPYTFGGGTKVEIK |
| SEQ ID NO: 92 | DNA VL | GAAATCGTGCTGACCCAGAGCCCTGCCACCCTGAGTG |
| | | TGTCTCCAGGCGAGAGAGCCACACTGAGCTGTAGAGC |
| | | CAGCCAGAGCGTGTCCTACGACGCCGACAGCTACATG |
| | | AACTGGTATCAGCAGAAGCCCGGCCAGGCCCCCAGAC |
| | | TGCTGATCTACGCCGCTTCCAATCTGGCCAGCGGCATC |
| | | CCCGCCAGATTTTCCGGCTCTGGCTCCGGCACCGAGTT |
| | | CACCCTGACAATCAGCAGCCTCCAGAGCGAGGACGCC |
| | | GCCGTGTACTACTGCCAGCAGAGCAACGAGGACCCCT |
| | | ACACCTTTGGCGGAGGCACCAAGGTGGAAATCAAG |
| >hIgG1 | | |
| SEQ ID NO: 93 | Constant HC | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNY |
| | | KTTPPVLDSDGSFFLYSKLTVDKSRWQQGNVFSCSVMH |
| | | EALHNHYTQKSLSLSPGK |
| SEQ ID NO: 94 | DNA Constant | GCGTCGACCAAGGGCCCCAGCGTGTTCCCCCTGGCCC |
| | HC | CCAGCAGCAAGAGCACCAGCGGCGGCACAGCCGCCC |
| | | TGGGCTGCCTGGTGAAGGACTACTTCCCCGAGCCCGT |
| | | GACCGTGTCCTGGAACAGCGGAGCCCTGACCTCCGGC |
| | | GTGCACACCTTCCCCGCCGTGCTGCAGAGCAGCGGCC |
| | | TGTACAGCCTGTCCAGCGTGGTGACAGTGCCCAGCAG |
| | | CAGCCTGGGCACCCAGACCTACATCTGCAACGTGAAC |
| | | CACAAGCCCAGCAACACCAAGGTGGACAAGAGAGTG |
| | | GAGCCCAAGAGCTGCGACAAGACCCACACCTGCCCCC |
| | | CCTGCCCAGCCCCAGAGCTGCTGGGCGGACCCTCCGT |
| | | GTTCCTGTTCCCCCCCAAGCCCAAGGACACCCTGATGA |
| | | TCAGCAGGACCCCCGAGGTGACCTGCGTGGTGGTGG |
| | | ACGTGAGCCACGAGGACCCAGAGGTGAAGTTCAACT |
| | | GGTACGTGGACGGCGTGGAGGTGCACAACGCCAAGA |
| | | CCAAGCCCAGAGAGGAGCAGTACAACAGCACCTACA |
| | | GGGTGGTGTCCGTGCTGACCGTGCTGCACCAGGACTG |
| | | GCTGAACGGCAAGGAATACAAGTGCAAGGTCTCCAAC |
| | | AAGGCCCTGCCAGCCCCCATCGAAAAGACCATCAGCA |
| | | AGGCCAAGGGCCAGCCACGGGAGCCCCAGGTGTACA |
| | | CCCTGCCCCCCTCCCGGGAGGAGATGACCAAGAACCA |
| | | GGTGTCCCTGACCTGTCTGGTGAAGGGCTTCTACCCCA |
| | | GCGACATCGCCGTGGAGTGGGAGAGCAACGGCCAGC |
| | | CCGAGAACAACTACAAGACCACCCCCCCAGTGCTGGA |
| | | CAGCGACGGCAGCTTCTTCCTGTACAGCAAGCTGACC |
| | | GTGGACAAGTCCAGGTGGCAGCAGGGCAACGTGTTC |
| | | AGCTGCAGCGTGATGCACGAGGCCCTGCACAACCACT |
| | | ACACCCAGAAGAGCCTGAGCCTGTCCCCCGGCAAG |
| >hIgG2 | | |
| SEQ ID NO: 95 | Constant HC | ASTKGPSVFPLAPCSRSTSESTAALGCLVKDYFPEPVTVS |
| | | WNSGALTSGVHTFPAVLQSSGLYSLSSVVTVPSSNFGTQ |
| | | TYTCNVDHKPSNTKVDKTVERKCCVECPPCPAPPVAGPS |
| | | VFLFPPKPKDTLMISRTPEVTCVVVDVSHEDPEVQFNWY |
| | | VDGVEVHNAKTKPREEQFNSTFRVVSVLTVVHQDWLN |
| | | GKEYKCKVSNKGLPAPIEKTISKTKGQPREPQVYTLPPSRE |
| | | EMTKNQVSLTCLVKGFYPSDIAVEWESNGQPENNYKTT |
| | | PPMLDSDGSFFLYSKLTVDKSRWQQGNVFSCSVMHEAL |
| | | HNHYTQKSLSLSPGK |
| SEQ ID NO: 96 | DNA Constant | GCCAGCACCAAGGGCCCCAGCGTGTTCCCCCTGGCCC |
| | HC | CCTGCAGCAGAAGCACCAGCGAGAGCACAGCCGCCCT |
| | | GGGCTGCCTGGTGAAGGACTACTTCCCCGAGCCAGTG |
| | | ACCGTGTCCTGGAACAGCGGAGCCCTGACCAGCGGC |
| | | GTGCACACCTTCCCCGCCGTGCTGCAGAGCAGCGGCC |
| | | TGTACAGCCTGTCCAGCGTGGTGACCGTGCCCAGCAG |
| | | CAACTTCGGCACCCAGACCTACACCTGCAACGTGGAC |
| | | CACAAGCCCAGCAACACCAAGGTGGACAAGACCGTG |
| | | GAGAGGAAGTGCTGCGTGGAGTGCCCCCCCTGCCCA |
| | | GCCCCCCCAGTGGCCGGACCCTCCGTGTTCCTGTTCCC |
| | | CCCCAAGCCCAAGGACACCCTGATGATCAGCAGGACC |
| | | CCCGAGGTGACCTGCGTGGTGGTGGACGTGAGCCAC |
| | | GAGGACCCAGAGGTGCAGTTCAACTGGTACGTGGAC |
| | | GGCGTGGAGGTGCACAACGCCAAGACCAAGCCCAGA |
| | | GAGGAACAGTTTAACAGCACCTTCAGGGTGGTGTCCG |
| | | TGCTGACCGTGGTGCACCAGGACTGGCTGAACGGCAA |
| | | AGAGTACAAGTGCAAGGTCTCCAACAAGGGCCTGCCA |
| | | GCCCCCATCGAGAAAACCATCAGCAAGACCAAGGGCC |
| | | AGCCACGGGAGCCCCAGGTGTACACCCTGCCCCCCAG |
| | | CCGGGAGGAAATGACCAAGAACCAGGTGTCCCTGAC |
| | | CTGTCTGGTGAAGGGCTTCTACCCCAGCGACATCGCC |
| | | GTGGAGTGGGAGAGCAACGGCCAGCCCGAGAACAAC |
| | | TACAAGACCACCCCCCCCATGCTGGACAGCGACGGCA |
| | | GCTTCTTCCTGTACAGCAAGCTGACAGTGGACAAGAG |
| | | CAGGTGGCAGCAGGGCAACGTGTTCAGCTGCAGCGT |
| | | GATGCACGAGGCCCTGCACAACCACTACACCCAGAAG |
| | | AGCCTGAGCCTGTCCCCCGGCAAG |
| >hIgG3 | | |
| SEQ ID NO: 97 | Constant HC | ASTKGPSVFPLAPCSRSTSGGTAALGCLVKDYFPEPVTVS |
| | | WNSGALTSGVHTFPAVLQSSGLYSLSSVVTVPSSSLGTQ |
| | | TYTCNVNHKPSNTKVDKRVELKTPLGDTTHTCPRCPEPK |
| | | SCDTPPPCPRCPEPKSCDTPPPCPRCPEPKSCDTPPPCPR |
| | | CPAPELLGGPSVFLFPPKPKDTLMISRTPEVTCVVVDVSH |
| | | EDPEVQFKWYVDGVEVHNAKTKPREEQYNSTFRVVSVL |
| | | TVLHQDWLNGKEYKCKVSNKALPAPIEKTISKTKGQPRE |
| | | PQVYTLPPSREEMTKNQVSLTCLVKGFYPSDIAVEWESS |
| | | GQPENNYNTTPPMLDSDGSFFLYSKLTVDKSRWQQGNI |
| | | FSCSVMHEALHNRFTQKSLSLSPGK |
| SEQ ID NO: 98 | DNA Constant | GCCAGCACCAAGGGCCCCAGCGTGTTCCCCCTGGCCC |
| | HC | CCTGCAGCCGGAGCACCAGCGGCGGCACCGCCGCCCT |
| | | GGGCTGCCTGGTGAAGGACTACTTCCCCGAGCCCGTG |
| | | ACCGTGAGCTGGAACAGCGGCGCCCTGACCAGCGGC |
| | | GTGCACACCTTCCCCGCCGTGCTGCAGAGCAGCGGCC |
| | | TGTACAGCCTGAGCAGCGTGGTGACCGTGCCCAGCAG |
| | | CAGCCTGGGCACCCAGACCTACACCTGCAACGTGAAC |
| | | CACAAGCCCAGCAACACCAAGGTGGACAAGCGGGTG |
| | | GAGCTGAAGACCCCCCTGGGCGACACCACCCACACCT |
| | | GCCCCCGGTGCCCCGAGCCCAAGAGCTGCGACACCCC |
| | | CCCCCCCTGCCCCCGGTGCCCCGAGCCCAAGAGCTGC |
| | | GACACCCCCCCCCCCTGCCCCCGGTGCCCCGAGCCCAA |
| | | GAGCTGCGACACCCCCCCCCCCTGCCCCCGGTGCCCC |
| | | GCCCCCGAGCTGCTGGGCGGCCCCAGCGTGTTCCTGT |
| | | TCCCCCCCAAGCCCAAGGACACCCTGATGATCAGCCG |
| | | GACCCCCGAGGTGACCTGCGTGGTGGTGGACGTGAG |
| | | CCACGAGGACCCCGAGGTGCAGTTCAAGTGGTACGTG |
| | | GACGGCGTGGAGGTGCACAACGCCAAGACCAAGCCC |
| | | CGGGAGGAGCAGTACAACAGCACCTTCCGGGTGGTG |
| | | AGCGTGCTGACCGTGCTGCACCAGGACTGGCTGAACG |
| | | GCAAGGAGTACAAGTGCAAGGTGAGCAACAAGGCCC |
| | | TGCCCGCCCCCATCGAGAAGACCATCAGCAAGACCAA |
| | | GGGCCAGCCCCGGGAGCCCCAGGTGTACACCCTGCCC |
| | | CCCAGCCGGGAGGAGATGACCAAGAACCAGGTGAGC |
| | | CTGACCTGCCTGGTGAAGGGCTTCTACCCCAGCGACA |
| | | TCGCCGTGGAGTGGGAGAGCAGCGGCCAGCCCGAGA |
| | | ACAACTACAACACCACCCCCCCCATGCTGGACAGCGA |
| | | CGGCAGCTTCTTCCTGTACAGCAAGCTGACCGTGGAC |
| | | AAGAGCCGGTGGCAGCAGGGCAACATCTTCAGCTGC |
| | | AGCGTGATGCACGAGGCCCTGCACAACCGGTTCACCC |
| | | AGAAGAGCCTGAGCCTGAGCCCCGGCAAG |
| >hIgG4 | | |
| SEQ ID NO: 99 | Constant HC | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPSCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLN |
| | | GKEYKCKVSNKGLPSSIEKTISKAKGQPREPQVYTLPPSQE |
| | | EMTKNQVSLTCLVKGFYPSDIAVEWESNGQPENNYKTT |
| | | PPVLDSDGSFFLYSRLTVDKSRWQEGNVFSCSVMHEAL |
| | | HNHYTQKSLSLSLGK |
| SEQ ID NO: 10 | DNA Constant | GCCTCTACCAAGGGCCCCAGCGTGTTCCCCCTGGCCCC |
| | HC | CTGCAGCAGAAGCACCAGCGAGAGCACAGCCGCCCT |
| | | GGGCTGCCTGGTGAAGGACTACTTCCCCGAGCCAGTG |
| | | ACCGTGTCCTGGAACAGCGGAGCCCTGACCAGCGGC |
| | | GTGCACACCTTCCCCGCCGTGCTGCAGAGCAGCGGCC |
| | | TGTACAGCCTGTCCAGCGTGGTGACCGTGCCCAGCAG |
| | | CAGCCTGGGCACCAAGACCTACACCTGCAACGTGGAC |
| | | CACAAGCCCAGCAACACCAAGGTGGACAAGAGGGTG |
| | | GAGAGCAAGTACGGCCCACCCTGCCCCTCTTGCCCAG |
| | | CCCCCGAGTTCCTGGGCGGACCCTCCGTGTTCCTGTTC |
| | | CCCCCCAAGCCCAAGGACACCCTGATGATCAGCAGGA |
| | | CCCCCGAGGTGACCTGCGTGGTGGTGGACGTGAGCC |
| | | AGGAAGATCCAGAGGTCCAGTTCAACTGGTACGTGGA |
| | | CGGCGTGGAGGTGCACAACGCCAAGACCAAGCCCAG |
| | | AGAGGAACAGTTTAACAGCACCTACAGGGTGGTGTCC |
| | | GTGCTGACCGTGCTGCACCAGGACTGGCTGAACGGCA |
| | | AAGAGTACAAGTGCAAGGTCTCCAACAAGGGCCTGCC |
| | | CAGCTCCATCGAGAAAACCATCAGCAAGGCCAAGGGC |
| | | CAGCCACGGGAGCCCCAGGTGTACACCCTGCCACCCT |
| | | CCCAGGAAGAGATGACCAAGAACCAGGTGTCCCTGAC |
| | | CTGTCTGGTGAAGGGCTTCTACCCCAGCGACATCGCC |
| | | GTGGAGTGGGAGAGCAACGGCCAGCCCGAGAACAAC |
| | | TACAAGACCACCCCCCCAGTGCTGGACAGCGACGGCA |
| | | GCTTCTTCCTGTACAGCAGGCTGACCGTGGACAAGTC |
| | | CAGGTGGCAGGAAGGCAACGTCTTTAGCTGCAGCGT |
| | | GATGCACGAGGCCCTGCACAACCACTACACCCAGAAG |
| | | AGCCTGAGCCTGTCCCTGGGCAAG |
| >hIgG1 LALA | | |
| SEQ ID NO: 101 | Constant HC | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPEAA |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTLP |
| | | PSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| SEQ ID NO: 102 | DNA Constant | GCCTCCACCAAGGGTCCATCGGTCTTCCCCCTGGCACC |
| | HC | CTCCTCCAAGAGCACCTCTGGGGGCACAGCGGCCCTG |
| | | GGCTGCCTGGTCAAGGACTACTTCCCCGAACCGGTGA |
| | | CGGTGTCGTGGAACTCAGGCGCCCTGACCAGCGGCGT |
| | | GCACACCTTCCCGGCTGTCCTACAGTCCTCAGGACTCT |
| | | ACTCCCTCAGCAGCGTGGTGACCGTGCCCTCCAGCAG |
| | | CTTGGGCACCCAGACCTACATCTGCAACGTGAATCAC |
| | | AAGCCCAGCAACACCAAGGTGGACAAGAGAGTTGAG |
| | | CCCAAATCTTGTGACAAAACTCACACATGCCCACCGTG |
| | | CCCAGCACCTGAAGCAGCGGGGGGACCGTCAGTCTTC |
| | | CTCTTCCCCCCAAAACCCAAGGACACCCTCATGATCTC |
| | | CCGGACCCCTGAGGTCACATGCGTGGTGGTGGACGTG |
| | | AGCCACGAAGACCCTGAGGTCAAGTTCAACTGGTACG |
| | | TGGACGGCGTGGAGGTGCATAATGCCAAGACAAAGC |
| | | CGCGGGAGGAGCAGTACAACAGCACGTACCGGGTGG |
| | | TCAGCGTCCTCACCGTCCTGCACCAGGACTGGCTGAAT |
| | | GGCAAGGAGTACAAGTGCAAGGTCTCCAACAAAGCCC |
| | | TCCCAGCCCCCATCGAGAAAACCATCTCCAAAGCCAAA |
| | | GGGCAGCCCCGAGAACCACAGGTGTACACCCTGCCCC |
| | | CATCCCGGGAGGAGATGACCAAGAACCAGGTCAGCC |
| | | TGACCTGCCTGGTCAAAGGCTTCTATCCCAGCGACATC |
| | | GCCGTGGAGTGGGAGAGCAATGGGCAGCCGGAGAA |
| | | CAACTACAAGACCACGCCTCCCGTGCTGGACTCCGAC |
| | | GGCTCCTTCTTCCTCTACAGCAAGCTCACCGTGGACAA |
| | | GAGCAGGTGGCAGCAGGGGAACGTCTTCTCATGCTCC |
| | | GTGATGCATGAGGCTCTGCACAACCACTACACGCAGA |
| | | AGAGCCTCTCCCTGTCTCCGGGTAAA |
| >hIgG1 N297A | | |
| SEQ ID NO: 103 | Constant HC | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYASTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNY |
| | | KTTPPVLDSDGSFFLYSKLTVDKSRWQQGNVFSCSVMH |
| | | EALHNHYTQKSLSLSPGK |
| SEQ ID NO: 104 | DNA Constant | GCTAGCACCAAGGGCCCCTCCGTGTTCCCTCTGGCCCC |
| | HC | CTCCAGCAAGTCCACCTCTGGCGGCACCGCCGCTCTG |
| | | GGCTGCCTGGTGAAAGACTACTTCCCCGAGCCCGTGA |
| | | CCGTGTCCTGGAACTCTGGCGCCCTGACCTCCGGCGT |
| | | GCACACCTTTCCAGCCGTGCTGCAGTCCTCCGGCCTGT |
| | | ACTCCCTGTCCTCCGTGGTGACCGTGCCCTCTAGCTCT |
| | | CTGGGCACCCAGACCTACATCTGCAACGTGAACCACA |
| | | AGCCCTCCAACACCAAGGTGGACAAGCGGGTGGAAC |
| | | CCAAGTCCTGCGACAAGACCCACACCTGTCCCCCCTGC |
| | | CCTGCCCCTGAACTGCTGGGCGGACCTTCCGTGTTCCT |
| | | GTTCCCCCCAAAGCCCAAGGACACCCTGATGATCTCCC |
| | | GGACCCCCGAAGTGACCTGCGTGGTGGTGGACGTGTC |
| | | CCACGAGGACCCTGAAGTGAAGTTCAATTGGTACGTG |
| | | GACGGCGTGGAAGTGCACAACGCCAAGACCAAGCCC |
| | | AGAGAGGAACAGTACGCCTCCACCTACCGGGTGGTGT |
| | | CTGTGCTGACCGTGCTGCACCAGGACTGGCTGAACGG |
| | | CAAAGAGTACAAGTGCAAGGTCTCCAACAAGGCCCTG |
| | | CCTGCCCCCATCGAAAAGACCATCTCCAAGGCCAAGG |
| | | GCCAGCCCCGCGAGCCACAGGTGTACACACTGCCCCC |
| | | CAGCCGGGAAGAGATGACCAAGAACCAGGTGTCCCT |
| | | GACCTGTCTGGTCAAAGGCTTCTACCCCTCCGATATCG |
| | | CCGTGGAGTGGGAGTCCAACGGACAGCCCGAGAACA |
| | | ACTACAAGACCACCCCCCCTGTGCTGGACTCCGACGG |
| | | CTCATTCTTCCTGTACTCCAAGCTGACCGTGGACAAGT |
| | | CCCGGTGGCAGCAGGGCAACGTGTTCTCCTGCTCCGT |
| | | GATGCACGAGGCCCTGCACAACCACTACACCCAGAAG |
| | | TCCCTGTCCCTGAGCCCCGGCAAG |
| >hIgG1 DAPA | | |
| SEQ ID NO: 105 | Constant HC | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVAVSHEDPEVKFN |
| | | WYVDGVEVHNAKTKPREEQYNSTYRVVSVLTVLHQDW |
| | | LNGKEYKCKVSNKALAAPIEKTISKAKGQPREPQVYTLPP |
| | | SREEMTKNQVSLTCLVKGFYPSDIAVEWESNGQPENNY |
| | | KTTPPVLDSDGSFFLYSKLTVDKSRWQQGNVFSCSVMH |
| | | EALHNHYTQKSLSLSPGK |
| SEQ ID NO: 106 | DNA Constant | GCTAGCACCAAGGGCCCCAGCGTGTTCCCCCTGGCCC |
| | HC | CCAGCAGCAAGAGCACCAGCGGCGGCACAGCCGCCC |
| | | TGGGCTGCCTGGTGAAGGACTACTTCCCCGAGCCCGT |
| | | GACCGTGTCCTGGAACAGCGGAGCCCTGACCTCCGGC |
| | | GTGCACACCTTCCCCGCCGTGCTGCAGAGCAGCGGCC |
| | | TGTACAGCCTGTCCAGCGTGGTGACAGTGCCCAGCAG |
| | | CAGCCTGGGCACCCAGACCTACATCTGCAACGTGAAC |
| | | CACAAGCCCAGCAACACCAAGGTGGACAAGAGAGTG |
| | | GAGCCCAAGAGCTGCGACAAGACCCACACCTGCCCCC |
| | | CCTGCCCAGCCCCAGAGCTGCTGGGCGGACCCTCCGT |
| | | GTTCCTGTTCCCCCCCAAGCCCAAGGACACCCTGATGA |
| | | TCAGCAGGACCCCCGAGGTGACCTGCGTGGTGGTGG |
| | | CCGTGAGCCACGAGGACCCAGAGGTGAAGTTCAACT |
| | | GGTACGTGGACGGCGTGGAGGTGCACAACGCCAAGA |
| | | CCAAGCCCAGAGAGGAGCAGTACAACAGCACCTACA |
| | | GGGTGGTGTCCGTGCTGACCGTGCTGCACCAGGACTG |
| | | GCTGAACGGCAAGGAATACAAGTGCAAGGTCTCCAAC |
| | | AAGGCCCTGGCAGCCCCCATCGAAAAGACCATCAGCA |
| | | AGGCCAAGGGCCAGCCACGGGAGCCCCAGGTGTACA |
| | | CCCTGCCCCCCTCCCGGGAGGAGATGACCAAGAACCA |
| | | GGTGTCCCTGACCTGTCTGGTGAAGGGCTTCTACCCCA |
| | | GCGACATCGCCGTGGAGTGGGAGAGCAACGGCCAGC |
| | | CCGAGAACAACTACAAGACCACCCCCCCAGTGCTGGA |
| | | CAGCGACGGCAGCTTCTTCCTGTACAGCAAGCTGACC |
| | | GTGGACAAGTCCAGGTGGCAGCAGGGCAACGTGTTC |
| | | AGCTGCAGCGTGATGCACGAGGCCCTGCACAACCACT |
| | | ACACCCAGAAGAGCCTGAGCCTGTCCCCCGGCAAG |
| >hkappa | | |
| SEQ ID NO: 107 | Constant LC | RTVAAPSVFIFPPSDEQLKSGTASVVCLLNNFYPREAKVQ |
| | | WKVDNALQSGNSQESVTEQDSKDSTYSLSSTLTLSKADY |
| | | EKHKVYACEVTHQGLSSPVTKSFNRGEC |
| SEQ ID NO: 108 | DNA Constant | CGTACGGTGGCCGCTCCCAGCGTGTTCATCTTCCCCCC |
| | LC | CAGCGACGAGCAGCTGAAGAGCGGCACCGCCAGCGT |
| | | GGTGTGCCTGCTGAACAACTTCTACCCCCGGGAGGCC |
| | | AAGGTGCAGTGGAAGGTGGACAACGCCCTGCAGAGC |
| | | GGCAACAGCCAGGAGAGCGTCACCGAGCAGGACAGC |
| | | AAGGACTCCACCTACAGCCTGAGCAGCACCCTGACCC |
| | | TGAGCAAGGCCGACTACGAGAAGCATAAGGTGTACG |
| | | CCTGCGAGGTGACCCACCAGGGCCTGTCCAGCCCCGT |
| | | GACCAAGAGCTTCAACAGGGGCGAGTGC |
| >IL-2 | | |
| SEQ ID NO: 109 | Human IL-2 | MYRMQLLSCIALSLALVTNSAPTSSSTKKTQLQLEHLLLDL |
| | | QMILNGINNYKNPKLTRMLTFKFYMPKKATELKHLQCLE |
| | | EELKPLEEVLNLAQSKNFHLRPRDLISNINVIVLELKGSETT |
| | | FMCEYADETATIVEFLNRWITFCQSIISTLT |
| SEQ ID NO: 110 | Proleukin ® | MAPTSSSTKKTQLQLEHLLLDLQMILNGINNYKNPKLTR |
| | (aldesleukin) | MLTFKFYMPKKATELKHLQCLEEELKPLEEVLNLAQSKNF |
| | | HLRPRDLISNINVIVLELKGSETTFMCEYADETATIVEFLN |
| | | RWITFSQSIISTLT |
| >NARA1 | | |
| SEQ ID NO: 4 (Kabat) | HCDR1 | NYLIE |
| SEQ ID NO: 2 (Kabat) | HCDR2 | VINPGSGGTNYNEKFKG |
| SEQ ID NO: 3 (Kabat) | HCDR3 | WRGDGYYAYFDV |
| SEQ ID NO: 19 (Kabat) | LCDR1 | KASQSVDYDGDSYMN |
| SEQ ID NO: 20 (Kabat) | LCDR2 | AASNLES |
| SEQ ID NO: 21 (Kabat) | LCDR3 | QQSNEDPYT |
| SEQ ID NO: 111 | VH | QVQLQQSGAELVRPGTSVKVSCKASGYAFTNYLIEWVK |
| | | QRPGQGLEWIGVINPGSGGTNYNEKFKGKATLTADKSS |
| | | STAYMQLSSLTSDDSAVYFCARWRGDGYYAYFDVWGA |
| | | GTTVTVSS |
| SEQ ID NO: 112 | DNA VH | CAGGTCCAGCTGCAGCAGTCTGGAGCTGAGCTGGTAA |
| | | GGCCTGGGACTTCAGTGAAGGTGTCCTGCAAGGCTTC |
| | | TGGATACGCCTTCACTAATTACTTGATAGAGTGGGTAA |
| | | AGCAGAGGCCTGGACAGGGCCTTGAGTGGATTGGAG |
| | | TGATTAATCCTGGAAGTGGTGGTACTAACTACAATGA |
| | | GAAGTTCAAGGGCAAGGCAACACTGACTGCAGACAA |
| | | ATCCTCCAGCACTGCCTACATGCAGCTCAGCAGCCTGA |
| | | CATCTGATGACTCTGCGGTCTATTTCTGTGCAAGATGG |
| | | AGGGGGGATGGTTACTACGCGTACTTCGATGTCTGGG |
| | | GCGCAGGGACCACGGTCACCGTCTCCTCA |
| SEQ ID NO: 113 | VL | DIVLTQSPASLAVSLGQRATISCKASQSVDYDGDSYMN |
| | | WYQQKPGQPPKLLIYAASNLESGIPARFSGSGSGTDFTL |
| | | NIHPVEEEDAATYYCQQSNEDPYTFGGGTKLEIK |
| SEQ ID NO: 114 | DNA VL | GACATTGTGCTGACCCAATCTCCAGCTTCTTTGGCTGT |
| | | GTCTCTAGGGCAGAGGGCCACCATCTCCTGCAAGGCC |
| | | AGCCAAAGTGTTGATTATGATGGTGATAGTTATATGA |
| | | ACTGGTACCAACAGAAACCAGGACAGCCACCCAAACT |
| | | CCTCATCTATGCTGCATCCAATCTAGAATCTGGGATCC |
| | | CAGCCAGGTTTAGTGGCAGTGGGTCTGGGACAGACTT |
| | | CACCCTCAACATCCATCCTGTGGAGGAGGAGGATGCT |
| | | GCAACCTATTACTGTCAGCAAAGTAATGAGGATCCGT |
| | | ACACGTTCGGAGGGGGGACCAAGCTGGAAATAAAA |
| SEQ ID NO: 115 | Heavy Chain | QVQLQQSGAELVRPGTSVKVSCKASGYAFTNYLIEWVK |
| | | QRPGQGLEWIGVINPGSGGTNYNEKFKGKATLTADKSS |
| | | STAYMQLSSLTSDDSAVYFCARWRGDGYYAYFDVWGA |
| | | GTTVTVSSAKTTAPSVYPLAPVCGDTTGSSVTLGCLVKGY |
| | | FPEPVTLTWNSGSLSSGVHTFPAVLQSDLYTLSSSVTVTS |
| | | STWPSQSITCNVAHPASSTKVDKKIEPRGPTIKPCPPCKC |
| | | PAPNLLGGPSVFIFPPKIKDVLMISLSPIVTCVVVDVSEDD |
| | | PDVQISWFVNNVEVHTAQTQTHREDYNSTLRVVSALPI |
| | | QHQDWMSGKEFKCKVNNKDLPAPIERTISKPKGSVRAP |
| | | QVYVLPPPEEEMTKKQVTLTCMVTDFMPEDIYVEWTN |
| | | NGKTELNYKNTEPVLDSDGSYFMYSKLRVEKKNWVERN |
| | | SYSCSVVHEGLHNHHTTKSFSRTPGK |
| SEQ ID NO: 116 | DNA Heavy | CAGGTCCAGCTGCAGCAGTCTGGAGCTGAGCTGGTAA |
| | Chain | GGCCTGGGACTTCAGTGAAGGTGTCCTGCAAGGCTTC |
| | | TGGATACGCCTTCACTAATTACTTGATAGAGTGGGTAA |
| | | AGCAGAGGCCTGGACAGGGCCTTGAGTGGATTGGAG |
| | | TGATTAATCCTGGAAGTGGTGGTACTAACTACAATGA |
| | | GAAGTTCAAGGGCAAGGCAACACTGACTGCAGACAA |
| | | ATCCTCCAGCACTGCCTACATGCAGCTCAGCAGCCTGA |
| | | CATCTGATGACTCTGCGGTCTATTTCTGTGCAAGATGG |
| | | AGGGGGGATGGTTACTACGCGTACTTCGATGTCTGGG |
| | | GCGCAGGGACCACGGTCACCGTCTCCTCAGCCAAAAC |
| | | AACAGCCCCATCGGTCTATCCACTGGCCCCTGTGTGTG |
| | | GAGATACAACTGGCTCCTCGGTGACTCTAGGATGCCT |
| | | GGTCAAGGGTTATTTCCCTGAGCCAGTGACCTTGACCT |
| | | GGAACTCTGGATCCCTGTCCAGTGGTGTGCACACCTTC |
| | | CCAGCTGTCCTGCAGTCTGACCTCTACACCCTCAGCAG |
| | | CTCAGTGACTGTAACCTCGAGCACCTGGCCCAGCCAG |
| | | TCCATCACCTGCAATGTGGCCCACCCGGCAAGCAGCA |
| | | CCAAGGTGGACAAGAAAATTGAGCCCAGAGGGCCCA |
| | | CAATCAAGCCCTGTCCTCCATGCAAATGCCCAGCACCT |
| | | AACCTCTTGGGTGGACCATCCGTCTTCATCTTCCCTCCA |
| | | AAGATCAAGGATGTACTCATGATCTCCCTGAGCCCCAT |
| | | AGTCACATGTGTGGTGGTGGATGTGAGCGAGGATGA |
| | | CCCAGATGTCCAGATCAGCTGGTTTGTGAACAACGTG |
| | | GAAGTACACACAGCTCAGACACAAACCCATAGAGAGG |
| | | ATTACAACAGTACTCTCCGGGTGGTCAGTGCCCTCCCC |
| | | ATCCAGCACCAGGACTGGATGAGTGGCAAGGAGTTC |
| | | AAATGCAAGGTCAACAACAAAGACCTCCCAGCGCCCA |
| | | TCGAGAGAACCATCTCAAAACCCAAAGGGTCAGTAAG |
| | | AGCTCCACAGGTATATGTCTTGCCTCCACCAGAAGAA |
| | | GAGATGACTAAGAAACAGGTCACTCTGACCTGCATGG |
| | | TCACAGACTTCATGCCTGAAGACATTTACGTGGAGTG |
| | | GACCAACAACGGGAAAACAGAGCTAAACTACAAGAA |
| | | CACTGAACCAGTCCTGGACTCTGATGGTTCTTACTTCA |
| | | TGTACAGCAAGCTGAGAGTGGAAAAGAAGAACTGGG |
| | | TGGAAAGAAATAGCTACTCCTGTTCAGTGGTCCACGA |
| | | GGGTCTGCACAATCACCACACGACTAAGAGCTTCTCCC |
| | | GGACTCCGGGTAAA |
| SEQ ID NO: 117 | Light Chain | DIVLTQSPASLAVSLGQRATISCKASQSVDYDGDSYMN |
| | | WYQQKPGQPPKLLIYAASNLESGIPARFSGSGSGTDFTL |
| | | NIHPVEEEDAATYYCQQSNEDPYTFGGGTKLEIKRADAA |
| | | PTVSIFPPSSEQLTSGGASVVCFLNNFYPKDINVKWKIDG |
| | | SERQNGVLNSWTDQDSKDSTYSMSSTLTLTKDEYERHN |
| | | SYTCEATHKTSTSPIVKSFNRNEC |
| SEQ ID NO: 118 | DNA Light | GACATTGTGCTGACCCAATCTCCAGCTTCTTTGGCTGT |
| | Chain | GTCTCTAGGGCAGAGGGCCACCATCTCCTGCAAGGCC |
| | | AGCCAAAGTGTTGATTATGATGGTGATAGTTATATGA |
| | | ACTGGTACCAACAGAAACCAGGACAGCCACCCAAACT |
| | | CCTCATCTATGCTGCATCCAATCTAGAATCTGGGATCC |
| | | CAGCCAGGTTTAGTGGCAGTGGGTCTGGGACAGACTT |
| | | CACCCTCAACATCCATCCTGTGGAGGAGGAGGATGCT |
| | | GCAACCTATTACTGTCAGCAAAGTAATGAGGATCCGT |
| | | ACACGTTCGGAGGGGGGACCAAGCTGGAAATAAAAC |
| | | GGGCTGATGCTGCACCAACTGTATCCATCTTCCCACCA |
| | | TCCAGTGAGCAGTTAACATCTGGAGGTGCCTCAGTCG |
| | | TGTGCTTCTTGAACAACTTCTACCCCAAAGACATCAAT |
| | | GTCAAGTGGAAGATTGATGGCAGTGAACGACAAAAT |
| | | GGCGTCCTGAACAGTTGGACTGATCAGGACAGCAAA |
| | | GACAGCACCTACAGCATGAGCAGCACCCTCACGTTGA |
| | | CCAAGGACGAGTATGAACGACATAACAGCTATACCTG |
| | | TGAGGCCACTCACAAGACATCAACTTCACCCATTGTCA |
| | | AGAGCTTCAACAGGAATGAGTGT |
| SEQ ID NO: 119 | HCDR1 | X1YLIE |
| | consensus | |
| | HCDR1 | X1 = N or S |
| | variable | |
| | amino acids | |
| SEQ ID NO: 120 | HCDR2 | VINPGSGGTNYX1X2X3X4KG |
| | consensus | |
| | HCDR2 | X1 = N or A; X2 = E or D; X3= K or S; |
| | variable | X4 = F or V |
| | amino acids | |
| SEQ ID NO: 121 | HCDR3 | WRGX1X2YYAYFDV |
| | consensus | |
| | HCDR3 | X1 = E, D, A, G, or T; X2 = G, A, T, or S |
| | variable | |
| | amino acids | |
| SEQ ID NO: 122 | LCDR1 | X1ASQSVX2YX3X4DSYMN |
| | consensus | |
| | LCDR1 | X1 = R or K; X2 = S or D; X3 = D, Q, A, G or T; |
| | variable | X4 = G, T or S |
| | amino acids | |
| SEQ ID NO: 123 | LCDR2 | AASNLX1S |
| | consensus | |
| | LCDR2 | X1 = E or A |
| | variable | |
| | amino acids | |
| >104343 | | |
| SEQ ID NO: 124 = | Light chain | DIVLTQSPLSLPVTLGQPASISCKASQSVDYDGDSYMNW |
| SEQ ID NO: 27 + | | YQQRPGQSPRLLIYAASNLESGVPDRFSGSGSGTDFTLKI |
| SEQ ID NO: 107 | | SRVEAEDVGVYYCQQSNEDPYTFGGGTKVEIK |
| | | RTVAAPSVFIFPPSDEQLKSGTASVVCLLNNFYPREAKVQ |
| | | WKVDNALQSGNSQESVTEQDSKDSTYSLSSTLTLSKADY |
| | | EKHKVYACEVTHQGLSSPVTKSFNRGEC |
| SEQ ID NO: 125 = | DNA encoding | GACATCGTGCTGACACAGAGCCCTCTGTCCCTGCCCGT |
| SEQ ID NO: 28 + | light chain | GACCCTGGGCCAGCCTGCCTCCATCTCCTGCAAGGCCT |
| SEQ ID NO: 108 | | CCCAGTCCGTGGACTACGACGGCGACTCCTACATGAA |
| | | CTGGTATCAGCAGCGGCCTGGCCAGTCCCCTCGGCTG |
| | | CTGATCTACGCCGCCTCCAACCTGGAATCCGGCGTGCC |
| | | CGACAGATTCTCCGGCTCCGGCTCTGGCACCGACTTCA |
| | | CCCTGAAGATCTCCCGGGTGGAAGCCGAGGACGTGG |
| | | GCGTGTACTACTGCCAGCAGTCCAACGAGGACCCCTA |
| | | CACCTTCGGCGGAGGCACCAAAGTGGAAATCAAGCGT |
| | | ACGGTGGCCGCTCCCAGCGTGTTCATCTTCCCCCCCAG |
| | | CGACGAGCAGCTGAAGAGCGGCACCGCCAGCGTGGT |
| | | GTGCCTGCTGAACAACTTCTACCCCCGGGAGGCCAAG |
| | | GTGCAGTGGAAGGTGGACAACGCCCTGCAGAGCGGC |
| | | AACAGCCAGGAGAGCGTCACCGAGCAGGACAGCAAG |
| | | GACTCCACCTACAGCCTGAGCAGCACCCTGACCCTGA |
| | | GCAAGGCCGACTACGAGAAGCATAAGGTGTACGCCT |
| | | GCGAGGTGACCCACCAGGGCCTGTCCAGCCCCGTGAC |
| | | CAAGAGCTTCAACAGGGGCGAGTGC |
| SEQ ID NO: 126 = | Heavy chain | QVQLVQSGAEVKKPGSSVKVSCKASGYAFTNYLIEWVR |
| SEQ ID NO: 7 + | | QAPGQGLEWMGVINPGSGGTNYNEKFKGRVTITADKS |
| SEQ ID NO: 103 | | TSTAYMELSSLRSEDTAVYYCARWRGDGYYAYFDVWG |
| | | QGTTVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSVL |
| | | TVLHQDWLNGKEYKCKVSNKALPAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGK |
| SEQ ID NO: 127 = | DNA encoding | CAAGTGCAGCTGGTGCAGTCTGGCGCTGAAGTGAAG |
| SEQ ID NO: 8 + | heavy chain | AAACCCGGCTCCTCCGTGAAAGTGTCCTGCAAGGCCT |
| SEQ ID NO: 104 | | CCGGCTACGCCTTCACCAACTACCTGATCGAGTGGGTC |
| | | CGACAGGCCCCAGGCCAGGGCCTGGAGTGGATGGGC |
| | | GTGATCAACCCTGGCTCCGGCGGCACCAACTACAACG |
| | | AGAAGTTCAAGGGCAGAGTGACCATCACCGCCGACAA |
| | | GTCCACCTCCACCGCCTACATGGAACTGTCCTCCCTGC |
| | | GGAGCGAGGACACCGCCGTGTACTACTGTGCCCGGTG |
| | | GCGGGGAGATGGCTACTACGCCTACTTCGACGTGTGG |
| | | GGCCAGGGCACCACCGTGACCGTGTCCTCTGCTAGCA |
| | | CCAAGGGCCCCTCCGTGTTCCCTCTGGCCCCCTCCAGC |
| | | AAGTCCACCTCTGGCGGCACCGCCGCTCTGGGCTGCC |
| | | TGGTGAAAGACTACTTCCCCGAGCCCGTGACCGTGTC |
| | | CTGGAACTCTGGCGCCCTGACCTCCGGCGTGCACACC |
| | | TTTCCAGCCGTGCTGCAGTCCTCCGGCCTGTACTCCCT |
| | | GTCCTCCGTGGTGACCGTGCCCTCTAGCTCTCTGGGCA |
| | | CCCAGACCTACATCTGCAACGTGAACCACAAGCCCTCC |
| | | AACACCAAGGTGGACAAGCGGGTGGAACCCAAGTCC |
| | | TGCGACAAGACCCACACCTGTCCCCCCTGCCCTGCCCC |
| | | TGAACTGCTGGGCGGACCTTCCGTGTTCCTGTTCCCCC |
| | | CAAAGCCCAAGGACACCCTGATGATCTCCCGGACCCC |
| | | CGAAGTGACCTGCGTGGTGGTGGACGTGTCCCACGA |
| | | GGACCCTGAAGTGAAGTTCAATTGGTACGTGGACGGC |
| | | GTGGAAGTGCACAACGCCAAGACCAAGCCCAGAGAG |
| | | GAACAGTACGCCTCCACCTACCGGGTGGTGTCTGTGC |
| | | TGACCGTGCTGCACCAGGACTGGCTGAACGGCAAAG |
| | | AGTACAAGTGCAAGGTCTCCAACAAGGCCCTGCCTGC |
| | | CCCCATCGAAAAGACCATCTCCAAGGCCAAGGGCCAG |
| | | CCCCGCGAGCCACAGGTGTACACACTGCCCCCCAGCC |
| | | GGGAAGAGATGACCAAGAACCAGGTGTCCCTGACCT |
| | | GTCTGGTCAAAGGCTTCTACCCCTCCGATATCGCCGTG |
| | | GAGTGGGAGTCCAACGGACAGCCCGAGAACAACTAC |
| | | AAGACCACCCCCCCTGTGCTGGACTCCGACGGCTCATT |
| | | CTTCCTGTACTCCAAGCTGACCGTGGACAAGTCCCGGT |
| | | GGCAGCAGGGCAACGTGTTCTCCTGCTCCGTGATGCA |
| | | CGAGGCCCTGCACAACCACTACACCCAGAAGTCCCTG |
| | | TCCCTGAGCCCCGGCAAG |
| >104348 | | |
| SEQ ID NO: 128 = | Light chain | AIRLTQSPSSFSASTGDRVTITCKASQSVDYDGDSYMNW |
| SEQ ID NO: 25 + | | YQQKPGKAPKLLIYAASNLESGVPSRFSGSGSGTDFTLTIS |
| SEQ ID NO: 107 | | SLQSEDFATYYCQQSNEDPYTFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| SEQ ID NO: 129 = | DNA encoding | GCCATCAGACTGACCCAGAGCCCCTCCAGCTTCTCCGC |
| SEQ ID NO: 26 + | light chain | CTCCACCGGCGACAGAGTGACCATCACATGCAAGGCC |
| SEQ ID NO: 108 | | TCCCAGTCCGTGGACTACGACGGCGACTCCTACATGA |
| | | ACTGGTATCAGCAGAAGCCCGGCAAGGCCCCTAAGCT |
| | | GCTGATCTACGCCGCCTCCAACCTGGAATCCGGCGTG |
| | | CCCTCCCGGTTCTCCGGCTCTGGCTCTGGCACCGACTT |
| | | CACCCTGACCATCTCCAGCCTGCAGTCCGAGGACTTCG |
| | | CCACCTACTACTGCCAGCAGTCCAACGAGGACCCCTAC |
| | | ACCTTCGGCGGAGGCACCAAAGTGGAAATCAAGCGT |
| | | ACGGTGGCCGCTCCCAGCGTGTTCATCTTCCCCCCCAG |
| | | CGACGAGCAGCTGAAGAGCGGCACCGCCAGCGTGGT |
| | | GTGCCTGCTGAACAACTTCTACCCCCGGGAGGCCAAG |
| | | GTGCAGTGGAAGGTGGACAACGCCCTGCAGAGCGGC |
| | | AACAGCCAGGAGAGCGTCACCGAGCAGGACAGCAAG |
| | | GACTCCACCTACAGCCTGAGCAGCACCCTGACCCTGA |
| | | GCAAGGCCGACTACGAGAAGCATAAGGTGTACGCCT |
| | | GCGAGGTGACCCACCAGGGCCTGTCCAGCCCCGTGAC |
| | | CAAGAGCTTCAACAGGGGCGAGTGC |
| SEQ ID NO: 130 = | Heavy chain | EVQLVQSGAEVKKPGESLKISCKGSGYAFTNYLIEWVRQ |
| SEQ ID NO: 17 + | | MPGKGLEWMGVINPGSGGTNYNEKFKGQVTISADKSIS |
| SEQ ID NO: 103 | | TAYLQWSSLKASDTAMYYCARWRGDGYYAYFDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKKVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSVL |
| | | TVLHQDWLNGKEYKCKVSNKALPAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGK |
| SEQ ID NO: 131 = | DNA encoding | GAAGTGCAGCTGGTGCAGTCTGGCGCTGAAGTGAAG |
| SEQ ID NO: 18 + | heavy chain | AAGCCCGGCGAGTCCCTGAAGATCTCCTGCAAGGGCT |
| SEQ ID NO: 104 | | CCGGCTACGCCTTCACCAACTACCTGATCGAGTGGGTC |
| | | CGACAGATGCCCGGCAAGGGCCTGGAGTGGATGGGC |
| | | GTGATCAACCCCGGCTCCGGCGGCACCAACTACAACG |
| | | AGAAGTTCAAGGGCCAAGTCACAATCTCCGCCGACAA |
| | | GTCCATCTCCACCGCCTACCTGCAGTGGTCCTCCCTGA |
| | | AGGCCTCCGACACCGCCATGTACTACTGCGCCAGATG |
| | | GCGGGGAGATGGCTACTACGCCTACTTCGACGTGTGG |
| | | GGCCAGGGCACCACCGTGACCGTGTCCTCTGCTAGCA |
| | | CCAAGGGCCCCTCCGTGTTCCCTCTGGCCCCCTCCAGC |
| | | AAGTCCACCTCTGGCGGCACCGCCGCTCTGGGCTGCC |
| | | TGGTGAAAGACTACTTCCCCGAGCCCGTGACCGTGTC |
| | | CTGGAACTCTGGCGCCCTGACCTCCGGCGTGCACACC |
| | | TTTCCAGCCGTGCTGCAGTCCTCCGGCCTGTACTCCCT |
| | | GTCCTCCGTGGTGACCGTGCCCTCTAGCTCTCTGGGCA |
| | | CCCAGACCTACATCTGCAACGTGAACCACAAGCCCTCC |
| | | AACACCAAGGTGGACAAGCGGGTGGAACCCAAGTCC |
| | | TGCGACAAGACCCACACCTGTCCCCCCTGCCCTGCCCC |
| | | TGAACTGCTGGGCGGACCTTCCGTGTTCCTGTTCCCCC |
| | | CAAAGCCCAAGGACACCCTGATGATCTCCCGGACCCC |
| | | CGAAGTGACCTGCGTGGTGGTGGACGTGTCCCACGA |
| | | GGACCCTGAAGTGAAGTTCAATTGGTACGTGGACGGC |
| | | GTGGAAGTGCACAACGCCAAGACCAAGCCCAGAGAG |
| | | GAACAGTACGCCTCCACCTACCGGGTGGTGTCTGTGC |
| | | TGACCGTGCTGCACCAGGACTGGCTGAACGGCAAAG |
| | | AGTACAAGTGCAAGGTCTCCAACAAGGCCCTGCCTGC |
| | | CCCCATCGAAAAGACCATCTCCAAGGCCAAGGGCCAG |
| | | CCCCGCGAGCCACAGGTGTACACACTGCCCCCCAGCC |
| | | GGGAAGAGATGACCAAGAACCAGGTGTCCCTGACCT |
| | | GTCTGGTCAAAGGCTTCTACCCCTCCGATATCGCCGTG |
| | | GAGTGGGAGTCCAACGGACAGCCCGAGAACAACTAC |
| | | AAGACCACCCCCCCTGTGCTGGACTCCGACGGCTCATT |
| | | CTTCCTGTACTCCAAGCTGACCGTGGACAAGTCCCGGT |
| | | GGCAGCAGGGCAACGTGTTCTCCTGCTCCGTGATGCA |
| | | CGAGGCCCTGCACAACCACTACACCCAGAAGTCCCTG |
| | | TCCCTGAGCCCCGGCAAG |
| SEQ ID NO: 132 | Epitope | GINNYKNPKLTRMLTFKFYMPKKATELKHLQCLEEELKPL |
| | section | EEVLNLAQSKNF |
| >VH-F100dY/N58Y/T30S | | |
| SEQ ID NO: 133 (Combined) | HCDR1 | GYAFSNYLIE |
| SEQ ID NO: 134 (Combined) | HCDR2 | VINPGSGGTYYNEKFKG |
| SEQ ID NO: 135 (Combined) | HCDR3 | WRGEGYYAYYDV |
| SEQ ID NO: 136 (Kabat) | HCDR1 | NYLIE |
| SEQ ID NO: 137 (Kabat) | HCDR2 | VINPGSGGTYYNEKFKG |
| SEQ ID NO: 138 (Kabat) | HCDR3 | WRGEGYYAYYDV |
| SEQ ID NO: 139 (Chothia) | HCDR1 | GYAFSNY |
| SEQ ID NO: 140 (Chothia) | HCDR2 | NPGSGG |
| SEQ ID NO: 141 (Chothia) | HCDR3 | WRGEGYYAYYDV |
| SEQ ID NO: 142 (IMGT) | HCDR1 | GYAFSNYL |
| SEQ ID NO: 143 (IMGT) | HCDR2 | INPGSGGT |
| SEQ ID NO: 144 (IMGT) | HCDR3 | ARWRGEGYYAYYDV |
| SEQ ID NO: 145 | VH | EVQLVQSGAEVKKPGESLKISCKGSGYAFSNYLIEWVRQ |
| | | MPGKGLEWMGVINPGSGGTYYNEKFKGQVTISADKSIS |
| | | TAYLQWSSLKASDTAMYYCARWRGEGYYAYYDVWGQ |
| | | GTTVTVSS |
| SEQ ID NO: 146 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCA |
| SEQ ID NO: 147 | Heavy Chain | EVQLVQSGAEVKKPGESLKISCKGSGYAFSNYLIEWVRQ |
| | | MPGKGLEWMGVINPGSGGTYYNEKFKGQVTISADKSIS |
| | | TAYLQWSSLKASDTAMYYCARWRGEGYYAYYDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 148 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VH-F100dY/N58Y | | |
| SEQ ID NO: 149 (Combined) | HCDR1 | GYAFTNYLIE |
| SEQ ID NO: 150 (Combined) | HCDR2 | VINPGSGGTYYNEKFKG |
| SEQ ID NO: 151 (Combined) | HCDR3 | WRGEGYYAYYDV |
| SEQ ID NO: 152 (Kabat) | HCDR1 | NYLIE |
| SEQ ID NO: 153 (Kabat) | HCDR2 | VINPGSGGTYYNEKFKG |
| SEQ ID NO: 154 (Kabat) | HCDR3 | WRGEGYYAYYDV |
| SEQ ID NO: 155 (Chothia) | HCDR1 | GYAFTNY |
| SEQ ID NO: 156 (Chothia) | HCDR2 | NPGSGG |
| SEQ ID NO: 157 (Chothia) | HCDR3 | WRGEGYYAYYDV |
| SEQ ID NO: 158 (IMGT) | HCDR1 | GYAFTNYL |
| SEQ ID NO: 159 (IMGT) | HCDR2 | INPGSGGT |
| SEQ ID NO: 160 (IMGT) | HCDR3 | ARWRGEGYYAYYDV |
| SEQ ID NO: 161 | VH | EVQLVQSGAEVKKPGESLKISCKGSGYAFTNYLIEWVRQ |
| | | MPGKGLEWMGVINPGSGGTYYNEKFKGQVTISADKSIS |
| | | TAYLQWSSLKASDTAMYYCARWRGEGYYAYYDVWGQ |
| | | GTTVTVSS |
| SEQ ID NO: 162 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCA |
| SEQ ID NO: 163 | Heavy Chain | EVQLVQSGAEVKKPGESLKISCKGSGYAFTNYLIEWVRQ |
| | | MPGKGLEWMGVINPGSGGTYYNEKFKGQVTISADKSIS |
| | | TAYLQWSSLKASDTAMYYCARWRGEGYYAYYDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 164 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VH-F100dY/T30S | | |
| SEQ ID NO: 165 (Combined) | HCDR1 | GYAFSNYLIE |
| SEQ ID NO: 166 (Combined) | HCDR2 | VINPGSGGTNYNEKFKG |
| SEQ ID NO: 167 (Combined) | HCDR3 | WRGEGYYAYYDV |
| SEQ ID NO: 168 (Kabat) | HCDR1 | NYLIE |
| SEQ ID NO: 169 (Kabat) | HCDR2 | VINPGSGGTNYNEKFKG |
| SEQ ID NO: 170 (Kabat) | HCDR3 | WRGEGYYAYYDV |
| SEQ ID NO: 171 (Chothia) | HCDR1 | GYAFSNY |
| SEQ ID NO: 172 (Chothia) | HCDR2 | NPGSGG |
| SEQ ID NO: 173 (Chothia) | HCDR3 | WRGEGYYAYYDV |
| SEQ ID NO: 174 (IMGT) | HCDR1 | GYAFSNYL |
| SEQ ID NO: 175 (IMGT) | HCDR2 | INPGSGGT |
| SEQ ID NO: 176 (IMGT) | HCDR3 | ARWRGEGYYAYYDV |
| SEQ ID NO: 177 | VH | EVQLVQSGAEVKKPGESLKISCKGSGYAFSNYLIEWVRQ |
| | | MPGKGLEWMGVINPGSGGTNYNEKFKGQVTISADKSIS |
| | | TAYLQWSSLKASDTAMYYCARWRGEGYYAYYDVWGQ |
| | | GTTVTVSS |
| SEQ ID NO: 178 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCA |
| SEQ ID NO: 179 | Heavy Chain | EVQLVQSGAEVKKPGESLKISCKGSGYAFSNYLIEWVRQ |
| | | MPGKGLEWMGVINPGSGGTNYNEKFKGQVTISADKSIS |
| | | TAYLQWSSLKASDTAMYYCARWRGEGYYAYYDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 180 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VH-F100dY | | |
| SEQ ID NO: 181 (Combined) | HCDR1 | GYAFTNYLIE |
| SEQ ID NO: 182 (Combined) | HCDR2 | VINPGSGGTNYNEKFKG |
| SEQ ID NO: 183 (Combined) | HCDR3 | WRGEGYYAYYDV |
| SEQ ID NO: 184 (Kabat) | HCDR1 | NYLIE |
| SEQ ID NO: 185 (Kabat) | HCDR2 | VINPGSGGTNYNEKFKG |
| SEQ ID NO: 186 (Kabat) | HCDR3 | WRGEGYYAYYDV |
| SEQ ID NO: 187 (Chothia) | HCDR1 | GYAFTNY |
| SEQ ID NO: 188 (Chothia) | HCDR2 | NPGSGG |
| SEQ ID NO: 189 (Chothia) | HCDR3 | WRGEGYYAYYDV |
| SEQ ID NO: 190 (IMGT) | HCDR1 | GYAFTNYL |
| SEQ ID NO: 191 (IMGT) | HCDR2 | INPGSGGT |
| SEQ ID NO: 192 (IMGT) | HCDR3 | ARWRGEGYYAYYDV |
| SEQ ID NO: 193 | VH | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGEGYYAYYDVWGQ |
| | | GTTVTVSS |
| SEQ ID NO: 194 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCA |
| SEQ ID NO: 195 | Heavy Chain | EVOLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGEGYYAYYDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 196 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTACGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VH-N58Y/T30S | | |
| SEQ ID NO: 197 (Combined) | HCDR1 | GYAFSNYLIE |
| SEQ ID NO: 198 (Combined) | HCDR2 | VINPGSGGTYYNEKFKG |
| SEQ ID NO: 199 (Combined) | HCDR3 | WRGEGYYAYFDV |
| SEQ ID NO: 200 (Kabat) | HCDR1 | NYLIE |
| SEQ ID NO: 201 (Kabat) | HCDR2 | VINPGSGGTYYNEKFKG |
| SEQ ID NO: 202 (Kabat) | HCDR3 | WRGEGYYAYFDV |
| SEQ ID NO: 203 (Chothia) | HCDR1 | GYAFSNY |
| SEQ ID NO: 204 (Chothia) | HCDR2 | NPGSGG |
| SEQ ID NO: 205 (Chothia) | HCDR3 | WRGEGYYAYFDV |
| SEQ ID NO: 206 (IMGT) | HCDR1 | GYAFSNYL |
| SEQ ID NO: 207 (IMGT) | HCDR2 | INPGSGGT |
| SEQ ID NO: 208 (IMGT) | HCDR3 | ARWRGEGYYAYFDV |
| SEQ ID NO: 209 | VH | EVQLVQSGAEVKKPGESLKISCKGSGYAFSNYLIEWVRQ |
| | | MPGKGLEWMGVINPGSGGTYYNEKFKGQVTISADKSIS |
| | | TAYLQWSSLKASDTAMYYCARWRGEGYYAYFDVWGQ |
| | | GTTVTVSS |
| SEQ ID NO: 210 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTTCGACG |
| | | TGTGGGGCCAGGGCACCACAGTGACCGTCAGCTCA |
| SEQ ID NO: 211 | Heavy Chain | EVQLVQSGAEVKKPGESLKISCKGSGYAFSNYLIEWVRQ |
| | | MPGKGLEWMGVINPGSGGTYYNEKFKGQVTISADKSIS |
| | | TAYLQWSSLKASDTAMYYCARWRGEGYYAYFDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 212 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCAGCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTTCGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VH-N58Y | | |
| SEQ ID NO: 213 (Combined) | HCDR1 | GYAFTNYLIE |
| SEQ ID NO: 214 (Combined) | HCDR2 | VINPGSGGTYYNEKFKG |
| SEQ ID NO: 215 (Combined) | HCDR3 | WRGEGYYAYFDV |
| SEQ ID NO: 216 (Kabat) | HCDR1 | NYLIE |
| SEQ ID NO: 217 (Kabat) | HCDR2 | VINPGSGGTYYNEKFKG |
| SEQ ID NO: 218 (Kabat) | HCDR3 | WRGEGYYAYFDV |
| SEQ ID NO: 219 (Chothia) | HCDR1 | GYAFTNY |
| SEQ ID NO: 220 (Chothia) | HCDR2 | NPGSGG |
| SEQ ID NO: 221 (Chothia) | HCDR3 | WRGEGYYAYFDV |
| SEQ ID NO: 222 (IMGT) | HCDR1 | GYAFTNYL |
| SEQ ID NO: 223 (IMGT) | HCDR2 | INPGSGGT |
| SEQ ID NO: 224 (IMGT) | HCDR3 | ARWRGEGYYAYFDV |
| SEQ ID NO: 225 | VH | EVQLVQSGAEVKKPGESLKISCKGSGYAFTNYLIEWVRQ |
| | | MPGKGLEWMGVINPGSGGTYYNEKFKGQVTISADKSIS |
| | | TAYLQWSSLKASDTAMYYCARWRGEGYYAYFDVWGQ |
| | | GTTVTVSS |
| SEQ ID NO: 226 | DNA VH | GAGGTGCAATTGGTGCAGAGCGGAGCCGAAGTGAAG |
| | | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTTCGACG |
| | | TGTGGGGCCAGGGCACCACAGTGACCGTCAGCTCA |
| SEQ ID NO: 227 | Heavy Chain | EVQLVQSGAEVKKPGESLKISCKGSGYAFTNYLIEWVRQ |
| | | MPGKGLEWMGVINPGSGGTYYNEKFKGQVTISADKSIS |
| | | TAYLQWSSLKASDTAMYYCARWRGEGYYAYFDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 228 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCTACTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTTCGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >104348_VH5D98E_VK1D28Q | | |
| heavy | | |
| SEQ ID NO: 229 | Heavy Chain | EVQLVQSGAEVKKPGESLKISCKGSGYAFTNYLIEWVRQ |
| | | MPGKGLEWMGVINPGSGGTNYNEKFKGQVTISADKSIS |
| | | TAYLQWSSLKASDTAMYYCARWRGEGYYAYFDVWGQ |
| | | GTTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYASTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 230 | DNA Heavy | GAGGTGCAATTGGTGCAGAGCGGAGCCGAAGTGAAG |
| | Chain | AAGCCCGGCGAGAGCCTGAAGATCAGCTGCAAGGGC |
| | | AGCGGCTACGCCTTCACCAACTACCTGATCGAGTGGG |
| | | TGCGCCAGATGCCCGGCAAGGGCCTGGAATGGATGG |
| | | GCGTGATCAATCCTGGCAGCGGCGGCACCAATTACAA |
| | | CGAGAAGTTCAAGGGCCAAGTGACCATCAGCGCCGA |
| | | CAAGAGCATCAGCACCGCCTACCTCCAGTGGTCCAGC |
| | | CTGAAGGCCAGCGACACCGCCATGTACTACTGCGCCA |
| | | GGTGGCGGGGAGAGGGCTACTACGCCTACTTCGACG |
| | | TGTGGGGCCAGGGCACCACAGTGACCGTCAGCTCAGC |
| | | TAGCACCAAGGGCCCCAGCGTGTTCCCCCTGGCCCCC |
| | | AGCAGCAAGAGCACCAGCGGCGGCACAGCCGCCCTG |
| | | GGCTGCCTGGTGAAGGACTACTTCCCCGAGCCCGTGA |
| | | CCGTGTCCTGGAACAGCGGAGCCCTGACCTCCGGCGT |
| | | GCACACCTTCCCCGCCGTGCTGCAGAGCAGCGGCCTG |
| | | TACAGCCTGTCCAGCGTGGTGACAGTGCCCAGCAGCA |
| | | GCCTGGGCACCCAGACCTACATCTGCAACGTGAACCA |
| | | CAAGCCCAGCAACACCAAGGTGGACAAGAGAGTGGA |
| | | GCCCAAGAGCTGCGACAAGACCCACACCTGCCCCCCC |
| | | TGCCCAGCCCCAGAGCTGCTGGGCGGACCCTCCGTGT |
| | | TCCTGTTCCCCCCCAAGCCCAAGGACACCCTGATGATC |
| | | AGCAGGACCCCCGAGGTGACCTGCGTGGTGGTGGAC |
| | | GTGAGCCACGAGGACCCAGAGGTGAAGTTCAACTGG |
| | | TACGTGGACGGCGTGGAGGTGCACAACGCCAAGACC |
| | | AAGCCCAGAGAGGAGCAGTACGCCAGCACCTACAGG |
| | | GTGGTGTCCGTGCTGACCGTGCTGCACCAGGACTGGC |
| | | TGAACGGCAAGGAATACAAGTGCAAGGTCTCCAACAA |
| | | GGCCCTGCCAGCCCCCATCGAAAAGACCATCAGCAAG |
| | | GCCAAGGGCCAGCCACGGGAGCCCCAGGTGTACACC |
| | | CTGCCCCCCTCCCGGGAGGAGATGACCAAGAACCAGG |
| | | TGTCCCTGACCTGTCTGGTGAAGGGCTTCTACCCCAGC |
| | | GACATCGCCGTGGAGTGGGAGAGCAACGGCCAGCCC |
| | | GAGAACAACTACAAGACCACCCCCCCAGTGCTGGACA |
| | | GCGACGGCAGCTTCTTCCTGTACAGCAAGCTGACCGT |
| | | GGACAAGTCCAGGTGGCAGCAGGGCAACGTGTTCAG |
| | | CTGCAGCGTGATGCACGAGGCCCTGCACAACCACTAC |
| | | ACCCAGAAGAGCCTGAGCCTGTCCCCCGGCAAG |
| >VK-A50S | | |
| SEQ ID NO: 231 (Combined) | LCDR1 | KASQSVDYQGDSYMN |
| SEQ ID NO: 232 (Combined) | LCDR2 | SASNLES |
| SEQ ID NO: 233 (Combined) | LCDR3 | QQSNEDPYT |
| SEQ ID NO: 234 (Kabat) | LCDR1 | KASQSVDYQGDSYMN |
| SEQ ID NO: 235 (Kabat) | LCDR2 | SASNLES |
| SEQ ID NO: 236 (Kabat) | LCDR3 | QQSNEDPYT |
| SEQ ID NO: 237 (Chothia) | LCDR1 | SQSVDYQGDSY |
| SEQ ID NO: 238 (Chothia) | LCDR2 | SAS |
| SEQ ID NO: 239 (Chothia) | LCDR3 | SNEDPY |
| SEQ ID NO: 240 (IMGT) | LCDR1 | QSVDYQGDSY |
| SEQ ID NO: 241 (IMGT) | LCDR2 | SAS |
| SEQ ID NO: 242 (IMGT) | LCDR3 | QQSNEDPYT |
| SEQ ID NO: 243 | VL | AIRLTQSPSSFSASTGDRVTITCKASQSVDYQGDSYMNW |
| | | YQQKPGKAPKLLIYSASNLESGVPSRFSGSGSGTDFTLTIS |
| | | SLQSEDFATYYCQQSNEDPYTFGGGTKVEIK |
| SEQ ID NO: 244 | DNA VL | GCCATCAGACTGACCCAGAGCCCCAGCAGCTTTAGCG |
| | | CCAGCACCGGCGACAGAGTGACCATCACATGCAAGGC |
| | | CAGCCAGAGCGTGGACTACCAGGGCGACAGCTACAT |
| | | GAACTGGTATCAGCAGAAGCCCGGCAAGGCCCCCAA |
| | | GCTGCTGATCTACTCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AG |
| SEQ ID NO: 245 | Light Chain | AIRLTQSPSSFSASTGDRVTITCKASQSVDYQGDSYMNW |
| | | YQQKPGKAPKLLIYSASNLESGVPSRFSGSGSGTDFTLTIS |
| | | SLQSEDFATYYCQQSNEDPYTFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| SEQ ID NO: 246 | DNA Light | GCCATCAGACTGACCCAGAGCCCCAGCAGCTTTAGCG |
| | Chain | CCAGCACCGGCGACAGAGTGACCATCACATGCAAGGC |
| | | CAGCCAGAGCGTGGACTACCAGGGCGACAGCTACAT |
| | | GAACTGGTATCAGCAGAAGCCCGGCAAGGCCCCCAA |
| | | GCTGCTGATCTACTCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AGCGTACGGTGGCCGCTCCCAGCGTGTTCATCTTCCCC |
| | | CCCAGCGACGAGCAGCTGAAGAGCGGCACCGCCAGC |
| | | GTGGTGTGCCTGCTGAACAACTTCTACCCCCGGGAGG |
| | | CCAAGGTGCAGTGGAAGGTGGACAACGCCCTGCAGA |
| | | GCGGCAACAGCCAGGAGAGCGTCACCGAGCAGGACA |
| | | GCAAGGACTCCACCTACAGCCTGAGCAGCACCCTGAC |
| | | CCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| >VK-A50T | | |
| SEQ ID NO: 247 (Combined) | LCDR1 | KASQSVDYQGDSYMN |
| SEQ ID NO: 248 (Combined) | LCDR2 | TASNLES |
| SEQ ID NO: 249 (Combined) | LCDR3 | QQSNEDPYT |
| SEQ ID NO: 250 (Kabat) | LCDR1 | KASQSVDYQGDSYMN |
| SEQ ID NO: 251 (Kabat) | LCDR2 | TASNLES |
| SEQ ID NO: 252 (Kabat) | LCDR3 | QQSNEDPYT |
| SEQ ID NO: 253 (Chothia) | LCDR1 | SQSVDYQGDSY |
| SEQ ID NO: 254 (Chothia) | LCDR2 | TAS |
| SEQ ID NO: 255 (Chothia) | LCDR3 | SNEDPY |
| SEQ ID NO: 256 (IMGT) | LCDR1 | QSVDYQGDSY |
| SEQ ID NO: 257 (IMGT) | LCDR2 | TAS |
| SEQ ID NO: 258 (IMGT) | LCDR3 | QQSNEDPYT |
| SEQ ID NO: 259 | VL | AIRLTQSPSSFSASTGDRVTITCKASQSVDYQGDSYMNW |
| | | YQQKPGKAPKLLIYTASNLESGVPSRFSGSGSGTDFTLTIS |
| | | SLQSEDFATYYCQQSNEDPYTFGGGTKVEIK |
| SEQ ID NO: 260 | DNA VL | GCCATCAGACTGACCCAGAGCCCCAGCAGCTTTAGCG |
| | | CCAGCACCGGCGACAGAGTGACCATCACATGCAAGGC |
| | | CAGCCAGAGCGTGGACTACCAGGGCGACAGCTACAT |
| | | GAACTGGTATCAGCAGAAGCCCGGCAAGGCCCCCAA |
| | | GCTGCTGATCTACACCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AG |
| SEQ ID NO: 261 | Light Chain | AIRLTQSPSSFSASTGDRVTITCKASQSVDYQGDSYMNW |
| | | YQQKPGKAPKLLIYTASNLESGVPSRFSGSGSGTDFTLTIS |
| | | SLQSEDFATYYCQQSNEDPYTFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| SEQ ID NO: 262 | DNA Light | GCCATCAGACTGACCCAGAGCCCCAGCAGCTTTAGCG |
| | Chain | CCAGCACCGGCGACAGAGTGACCATCACATGCAAGGC |
| | | CAGCCAGAGCGTGGACTACCAGGGCGACAGCTACAT |
| | | GAACTGGTATCAGCAGAAGCCCGGCAAGGCCCCCAA |
| | | GCTGCTGATCTACACCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AGCGTACGGTGGCCGCTCCCAGCGTGTTCATCTTCCCC |
| | | CCCAGCGACGAGCAGCTGAAGAGCGGCACCGCCAGC |
| | | GTGGTGTGCCTGCTGAACAACTTCTACCCCCGGGAGG |
| | | CCAAGGTGCAGTGGAAGGTGGACAACGCCCTGCAGA |
| | | GCGGCAACAGCCAGGAGAGCGTCACCGAGCAGGACA |
| | | GCAAGGACTCCACCTACAGCCTGAGCAGCACCCTGAC |
| | | CCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| >VK-M33L/A50S | | |
| SEQ ID NO: 263 (Combined) | LCDR1 | KASQSVDYQGDSYLN |
| SEQ ID NO: 264 (Combined) | LCDR2 | SASNLES |
| SEQ ID NO: 265 (Combined) | LCDR3 | QQSNEDPYT |
| SEQ ID NO: 266 (Kabat) | LCDR1 | KASQSVDYQGDSYLN |
| SEQ ID NO: 267 (Kabat) | LCDR2 | SASNLES |
| SEQ ID NO: 268 (Kabat) | LCDR3 | QQSNEDPYT |
| SEQ ID NO: 269 (Chothia) | LCDR1 | SQSVDYQGDSY |
| SEQ ID NO: 270 (Chothia) | LCDR2 | SAS |
| SEQ ID NO: 271 (Chothia) | LCDR3 | SNEDPY |
| SEQ ID NO: 272 (IMGT) | LCDR1 | QSVDYQGDSY |
| SEQ ID NO: 273 (IMGT) | LCDR2 | SAS |
| SEQ ID NO: 274 (IMGT) | LCDR3 | QQSNEDPYT |
| SEQ ID NO: 275 | VL | AIRLTQSPSSFSASTGDRVTITCKASQSVDYQGDSYLNWY |
| | | QQKPGKAPKLLIYSASNLESGVPSRFSGSGSGTDFTLTISS |
| | | LQSEDFATYYCQQSNEDPYTFGGGTKVEIK |
| SEQ ID NO: 276 | DNA VL | GCCATCAGACTGACCCAGAGCCCCAGCAGCTTTAGCG |
| | | CCAGCACCGGCGACAGAGTGACCATCACATGCAAGGC |
| | | CAGCCAGAGCGTGGACTACCAGGGCGACAGCTACCT |
| | | GAACTGGTATCAGCAGAAGCCCGGCAAGGCCCCCAA |
| | | GCTGCTGATCTACTCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AG |
| SEQ ID NO: 277 | Light Chain | AIRLTQSPSSFSASTGDRVTITCKASQSVDYQGDSYLNWY |
| | | QQKPGKAPKLLIYSASNLESGVPSRFSGSGSGTDFTLTISS |
| | | LQSEDFATYYCQQSNEDPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSFNRGEC |
| SEQ ID NO: 278 | DNA Light | GCCATCAGACTGACCCAGAGCCCCAGCAGCTTTAGCG |
| | Chain | CCAGCACCGGCGACAGAGTGACCATCACATGCAAGGC |
| | | CAGCCAGAGCGTGGACTACCAGGGCGACAGCTACCT |
| | | GAACTGGTATCAGCAGAAGCCCGGCAAGGCCCCCAA |
| | | GCTGCTGATCTACTCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AGCGTACGGTGGCCGCTCCCAGCGTGTTCATCTTCCCC |
| | | CCCAGCGACGAGCAGCTGAAGAGCGGCACCGCCAGC |
| | | GTGGTGTGCCTGCTGAACAACTTCTACCCCCGGGAGG |
| | | CCAAGGTGCAGTGGAAGGTGGACAACGCCCTGCAGA |
| | | GCGGCAACAGCCAGGAGAGCGTCACCGAGCAGGACA |
| | | GCAAGGACTCCACCTACAGCCTGAGCAGCACCCTGAC |
| | | CCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| >VK-M33L | | |
| SEQ ID NO: 279 (Combined) | LCDR1 | KASQSVDYQGDSYLN |
| SEQ ID NO: 280 (Combined) | LCDR2 | AASNLES |
| SEQ ID NO: 281 (Combined) | LCDR3 | QQSNEDPYT |
| SEQ ID NO: 282 (Kabat) | LCDR1 | KASQSVDYQGDSYLN |
| SEQ ID NO: 283 (Kabat) | LCDR2 | AASNLES |
| SEQ ID NO: 284 (Kabat) | LCDR3 | QQSNEDPYT |
| SEQ ID NO: 285 (Chothia) | LCDR1 | SQSVDYQGDSY |
| SEQ ID NO: 386 (Chothia) | LCDR2 | AAS |
| SEQ ID NO: 387 (Chothia) | LCDR3 | SNEDPY |
| SEQ ID NO: 388 (IMGT) | LCDR1 | QSVDYQGDSY |
| SEQ ID NO: 389 (IMGT) | LCDR2 | AAS |
| SEQ ID NO: 390 (IMGT) | LCDR3 | QQSNEDPYT |
| SEQ ID NO: 391 | VL | AIRLTQSPSSFSASTGDRVTITCKASQSVDYQGDSYLNWY |
| | | QQKPGKAPKLLIYAASNLESGVPSRFSGSGSGTDFTLTISS |
| | | LQSEDFATYYCQQSNEDPYTFGGGTKVEIK |
| SEQ ID NO: 392 | DNA VL | GCCATCAGACTGACCCAGAGCCCCAGCAGCTTTAGCG |
| | | CCAGCACCGGCGACAGAGTGACCATCACATGCAAGGC |
| | | CAGCCAGAGCGTGGACTACCAGGGCGACAGCTACCT |
| | | GAACTGGTATCAGCAGAAGCCCGGCAAGGCCCCCAA |
| | | GCTGCTGATCTACGCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AG |
| SEQ ID NO: 393 | Light Chain | AIRLTQSPSSFSASTGDRVTITCKASQSVDYQGDSYLNWY |
| | | QQKPGKAPKLLIYAASNLESGVPSRFSGSGSGTDFTLTISS |
| | | LQSEDFATYYCQQSNEDPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSFNRGEC |
| SEQ ID NO: 394 | DNA Light | GCCATCAGACTGACCCAGAGCCCCAGCAGCTTTAGCG |
| | Chain | CCAGCACCGGCGACAGAGTGACCATCACATGCAAGGC |
| | | CAGCCAGAGCGTGGACTACCAGGGCGACAGCTACCT |
| | | GAACTGGTATCAGCAGAAGCCCGGCAAGGCCCCCAA |
| | | GCTGCTGATCTACGCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AGCGTACGGTGGCCGCTCCCAGCGTGTTCATCTTCCCC |
| | | CCCAGCGACGAGCAGCTGAAGAGCGGCACCGCCAGC |
| | | GTGGTGTGCCTGCTGAACAACTTCTACCCCCGGGAGG |
| | | CCAAGGTGCAGTGGAAGGTGGACAACGCCCTGCAGA |
| | | GCGGCAACAGCCAGGAGAGCGTCACCGAGCAGGACA |
| | | GCAAGGACTCCACCTACAGCCTGAGCAGCACCCTGAC |
| | | CCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| >104348_VH5D98E_VK1D28Q | | |
| light | | |
| SEQ ID NO: 395 = | Light Chain | AIRLTQSPSSFSASTGDRVTITCKASQSVDYQGDSYMNW |
| SEQ ID NO: 70 + | | YQQKPGKAPKLLIYAASNLESGVPSRFSGSGSGTDFTLTIS |
| SEQ ID NO: 107 | | SLQSEDFATYYCQQSNEDPYTFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |
| SEQ ID NO: 396 | DNA Light | GCCATCAGACTGACCCAGAGCCCCAGCAGCTTTAGCG |
| | Chain | CCAGCACCGGCGACAGAGTGACCATCACATGCAAGGC |
| | | CAGCCAGAGCGTGGACTACCAGGGCGACAGCTACAT |
| | | GAACTGGTATCAGCAGAAGCCCGGCAAGGCCCCCAA |
| | | GCTGCTGATCTACGCCGCCAGCAATCTGGAAAGCGGC |
| | | GTGCCCAGCAGATTCAGCGGCTCTGGCAGCGGCACCG |
| | | ACTTCACCCTGACAATCAGCAGCCTCCAGTCCGAGGA |
| | | CTTCGCCACCTACTACTGCCAGCAGAGCAACGAGGAC |
| | | CCCTACACCTTTGGCGGAGGCACCAAGGTGGAAATCA |
| | | AGCGTACGGTGGCCGCTCCCAGCGTGTTCATCTTCCCC |
| | | CCCAGCGACGAGCAGCTGAAGAGCGGCACCGCCAGC |
| | | GTGGTGTGCCTGCTGAACAACTTCTACCCCCGGGAGG |
| | | CCAAGGTGCAGTGGAAGGTGGACAACGCCCTGCAGA |
| | | GCGGCAACAGCCAGGAGAGCGTCACCGAGCAGGACA |
| | | GCAAGGACTCCACCTACAGCCTGAGCAGCACCCTGAC |
| | | CCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| >Linkers | | |
| SEQ ID NO: 397 | (G4S)3 | GGGGSGGGGSGGGGS |
| SEQ ID NO: 398 | (G4S)4 | GGGGSGGGGSGGGGSGGGGS |
| SEQ ID NO: 399 | (G4S)5 | GGGGSGGGGSGGGGSGGGGSGGGGS |
| SEQ ID NO: 400 | (G4S)6 | GGGGSGGGGSGGGGSGGGGSGGGGSGGGGS |
| SEQ ID NO: 401 | (G4S)7 | GGGGSGGGGSGGGGSGGGGSGGGGSGGGGSGGGGS |
| SEQ ID NO: 402 | (G4S)8 | GGGGSGGGGSGGGGSGGGGSGGGGSGGGGSGGGGS |
| | | GGGGS |
| SEQ ID NO: 403 | (G4S)9 | GGGGSGGGGSGGGGSGGGGSGGGGSGGGGSGGGGS |
| | | GGGGSGGGGS |
| SEQ ID NO: 404 | (G3S)4 | GGGSGGGSGGGSGGGS |
| SEQ ID NO: 405 | (G3S)5 | GGGSGGGSGGGSGGGSGGGS |
| SEQ ID NO: 406 | (G3S)6 | GGGSGGGSGGGSGGGSGGGSGGGS |
| SEQ ID NO: 407 | (G3S)7 | GGGSGGGSGGGSGGGSGGGSGGGSGGGS |
| SEQ ID NO: 408 | (G3S)8 | GGGSGGGSGGGSGGGSGGGSGGGSGGGSGGGS |
| SEQ ID NO: 409 | (G3S)9 | GGGSGGGSGGGSGGGSGGGSGGGSGGGSGGGSGGG |
| | | S |
| SEQ ID NO: 410 | (G3S)10 | GGGSGGGSGGGSGGGSGGGSGGGSGGGSGGGSGGG |
| | | SGGGS |
| SEQ ID NO: 411 | (G3S)11 | GGGSGGGSGGGSGGGSGGGSGGGSGGGSGGGSGGG |
| | | SGGGSGGGS |
| SEQ ID NO: 412 | G4 | GGGG |
| >Fusions | | |
| SEQ ID NO: 413 (DNA | IL-2-(G4S)₃-L | ATGTACAGGATGCAACTCCTGTCTTGCATTGCACTAAG |
| without signal peptide) | (NARA1) | TCTTGCACTTGTCACAAACAGTGCACCTACTTCAAGTT |
| | | CTACAAAGAAAACACAGCTACAACTGGAGCATTTACTT |
| | | CTGGATTTACAGATGATTTTGAATGGAATTAATAATTA |
| | | CAAGAATCCCAAACTCACCAGGATGCTCACATTTAAGT |
| | | TTTACATGCCCAAGAAGGCCACAGAACTGAAACATCT |
| | | TCAGTGTCTAGAAGAAGAACTCAAACCTCTGGAGGAA |
| | | GTGCTAAATTTAGCTCAAAGCAAAAACTTTCACTTAAG |
| | | ACCCAGGGACTTAATCAGCAATATCAACGTAATAGTTC |
| | | TGGAACTAAAGGGATCTGAAACAACATTCATGTGTGA |
| | | ATATGCTGATGAGACAGCAACCATTGTAGAATTTCTG |
| | | AACAGATGGATTACCTTTTGTCAAAGCATCATCTCAAC |
| | | ACTGACTGGCGGGGGAGGTTCTGGCGGTGGGGGATC |
| | | GGGCGGTGGAGGGAGCGACATTGTGCTGACCCAATC |
| | | TCCAGCTTCTTTGGCTGTGTCTCTAGGGCAGAGGGCC |
| | | ACCATCTCCTGCAAGGCCAGCCAAAGTGTTGATTATG |
| | | ATGGTGATAGTTATATGAACTGGTACCAACAGAAACC |
| | | AGGACAGCCACCCAAACTCCTCATCTATGCTGCATCCA |
| | | ATCTAGAATCTGGGATCCCAGCCAGGTTTAGTGGCAG |
| | | TGGGTCTGGGACAGACTTCACCCTCAACATCCATCCTG |
| | | TGGAGGAGGAGGATGCTGCAACCTATTACTGTCAGCA |
| | | AAGTAATGAGGATCCGTACACGTTCGGAGGGGGGAC |
| | | CAAGCTGGAAATAAAACGGGCTGATGCTGCACCAACT |
| | | GTATCCATCTTCCCACCATCCAGTGAGCAGTTAACATC |
| | | TGGAGGTGCCTCAGTCGTGTGCTTCTTGAACAACTTCT |
| | | ACCCCAAAGACATCAATGTCAAGTGGAAGATTGATGG |
| | | CAGTGAACGACAAAATGGCGTCCTGAACAGTTGGACT |
| | | GATCAGGACAGCAAAGACAGCACCTACAGCATGAGC |
| | | AGCACCCTCACGTTGACCAAGGACGAGTATGAA |
| SEQ ID NO: 414 (DNA | IL-2-(G4S)₄-L | ATGTACAGGATGCAACTCCTGTCTTGCATTGCACTAAG |
| without signal peptide) | (NARA1) | TCTTGCACTTGTCACAAACAGTGCACCTACTTCAAGTT |
| | | CTACAAAGAAAACACAGCTACAACTGGAGCATTTACTT |
| | | CTGGATTTACAGATGATTTTGAATGGAATTAATAATTA |
| | | CAAGAATCCCAAACTCACCAGGATGCTCACATTTAAGT |
| | | TTTACATGCCCAAGAAGGCCACAGAACTGAAACATCT |
| | | TCAGTGTCTAGAAGAAGAACTCAAACCTCTGGAGGAA |
| | | GTGCTAAATTTAGCTCAAAGCAAAAACTTTCACTTAAG |
| | | ACCCAGGGACTTAATCAGCAATATCAACGTAATAGTTC |
| | | TGGAACTAAAGGGATCTGAAACAACATTCATGTGTGA |
| | | ATATGCTGATGAGACAGCAACCATTGTAGAATTTCTG |
| | | AACAGATGGATTACCTTTTGTCAAAGCATCATCTCAAC |
| | | ACTGACTGGCGGTGGGGGATCAGGGGGCGGAGGTTC |
| | | TGGAGGTGGCGGGTCGGGGGGAGGTGGGAGCGACA |
| | | TTGTGCTGACCCAATCTCCAGCTTCTTTGGCTGTGTCTC |
| | | TAGGGCAGAGGGCCACCATCTCCTGCAAGGCCAGCCA |
| | | AAGTGTTGATTATGATGGTGATAGTTATATGAACTGG |
| | | TACCAACAGAAACCAGGACAGCCACCCAAACTCCTCA |
| | | TCTATGCTGCATCCAATCTAGAATCTGGGATCCCAGCC |
| | | AGGTTTAGTGGCAGTGGGTCTGGGACAGACTTCACCC |
| | | TCAACATCCATCCTGTGGAGGAGGAGGATGCTGCAAC |
| | | CTATTACTGTCAGCAAAGTAATGAGGATCCGTACACG |
| | | TTCGGAGGGGGGACCAAGCTGGAAATAAAACGGGCT |
| | | GATGCTGCACCAACTGTATCCATCTTCCCACCATCCAG |
| | | TGAGCAGTTAACATCTGGAGGTGCCTCAGTCGTGTGC |
| | | TTCTTGAACAACTTCTACCCCAAAGACATCAATGTCAA |
| | | GTGGAAGATTGATGGCAGTGAACGACAAAATGGCGT |
| | | CCTGAACAGTTGGACTGATCAGGACAGCAAAGACAGC |
| | | ACCTACAGCATGAGCAGCACCCTCACGTTGACCAAGG |
| | | ACGAGTATGAACGACATAACAGCTATACCTGTGAGGC |
| | | CACTCACAAGACATCAACTTCACCCATTGTCAAGAGCT |
| | | TCAACAGGAATGAGTGT |
| SEQ ID NO: 415 (DNA | IL-2-(G4S)₅-L | ATGTACAGGATGCAACTCCTGTCTTGCATTGCACTAAG |
| without signal peptide) | (NARA1) | TCTTGCACTTGTCACAAACAGTGCACCTACTTCAAGTT |
| | | CTACAAAGAAAACACAGCTACAACTGGAGCATTTACTT |
| | | CTGGATTTACAGATGATTTTGAATGGAATTAATAATTA |
| | | CAAGAATCCCAAACTCACCAGGATGCTCACATTTAAGT |
| | | TTTACATGCCCAAGAAGGCCACAGAACTGAAACATCT |
| | | TCAGTGTCTAGAAGAAGAACTCAAACCTCTGGAGGAA |
| | | GTGCTAAATTTAGCTCAAAGCAAAAACTTTCACTTAAG |
| | | ACCCAGGGACTTAATCAGCAATATCAACGTAATAGTTC |
| | | TGGAACTAAAGGGATCTGAAACAACATTCATGTGTGA |
| | | ATATGCTGATGAGACAGCAACCATTGTAGAATTTCTG |
| | | AACAGATGGATTACCTTTTGTCAAAGCATCATCTCAAC |
| | | ACTGACTGGCGGTGGGGGATCAGGGGGCGGAGGTTC |
| | | TGGAGGTGGCGGGTCGGGGGGAGGTGGGAGCGGTG |
| | | GCGGGGGATCAGACATTGTGCTGACCCAATCTCCAGC |
| | | TTCTTTGGCTGTGTCTCTAGGGCAGAGGGCCACCATCT |
| | | CCTGCAAGGCCAGCCAAAGTGTTGATTATGATGGTGA |
| | | TAGTTATATGAACTGGTACCAACAGAAACCAGGACAG |
| | | CCACCCAAACTCCTCATCTATGCTGCATCCAATCTAGA |
| | | ATCTGGGATCCCAGCCAGGTTTAGTGGCAGTGGGTCT |
| | | GGGACAGACTTCACCCTCAACATCCATCCTGTGGAGG |
| | | AGGAGGATGCTGCAACCTATTACTGTCAGCAAAGTAA |
| | | TGAGGATCCGTACACGTTCGGAGGGGGGACCAAGCT |
| | | GGAAATAAAACGGGCTGATGCTGCACCAACTGTATCC |
| | | ATCTTCCCACCATCCAGTGAGCAGTTAACATCTGGAGG |
| | | TGCCTCAGTCGTGTGCTTCTTGAACAACTTCTACCCCA |
| | | AAGACATCAATGTCAAGTGGAAGATTGATGGCAGTGA |
| | | ACGACAAAATGGCGTCCTGAACAGTTGGACTGATCAG |
| | | GACAGCAAAGACAGCACCTACAGCATGAGCAGCACCC |
| | | TCACGTTGACCAAGGACGAGTATGAACGACATAACAG |
| | | CTATACCTGTGAGGCCACTCACAAGACATCAACTTCAC |
| | | CCATTGTCAAGAGCTTCAACAGGAATGAGTGT |
| SEQ ID NO: 416 | IL-2-(G4S)₃-L | APTSSSTKKTQLQLEHLLLDLQMILNGI |
| | (NARA1) | NNYKNPKLTRMLTFKFYMPKKATELKH |
| | | LQCLEEELKPLEEVLNLAQSKNFHLRPR |
| | | DLISNINVIVLELKGSETTFMCEYADETA |
| | | TIVEFLNRWITFCQSIISTLTGGGGSGG |
| | | GGSGGGGSDIVLTQSPASLAVSLGQRA |
| | | TISCKASQSVDYDGDSYMNWYQQKPG |
| | | QPPKLLIYAASNLESGIPARFSGSGSGT |
| | | DFTLNIHPVEEEDAATYYCQQSNEDPYT |
| | | FGGGTKLEIKRADAAPTVSIFPPSSEQLT |
| | | SGGASVVCFLNNFYPKDINVKWKIDGS |
| | | ERQNGVLNSWTDQDSKDSTYSMSSTLT |
| | | LTKDEYERHNSYTCEATHKTSTSPIVKSF |
| | | NRNEC |
| SEQ ID NO: 417 | IL-2-(G4S)₄-L | APTSSSTKKTQLQLEHLLLDLQMILNGI |
| | (NARA1) | NNYKNPKLTRMLTFKFYMPKKATELKH |
| | | LQCLEEELKPLEEVLNLAQSKNFHLRPR |
| | | DLISNINVIVLELKGSETTFMCEYADETA |
| | | TIVEFLNRWITFCQSIISTLTGGGGSGG |
| | | GGSGGGGSGGGGSDIVLTQSPASLAVS |
| | | LGQRATISCKASQSVDYDGDSYMNWY |
| | | QQKPGQPPKLLIYAASNLESGIPARFSG |
| | | SGSGTDFTLNIHPVEEEDAATYYCQQSN |
| | | EDPYTFGGGTKLEIKRADAAPTVSIFPPS |
| | | SEQLTSGGASVVCFLNNFYPKDINVKW |
| | | KIDGSERQNGVLNSWTDQDSKDSTYSM |
| | | SSTLTLTKDEYERHNSYTCEATHKTSTSP |
| | | IVKSFNRNEC |
| SEQ ID NO: 418 | IL-2-(G4S)₅-L | APTSSSTKKTQLQLEHLLLDLQMILNGI |
| | (NARA1) | NNYKNPKLTRMLTFKFYMPKKATELKH |
| | | LQCLEEELKPLEEVLNLAQSKNFHLRPR |
| | | DLISNINVIVLELKGSETTFMCEYADETA |
| | | TIVEFLNRWITFCQSIISTLTGGGGSGG |
| | | GGSGGGGSGGGGSGGGGSDIVLTQSP |
| | | ASLAVSLGQRATISCKASQSVDYDGDSY |
| | | MNWYQQKPGQPPKLLIYAASNLESGIP |
| | | ARFSGSGSGTDFTLNIHPVEEEDAATYY |
| | | CQQSNEDPYTFGGGTKLEIKRADAAPT |
| | | VSIFPPSSEQLTSGGASVVCFLNNFYPK |
| | | DINVKWKIDGSERQNGVLNSWTDQDS |
| | | KDSTYSMSSTLTLTKDEYERHNSYTCEA |
| | | THKTSTSPIVKSFNRNEC |

1-31. (canceled)

32. A method of treating cancer or viral infection by (a) selecting a patient having cancer or viral infection and (b) administering a therapeutically effective amount of an antibody/IL-2 fusion protein comprising:
(i) a first polypeptide comprising a VH domain, wherein the VH domain comprises the amino acid sequences of SEQ ID NOs: 119, 120, and 121;
(ii) a second polypeptide comprising a VL domain, wherein the VL domain comprises the amino acid sequences SEQ ID NOs: 122, 123, and 21; and
(iii) a third polypeptide comprising an amino acid sequence encoding a human interleukin-2 protein (hIL-2),
wherein the third polypeptide is inserted into at least one of the VH domain or the VL domain, and
wherein said antibody/IL-2 fusion protein has hIL-2 activity.

33. The method of treating cancer or viral infection according to claim 32, wherein
(a) the VL domain comprises:
(i) a LCDR1 comprising an amino acid sequence selected from the group consisting of SEQ ID NO: 19, SEQ ID NO: 31, SEQ ID NO: 69, SEQ ID NO: 72, SEQ ID NO: 86 and SEQ ID NO: 90;
(ii) a LCDR2 comprising an amino acid sequence selected from the group consisting of SEQ ID NO: 20 and SEQ ID NO: 32; and
(iii) a LCDR3 comprising an amino acid sequence selected from SEQ ID NO: 21, and
(b) the VH domain comprises:
(i) a HCDR1 comprising an amino acid sequence selected from the group consisting of SEQ ID NO: 1, SEQ ID NO: 4 and SEQ ID NO: 13;
(ii) a HCDR2 comprising an amino acid sequence selected from the group consisting of SEQ ID NO: 2 and SEQ ID NO: 12; and
(iii) a HCDR3 comprising an amino acid sequence selected from the group consisting of SEQ ID NO: 3, SEQ ID NO: 36, SEQ ID NO: 39, SEQ ID NO: 42, and SEQ ID NO: 45.

34. The method of treating cancer or viral infection according to claim 33, wherein:
(a) the LCDR1, LCDR2 and LCDR3 comprise the amino acid sequences of SEQ ID NO: 19, 20 and 21, respectively and the HCDR1, HCDR2 and HCDR3 comprise the amino acid sequences of SEQ ID NO: 4, 2 and 3, respectively;
(b) the LCDR1, LCDR2 and LCDR3 comprise the amino acid sequences of SEQ ID NO: 31, 32 and 21, respectively and the HCDR1, HCDR2 and HCDR3 comprise the amino acid sequences of SEQ ID NO: 4, 2 and 3, respectively;
(c) the LCDR1, LCDR2 and LCDR3 comprise the amino acid sequences of SEQ ID NO: 19, 20 and 21, respectively and the HCDR1, HCDR2 and HCDR3 comprise the amino acid sequences of SEQ ID NO: 13, 12 and 3, respectively;
(d) the LCDR1, LCDR2 and LCDR3 comprise the amino acid sequences of SEQ ID NO: 31, 32 and 21, respectively and the HCDR1, HCDR2 and HCDR3 comprise the amino acid sequences of SEQ ID NO: 13, 12 and 3, respectively;
(e) the LCDR1, LCDR2 and LCDR3 comprise the amino acid sequences of SEQ ID NO: 69, 20 and 21, respectively and the HCDR1, HCDR2 and HCDR3 comprise the amino acid sequences of SEQ ID NO: 4, 2, and 3, respectively;
(f) the LCDR1, LCDR2 and LCDR3 comprise the amino acid sequences of SEQ ID NO: 19, 20, and 21, respectively, and the HCDR1, HCDR2 and HCDR3 comprise the amino acid sequences of 4, 2, and 36, respectively;
(g) the LCDR1, LCDR2 and LCDR3 comprise the amino acid sequences of SEQ ID NO: 69, 20, and 21, respectively, and the HCDR1, a HCDR2 and HCDR3 comprise the amino acid sequences of 4, 2, and 36, respectively; or
(h) the LCDR1, LCDR2 and LCDR3 comprise the amino acid sequences of SEQ ID NO: 19, 20, 21, respectively, and the HCDR1, HCDR2 and HCDR3 comprise the amino acid sequences of 1, 2, and 36, respectively.

35. The method of treating cancer or viral infection according to claim 32, wherein the therapeutically effective amount of the antibody/IL-2 fusion protein is administered with another agent as a combination therapy.

36. The method of treating cancer or viral infection according to claim 35, wherein the agent comprises an immunomodulating agent, a cytotoxic agent, an anti-inflammatory agent, or a chemotherapeutic agent.

37. The method of treating cancer or viral infection according to claim 35, wherein the combination therapy is administered sequentially or simultaneously.

38. The method of treating cancer or viral infection according to claim 32, wherein the therapeutically effective amount of the antibody/IL-2 fusion protein is administered in a pharmaceutical composition comprising the antibody/IL-2 fusion protein and a pharmaceutically acceptable carrier.

39. The method of treating cancer or viral infection according to claim 38, wherein the pharmaceutical composition further comprises an immunomodulating agent, a cytotoxic agent, an anti-inflammatory agent, or a chemotherapeutic agent.

40. The method of treating cancer or viral infection according to claim 32, wherein the cancer is a solid tumor, a soft tissue tumor or a metastatic lesion.

41. The method of treating cancer or viral infection according to claim 32, wherein the cancer is melanoma, myeloma, renal cancer, prostate cancer, breast cancer, colorectal cancer, anal cancer, liver cancer, pancreatic cancer, gastro-esophageal cancer, thyroid cancer, cervical cancer, lung cancer or hematological cancer.

42. The method of treating cancer or viral infection according to claim 32, wherein the viral infection is an infection that can lead to cancer.

43. The method of treating cancer or viral infection according to claim 42, wherein the viral infection is viral hepatitis.
